(19) United States
(12) Patent Application Publication
(10) Pub. No.: US 2024/0277833 A1
(43) Pub. Date: Aug. 22, 2024

(54) NON-VIRAL DNA VECTORS FOR VACCINE DELIVERY

(71) Applicant: Generation Bio Co., Cambridge, MA (US)

(72) Inventors: Phillip Samayoa, Cambridge, MA (US); Matthew G. Stanton, Cambridge, MA (US); Raj Rajendran, Cambridge, MA (US); Debra Klatte, Cambridge, MA (US); Nathaniel Silver, Cambridge, MA (US); Luke S. Hamm, Cambridge, MA (US); Matthew Manganiello, Cambridge, MA (US); Jeffrey Moffit, Cambridge, MA (US)

(21) Appl. No.: 18/289,622

(22) PCT Filed: May 6, 2022
(86) PCT No.: PCT/US2022/028019

§ 371 Date: Nov. 6, 2023

Related U.S. Application Data
(60) Provisional application No. 63/185,823, filed on May 7, 2021.

(51) Int. Cl. A61K 39/215; A61K 9/51; A61K 39/00; A61P 37/04; C12N 7/00; C12N 15/85; C12N 15/88

(52) U.S. Cl. CPC A61K 39/215; A61K 9/5123; A61P 37/04; C12N 7/00; C12N 15/85; C12N 15/88; A61K 2039/53; C12N 2770/20022; C12N 2770/20034; C12N 2830/50

(57) ABSTRACT

The application describes methods and compositions comprising ceDNA vectors useful for the expression of antigens and immunogenic peptides in a cell, tissue or subject, and methods of treatment and/or prevention of various infectious diseases, autoimmune disorders and cancers.

### RELATED APPLICATIONS

This application claims priority to U.S. Provisional Application No. 63/185,823, filed on May 7, 2021. The entire contents of the foregoing application are expressly incorporated by reference herein.

### TECHNICAL FIELD

The present disclosure relates generally to vaccines against immunogens of pathogenic organisms and cancer. The present disclosure relates, more particularly, to non-viral vectors for expressing antigens, or antigenic proteins, in a subject or a cell. The disclosure also relates to nucleic acid constructs, promoters, vectors, and host cells comprising the nucleic acids, as well as methods of delivering transgenes encoding the antigens, or antigenic proteins, to a target cell, tissue, organ or organism.

### BACKGROUND

Infectious existing and emerging pathogens continue to cause significant morbidity, mortality and economic burden worldwide. The majority of deaths are caused by just a few pathogens: among the 1400 or so recognized human pathogens and parasites, the majority of deaths are caused by respiratory illness, diarrhea, HIV/AIDS, TB, malaria, meningitis, pertussis, measles, hepatitis B, and sexually transmitted diseases (STDs) (Dye C. After 2015: Infectious diseases in a new era of health and development. (2014) Philosophical Transactions of the Royal Society of London. Series B, Biological Sciences, 369(1645), 20130426. doi:10.1098/rstb.2013.0426). Certain diseases are considered particularly important, e.g., because they had a 100% lethality rate when they emerged, for example, HIV/AIDS; or because the infectious viral agent causes disease beyond the principal person of infection, for example the emergence of birth defects from infection with zika virus.

Therapeutic products used to fight pathogens include preventative immunizations, such as vaccines, and post-infection therapeutics, such as anti-bacterials and anti-virals. Vaccines are therapeutics composed of one or a few specific antigens of the causative microbial agent or the microbial or viral body with its whole set of antigens that induce an immune response in the receiving individual and/or a cellular response in the pathogen itself (Cassone, A., & Rappuoli, R. (2010). Universal vaccines: shifting to one for many. Bio. 1(1), e00042-10. doi:10.1128/mBio.00042-10).

Vaccines protect by inducing effector mechanisms capable of rapidly controlling replicating pathogens or inactivating their toxic components.

While vaccination provides a cost-effective measure to prevent disease and to control outbreaks of infection at herd level, vaccines currently on the market have significant shortcomings and sometimes failures.

Recombinant AAV (rAAV) is perhaps the best studied vector for gene transfer in humans, with hundreds of clinical trials demonstrating safety of transduction. Adeno-associated viruses (AAVs) belong to the Parvoviridae family and more specifically constitute the Dependoparvovirus genus. Vectors derived from AAV (i.e., rAVV or AAV vectors) are attractive for delivering genetic material because (i) they are able to infect (transduce) a wide variety of non-dividing and dividing cell types including myocytes and neurons; (ii) they are devoid of the virus structural genes, thereby diminishing the host cell responses to virus infection, e.g., interferon-mediated responses; (iii) wild-type viruses are considered non-pathologic in humans; (iv) in contrast to wild type AAV, which are capable of integrating into the host cell genome, replication-deficient AAV vectors lack the replication (rep) gene and generally persist as episomes, thus limiting the risk of insertional mutagenesis or genotoxicity; and (v) in comparison to other vector systems, AAV vectors are generally considered to be relatively poor immunogens and therefore do not trigger a significant immune response (see (ii)), thus gaining persistence of the vector DNA and potentially, long-term expression of the therapeutic transgenes.

However, there are several major deficiencies in using AAV particles as a gene delivery vector. One major drawback associated with rAAV is its limited viral packaging capacity of about 4.5 kb of heterologous DNA (Dong et al., 1996; Athanasopoulos et al., 2004; Lai et al., 2010), and as a result, use of AAV vectors has been limited to less than 150,000 Da protein coding capacity. Particularly related to antibody delivery, the packaging limitation of AAV represents a significant challenge for the efficient delivery of both heavy and light chains that form the natural antibody structure. The second drawback is that as a result of the prevalence of wild-type AAV infection in the population, candidates for rAAV gene therapy have to be screened for the presence of neutralizing antibodies that eliminate the vector from the patient. A third drawback is related to the capsid immunogenicity that prevents re-administration to patients that were not excluded from an initial treatment. The immune system in the patient can respond to the vector which effectively acts as a “booster” shot to stimulate the immune system generating high titer anti-AAV antibodies that preclude future treatments. Preexisting immunity can severely limit the efficiency of transduction. Some recent reports indicate concerns with immunogenicity in high dose situations. Another notable drawback is that the onset of AAV-mediated gene expression is relatively slow, given that single-stranded AAV DNA must be converted to double-stranded DNA prior to heterologous gene expression.

Adenovirus vectors whereby the vector expresses an unknown antigenic protein have been well studied for gene and cancer therapy and vaccines. Apart from its extensive safety profile, the advantages of utilizing an adenovirus vector are that it is relatively stable, easy to attain high titers and able to infect multiple cell lines which attributes to its potency. Even though recombinant adenoviral vectors are widely used today thanks to its high transduction efficiency and transgene expression, there is likelihood for pre-existing immunity against the vector, because most of the population has been exposed to adenovirus (Id). This has been proven detrimental in a human immunodeficiency virus (HIV-1) phase IIb vaccine trial in which the vector-based vaccines provided favorable conditions for HIV-1 replication (Smaill, F. et al., Sci. Transl. Med. (2013) 5: 205).

There remains a need in the art for the development of improved vaccine therapeutics.

### BRIEF DESCRIPTION

The technology described herein relates to capsid-free (e.g., non-viral) DNA vectors with covalently-closed ends (referred to herein as a “closed-ended DNA vector” or a “ceDNA vector”), where the ceDNA vector comprises a nucleic acid sequence that encodes an antigen, or an immunogenic peptide. The application of one or more ceDNA vectors expressing one or more nucleic acid sequences that encode one or more antigens, or immunogenic peptides, is useful to: treat, prevent or reduce the severity of a disease or disorder in a subject, be minimally invasive in delivery, be repeatable and dosed-to-effect, have rapid onset of therapeutic effect, and/or result in sustained expression of antigen, or immunogenic peptide.

Unlike traditional vaccines, which are manufactured ex vivo and may trigger unwanted cellular responses, the ceDNA vaccines described herein are presented to the cellular system in a more native fashion. By employing a ceDNA vector to deliver a transgene (e.g., a nucleic acid sequence) encoding antigen, or immunogenic peptide to cells or tissues, the adaptive immune response is bypassed, and the desired antibody specificities are produced without the use of immunization or passive transfer. That is, the ceDNA vector enters the cell via endocytosis, then escapes from the endosomal compartment and is transported to the nucleus. The transcriptionally active ceDNA episome results in the expression of antigens that may then be secreted from the cell into the circulation. The ceDNA vector may therefore enable continuous, sustained and long-term delivery of antibodies (e.g., the therapeutic antibodies, or antigen-binding fragments therein, described herein) administered by a single injection. This is particularly advantageous in the context of nucleic acid vaccine compositions, where DNA vaccines show a slower increase in expression and a more sustained expression as compared to mRNA vaccines which may show more increased initial expression, that decreased more quickly.

According to some aspects, the disclosure provides a capsid-free closed ended DNA (ceDNA) vector comprising at least one nucleic acid sequence between flanking inverted terminal (ITRs), wherein the at least one nucleic acid sequence encodes an antigen, or an immunogenic peptide. According to some embodiments, the antigen, or the immunogenic peptide, is derived from a bacterial, a viral, a fungal or a parasitic infectious agent. According to some embodiments, the antigen, or the immunogenic peptide, is a tumor associated antigen. According to some embodiments, the antigen, or the immunogenic peptide, is associated with an autoimmune condition. According to some embodiments of the foregoing aspects and embodiments, the antigen, or the immunogenic peptide, is selected from one or more of those set forth in Tables 1-8. According to some embodiments of the foregoing aspects and embodiments, the ceDNA vector comprises a promoter sequence operatively linked to the at least one nucleic acid sequence. According to some embodiments of the foregoing aspects and embodiments, the ceDNA vector comprises at least one poly A sequence. According to some embodiments of the foregoing aspects and embodiments, the ceDNA vector comprises a 5′ UTR and/or intron sequence. According to some embodiments of the foregoing aspects and embodiments, the ceDNA vector comprises a 3′ UTR sequence. According to some embodiments of the foregoing aspects and embodiments, the ceDNA vector comprises an enhancer sequence. According to some embodiments of the foregoing aspects and embodiments, at least one ITR comprises a functional terminal resolution site and a Rep binding site. According to some embodiments of the foregoing aspects and embodiments, one or both of the ITRs are from a virus selected from a Parvovirus, a Dependovirus, and an adeno-associated virus (AAV). According to some embodiments of the foregoing aspects and embodiments, the flanking ITRs are symmetric or asymmetric with respect to one another. According to some embodiments, the flanking ITRs are symmetrical or substantially symmetrical. According to some embodiments, the flanking ITRs are asymmetric. According to some embodiments of the foregoing aspects and embodiments, one or both of the ITRs are wild type, or wherein both of the ITRs are wild-type ITRs. According to some embodiments of the foregoing aspects and embodiments, the flanking ITRs are from different viral serotypes. According to some embodiments of the foregoing aspects and embodiments, the flanking ITRs are selected from any pair of viral serotypes shown in Table 8. According to some embodiments of the foregoing aspects and embodiments, one or both of the ITRs comprises a sequence selected from one or more of the sequences in Table 9. According to some embodiments of the foregoing aspects and embodiments, at least one of the ITRs is altered from a wild-type AAV ITR sequence by a deletion, addition, or substitution that affects the overall three-dimensional conformation of the ITR.

According to some embodiments of the foregoing aspects and embodiments, one or both of the ITRs are derived from an AAV serotype selected from AAV1, AAV2, AAV3, AAV4, AAV5, AAV6, AAV7, AAV8, AAV9, AAV10, AAV11, and AAV12. According to some embodiments of the foregoing aspects and embodiments, one or both of the ITRs are synthetic. According to some embodiments of the foregoing aspects and embodiments, one or both of the ITRs are not a wild type ITR, or wherein both of the ITRs are not wild-type ITRs. According to some embodiments of the foregoing aspects and embodiments, one or both of the ITRs are modified by a deletion, insertion, and/or substitution in at least one of the ITR regions selected from A, A′, B, B′, C, C′, D, and D′. According to some embodiments, the deletion, insertion, and/or substitution results in the deletion of all or part of a stem-loop structure normally formed by the A, A′, B, B′, C, or C′ regions. According to some embodiments of the foregoing aspects and embodiments, one or both of the ITRs are modified by a deletion, insertion, and/or substitution that results in the deletion of all or part of a stem-loop structure normally formed by the B and B′ regions. According to some embodiments of the foregoing aspects and embodiments, one or both of the ITRs are modified by a deletion, insertion, and/or substitution that results in the deletion of all or part of a stem-loop structure normally formed by the C and C′ regions. According to some embodiments of the foregoing aspects and embodiments, one or both of the ITRs are modified by a deletion, insertion, and/or substitution that results in the deletion of part of a stem-loop structure normally formed by the B and B′ regions and/or part of a stem-loop structure normally formed by the C and C′ regions. According to some embodiments of the foregoing aspects and embodiments, one or both of the ITRs comprise a single stem-loop structure in the region that normally comprises a first stem-loop structure formed by the B and B′ regions and a second stem-loop structure formed by the C and C′ regions. According to some embodiments of the foregoing aspects and embodiments, one or both of the ITRs comprise a single stem and two loops in the region that normally comprises a first stem-loop structure formed by the B and B′ regions and a second stem-loop structure formed by the C and C′ regions. According to some embodiments of the foregoing aspects and embodiments, one or both of the ITRs comprise a single stem and a single loop in the region that normally comprises a first stem-loop structure formed by the B and B′ regions and a second stem-loop structure formed by the C and C′ regions. According to some embodiments of the foregoing aspects and embodiments, both ITRs are altered in a manner that results in an overall three-dimensional symmetry when the ITRs are inverted relative to each other. According to some embodiments of the foregoing aspects and embodiments, the ceDNA vector is encapsulated in a lipid nanoparticle (LNP).

According to some aspects, the ceDNA vector as described in the aspects and embodiments herein is used as a vaccine.

According to some aspects, the disclosure provides a method of expressing an antigen, or an immunogenic peptide, in a cell comprising contacting the cell with the ceDNA vector of any of the foregoing aspects and embodiments. According to some embodiments, the cell is in vitro or in vivo.

According to some embodiments, the at least one nucleic acid sequence is codon optimized for expression in the cell.

According to some aspects, the disclosure provides a method of treating a subject with a bacterial, a viral, a parasitic or a fungal infection, comprising administering to the subject the ceDNA vector of any of the foregoing aspects and embodiments.

According to some aspects, the disclosure provides a method of treating a subject with a cancer, comprising administering to the subject the ceDNA vector of any of the foregoing aspects and embodiments.

According to some aspects, the disclosure provides a method of treating a subject with an autoimmune disease or disorder, comprising administering to the subject the ceDNA vector of any of the foregoing aspects and embodiments.

According to some aspects, the disclosure provides a method of preventing a bacterial, a viral, a parasitic or a fungal infection in a subject, comprising administering to the subject the ceDNA vector of any of the foregoing aspects and embodiments.

According to some aspects, the disclosure provides a method of preventing cancer in a subject, comprising administering to the subject the ceDNA vector of any of the foregoing aspects and embodiments.

According to some aspects, the disclosure provides a method of preventing an autoimmune disease in a subject, comprising administering to the subject the ceDNA vector of any of the foregoing aspects and embodiments.

According to some embodiments of the foregoing aspects and embodiments, the method further comprises administering to the subject one or more additional therapeutic agents. According to some embodiments of the foregoing aspects and embodiments the ceDNA vector is administered by intravenous, subcutaneous, intratumoral or intramuscular injection.

According to some aspects, the disclosure provides a pharmaceutical composition comprising the ceDNA vector of any of the foregoing aspects and embodiments. According to some embodiments, the pharmaceutical composition further comprises one or more additional therapeutic agents.

According to some aspects, the disclosure provides a vaccine composition comprising the ceDNA vector of any of the foregoing aspects and embodiments.

According to some aspects, the disclosure provides a composition comprising the ceDNA vector of any of the foregoing aspects and embodiments and a lipid. According to some embodiment, the lipid is a lipid nanoparticle (LNP). According to some embodiments, the composition is lyophilized.

According to some aspects, the disclosure provides a kit comprising the ceDNA vector of any of the foregoing aspects and embodiments, the pharmaceutical composition of any of the foregoing aspects and embodiments or the composition of any of the foregoing aspects and embodiments.

These and other aspects of the disclosure are described in further detail below.

### DESCRIPTION OF DRAWINGS

Embodiments of the present disclosure, briefly summarized above and discussed in greater detail below, can be understood by reference to the illustrative embodiments of the disclosure depicted in the appended drawings. However, the appended drawings illustrate only typical embodiments of the disclosure and are therefore not to be considered limiting of scope, for the disclosure may admit to other equally effective embodiments.

FIG. 1A illustrates an exemplary structure of a ceDNA vector for expression of an antigen, or immunogenic peptide, as disclosed herein, comprising asymmetric ITRs. In this embodiment, the exemplary ceDNA vector comprises an expression cassette containing CAG promoter, WPRE, and BGHpA. An open reading frame (ORF) encoding the transgene (e.g., nucleic acid sequence encoding the antibody or the antigen-binding fragment thereof) can be inserted into the cloning site (R3/R4) between the CAG promoter and WPRE. The expression cassette is flanked by two inverted terminal repeats (ITRs)—the wild-type AAV2 ITR on the upstream (5′-end) and the modified ITR on the downstream (3′-end) of the expression cassette, therefore the two ITRs flanking the expression cassette are asymmetric with respect to each other.

FIG. 1B illustrates an exemplary structure of a ceDNA vector for expression of an antigen, or immunogenic peptide, as disclosed herein comprising asymmetric ITRs with an expression cassette containing CAG promoter, WPRE, and BGHpA. An open reading frame (ORF) encoding the transgene (e.g., nucleic acid sequence encoding the antibody or the antigen-binding fragment thereof) can be inserted into the cloning site between CAG promoter and WPRE. The expression cassette is flanked by two inverted terminal repeats (ITRs)—a modified ITR on the upstream (5′-end) and a wild-type ITR on the downstream (3′-end) of the expression cassette.

FIG. 1C illustrates an exemplary structure of a ceDNA vector for expression of an antigen, or immunogenic peptide, as disclosed herein comprising asymmetric ITRs, with an expression cassette containing an enhancer/promoter, the transgene (e.g., nucleic acid sequence encoding the antibody or the antigen-binding fragment thereof), a post transcriptional element (WPRE), and a polyA signal. An open reading frame (ORF) allows insertion of the transgene into the cloning site between CAG promoter and WPRE. The expression cassette is flanked by two inverted terminal repeats (ITRs) that are asymmetrical with respect to each other; a modified ITR on the upstream (5′-end) and a modified ITR on the downstream (3′-end) of the expression cassette, where the 5′ ITR and the 3′ITR are both modified ITRs but have different modifications (i.e., they do not have the same modifications).

FIG. 1D illustrates an exemplary structure of a ceDNA vector for expression of an antigen, or immunogenic peptide (e.g., HC or LC), as disclosed herein, comprising symmetric modified ITRs, or substantially symmetrical modified ITRs as defined herein, with an expression cassette containing CAG promoter, WPRE, and BGHpA. An open reading frame (ORF) encoding the transgene (e.g., nucleic acid sequence encoding the antibody or antigen-binding fragment thereof) is inserted into the cloning site between CAG promoter and WPRE. The expression cassette is flanked by two modified inverted terminal repeats (ITRs), where the 5′ modified ITR and the 3′ modified ITR are symmetrical or substantially symmetrical.

FIG. 1E illustrates an exemplary structure of a ceDNA vector for expression of an antigen, or immunogenic peptide, as disclosed herein comprising symmetric modified ITRs, or substantially symmetrical modified ITRs as defined herein, with an expression cassette containing an enhancer/promoter, a transgene, a post transcriptional element (WPRE), and a polyA signal. An open reading frame (ORF) allows insertion of a transgene (e.g., nucleic acid sequence encoding the antibody or antigen-binding fragment thereof) into the cloning site between CAG promoter and WPRE. The expression cassette is flanked by two modified inverted terminal repeats (ITRs), where the 5′ modified ITR and the 3′ modified ITR are symmetrical or substantially symmetrical.

FIG. 1F illustrates an exemplary structure of a ceDNA vector for expression of an antigen, or immunogenic peptide, as disclosed herein, comprising symmetric WT-ITRs, or substantially symmetrical WT-ITRs as defined herein, with an expression cassette containing CAG promoter, WPRE, and BGHpA. An open reading frame (ORF) encoding a transgene (e.g., nucleic acid sequence encoding the antibody or antigen-binding fragment thereof) is inserted into the cloning site between CAG promoter and WPRE. The expression cassette is flanked by two wild type inverted terminal repeats (WT-ITRs), where the 5′ WT-ITR and the 3′ WT ITR are symmetrical or substantially symmetrical.

FIG. 1G illustrates an exemplary structure of a ceDNA vector for expression of an antigen, or immunogenic peptide, as disclosed herein, comprising symmetric modified ITRs, or substantially symmetrical modified ITRs as defined herein, with an expression cassette containing an enhancer/promoter, a transgene, a post transcriptional element (WPRE), and a polyA signal. An open reading frame (ORF) allows insertion of a transgene into the cloning site between CAG promoter and WPRE. The expression cassette is flanked by two wild type inverted terminal repeats (WT-ITRs), where the 5′ WT-ITR and the 3′ WT ITR are symmetrical or substantially symmetrical.

FIG. 2A provides the T-shaped stem-loop structure of a wild-type left ITR of AAV2 with identification of A-A′ arm, B-B′ arm, C-C′ arm, two Rep binding sites (RBE and RBE′) and also shows the terminal resolution site (trs). The RBE contains a series of 4 duplex tetramers that are believed to interact with either Rep 78 or Rep 68. In addition, the RBE′ is also believed to interact with Rep complex assembled on the wild-type ITR or mutated ITR in the construct. The D and D′ regions contain transcription factor binding sites and other conserved structure. FIG. 2B shows proposed Rep-catalyzed nicking and ligating activities in a wild-type left ITR, including the T-shaped stem-loop structure of the wild-type left ITR of AAV2 with identification of A-A′ arm, B-B′ arm, C-C′ arm, two Rep Binding sites (RBE and RBE′) and also shows the terminal resolution site (trs), and the D and D′ region comprising several transcription factor binding sites and other conserved structure.

FIG. 3A provides the primary structure (polynucleotide sequence) (left) and the secondary structure (right) of the RBE-containing portions of the A-A′ arm, and the C-C′ and B-B′ arm of the wild type left AAV2 ITR. FIG. 3B shows an exemplary mutated ITR (also referred to as a modified ITR) sequence for the left ITR. Shown is the primary structure (left) and the predicted secondary structure (right) of the RBE portion of the A-A′ arm, the C arm and B-B′ arm of an exemplary mutated left ITR (ITR-1, left). FIG. 3C shows the primary structure (left) and the secondary structure (right) of the RBE-containing portion of the A-A′ loop, and the B-B′ and C-C′ arms of wild type right AAV2 ITR. FIG. 3D shows an exemplary right modified ITR. Shown is the primary structure (left) and the predicted secondary structure (right) of the RBE containing portion of the A-A′ arm, the B-B′ and the C arm of an exemplary mutant right ITR (ITR-1, right). Any combination of left and right ITR (e.g., AAV2 ITRs or other viral serotype or synthetic ITRs) can be used as taught herein. Each of FIGS. 3A-3D polynucleotide sequences refer to the sequence used in the plasmid or bacmid/baculovirus genome used to produce the ceDNA as described herein. Also included in each of FIGS. 3A-3D are corresponding ceDNA secondary structures inferred from the ceDNA vector configurations in the plasmid or bacmid/baculovirus genome and the predicted Gibbs free energy values.

FIG. 4A is a schematic illustrating an upstream process for making baculovirus infected insect cells (BIICs) that are useful in the production of a ceDNA vector for expression of the antigen, or immunogenic protein, disclosed herein in the process described in the schematic in FIG. 4B. FIG. 4B is a schematic of an exemplary method of ceDNA production and FIG. 4C illustrates a biochemical method and process to confirm ceDNA vector production. FIG. 4D and FIG. 4E are schematic illustrations describing a process for identifying the presence of ceDNA in DNA harvested from cell pellets obtained during the ceDNA production processes in FIG. 4B. FIG. 4D shows schematic expected bands for an exemplary ceDNA either left uncut or digested with a restriction endonuclease and then subjected to electrophoresis on either a native gel or a denaturing gel. The leftmost schematic is a native gel, and shows multiple bands suggesting that in its duplex and uncut form ceDNA exists in at least monomeric and dimeric states, visible as a faster-migrating smaller monomer and a slower-migrating dimer that is twice the size of the monomer. The schematic second from the left shows that when ceDNA is cut with a restriction endonuclease, the original bands are gone and faster-migrating (e.g., smaller) bands appear, corresponding to the expected fragment sizes remaining after the cleavage. Under denaturing conditions, the original duplex DNA is single-stranded and migrates as a species twice as large as observed on native gel because the complementary strands are covalently linked. Thus, in the second schematic from the right, the digested ceDNA shows a similar banding distribution to that observed on native gel, but the bands migrate as fragments twice the size of their native gel counterparts. The rightmost schematic shows that uncut ceDNA under denaturing conditions migrates as a single-stranded open circle, and thus the observed bands are twice the size of those observed under native conditions where the circle is not open. In this figure “kb” is used to indicate relative size of nucleotide molecules based, depending on context, on either nucleotide chain length (e.g., for the single stranded molecules observed in denaturing conditions) or number of basepairs (e.g., for the double-stranded molecules observed in native conditions). FIG. 4E shows DNA having a non-continuous structure. The ceDNA can be cut by a restriction endonuclease, having a single recognition site on the ceDNA vector, and generate two DNA fragments with different sizes (1 kb and 2 kb) in both neutral and denaturing conditions. FIG. 4E also shows a ceDNA having a linear and continuous structure. The ceDNA vector can be cut by the restriction endonuclease, and generate two DNA fragments that migrate as 1 kb and 2 kb in neutral conditions, but in denaturing conditions, the stands remain connected and produce single strands that migrate as 2 kb and 4 kb.

FIG. 5 is an exemplary picture of a denaturing gel running examples of ceDNA vectors with (+) or without (−) digestion with endonucleases (EcoRI for ceDNA construct 1 and 2; BamHI for ceDNA construct 3 and 4; SpeI for ceDNA construct 5 and 6; and XhoI for ceDNA construct 7 and 8) Constructs 1-8 are described in Example 1 of International Application PCT PCT/US18/49996, which is incorporated herein in its entirety by reference. Sizes of bands highlighted with an asterisk were determined and provided on the bottom of the picture.

FIG. 6 is a graph that shows spike protein antibody titer as determined at day 29 and day 49 of the study described in Example 6.

FIG. 7 shows the results of a neutralization assay.

FIG. 8 is a graph that shows spike protein antibody titer as determined at day 21 and day 41 of the study described in Example 7.

FIG. 9 depicts a chart that demonstrates spike protein antibody titer as determined at day 21 and day 49 of the study described in Example 8.

### DETAILED DESCRIPTION

According to embodiments of the present disclosure, compositions for delivering one or more antigens, or immunogenic peptides, by ceDNA vectors are provided.

### I. Definitions

Unless otherwise defined herein, scientific and technical terms used in connection with the present application shall have the meanings that are commonly understood by those of ordinary skill in the art to which this disclosure belongs. It should be understood that this disclosure is not limited to the particular methodology, protocols, and reagents, etc., described herein and as such can vary. The terminology used herein is for the purpose of describing particular embodiments only and is not intended to limit the scope of the present disclosure, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), Fields Virology, 6^{th} Edition, published by Lippincott Williams & Wilkins, Philadelphia, PA, USA (2013), Knipe, D. M. and Howley, P. M. (ed.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4^{th} ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

The term “immunization” or “active immunization” as used herein refers to the production of active immunity, meaning immunity resulting from a naturally acquired infection or intentional vaccination (artificial active immunity).

The term “adjuvant” as used herein, is meant to refer to an agent that, when used in combination with a specific immunogen in a formulation, will augment or otherwise alter or modify the resultant immune response. Modification of the immune response includes intensification or broadening the specificity of the immune response (e.g., either or both the antibody and cellular immune responses). Modification of the immune response can also mean decreasing or suppressing certain antigen-specific immune responses.

The term “antigen” as used herein, is meant to refer to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, inclusive, such as, 9, 10, 11, 12, 13, 14 or 15 amino acids. The term includes polypeptides which include modifications, such as deletions, additions and substitutions (generally conservative in nature) as compared to a native sequence, as long as the protein maintains the ability to elicit an immunological response, as defined herein. These modifications may be deliberate, as through site-directed mutagenesis, or may be accidental, such as through mutations of hosts which produce the antigens.

The term “epitope” may be also referred to as an antigenic determinant, is a molecular determinant (e.g., polypeptide determinant) that can be specifically bound by a binding agent, immunoglobulin or T-cell receptor. Epitope determinants include chemically active surface groupings of molecules, such as amino acids, sugar side chains, phosphoryl, or sulfonyl, and, in certain embodiments, may have specific three-dimensional structural characteristics, and/or specific charge characteristics. Epitopes may be defined as structural or functional. Functional epitopes are generally a subset of the structural epitopes and have those residues that directly contribute to the affinity of the interaction. Epitopes may be linear or conformational, that is, composed of non-linear amino acids. An epitope recognized by an antibody or an antigen-binding fragment of an antibody is a structural element of an antigen that interacts with CDRs (e.g., the complementary site) of the antibody or the fragment. An epitope may be formed by contributions from several amino acid residues, which interact with the CDRs of the antibody to produce specificity. An antigenic fragment can contain more than one epitope. In certain embodiments, an antibody specifically binds an antigen when it recognizes its target antigen in a complex mixture of proteins and/or macromolecules. For example, antibodies are said to “bind to the same epitope” if the antibodies cross-compete (one prevents the binding or modulating effect of the other).

As used herein, the term “autoimmune disorders” refers generally to conditions in which a subject's immune system attacks the body's own cells, causing tissue destruction. Autoimmune disorders may be diagnosed using blood tests, cerebrospinal fluid analysis, electromyogram (measures muscle function), and magnetic resonance imaging of the brain, but antibody testing in the blood, for self-antibodies (or auto-antibodies) is particularly useful. Usually, IgG class antibodies are associated with autoimmune diseases.

The terms “B lymphocyte” or “B cell” are used interchangeably to refer to a broad class of lymphocytes, which are precursors of antibody-secreting cells, that express clonally diverse cell surface immunoglobulin (Ig) receptors (BCRs) recognizing specific antigenic epitopes. Mammalian B-cell development encompasses a continuum of stages that begin in primary lymphoid tissue (e.g., human fetal liver and fetal/adult marrow), with subsequent functional maturation in secondary lymphoid tissue (e.g., human lymph nodes and spleen). The functional/protective end point is antibody production by terminally differentiated plasma cells. A mature B cell can be activated by an encounter with an antigen that expresses epitopes that are recognized by its cell surface immunoglobulin (Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). (LeBien, T W & T F Tedder, B lymphocytes: how they develop and function. Blood (2008) 112 (5): 1570-80).

As used herein, the term “cancer” refers to diseases in which abnormal cells divide without control and are able to invade other tissues. There are more than 100 different types of cancer. Most cancers are named for the organ or type of cell in which they start—for example, cancer that begins in the colon is called colon cancer; cancer that begins in melanocytes of the skin is called melanoma. Cancer types can be grouped into broader categories. The main categories of cancer include: carcinoma (meaning a cancer that begins in the skin or in tissues that line or cover internal organs, and its subtypes, including adenocarcinoma, basal cell carcinoma, squamous cell carcinoma, and transitional cell carcinoma); sarcoma (meaning a cancer that begins in bone, cartilage, fat, muscle, blood vessels, or other connective or supportive tissue); leukemia (meaning a cancer that starts in blood-forming tissue (e.g., bone marrow) and causes large numbers of abnormal blood cells to be produced and enter the blood; lymphoma and myeloma (meaning cancers that begin in the cells of the immune system); and central nervous system (CNS) cancers (meaning cancers that begin in the tissues of the brain and spinal cord). The term “myelodysplastic syndrome” refers to a type of cancer in which the bone marrow does not make enough healthy blood cells (white blood cells, red blood cells, and platelets) and there are abnormal cells in the blood and/or bone marrow. Myelodysplastic syndrome may become acute myeloid leukemia (AML). In certain embodiments, the cancer is selected from cancers including, but not limited to, ACUTE lymphoblastic leukemia (ALL), ACUTE myeloid leukemia (AML), anal cancer, bile duct cancer, bladder cancer, bone cancer, bowel cancer, brain tumor, breast cancer, cancer of unknown primary, cancer spread to bone, cancer spread to brain, cancer spread to liver, cancer spread to lung, carcinoid, cervical cancer, choriocarcinoma, chronic lymphocytic leukemia (CLL), chronic myeloid leukemia (CML), colon cancer, colorectal cancer, endometrial cancer, eye cancer, gallbladder cancer, gastric cancer, gestational trophoblastic tumor (GTT), hairy cell leukemia, head and neck cancer, Hodgkin lymphoma, kidney cancer, laryngeal cancer, leukemia, liver cancer, lung cancer, lymphoma, melanoma skin cancer, mesothelioma, men's cancer, molar pregnancy, mouth and oropharyngeal cancer, myeloma, nasal and sinus cancers, nasopharyngeal cancer, non hodgkin lymphoma (NHL), esophageal cancer, ovarian cancer, pancreatic cancer, penile cancer, prostate cancer, rare cancers, rectal cancer, salivary gland cancer, secondary cancers, skin cancer (non melanoma), soft tissue sarcoma, stomach cancer, testicular cancer, thyroid cancer, unknown primary cancer, uterine cancer, vaginal cancer, and vulval cancer.

As used herein, the term “cross-protection” is used to describe immunity against at least two subgroups, subtypes, strains and/or variants of a virus, bacteria, parasite or other pathogen with a single inoculation with one subgroup, subtype, strain and/or variant thereof.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “detectable response” as used herein, is meant to refer to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The term “effector cell” as used herein refers to a cell that carries out a final response or function. The main effector cells of the immune system, for example, are activated lymphocytes and phagocytes.

The term “herd immunity” as used herein refers to protection conferred to unvaccinated individuals in a population produced by vaccination of others and reduction in the natural reservoir for infection.

The term “heterosubtypic immunity” (“HSI”) as used herein refers to immunity based on immune recognition of antigens conserved across all viral strains.

The term “heterotypic” as used herein is used to refer to being of a different or unusual type or form (e.g., different subgroup, subtype, strain and/or variant of a virus, bacteria, parasite or other pathogen).

The term “homotypic” as used herein is used to refer to being of the same type or form, e.g., same subgroup, subtype, strain and/or variant of a virus, bacteria, parasite or other pathogen.

The terms “immune response” and “immune-mediated” as used herein, are used interchangeably herein to refer to any functional expression of a subject's immune system, against either foreign or self-antigens, whether the consequences of these reactions are beneficial or harmful to the subject. The term “immunological response” to an antigen or composition as used herein, is meant to refer to the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or γδ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The term “immune phenotype” or “immunotype” as used herein refers to the collective frequency of various immune cell populations and their functional responses to stimuli (cell signaling and antibody responses). (See Kaczorowski, K J et al. Proc. Nat. Acad. Sci. USA (2017)).

The term “immune system” as used herein refers to the body's system of defenses against disease, which comprises the innate immune system and the adaptive immune system. The innate immune system provides a non-specific first line of defense against pathogens. It comprises physical barriers (e.g., the skin) and both cellular (granulocytes, natural killer cells) and humoral (complement system) defense mechanisms. The reaction of the innate immune system is immediate, but unlike the adaptive immune system, it does not provide permanent immunity against pathogens. The adaptive immune response is the response of the vertebrate immune system to a specific antigen that typically generates immunological memory.

The term “immunodominant epitope” as used herein refers to the epitope against which the majority of antibodies is raised, or to which the majority of T cells responds.

The term “immunological repertoire” refers to the collection of transmembrane antigen-receptor proteins located on the surface of T and B cells. (Benichou, J. et al. Immunology (2011) 135: 183-191)) The combinatorial mechanism that is responsible for encoding the receptors does so by reshuffling the genetic code, with a potential to generate more than 1018 different T cell receptors (TCRs) in humans (Venturi, Y. et al. Nat. Rev. Immunol. (2008) 8: 231-8) and a much more diverse B-cell repertoire. These sequences, in turn, will be transcribed and then translated into protein to be presented on the cell surface. The recombination process that rearranges the gene segments for the construction of the receptors is key to the development of the immune response, and the correct formation of the rearranged receptors is critical to their future binding affinity to antigen.

A peptide, oligopeptide, polypeptide, protein, or polynucleotide coding for such a molecule is “immunogenic” and thus an immunogen within the present disclosure if it is capable of inducing an immune response. In the present disclosure, immunogenicity is more specifically defined as the ability to induce a CTL-mediated response. Thus, an immunogen would be a molecule that is capable of inducing an immune response, and in the present disclosure, a molecule capable of inducing a CTL response. An immunogen may have one or more isoforms, sequence variants, or splice variants that have equivalent biological and immunological activity, and are thus also considered for the purposes of this disclosure to be immunogenic equivalents of the original, natural polypeptide.

A peptide, oligopeptide, polypeptide, protein, or polynucleotide coding for such a molecule is “immunogenic” and thus an immunogen within the present disclosure if it is capable of inducing an immune response. In the present disclosure, immunogenicity is more specifically defined as the ability to induce a CTL-mediated response. Thus, an immunogen would be a molecule that is capable of inducing an immune response, and in the present disclosure, a molecule capable of inducing a CTL response. An immunogen may have one or more isoforms, sequence variants, or splice variants that have equivalent biological and immunological activity, and are thus also considered for the purposes of this disclosure to be immunogenic equivalents of the original, natural polypeptide.

The term “specifically binds,” as used herein refers to the ability of a polypeptide or polypeptide complex to recognize and bind to a ligand in vitro or in vivo while not substantially recognizing or binding to other molecules in the surrounding milieu. In some embodiments, specific binding can be characterized by an equilibrium dissociation constant of at least about 1×10⁶M or less (e.g., a smaller equilibrium dissociation constant denotes tighter binding). Methods for determining whether two molecules specifically bind are well known in the art and include, for example, equilibrium dialysis, surface plasmon resonance, and the like.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Example 1 of U.S. Pat. No. 6,258,562 and Jönsson et al. (1993) Ann. Biol. Clin. 51:19; Jönsson et al. (1991) Biotechniques 11:620-627; Johnsson et al. (1995) J. Mol. Recognit. 8:125; and Johnnson et al. (1991) Anal. Biochem. 198:268.

As used herein, the terms “heterologous nucleic acid sequence” and “transgene” are used interchangeably and refer to a nucleic acid of interest (other than a nucleic acid encoding a capsid polypeptide) that is incorporated into and may be delivered and expressed by a ceDNA vector as disclosed herein. According to some embodiments, the term “heterologous nucleic acid” is meant to refer to a nucleic acid (or transgene) that is not present in, expressed by, or derived from the cell or subject to which it is contacted.

As used herein, the terms “expression cassette” and “transcription cassette” are used interchangeably and refer to a linear stretch of nucleic acids that includes a transgene that is operably linked to one or more promoters or other regulatory sequences sufficient to direct transcription of the transgene, but which does not comprise capsid-encoding sequences, other vector sequences or inverted terminal repeat regions. An expression cassette may additionally comprise one or more cis-acting sequences (e.g., promoters, enhancers, or repressors), one or more introns, and one or more post-transcriptional regulatory elements.

The terms “polynucleotide” and “nucleic acid,” used interchangeably herein, refer to a polymeric form of nucleotides of any length, either ribonucleotides or deoxyribonucleotides. Thus, this term includes single, double, or multi-stranded DNA or RNA, genomic DNA, cDNA, DNA-RNA hybrids, or a polymer including purine and pyrimidine bases or other natural, chemically or biochemically modified, non-natural, or derivatized nucleotide bases. “Oligonucleotide” generally refers to polynucleotides of between about 5 and about 100 nucleotides of single- or double-stranded DNA. However, for the purposes of this disclosure, there is no upper limit to the length of an oligonucleotide. Oligonucleotides are also known as “oligomers” or “oligos” and may be isolated from genes, or chemically synthesized by methods known in the art. The terms “polynucleotide” and “nucleic acid” should be understood to include, as applicable to the embodiments being described, single-stranded (such as sense or antisense) and double-stranded polynucleotides.

DNA may be in the form of, e.g., antisense molecules, plasmid DNA, DNA-DNA duplexes, pre-condensed DNA, PCR products, vectors (P1, PAC, BAC, YAC, artificial chromosomes), expression cassettes, chimeric sequences, chromosomal DNA, or derivatives and combinations of these groups. DNA may be in the form of minicircle, plasmid, bacmid, minigene, ministring DNA (linear covalently closed DNA vector), closed-ended linear duplex DNA (CELiD or ceDNA), doggybone (dbDNA™) DNA, dumbbell shaped DNA, minimalistic immunological-defined gene expression (MIDGE)-vector, viral vector or nonviral vectors. RNA may be in the form of small interfering RNA (siRNA), Dicer-substrate dsRNA, small hairpin RNA (shRNA), asymmetrical interfering RNA (aiRNA), microRNA (miRNA), mRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs and/or modified residues include, without limitation, phosphorothioates, phosphorodiamidate morpholino oligomer (morpholino), phosphoramidates, methyl phosphonates, chiral-methyl phosphonates, 2′-O-methyl ribonucleotides, locked nucleic acid (LNA™), and peptide nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated.

“Nucleotides” contain a sugar deoxyribose (DNA) or ribose (RNA), a base, and a phosphate group. Nucleotides are linked together through the phosphate groups.

“Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence of the present disclosure. An “expression cassette” includes a DNA coding sequence operably linked to a promoter.

By “hybridizable” or “complementary” or “substantially complementary” it is meant that a nucleic acid (e.g., RNA) includes a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. As is known in the art, standard Watson-Crick base-pairing includes: adenine (A) pairing with thymidine (T), adenine (A) pairing with uracil (U), and guanine (G) pairing with cytosine (C). In addition, it is also known in the art that for hybridization between two RNA molecules (e.g., dsRNA), guanine (G) base pairs with uracil (U). For example, G/U base-pairing is partially responsible for the degeneracy (i.e., redundancy) of the genetic code in the context of tRNA anti-codon base-pairing with codons in mRNA. In the context of this disclosure, a guanine (G) of a protein-binding segment (dsRNA duplex) of a subject DNA-targeting RNA molecule is considered complementary to a uracil (U), and vice versa. As such, when a G/U base-pair can be made at a given nucleotide position a protein-binding segment (dsRNA duplex) of a subject DNA-targeting RNA molecule, the position is not considered to be non-complementary, but is instead considered to be complementary.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

A DNA sequence that “encodes” a particular antigen or immunogenic peptide, is a DNA nucleic acid sequence that is transcribed into the particular RNA and/or protein. A DNA polynucleotide may encode an RNA (mRNA) that is translated into protein, or a DNA polynucleotide may encode an RNA that is not translated into protein (e.g., tRNA, rRNA, or a DNA-targeting RNA; also called “non-coding” RNA or “ncRNA”).

As used herein, the term “terminal repeat” or “TR” includes any viral terminal repeat or synthetic sequence that comprises at least one minimal required origin of replication and a region comprising a palindrome hairpin structure. A Rep-binding sequence (“RBS”) (also referred to as RBE (Rep-binding element)) and a terminal resolution site (“TRS”) together constitute a “minimal required origin of replication” and thus the TR comprises at least one RBS and at least one TRS. TRs that are the inverse complement of one another within a given stretch of polynucleotide sequence are typically each referred to as an “inverted terminal repeat” or “ITR”. In the context of a virus, ITRs mediate replication, virus packaging, integration and provirus rescue. As was unexpectedly found, TRs that are not inverse complements across their full length can still perform the traditional functions of ITRs, and thus the term ITR is used herein to refer to a TR in a ceDNA genome or ceDNA vector that is capable of mediating replication of ceDNA vector. It will be understood by one of ordinary skill in the art that in complex ceDNA vector configurations more than two ITRs or asymmetric ITR pairs may be present. The ITR can be an AAV ITR or a non-AAV ITR, or can be derived from an AAV ITR or a non-AAV ITR. For example, the ITR can be derived from the family Parvoviridae, which encompasses Parvoviruses and Dependoviruses (e.g., canine parvovirus, bovine parvovirus, mouse parvovirus, porcine parvovirus, human parvovirus B-19), or the SV40 hairpin that serves as the origin of SV40 replication can be used as an ITR, which can further be modified by truncation, substitution, deletion, insertion and/or addition. Parvoviridae family viruses consist of two subfamilies: Parvovirinae, which infect vertebrates, and Densovirinae, which infect invertebrates. Dependoparvoviruses include the viral family of the adeno-associated viruses (AAV) which are capable of replication in vertebrate hosts including, but not limited to, human, primate, bovine, canine, equine and ovine species. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

A “wild-type ITR” or “WT-ITR” refers to the sequence of a naturally occurring ITR sequence in an AAV or other dependovirus that retains, e.g., Rep binding activity and Rep nicking ability. The nucleic acid sequence of a WT-ITR from any AAV serotype may slightly vary from the canonical naturally occurring sequence due to degeneracy of the genetic code or drift, and therefore WT-ITR sequences encompassed for use herein include WT-ITR sequences as result of naturally occurring changes taking place during the production process (e.g., a replication error).

As used herein, the term “substantially symmetrical WT-ITRs” or a “substantially symmetrical WT-ITR pair” refers to a pair of WT-ITRs within a single ceDNA genome or ceDNA vector that are both wild type ITRs that have an inverse complement sequence across their entire length. For example, an ITR can be considered to be a wild-type sequence, even if it has one or more nucleotides that deviate from the canonical naturally occurring sequence, so long as the changes do not affect the properties and overall three-dimensional structure of the sequence. According to some aspects, the deviating nucleotides represent conservative sequence changes. As one non-limiting example, a sequence that has at least 95%, 96%, 97%, 98%, or 99% sequence identity to the canonical sequence (as measured, e.g., using BLAST at default settings), and also has a symmetrical three-dimensional spatial organization to the other WT-ITR such that their 3D structures are the same shape in geometrical space. The substantially symmetrical WT-ITR has the same A, C-C′ and B-B′ loops in 3D space. A substantially symmetrical WT-ITR can be functionally confirmed as WT by determining that it has an operable Rep binding site (RBE or RBE′) and terminal resolution site (trs) that pairs with the appropriate Rep protein. One can optionally test other functions, including transgene expression under permissive conditions.

As used herein, the phrases of “modified ITR” or “mod-ITR” or “mutant ITR” are used interchangeably herein and refer to an ITR that has a mutation in at least one or more nucleotides as compared to the WT-ITR from the same serotype. The mutation can result in a change According to some or more of A, C, C′, B, B′ regions in the ITR, and can result in a change in the three-dimensional spatial organization (i.e., its 3D structure in geometric space) as compared to the 3D spatial organization of a WT-ITR of the same serotype.

As used herein, the term “asymmetric ITRs” also referred to as “asymmetric ITR pairs” refers to a pair of ITRs within a single ceDNA genome or ceDNA vector that are not inverse complements across their full length. As one non-limiting example, an asymmetric ITR pair does not have a symmetrical three-dimensional spatial organization to their cognate ITR such that their 3D structures are different shapes in geometrical space. Stated differently, an asymmetrical ITR pair have the different overall geometric structure, i.e., they have different organization of their A, C-C′ and B-B′ loops in 3D space (e.g., one ITR may have a short C-C′ arm and/or short B-B′ arm as compared to the cognate ITR). The difference in sequence between the two ITRs may be due to one or more nucleotide addition, deletion, truncation, or point mutation. According to some embodiments, one ITR of the asymmetric ITR pair may be a wild-type AAV ITR sequence and the other ITR a modified ITR as defined herein (e.g., a non-wild-type or synthetic ITR sequence). In another embodiment, neither ITRs of the asymmetric ITR pair is a wild-type AAV sequence and the two ITRs are modified ITRs that have different shapes in geometrical space (i.e., a different overall geometric structure). According to some embodiments, one mod-ITRs of an asymmetric ITR pair can have a short C-C′ arm and the other ITR can have a different modification (e.g., a single arm, or a short B-B′ arm etc.) such that they have different three-dimensional spatial organization as compared to the cognate asymmetric mod-ITR.

As used herein, the term “symmetric ITRs” refers to a pair of ITRs within a single ceDNA genome or ceDNA vector that are mutated or modified relative to wild-type dependoviral ITR sequences and are inverse complements across their full length. Neither ITRs are wild type ITR AAV2 sequences (i.e., they are a modified ITR, also referred to as a mutant ITR), and can have a difference in sequence from the wild type ITR due to nucleotide addition, deletion, substitution, truncation, or point mutation. For convenience herein, an ITR located 5′ to (upstream of) an expression cassette in a ceDNA vector is referred to as a “5′ ITR” or a “left ITR”, and an ITR located 3′ to (downstream of) an expression cassette in a ceDNA vector is referred to as a “3′ ITR” or a “right ITR”.

As used herein, the terms “substantially symmetrical modified-ITRs” or a “substantially symmetrical mod-ITR pair” refers to a pair of modified-ITRs within a single ceDNA genome or ceDNA vector that are both that have an inverse complement sequence across their entire length. For example, the modified ITR can be considered substantially symmetrical, even if it has some nucleotide sequences that deviate from the inverse complement sequence so long as the changes do not affect the properties and overall shape. As one non-limiting example, a sequence that has at least 85%, 90%, 91%, 92%, 93%, 94%, 95%, 96%, 97%, 98%, or 99% sequence identity to the canonical sequence (as measured using BLAST at default settings), and also has a symmetrical three-dimensional spatial organization to their cognate modified ITR such that their 3D structures are the same shape in geometrical space. Stated differently, a substantially symmetrical modified-ITR pair have the same A, C-C′ and B-B′ loops organized in 3D space. According to some embodiments, the ITRs from a mod-ITR pair may have different reverse complement nucleotide sequences but still have the same symmetrical three-dimensional spatial organization—that is both ITRs have mutations that result in the same overall 3D shape. For example, one ITR (e.g., 5′ ITR) in a mod-ITR pair can be from one serotype, and the other ITR (e.g., 3′ ITR) can be from a different serotype, however, both can have the same corresponding mutation (e.g., if the 5′ITR has a deletion in the C region, the cognate modified 3′ITR from a different serotype has a deletion at the corresponding position in the C′ region), such that the modified ITR pair has the same symmetrical three-dimensional spatial organization. In such embodiments, each ITR in a modified ITR pair can be from different serotypes (e.g., AAV1, 2, 3, 4, 5, 6, 7, 8, 9, 10, 11, and 12) such as the combination of AAV2 and AAV6, with the modification According to some ITR reflected in the corresponding position in the cognate ITR from a different serotype. According to some embodiments, a substantially symmetrical modified ITR pair refers to a pair of modified ITRs (mod-ITRs) so long as the difference in nucleotide sequences between the ITRs does not affect the properties or overall shape and they have substantially the same shape in 3D space. As a non-limiting example, a mod-ITR that has at least 90%, 91%, 92%, 93%, 94%, 95%, 96%, 97%, 98% or 99% sequence identity to the canonical mod-ITR as determined by standard means well known in the art such as BLAST (Basic Local Alignment Search Tool), or BLASTN at default settings, and also has a symmetrical three-dimensional spatial organization such that their 3D structure is the same shape in geometric space. A substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR.

As used herein, an “Internal ribosomal entry site” (IRES) is meant to refer to a nucleotide sequence (>500 nucleotides) that allows for initiation of translation in the middle of an mRNA sequence (Kirn, J I T. et al., 2011. PLoS One 6(4): 8556; the contents of which are herein incorporated by reference in its entirety). Use of an IRES sequence ensures co-expression of genes before and after the IRES, though the sequence following the IRES may be transcribed and translated at lower levels than the sequence preceding the IRES sequence.

As used herein, “2A peptides” are meant to refer to small self-cleaving peptides derived from viruses such as foot-and-mouth disease virus (F2A), porcine teschovirus-1 (P2A), osea asigna virus (T2A), or equine rhinitis A virus (E2A). The 2A designation refers specifically to a region of picomavirus poiyproteins that lead to a ribosomal skip at the glycyl-prolyl bond in the O terminus of the 2A peptide (Kim, J. I T. et al. 2011. PLoS One 6(4); the contents of which are herein incorporated by reference in its entirety). This skip results in a cleavage between the 2A peptide and its immediate downstream peptide.

The term “flanking” refers to a relative position of one nucleic acid sequence with respect to another nucleic acid sequence. Generally, in the sequence ABC, B is flanked by A and C. The same is true for the arrangement A×B×C. Thus, a flanking sequence precedes or follows a flanked sequence but need not be contiguous with, or immediately adjacent to the flanked sequence. According to some embodiments, the term flanking refers to terminal repeats at each end of the linear duplex ceDNA vector.

As used herein, the term “ceDNA genome” refers to an expression cassette that further incorporates at least one inverted terminal repeat region. A ceDNA genome may further comprise one or more spacer regions. According to some embodiments the ceDNA genome is incorporated as an intermolecular duplex polynucleotide of DNA into a plasmid or viral genome.

As used herein, the term “ceDNA spacer region” refers to an intervening sequence that separates functional elements in the ceDNA vector or ceDNA genome. According to some embodiments, ceDNA spacer regions keep two functional elements at a desired distance for optimal functionality. According to some embodiments, ceDNA spacer regions provide or add to the genetic stability of the ceDNA genome within e.g., a plasmid or baculovirus. According to some embodiments, ceDNA spacer regions facilitate ready genetic manipulation of the ceDNA genome by providing a convenient location for cloning sites and the like. For example, in certain aspects, an oligonucleotide “polylinker” containing several restriction endonuclease sites, or a non-open reading frame sequence designed to have no known protein (e.g., transcription factor) binding sites can be positioned in the ceDNA genome to separate the cis-acting factors, e.g., inserting a 6mer, 12mer, 18mer, 24mer, 48mer, 86mer, 176mer, etc. between the terminal resolution site and the upstream transcriptional regulatory element. Similarly, the spacer may be incorporated between the polyadenylation signal sequence and the 3′-terminal resolution site.

As used herein, the terms “Rep binding site, “Rep binding element, “RBE” and “RBS” are used interchangeably and refer to a binding site for Rep protein (e.g., AAV Rep 78 or AAV Rep 68) which upon binding by a Rep protein permits the Rep protein to perform its site-specific endonuclease activity on the sequence incorporating the RBS. An RBS sequence and its inverse complement together form a single RBS. RBS sequences are known in the art, and include, for example, 5′-GCGCGCTCGCTCGCTC-3′, an RBS sequence identified in AAV2. Any known RBS sequence may be used in the embodiments of the disclosure, including other known AAV RBS sequences and other naturally known or synthetic RBS sequences. Without being bound by theory it is thought that the nuclease domain of a Rep protein binds to the duplex nucleic acid sequence GCTC, and thus the two known AAV Rep proteins bind directly to and stably assemble on the duplex oligonucleotide, 5′-(GCGC)(GCTC)(GCTC)(GCTC)-3′. In addition, soluble aggregated conformers (i.e., undefined number of inter-associated Rep proteins) dissociate and bind to oligonucleotides that contain Rep binding sites. Each Rep protein interacts with both the nitrogenous bases and phosphodiester backbone on each strand. The interactions with the nitrogenous bases provide sequence specificity whereas the interactions with the phosphodiester backbone are non- or less-sequence specific and stabilize the protein-DNA complex.

As used herein, the terms “terminal resolution site” and “TRS” are used interchangeably herein and refer to a region at which Rep forms a tyrosine-phosphodiester bond with the 5′ thymidine generating a 3′ OH that serves as a substrate for DNA extension via a cellular DNA polymerase, e.g., DNA pol delta or DNA pol epsilon. Alternatively, the Rep-thymidine complex may participate in a coordinated ligation reaction. According to some embodiments, a TRS minimally encompasses a non-base-paired thymidine. According to some embodiments, the nicking efficiency of the TRS can be controlled at least in part by its distance within the same molecule from the RBS. When the acceptor substrate is the complementary ITR, then the resulting product is an intramolecular duplex. TRS sequences are known in the art, and include, for example, 5′-GGTTGA-3′, the hexanucleotide sequence identified in AAV2. Any known TRS sequence may be used in the embodiments of the disclosure, including other known AAV TRS sequences and other naturally known or synthetic TRS sequences such as AGTT, GGTTGG, AGTTGG, AGTTGA, and other motifs such as RRTTRR.

As used herein, the term “ceDNA” refers to capsid-free closed-ended linear double stranded (ds) duplex DNA for non-viral gene transfer, synthetic or otherwise. Detailed description of ceDNA is described in International application of PCT/US2017/020828, filed Mar. 3, 2017, the entire contents of which are expressly incorporated herein by reference. Certain methods for the production of ceDNA comprising various inverted terminal repeat (ITR) sequences and configurations using cell-based methods are described in Example 1 of International applications PCT/US18/49996, filed Sep. 7, 2018, and PCT/US2018/064242, filed Dec. 6, 2018 each of which is incorporated herein in its entirety by reference. Certain methods for the production of synthetic ceDNA vectors comprising various ITR sequences and configurations are described, e.g., in International application PCT/US2019/14122, filed Jan. 18, 2019, the entire content of which is incorporated herein by reference. As used herein, the terms “ceDNA vector” and “ceDNA” are used interchangeably and refer to a closed-ended DNA vector comprising at least one terminal palindrome. According to some embodiments, the ceDNA comprises two covalently-closed ends.

As used herein, the term “ceDNA-plasmid” refers to a plasmid that comprises a ceDNA genome as an intermolecular duplex.

As used herein, the term “ceDNA-bacmid” refers to an infectious baculovirus genome comprising a ceDNA genome as an intermolecular duplex that is capable of propagating in E. coli as a plasmid, and so can operate as a shuttle vector for baculovirus.

As used herein, the term “ceDNA-baculovirus” refers to a baculovirus that comprises a ceDNA genome as an intermolecular duplex within the baculovirus genome.

As used herein, the terms “ceDNA-baculovirus infected insect cell” and “ceDNA-BIIC” are used interchangeably, and refer to an invertebrate host cell (including, but not limited to an insect cell (e.g., an Sf9 cell)) infected with a ceDNA-baculovirus.

As used herein, the term “closed-ended DNA vector” refers to a capsid-free DNA vector with at least one covalently closed end and where at least part of the vector has an intramolecular duplex structure.

As defined herein, “reporters” refer to proteins that can be used to provide detectable read-outs. Reporters generally produce a measurable signal such as fluorescence, color, or luminescence.

Reporter protein coding sequences encode proteins whose presence in the cell or organism is readily observed. For example, fluorescent proteins cause a cell to fluoresce when excited with light of a particular wavelength, luciferases cause a cell to catalyze a reaction that produces light, and enzymes such as β-galactosidase convert a substrate to a colored product. Exemplary reporter polypeptides useful for experimental or diagnostic purposes include, but are not limited to β-lactamase, β-galactosidase (LacZ), alkaline phosphatase (AP), thymidine kinase (TK), green fluorescent protein (GFP) and other fluorescent proteins, chloramphenicol acetyltransferase (CAT), luciferase, and others well known in the art.

As used herein, the term “effector protein” refers to a polypeptide that provides a detectable read-out, either as, for example, a reporter polypeptide, or more appropriately, as a polypeptide that kills a cell, e.g., a toxin, or an agent that renders a cell susceptible to killing with a chosen agent or lack thereof. Effector proteins include any protein or peptide that directly targets or damages the host cell's DNA and/or RNA. For example, effector proteins can include, but are not limited to, a restriction endonuclease that targets a host cell DNA sequence (whether genomic or on an extrachromosomal element), a protease that degrades a polypeptide target necessary for cell survival, a DNA gyrase inhibitor, and a ribonuclease-type toxin. According to some embodiments, the expression of an effector protein controlled by a synthetic biological circuit as described herein can participate as a factor in another synthetic biological circuit to thereby expand the range and complexity of a biological circuit system's responsiveness.

Transcriptional regulators refer to transcriptional activators and repressors that either activate or repress transcription of a transgene (e.g., a nucleic acid encoding an antibody or antigen-binding fragment thereof as described herein). Promoters are regions of nucleic acid that initiate transcription of a particular gene. Transcriptional activators typically bind nearby to transcriptional promoters and recruit RNA polymerase to directly initiate transcription. Repressors bind to transcriptional promoters and sterically hinder transcriptional initiation by RNA polymerase. Other transcriptional regulators may serve as either an activator or a repressor depending on where they bind and cellular and environmental conditions. Non-limiting examples of transcriptional regulator classes include, but are not limited to homeodomain proteins, zinc-finger proteins, winged-helix (forkhead) proteins, and leucine-zipper proteins.

As used herein, a “repressor protein” or “inducer protein” is a protein that binds to a regulatory sequence element and represses or activates, respectively, the transcription of sequences operatively linked to the regulatory sequence element. Preferred repressor and inducer proteins as described herein are sensitive to the presence or absence of at least one input agent or environmental input. Preferred proteins as described herein are modular in form, comprising, for example, separable DNA-binding and input agent-binding or responsive elements or domains.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutically active substances is well known in the art. Supplementary active ingredients can also be incorporated into the compositions. The phrase “pharmaceutically-acceptable” refers to molecular entities and compositions that do not produce a toxic, an allergic, or similar untoward reaction when administered to a host.

As used herein, an “input agent responsive domain” is a domain of a transcription factor that binds to or otherwise responds to a condition or input agent in a manner that renders a linked DNA binding fusion domain responsive to the presence of that condition or input. According to some embodiments, the presence of the condition or input results in a conformational change in the input agent responsive domain, or in a protein to which it is fused, that modifies the transcription-modulating activity of the transcription factor.

The term “in vivo” refers to assays or processes that occur in or within an organism, such as a multicellular animal. According to some of the aspects described herein, a method or use can be said to occur “in vivo” when a unicellular organism, such as a bacterium, is used. The term “ex vivo” refers to methods and uses that are performed using a living cell with an intact membrane that is outside of the body of a multicellular animal or plant, e.g., explants, cultured cells, including primary cells and cell lines, transformed cell lines, and extracted tissue or cells, including blood cells, among others. The term “in vitro” refers to assays and methods that do not require the presence of a cell with an intact membrane, such as cellular extracts, and can refer to the introducing of a programmable synthetic biological circuit in a non-cellular system, such as a medium not comprising cells or cellular systems, such as cellular extracts.

The term “promoter,” as used herein, refers to any nucleic acid sequence that regulates the expression of another nucleic acid sequence by driving transcription of the nucleic acid sequence, which can be a heterologous target gene encoding a protein or an RNA. Promoters can be constitutive, inducible, repressible, tissue-specific, or any combination thereof. A promoter is a control region of a nucleic acid sequence at which initiation and rate of transcription of the remainder of a nucleic acid sequence are controlled. A promoter can also contain genetic elements at which regulatory proteins and molecules can bind, such as RNA polymerase and other transcription factors. According to some embodiments of the aspects described herein, a promoter can drive the expression of a transcription factor that regulates the expression of the promoter itself. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the expression of transgenes in the ceDNA vectors disclosed herein. A promoter sequence may be bounded at its 3′ terminus by the transcription initiation site and extends upstream (5′ direction) to include the minimum number of bases or elements necessary to initiate transcription at levels detectable above background. According to some embodiments, a promoter of the disclosure is a liver specific promoter.

The term “enhancer” as used herein refers to a cis-acting regulatory sequence (e.g., 10-1,500 base pairs) that binds one or more proteins (e.g., activator proteins, or transcription factor) to increase transcriptional activation of a nucleic acid sequence. Enhancers can be positioned up to 1,000,000 base pars upstream of the gene start site or downstream of the gene start site that they regulate. An enhancer can be positioned within an intronic region, or in the exonic region of an unrelated gene.

A promoter can be said to drive expression or drive transcription of the nucleic acid sequence that it regulates. The phrases “operably linked,” “operatively positioned,” “operatively linked,” “under control,” and “under transcriptional control” indicate that a promoter is in a correct functional location and/or orientation in relation to a nucleic acid sequence it regulates to control transcriptional initiation and/or expression of that sequence. An “inverted promoter,” as used herein, refers to a promoter in which the nucleic acid sequence is in the reverse orientation, such that what was the coding strand is now the non-coding strand, and vice versa. Inverted promoter sequences can be used in various embodiments to regulate the state of a switch. In addition, in various embodiments, a promoter can be used in conjunction with an enhancer.

A promoter can be one naturally associated with a gene or sequence, as can be obtained by isolating the 5′ non-coding sequences located upstream of the coding segment and/or exon of a given gene or sequence. Such a promoter can be referred to as “endogenous.” Similarly, according to some embodiments, an enhancer can be one naturally associated with a nucleic acid sequence, located either downstream or upstream of that sequence.

According to some embodiments, a coding nucleic acid segment is positioned under the control of a “recombinant promoter” or “heterologous promoter,” both of which refer to a promoter that is not normally associated with the encoded nucleic acid sequence it is operably linked to in its natural environment. A recombinant or heterologous enhancer refers to an enhancer not normally associated with a given nucleic acid sequence in its natural environment. Such promoters or enhancers can include promoters or enhancers of other genes; promoters or enhancers isolated from any other prokaryotic, viral, or eukaryotic cell; and synthetic promoters or enhancers that are not “naturally occurring,” i.e., comprise different elements of different transcriptional regulatory regions, and/or mutations that alter expression through methods of genetic engineering that are known in the art. In addition to producing nucleic acid sequences of promoters and enhancers synthetically, promoter sequences can be produced using recombinant cloning and/or nucleic acid amplification technology, including PCR, in connection with the synthetic biological circuits and modules disclosed herein (see, e.g., U.S. Pat. Nos. 4,683,202, 5,928,906, each incorporated herein by reference). Furthermore, it is contemplated that control sequences that direct transcription and/or expression of sequences within non-nuclear organelles such as mitochondria, chloroplasts, and the like, can be employed as well.

As described herein, an “inducible promoter” is one that is characterized by initiating or enhancing transcriptional activity when in the presence of, influenced by, or contacted by an inducer or inducing agent. An “inducer” or “inducing agent,” as defined herein, can be endogenous, or a normally exogenous compound or protein that is administered in such a way as to be active in inducing transcriptional activity from the inducible promoter. According to some embodiments, the inducer or inducing agent, i.e., a chemical, a compound or a protein, can itself be the result of transcription or expression of a nucleic acid sequence (i.e., an inducer can be an inducer protein expressed by another component or module), which itself can be under the control or an inducible promoter. According to some embodiments, an inducible promoter is induced in the absence of certain agents, such as a repressor. Examples of inducible promoters include but are not limited to, tetracycline, metallothionine, ecdysone, mammalian viruses (e.g., the adenovirus late promoter; and the mouse mammary tumor virus long terminal repeat (MMTV-LTR)) and other steroid-responsive promoters, rapamycin responsive promoters and the like.

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., DNA-targeting RNA) or a coding sequence (e.g., site-directed modifying polypeptide, or Cas9/Csn1 polypeptide) and/or regulate translation of an encoded polypeptide.

The term “open reading frame (ORF)” as used herein is meant to refer to a sequence of several nucleotide triplets which may be translated into a peptide or protein. An open reading frame preferably contains a start codon, i.e. a combination of three subsequent nucleotides coding usually for the amino acid methionine (ATG), at its 5′-end and a subsequent region which usually exhibits a length which is a multiple of 3 nucleotides. An ORF is preferably terminated by a stop-codon (e.g., TAA, TAG, TGA). Typically, this is the only stop-codon of the open reading frame. Thus, an open reading frame in the context of the present disclosure is preferably a nucleotide sequence, consisting of a number of nucleotides that may be divided by three, which starts with a start codon (e.g., ATG) and which preferably terminates with a stop codon (e.g., TAA, TGA, or TAG). The open reading frame may be isolated or it may be incorporated in a longer nucleic acid sequence, for example in a ceDNA vector as described herein.

“Operably linked” refers to a juxtaposition wherein the components so described are in a relationship permitting them to function in their intended manner. For instance, a promoter is operably linked to a coding sequence if the promoter affects its transcription or expression. An “expression cassette” includes a DNA sequence that is operably linked to a promoter or other regulatory sequence sufficient to direct transcription of the transgene in the ceDNA vector. Suitable promoters include, for example, tissue specific promoters. Promoters can also be of AAV origin.

The term “subject” as used herein refers to a human or animal, to whom treatment, including prophylactic treatment, with the ceDNA vector according to the present disclosure, is provided. As used herein, the term “subject” includes humans and other animals. Typically, the subject is a human. For example, the subject may be an adult, a teenager, a child (2 years to 14 years of age), an infant (birth to 2 year), or a neonate (up to 2 months). In particular aspects, the subject is up to 4 months old, or up to 6 months old. According to some aspects, the adults are seniors about 65 years or older, or about 60 years or older. According to some aspects, the subject is a pregnant woman or a woman intending to become pregnant. In other aspects, subject is not a human; for example a non-human primate; such as a baboon, a chimpanzee, a gorilla, or a macaque. In certain aspects, the subject may be a pet, such as a dog or a cat.

As used herein, the term “host cell”, includes any cell type that is susceptible to transformation, transfection, transduction, and the like with a nucleic acid construct or ceDNA expression vector of the present disclosure. As non-limiting examples, a host cell can be an isolated primary cell, pluripotent stem cells, CD34⁺ cells), induced pluripotent stem cells, or any of a number of immortalized cell lines (e.g., HepG2 cells). Alternatively, a host cell can be an in situ or in vivo cell in a tissue, organ or organism.

The term “exogenous” refers to a substance present in a cell other than its native source. The term “exogenous” when used herein can refer to a nucleic acid (e.g., a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell.

The term “sequence identity” refers to the relatedness between two nucleotide sequences. For purposes of the present disclosure, the degree of sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 3.0.0 or later. The optional parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows: (Identical Deoxyribonucleotides.times.100)/(Length of Alignment-Total Number of Gaps in Alignment). The length of the alignment is preferably at least 10 nucleotides, preferably at least 25 nucleotides more preferred at least 50 nucleotides and most preferred at least 100 nucleotides.

The term “homology” or “homologous” as used herein is defined as the percentage of nucleotide residues that are identical to the nucleotide residues in the corresponding sequence on the target chromosome, after aligning the sequences and introducing gaps, if necessary, to achieve the maximum percent sequence identity. Alignment for purposes of determining percent nucleotide sequence homology can be achieved in various ways that are within the skill in the art, for instance, using publicly available computer software such as BLAST, BLAST-2, ALIGN, ClustalW2 or Megalign (DNASTAR) software. Those skilled in the art can determine appropriate parameters for aligning sequences, including any algorithms needed to achieve maximal alignment over the full length of the sequences being compared. According to some embodiments, a nucleic acid sequence (e.g., DNA sequence), for example of a homology arm, is considered “homologous” when the sequence is at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to the corresponding native or unedited nucleic acid sequence (e.g., genomic sequence) of the host cell.

The term “heterologous,” as used herein, means a nucleotide or polypeptide sequence that is not found in the native nucleic acid or protein, respectively. A heterologous nucleic acid sequence may be linked to a naturally-occurring nucleic acid sequence (or a variant thereof) (e.g., by genetic engineering) to generate a chimeric nucleotide sequence encoding a chimeric polypeptide. A heterologous nucleic acid sequence may be linked to a variant polypeptide (e.g., by genetic engineering) to generate a nucleic acid sequence encoding a fusion variant polypeptide. Alternatively, the term “heterologous” may refer to a nucleic acid sequence which is not naturally present in a cell or subject.

A “vector” or “expression vector” is a replicon, such as plasmid, bacmid, phage, virus, virion, or cosmid, to which another DNA segment, i.e., an “insert”, may be attached so as to bring about the replication of the attached segment in a cell. A vector can be a nucleic acid construct designed for delivery to a host cell or for transfer between different host cells. As used herein, a vector can be viral or non-viral in origin and/or in final form, however for the purpose of the present disclosure, a “vector” generally refers to a ceDNA vector, as that term is used herein. The term “vector” encompasses any genetic element that is capable of replication when associated with the proper control elements and that can transfer gene sequences to cells. According to some embodiments, a vector can be an expression vector or recombinant vector.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g., 5′ untranslated (5′UTR) or “leader” sequences and 3′ UTR or “trailer” sequences, as well as intervening sequences (introns) between individual coding segments (exons).

By “recombinant vector” is meant a vector that includes a heterologous nucleic acid sequence, or “transgene” that is capable of expression in vivo. It should be understood that the vectors described herein can, according to some embodiments, be combined with other suitable compositions and therapies. According to some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms, “administration,” “administering” and variants thereof refers to introducing a composition or agent (e.g., a ceDNA as described herein) into a subject and includes concurrent and sequential introduction of one or more compositions or agents. “Administration” can refer, e.g., to therapeutic, pharmacokinetic, diagnostic, research, placebo, and experimental methods. “Administration” also encompasses in vitro and ex vivo treatments. The introduction of a composition or agent into a subject is by any suitable route, including orally, pulmonarily, intranasally, parenterally (intravenously, intramuscularly, intraperitoneally, or subcutaneously), rectally, intralymphatically, intratumorally, or topically. Administration includes self-administration and the administration by another. Administration can be carried out by any suitable route. A suitable route of administration allows the composition or the agent to perform its intended function. For example, if a suitable route is intravenous, the composition is administered by introducing the composition or agent into a vein of the subject.

The term “infection” as used herein refers to the initial entry of a pathogen into a host; and the condition in which the pathogen has become established in or on cells or tissues of a host; such a condition does not necessarily constitute or lead to a disease.

As used herein, the term “biological sample” refers to any type of material of biological origin isolated from a subject, including, for example, DNA, RNA, lipids, carbohydrates, and protein. The term “biological sample” includes tissues, cells and biological fluids isolated from a subject. Biological samples include, e.g., but are not limited to, whole blood, plasma, serum, semen, saliva, tears, urine, fecal material, sweat, buccal, skin, cerebrospinal fluid, bone marrow, bile, hair, muscle biopsy, organ tissue or other material of biological origin known by those of ordinary skill in the art. Biological samples can be obtained from subjects for diagnosis or research or can be obtained from healthy subjects, as controls or for basic research. The term “dose” as used herein refers to the quantity of a substance (e.g., a ceDNA as described herein) to be taken or administered to the subject at one time.

The term “dosing”, as used herein, refers to the administration of a substance (e.g., a ceDNA as described herein) to achieve a therapeutic objective (e.g., treatment).

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present disclosure, therefore, includes methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may to administer to the subject a second agent, and the administering steps may be executed at the same time, or nearly the same time, or at distant times, so long as the first agent (and additional agents) are after administration in the presence of the second agent (and additional agents). The actor and the subject may be the same entity (e.g., human).

The term “combination therapy”, as used herein, refers to the administration of two or more therapeutic substances, e.g., an antigen, or immunogenic protein, as described herein, and another drug. The other drug(s) may be administered concomitant with, prior to, or following the administration of the antigen, or immunogenic protein, as described herein.

As used herein, the phrases “nucleic acid therapeutic”, “therapeutic nucleic acid” and “TNA” are used interchangeably and refer to any modality of therapeutic using nucleic acids as an active component of therapeutic agent to treat a disease or disorder. As used herein, these phrases refer to RNA-based therapeutics and DNA-based therapeutics. Non-limiting examples of RNA-based therapeutics include mRNA, antisense RNA and oligonucleotides, ribozymes, aptamers, interfering RNAs (RNAi), Dicer-substrate dsRNA, small hairpin RNA (shRNA), asymmetrical interfering RNA (aiRNA), microRNA (miRNA). Non-limiting examples of DNA-based therapeutics include minicircle DNA, minigene, viral DNA (e.g., Lentiviral or AAV genome) or non-viral synthetic DNA vectors, closed-ended linear duplex DNA (ceDNA/CELiD), plasmids, bacmids, doggybone™ DNA vectors, minimalistic immunological-defined gene expression (MIDGE)-vector, nonviral ministring DNA vector (linear-covalently closed DNA vector), or dumbbell-shaped DNA minimal vector (“dumbbell DNA”). According to some embodiments, the therapeutic nucleic acid is a ceDNA.

As used herein the term “therapeutic effect” refers to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan. General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to therapeutic window, additional guidance for dosage modification can be obtained.

As used herein, “viral infection” is meant to refer to the invasion and multiplication of a virus in the body of a subject.

The term “treatment” as used herein is meant to refer to any of (i) the prevention of infection or reinfection, as in a traditional vaccine, (ii) the reduction or elimination of symptoms, and (iii) the substantial or complete elimination of the pathogen in question. Treatment may be effected prophylactically (prior to infection) or therapeutically (following infection). Treating may further refer to accomplishing one or more of the following: (a) reducing the severity of the disorder; ((b) limiting worsening of symptoms characteristic of the disorder(s) being treated; (c) limiting recurrence of the disorder(s) in patients that have previously had the disorder(s); and (d) limiting recurrence of symptoms in patients that were previously asymptomatic for the disorder(s).

Beneficial or desired clinical results, such as pharmacologic and/or physiologic effects include, but are not limited to, preventing the disease, disorder or condition from occurring in a subject that may be predisposed to the disease, disorder or condition but does not yet experience or exhibit symptoms of the disease (prophylactic treatment), alleviation of symptoms of the disease, disorder or condition, diminishment of extent of the disease, disorder or condition, stabilization (i.e., not worsening) of the disease, disorder or condition, preventing spread of the disease, disorder or condition, delaying or slowing of the disease, disorder or condition progression, amelioration or palliation of the disease, disorder or condition, and combinations thereof, as well as prolonging survival as compared to expected survival if not receiving treatment.

The term “vaccinated” as used herein is meant to refer to being treated with a vaccine.

The term “vaccination” as used herein is meant to refer to treatment with a vaccine.

The term “vaccine” as used herein is meant to refer to a formulation which is in a form that is capable of being administered to a vertebrate and which induces a protective immune response sufficient to induce immunity and/or to prevent and/or ameliorate an infection and/or to reduce at least one symptom of an infection and/or to enhance the efficacy of another dose of a formulation. Typically, the vaccine comprises a conventional saline or buffered aqueous solution medium in which the composition of the present disclosure is suspended or dissolved. In this form, the composition of the present disclosure can be used conveniently to prevent, ameliorate, or otherwise treat a viral infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

The term “vaccine therapy” as used herein is meant to refer to a type of treatment that uses a substance or group of substances to stimulate the immune system to destroy a tumor or infectious microorganisms.

Those “in need of treatment” include mammals, such as humans, already having a disease or disorder, an infection, or a cancer.

As used herein, the term “increase,” “enhance,” “raise” (and like terms) generally refers to the act of increasing, either directly or indirectly, a concentration, level, function, activity, or behavior relative to the natural, expected, or average, or relative to a control condition.

As used herein, the term “suppress,” “decrease,” “interfere,” “inhibit” and/or “reduce” (and like terms) generally refers to the act of reducing, either directly or indirectly, a concentration, level, function, activity, or behavior relative to the natural, expected, or average, or relative to a control condition.

As used herein, a “control” is meant to refer to a reference standard. According to some embodiments, the control is a negative control sample obtained from a healthy patient. In other embodiments, the control is a positive control sample obtained from a patient diagnosed with a disease or disorder, an infection or a cancer. In still other embodiments, the control is a historical control or standard reference value or range of values (such as a previously tested control sample, or group of samples that represent baseline or normal values). A difference between a test sample and a control can be an increase or conversely a decrease. The difference can be a qualitative difference or a quantitative difference, for example a statistically significant difference. According to some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, at least about 500%, or greater than 500%.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment. The use of “comprising” indicates inclusion rather than limitation.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus, for example, references to “the method” includes one or more methods, and/or steps of the type described herein and/or which will become apparent to those persons skilled in the art upon reading this disclosure and so forth. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.” The term “about” when used in connection with percentages can mean±±1%. The present disclosure is further explained in detail by the following examples, but the scope of the disclosure should not be limited thereto.

Groupings of alternative elements or embodiments of the disclosure disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Other terms are defined herein within the description of the various aspects of the disclosure.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting. It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims.

### II. Cells of the Immune System

There are a large number of cellular interactions that comprise the immune system. These interactions occur through specific receptor-ligand pairs that signal in both directions so that each cell receives instructions based on the temporal and spatial distribution of those signals.

Murine models have been highly useful in discovering immunomodulatory pathways, but clinical utility of these pathways does not always translate from an inbred mouse strain to an outbred human population, since an outbred human population may have individuals that rely to varying extents on individual immunomodulatory pathways.

Cells of the immune system include lymphocytes, monocytes/macrophages, dendritic cells, the closely related Langerhans cells, natural killer (NK) cells, mast cells, basophils, and other members of the myeloid lineage of cells. In addition, a series of specialized epithelial and stromal cells provide the anatomic environment in which immunity occurs, often by secreting critical factors that regulate growth and/or gene activation in cells of the immune system, which also play direct roles in the induction and effector phases of the response. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

The cells of the immune system are found in peripheral organized tissues, such as the spleen, lymph nodes, Peyer's patches of the intestine and tonsils. Lymphocytes also are found in the central lymphoid organs, the thymus, and bone marrow where they undergo developmental steps that equip them to mediate the myriad responses of the mature immune system. A substantial portion of lymphocytes and macrophages comprise a recirculating pool of cells found in the blood and lymph, providing the means to deliver immunocompetent cells to sites where they are needed and to allow immunity that is generated locally to become generalized. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens through recombination of their genetic material (e.g., to create a T cell receptor and a B cell receptor). This commitment, which exists before the first contact of the immune system with a given antigen, is expressed by the presence of receptors specific for determinants (epitopes) on the antigen on the lymphocyte's surface membrane. Each lymphocyte possesses a unique population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. (Paul, W. E., “Chapter 1: The immune system: an introduction,” Fundamental Immunology, 4th Edition, Ed. Paul, W. E., Lippicott-Raven Publishers, Philadelphia, (1999), at p. 102).

Two broad classes of lymphocytes are recognized: the B-lymphocytes (B-cells), which are precursors of antibody-secreting cells, and T-lymphocytes (T-cells).

### B-Lymphocytes

B-lymphocytes are derived from hematopoietic cells of the bone marrow. A mature B-cell can be activated with an antigen that expresses epitopes that are recognized by its cell surface. The activation process may be direct, dependent on cross-linkage of membrane Ig molecules by the antigen (cross-linkage-dependent B-cell activation), or indirect, via interaction with a helper T-cell, in a process referred to as cognate help. In many physiological situations, receptor cross-linkage stimuli and cognate help synergize to yield more vigorous B-cell responses (Paul, W. E., “Chapter 1: The immune system: an introduction,” Fundamental Immunology, 4th Edition, Ed. Paul, W. E., Lippicott-Raven Publishers, Philadelphia, (1999)).

Cross-linkage dependent B-cell activation requires that the antigen express multiple copies of the epitope complementary to the binding site of the cell surface receptors, because each B-cell expresses Ig molecules with identical variable regions. Such a requirement is fulfilled by other antigens with repetitive epitopes, such as capsular polysaccharides of microorganisms or viral envelope proteins. Cross-linkage-dependent B-cell activation is a major protective immune response mounted against these microbes (Paul, W. E., “Chapter 1: The immune system: an introduction”, Fundamental Immunology, 4th Edition, Ed. Paul, W. E., Lippicott-Raven Publishers, Philadelphia, (1999)).

Cognate help allows B-cells to mount responses against antigens that cannot cross-link receptors and, at the same time, provides costimulatory signals that rescue B cells from inactivation when they are stimulated by weak cross-linkage events. Cognate help is dependent on the binding of antigen by the B-cell's membrane immunoglobulin (Ig), the endocytosis of the antigen, and its fragmentation into peptides within the endosomal/lysosomal compartment of the cell. Some of the resultant peptides are loaded into a groove in a specialized set of cell surface proteins known as class II major histocompatibility complex (MHC) molecules. The resultant class II/peptide complexes are expressed on the cell surface and act as ligands for the antigen-specific receptors of a set of T-cells designated as CD4+ T-cells. The CD4+ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. In addition, T helper cells secrete several cytokines that regulate the growth and differentiation of the stimulated B-cell by binding to cytokine receptors on the B cell (Paul, W. E., “Chapter 1: The immune system: an introduction, “Fundamental Immunology, 4th Edition, Ed. Paul, W. E., Lippicott-Raven Publishers, Philadelphia, (1999)).

During cognate help for antibody production, the CD40 ligand is transiently expressed on activated CD4+T helper cells, and it binds to CD40 on the antigen-specific B cells, thereby transducing a second costimulatory signal. The latter signal is essential for B cell growth and differentiation and for the generation of memory B cells by preventing apoptosis of germinal center B cells that have encountered antigen. Hyperexpression of the CD40 ligand in both B and T cells is implicated in pathogenic autoantibody production in human SLE patients (Desai-Mehta, A. et al., “Hyperexpression of CD40 ligand by B and T cells in human lupus and its role in pathogenic autoantibody production,” J. Clin. Invest. Vol. 97(9), 2063-2073, (1996)).

### T-Lymphocytes

T-lymphocytes derived from precursors in hematopoietic tissue, undergo differentiation in the thymus, and are then seeded to peripheral lymphoid tissue and to the recirculating pool of lymphocytes. T-lymphocytes or T cells mediate a wide range of immunologic functions. These include the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on T cell expression of specific cell surface molecules and the secretion of cytokines (Paul, W. E., “Chapter 1: The immune system: an introduction”, Fundamental Immunology, 4th Edition, Ed. Paul, W. E., Lippicott-Raven Publishers, Philadelphia, (1999)).

T cells differ from B cells in their mechanism of antigen recognition. Immunoglobulin, the B cell's receptor, binds to individual epitopes on soluble molecules or on particulate surfaces. B-cell receptors see epitopes expressed on the surface of native molecules. While antibody and B-cell receptors evolved to bind to and to protect against microorganisms in extracellular fluids, T cells recognize antigens on the surface of other cells and mediate their functions by interacting with, and altering, the behavior of these antigen-presenting cells (APCs). There are three main types of APCs in peripheral lymphoid organs that can activate T cells: dendritic cells, macrophages and B cells. The most potent of these are the dendritic cells, whose only function is to present foreign antigens to T cells. Immature dendritic cells are located in tissues throughout the body, including the skin, gut, and respiratory tract. When they encounter invading microbes at these sites, they endocytose the pathogens and their products, and carry them via the lymph to local lymph nodes or gut associated lymphoid organs. The encounter with a pathogen induces the dendritic cell to mature from an antigen-capturing cell to an APC that can activate T cells. APCs display three types of protein molecules on their surface that have a role in activating a T cell to become an effector cell: (1) MHC proteins, which present foreign antigen to the T cell receptor; (2) costimulatory proteins which bind to complementary receptors on the T cell surface; and (3) cell-cell adhesion molecules, which enable a T cell to bind to the APC for long enough to become activated (“Chapter 24: The adaptive immune system,” Molecular Biology of the Cell, Alberts, B. et al., Garland Science, NY, (2002)).

T-cells are subdivided into two distinct classes based on the cell surface receptors they express. The majority of T cells express T cell receptors (TCR) consisting of α and β-chains. A small group of T cells express receptors made of γ and δ chains. Among the α/β T cells are two sub-lineages: those that express the coreceptor molecule CD4 (CD4+ T cells); and those that express CD8 (CD8+ T cells). These cells differ in how they recognize antigen and in their effector and regulatory functions.

CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated.

T cells also mediate important effector functions, some of which are determined by the patterns of cytokines they secrete. The cytokines can be directly toxic to target cells and can mobilize potent inflammatory mechanisms.

In addition, T cells, particularly CD8+ T cells, can develop into cytotoxic T-lymphocytes (CTLs) capable of efficiently lysing target cells that express antigens recognized by the CTLs (Paul, W. E., “Chapter 1: The immune system: an introduction,” Fundamental Immunology, 4th Edition, Ed. Paul, W. E., Lippicott-Raven Publishers, Philadelphia, (1999)).

T cell receptors (TCRs) recognize a complex consisting of a peptide derived by proteolysis of the antigen bound to a specialized groove of a class II or class I MHC protein. CD4+ T cells recognize only peptide/class II complexes while CD8+ T cells recognize peptide/class I complexes (Paul, W. E., “Chapter 1: The immune system: an introduction,” Fundamental Immunology, 4th Edition, Ed. Paul, W. E., Lippicott-Raven Publishers, Philadelphia, (1999)).

The TCR's ligand (i.e., the peptide/MHC protein complex) is created within APCs. In general, class II MHC molecules bind peptides derived from proteins that have been taken up by the APC through an endocytic process. These peptide-loaded class II molecules are then expressed on the surface of the cell, where they are available to be bound by CD4+ T cells with TCRs capable of recognizing the expressed cell surface complex. Thus, CD4+ T cells are specialized to react with antigens derived from extracellular sources (Paul, W. E., “Chapter 1: The immune system: an introduction,” Fundamental Immunology, 4th Edition, Ed. Paul, W. E., Lippicott-Raven Publishers, Philadelphia, (1999)).

In contrast, class I MHC molecules are mainly loaded with peptides derived from internally synthesized proteins, such as viral proteins. These peptides are produced from cytosolic proteins by proteolysis by the proteosome and are translocated into the rough endoplasmic reticulum. Such peptides, generally composed of nine amino acids in length, are bound into the class I MHC molecules and are brought to the cell surface, where they can be recognized by CD8+ T cells expressing appropriate receptors. This gives the T cell system, particularly CD8+ T cells, the ability to detect cells expressing proteins that are different from, or produced in much larger amounts than, those of cells of the remainder of the organism (e.g., viral antigens) or mutant antigens (such as active oncogene products), even if these proteins in their intact form are neither expressed on the cell surface nor secreted (Paul, W. E., “Chapter 1: The immune system: an introduction,” Fundamental Immunology, 4th Edition, Ed. Paul, W. E., Lippicott-Raven Publishers, Philadelphia, (1999)).

T cells can also be classified based on their function as helper T cells; T cells involved in inducing cellular immunity; suppressor T cells; and cytotoxic T cells.

### Helper T Cells

Helper T cells are T cells that stimulate B cells to make antibody responses to proteins and other T cell-dependent antigens. T cell-dependent antigens are immunogens in which individual epitopes appear only once or a limited number of times such that they are unable to cross-link the membrane immunoglobulin (Ig) of B cells or do so inefficiently. B cells bind the antigen through their membrane Ig, and the complex undergoes endocytosis. Within the endosomal and lysosomal compartments, the antigen is fragmented into peptides by proteolytic enzymes, and one or more of the generated peptides are loaded into class II MHC molecules, which traffic through this vesicular compartment. The resulting peptide/class II MHC complex is then exported to the B-cell surface membrane. T cells with receptors specific for the peptide/class II molecular complex recognize this complex on the B-cell surface. (Paul, W. E., “Chapter 1: The immune system: an introduction,” Fundamental Immunology, 4th Edition, Ed. Paul, W. E., Lippicott-Raven Publishers, Philadelphia (1999)).

B-cell activation depends both on the binding of the T cell through its TCR and on the interaction of the T-cell CD40 ligand (CD40L) with CD40 on the B cell. T cells do not constitutively express CD40L. Rather, CD40L expression is induced as a result of an interaction with an APC that expresses both a cognate antigen recognized by the TCR of the T cell and CD80 or CD86. CD80/CD86 is generally expressed by activated, but not resting, B cells so that the helper interaction involving an activated B cell and a T cell can lead to efficient antibody production. In many cases, however, the initial induction of CD40L on T cells is dependent on their recognition of antigen on the surface of APCs that constitutively express CD80/86, such as dendritic cells. Such activated helper T cells can then efficiently interact with and help B cells. Cross-linkage of membrane Ig on the B cell, even if inefficient, may synergize with the CD40L/CD40 interaction to yield vigorous B-cell activation. The subsequent events in the B-cell response, including proliferation, Ig secretion, and class switching of the Ig class being expressed, either depend or are enhanced by the actions of T cell-derived cytokines (Paul, W. E., “Chapter 1: The immune system: an introduction,” Fundamental Immunology, 4th Edition, Ed. Paul, W. E., Lippicott-Raven Publishers, Philadelphia, (1999)).

CD4+ T cells tend to differentiate into cells that principally secrete the cytokines IL-4, IL-5, IL-6, and IL-10 (TH2 cells) or into cells that mainly produce IL-2, IFN-γ, and lymphotoxin (TH1 cells). The TH2 cells are very effective in helping B-cells develop into antibody-producing cells, whereas the TH1 cells are effective inducers of cellular immune responses, involving enhancement of microbicidal activity of monocytes and macrophages, and consequent increased efficiency in lysing microorganisms in intracellular vesicular compartments. Although CD4+ T cells with the phenotype of TH2 cells (i.e., IL-4, IL-5, IL-6 and IL-10) are efficient helper cells, TH1 cells also have the capacity to be helpers (Paul, W. E., “Chapter 1: The immune system: an introduction, “Fundamental Immunology, 4th Edition, Ed. Paul, W. E., Lippicott-Raven Publishers, Philadelphia, (1999)).

### T Cell Involvement in Cellular Immunity Induction

T cells also may act to enhance the capacity of monocytes and macrophages to destroy intracellular microorganisms. In particular, interferon-gamma (IFN-γ) produced by helper T cells enhances several mechanisms through which mononuclear phagocytes destroy intracellular bacteria and parasitism including the generation of nitric oxide and induction of tumor necrosis factor (TNF) production. TH1 cells are effective in enhancing the microbicidal action, because they produce IFN-γ. In contrast, two of the major cytokines produced by TH2 cells, IL-4 and IL-10, block these activities (Paul, W. E., “Chapter 1: The immune system: an introduction,” Fundamental Immunology, 4th Edition, Ed. Paul, W. E., Lippicott-Raven Publishers, Philadelphia, (1999)).

### Regulatory T (Treg) Cells

Immune homeostasis is maintained by a controlled balance between initiation and downregulation of the immune response. The mechanisms of both apoptosis and T cell anergy (a tolerance mechanism in which the T cells are intrinsically functionally inactivated following an antigen encounter (Scwartz, R. H., “T cell anergy”, Annu. Rev. Immunol., Vol. 21: 305-334 (2003)) contribute to the downregulation of the immune response. A third mechanism is provided by active suppression of activated T cells by suppressor or regulatory CD4+T (Treg) cells (Reviewed in Kronenberg, M. et al., “Regulation of immunity by self-reactive T cells”, Nature, Vol. 435: 598-604 (2005)). CD4+ Tregs that constitutively express the IL-2 receptor alpha (IL-2Ra) chain (CD4+CD25+) are a naturally occurring T cell subset that are anergic and suppressive (Taams, L. S. et al., “Human anergic/suppressive CD4+CD25+ T cells: a highly differentiated and apoptosis-prone population”, Eur. J. Immunol. Vol. 31: 1122-1131 (2001)). Depletion of CD4+CD25+ Tregs results in systemic autoimmune disease in mice. Furthermore, transfer of these Tregs prevents development of autoimmune disease. Human CD4+CD25+ Tregs, similar to their murine counterpart, are generated in the thymus and are characterized by the ability to suppress proliferation of responder T cells through a cell-cell contact-dependent mechanism, the inability to produce IL-2, and the anergic phenotype in vitro. Human CD4+CD25+ T cells can be split into suppressive (CD25high) and nonsuppressive (CD25low) cells, according to the level of CD25 expression. A member of the forkhead family of transcription factors, FOXP3, has been shown to be expressed in murine and human CD4+CD25+ Tregs and appears to be a master gene controlling CD4+CD25+ Treg development (Battaglia, M. et al., “Rapamycin promotes expansion of functional CD4+CD25+Foxp3+ regulator T cells of both healthy subjects and type 1 diabetic patients”, J. Immunol., Vol. 177: 8338-8347, (2006)).

### Cytotoxic T Lymphocytes

CD8+ T cells that recognize peptides from proteins produced within the target cell have cytotoxic properties in that they lead to lysis of the target cells. The mechanism of CTL-induced lysis involves the production by the CTL of perforin, a molecule that can insert into the membrane of target cells and promote the lysis of that cell. Perforin-mediated lysis is enhanced by granzymes, a series of enzymes produced by activated CTLs. Many active CTLs also express large amounts of fas ligand on their surface. The interaction of fas ligand on the surface of CTL with fas on the surface of the target cell initiates apoptosis in the target cell, leading to the death of these cells. CTL-mediated lysis appears to be a major mechanism for the destruction of virally infected cells.

### Lymphocyte Activation

The term “activation” or “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the APC.

### T-Memory Cells

Following the recognition and eradication of pathogens through adaptive immune responses, the vast majority (90-95%) of T cells undergo apoptosis with the remaining cells forming a pool of memory T cells, designated central memory T cells (TCM), effector memory T cells (TEM), and resident memory T cells (TRM) (Clark, R. A., “Resident memory T cells in human health and disease”, Sci. Transl. Med., 7, 269 rv1, (2015)). CD45RA is expressed on naïve T cells, as well as the effector cells in both CD4 and CD8. After antigen experience, central and effector memory T cells gain expression of CD45RO and lose expression of CD45RA. Thus either CD45RA or CD45RO is used to generally differentiate the naïve from memory populations. CCR7 and CD62L are two other markers that can be used to distinguish central and effector memory T cells. Naïve and central memory cells express CCR7 and CD62L in order to migrate to secondary lymphoid organs. Thus, naïve T cells are CD45RA+CD45RO-CCR7+CD62L+, central memory T cells are CD45RA-CD45RO+CCR7+CD62L+, and effector memory T cells are CD45RA-CD45RO+CCR7-CD62L−.

Compared to standard T cells, these memory T cells are long-lived with distinct phenotypes such as expression of specific surface markers, rapid production of different cytokine profiles, capability of direct effector cell function, and unique homing distribution patterns. Memory T cells exhibit quick reactions upon re-exposure to their respective antigens in order to eliminate the reinfection of the offender and thereby restore balance of the immune system rapidly. Increasing evidence substantiates that autoimmune memory T cells hinder most attempts to treat or cure autoimmune diseases (Clark, R. A., “Resident memory T cells in human health and disease”, Sci. Transl. Med., Vol. 7, 269rv1, (2015)).

III. Expression of Antigens or Immunogenic Peptides from a ceDNA Vector

The technology described herein is directed in general to the expression and/or production of an antigen, or immunogenic peptide, in a cell from one or more non-viral DNA vectors, e.g., ceDNA vectors as described herein. ceDNA vectors for expression of an antigen, or immunogenic peptide antigen, are described herein in the section entitled “ceDNA vectors in general”. As previously discussed, a distinct advantage of ceDNA vectors over traditional AAV vectors, and even lentiviral vectors, is that there is no size constraint for the one or more nucleic acid sequences that encode an antigen, or an immunogenic peptide. The skilled artisan would appreciate, based upon the disclosure provided herein, that numerous antigens or immunogenic peptides (i.e., immunoregulatory molecules) can be used to produce an almost limitless variety of ceDNA vectors once armed with the teachings provided herein.

In particular embodiments, ceDNA vectors for expression of an antigen, or an immunogenic peptide, comprise a pair of ITRs (e.g., symmetric or asymmetric as described herein) and between the ITR pair, a nucleic acid encoding an antigen, or an immunogenic peptide, as described herein, operatively linked to a promoter or regulatory sequence. A distinct advantage of ceDNA vectors for expression of an antigen, or an immunogenic peptide, over traditional AAV vectors, and even lentiviral vectors, is that there is no size constraint for the nucleic acid sequences encoding the desired antigen, or immunogenic peptide.

As one will appreciate, the ceDNA vector technologies described herein can be adapted to any level of complexity or can be used in a modular fashion, where expression of different components of the ceDNA vector can be controlled in an independent manner. The following embodiments are specifically contemplated herein and can adapted by one of skill in the art as desired.

According to some aspects, the present disclosure provides one or more ceDNA vectors comprising one or more nucleic acid sequences that encode an antigen, or an immunogenic peptide. According to some embodiments, the one or more nucleic acid sequences encode one or more antigens, or immunogenic peptides, from a variety of pathogens, including, e.g., bacterial, viral, fungal and parasitic infectious agents. According to some embodiments, the one or more nucleic acid sequences encode one or more antigens, or immunogenic peptides, that are cancer or cancer-associated antigens. According to some embodiments, the antigen or immunogenic peptide is a tumor antigen. According to some embodiments, the one or more nucleic acid sequences encode one or more antigens, or immunogenic peptides, that are associated with an autoimmune condition, such as rheumatoid arthritis (RA) or multiple sclerosis (MS). According to some embodiments, the antigen is an antigen relating to an autoimmune disorder or condition, such as an autoimmune disease triggered by an infectious agent, or to an infectious disease or pathogen.

### Cancer or Tumor-Associated Antigens

According to some embodiments, the ceDNA comprises a nucleic acid sequence that encodes is a cancer or a tumor-associated antigen. According to some embodiments, the ceDNA comprises a nucleic acid sequence that encodes one or more antigens selected from the Cancer Antigenic Peptide Database, publically available at caped.icp.ucl.ac.be/about. This database includes the peptide sequence and its position in the protein sequence, for each antigen identified.

According to some embodiments, the ceDNA comprises a nucleic acid sequence that encodes a tumor-associated antigen selected from one of more of the antigens set forth in Table 1 below:

TABLE 1

| | | |
|---|---|---|
| Mesothelin | alpha fetal protein (AFP) | kRAS |
| NY-ESO-1 | cancer embryo antigen | Ep-CAM |
| | (CEA) | |
| FBP | SOX2 | MUC1 |
| HER-2/neu | GOLGA | Survivin |
| IL-13 receptor α2 | TPR | hTERT |
| melanoma-related antigens- | U2AF1L | WT1 |
| MAGE-1, MAGE-2, MAGE-3 | | |
| EphA2 | CYNL2 | C13orf53 |
| P53 | NSEP1 | RBPSUH |
| NKTR | C13orf24 | IG4 |
| GLEA2 | TNKS2 | HSPH1 |
| BRAP | RTN4 | KIAA0376 |
| SART1 | C9orf112 | AIM-3 |
| IL-13R alpha | TRP-2 | HER-2 |
| TRP-1 | ras | PSMA |
| PSA | PAP | MUM-1 |
| MART-1 | gp100 | gp75 |
| tyrosinase (Tyr) | midkin (MK) | BAGE |
| CASP-8 | β-catenin | CA-125 |
| CDK-1 | ESO-1 | |

Recent analyses of The Cancer Genome Atlas (TCGA) datasets have linked the genomic landscape of tumors with tumor immunity, implicating neoantigen load in driving T cell responses (Brown et al., Genome Res. 2014 May; 24(5):743-50, 2014) and identifying somatic mutations associated with immune infiltrates (Rutledge et al., Clin Cancer Res. 2013 Sep. 15; 19(18):4951-60, 2013). Rooney et al. (2015 Jan. 15;160(1-2):48-61) suggest that neoantigens and viruses are likely to drive cytolytic activity, and reveal known and novel mutations that enable tumors to resist immune attack.

In some embodiments, the antigen is a neoantigen identified from a cancer cell in a subject. In some embodiments, the neoantigen is a shared neoantigen. Methods of identifying neoantigens are known in the art and described, e.g., in U.S. Pat. No. 10,055,540, incorporated by reference in its entirety herein. Neoantigenic polypeptides and shared neoantigenic polypeptides are described, for example, in PCT/US2016/033452, U.S. Publication No. 20180055922, Schumacher and Hacohen et al. (Curr Opin Immunol. 2016 August;41:98-103), Gubin, M M et al. (Nature. 2014 Nov. 27; 515(7528):577-81), Schumacher and Schreiber, Science. 2015 Apr. 3; 348(6230):69-74), Ott P A., et al., Nature. 2017 Jul. 13; 547(7662):217-221, all of which are incorporated by reference in their entireties herein.

Accordingly, in some embodiments, the antigen is a neoantigen polypeptide. In some embodiments, the antigen is a neoantigen polypeptide set forth in The Comprehensive Tumor-Specific Neoantigen Database (TSNAdb v1.0); available at biopharm.zju.edu.cn/tsnadb and described in Wu et al., Genomics Proteomics Bioinformatics 16 (2018) 276-282. In some embodiments, the antigen is a neoantigen polypeptide set forth in U.S. Pat. No. 10,055,540, incorporated by reference in its entirety herein.

### Autoimmune Disease Antigens

According to some embodiments, antigen, or immunogenic peptide is associated with an autoimmune disease. According to some embodiments, the ceDNA comprises a nucleic acid sequence that encodes one or more antigens selected from those in Table 2, below.

TABLE 2

| Disease | Antigen |
|---|---|
| Neuromyelitis Optica (NMO) | Aquaporin 4 (AQP4) |
| Myasthenia Gravis (MG) | Acetylcholine receptor (AchR) |
| Membranous glomerulonephritis | Phospholipase A2 receptor (PLA2R) |
| Pemphigus Vulgaris (PV) | desmoglein 3 (DSG3) |
| Pemphigus Foliaceus (PF) | desmoglein 1 (DSG1) |
| Type I diabetes mellitus (T1DM) | Insulin/proinsulin/preproinsulin |
| Type I diabetes mellitus (T1DM) | glutamate decarboxylase (GAD65) |
| Type I diabetes mellitus (T1DM) | insulinoma antigen-2 (IA-2) |
| Multiple Sclerosis (MS) | myelin oligodendrocyte glycoprotein (MOG) |
| Multiple Sclerosis (MS) | myelin basic protein (MBP) |
| Multiple Sclerosis (MS) | proteolipid protein (PLP) |
| anti-phospholipid syndrome (APS)/CAPS | beta-2 glycoprotein 1 (b2GP1) |
| celiac disease | A-gliadin |
| Acute rheumatic fever | cross reactive antibodies to cardiac muscle |
| alopecia areata | Trychohyalin, keratin 16 |
| ANCA-associated vasculitis | Neutrophil cytoplasmic antigen, proteinase 3, |
| | myeloperodixase, bacterial permiability |
| | increasing factor |
| autoimmune gastritis | H, K adenosine triphosphatase |
| Autoimmune hemolytic | Rh blood group antigens, 1 antigen |
| anemia | |
| autoimmune hepatitis | nuclear protein, liver-kidney microsome type 1, |
| | liver cytosol type 1 |
| autoimmune myocarditis | cardiac myosin |
| Autoimmune thyroiditis | Thyroid peroxidase, thyroglobulin, thyroid- |
| | stimulating hormone receptor |
| Autoimmune uveitis | Retinal arrestin (S-antigen) |
| dermatomyositis | Mi2 ATPase |
| diabetes (type 1) | Pancreatic beta cell antigen |
| good pasture's syndrome | Noncollagenous domain of basement membrane |
| | collagen type IV |
| Graves' disease | Thyroid stimulating hormone receptor |
| Guillain-Barré syndrome | Neurofascin-18G, gliomedin, nodal adhesion |
| | molecueles |
| Hypoglycemia | Insulin receptor |
| Idiopathic thrombocytopenic purpura | Platelet integrin Gpllb, Gpllla |
| Insulin resistant diabetes | Insulin receptor |
| Membranous nephritis | Phospholipase AZ |
| mixed essential cryoglobulinemia | rheumatoid factor IgG complexes |
| multiple sclerosis | Myelin basic protein, proteolipid protein, |
| | myelin oligodendrocyte glycoprotein |
| myasthenia gravis | Acetylcholine receptor |
| Myasthenia gravis-MUSC | Muscarinic receptor |
| pemphigus/pemphigoid | Epidermal cadherin |
| pernicious anemia | intrinsic factor (Gastric) |
| polymyositis | nuclear and nucleolar antigen |
| primary biliary cirrhosis | neutrophil nuclear antigen, mitochondrial |
| | multienzyme complex |
| psoriasis | PSO p27 |
| rheumatoid arthritis | rheumatoid factor IgG complexes, synovial joint |
| | antigen, citrullinated protein, carbamylated |
| | protein |
| scleroderma/systemic sclerosis | Scl-86, nucleolar scleroderma antigen |
| Sjogren's syndrome | SS-B, tupus La protein |
| systemic lupus erythematosus | DNAr histones, ribosomes, snRNP, scRNP |
| vitiligo | VIT-90, VIT-75, VIT-40 |
| Wegener's granulomatosis | neutrophil nuclear antigen |
| Antiphospholipid syndrome (APS) & | Beta-2 glycoprotein 1 |
| catastrophic APS | |
| Chemotherapy induced peripheral neuropathy | Neuronal antigens |
| Atypical hemolytic uremic syndrome | Complement factor H |
| Thrombotic thrombocytopenia purpura | ADAMTS13 |

According to some embodiments, the autoimmune disease is triggered by an infectious agent. According to some embodiments, the disclosure provides a ceDNA as described herein comprising a nucleic acid sequence that encodes one or more antigens, or immunogenic peptides, for treating an autoimmune disease or disorder associated with or triggered by an infectious agent. Exemplary autoimmune diseases or disorders associated with or triggered by infectious agents are provided in Table 3.

TABLE 3

| Autoimmune Disease | Infectious Agent(s) |
|---|---|
| Allergic encephalitis | Measles virus |
| Autoimmune kidney disease | Streptococcal infections |
| Chagas disease | Trypanosoma cruzi |
| Chronic autoimmune hepatitis | Hepatitis C virus |
| Guillain-Barré syndrome | Campylobacter jejuni, Cytomegalovirus, Zika virus |
| Herpetic stromal keratitis | Herpes simplex virus |
| HTLV-associated myelopathy | Human T-cell leukemia virus |
| Lyme arthritis | Borrelia burgdorferi |
| Mixed cryoglobulinemia | Hepatitis C virus |
| Myocarditis | Coxsackie virus B3 |
| Pediatric autoimmune neuropsychiatric | Streptococcal infections |
| disorders | |
| Polyarteritis nodosa | Hepatitis B virus |
| Primary biliary cirrhosis | Escherichia coli |
| Reactive arthritis | Yersinia enterocolitica |
| Reiter's syndrome | Chlamydia trachomatis, Shigella species |
| Rheumatic fever | Streptococcus pyogenes |
| Rheumatic heart disease | Sreptococci |
| Rheumatoid arthritis | Normal gut flora |
| Scleroderma | Cytomegalovirus |
| Tourette syndrome | Streptococcal infections |
| Type 1 diabetes | Enterovirus, Rotavirus |
| Type 1 diabetes mellitus | Coxsackie virus B4 |

### Infectious Diseases

According to some embodiments, the disclosure provides a ceDNA as described herein comprising a nucleic acid sequence that encodes one or more antigens, or immunogenic peptides, for treating an infectious disease. According to some embodiments, the antigen is an antigen of a pathogen or infectious agent (where “pathogen” and “infectious agent” are used interchangeably herein), e.g., a viral pathogen, a bacterial pathogen, a fungal pathogen, or a parasitic pathogen.

According to some embodiments, the antigen, or immunogenic peptide, is a viral antigen or immunogenic peptide. According to some embodiments, the disclosure provides a ceDNA as described herein comprising a nucleic acid sequence that encodes one or more viral antigens, or immunogenic peptides.

Viral infections include adenovirus, coxsackievirus, hepatitis A virus, poliovirus, Epstein-Barr virus, herpes simplex type 1, herpes simplex type 2, human cytomegalovirus, human herpesvirus type 8, varicella-zoster virus, hepatitis B virus, hepatitis C viruses, human immunodeficiency virus (HIV), influenza virus, measles virus, mumps virus, parainfluenza virus, respiratory syncytial virus, papillomavirus, rabies virus, and Rubella virus. Other viral targets include Paramyxoviridae (e.g., pneumovirus, morbillivirus, metapneumovirus, respirovirus or rubulavirus), Adenoviridae (e.g., adenovirus), Arenaviridae (e.g., arenavirus such as lymphocytic choriomeningitis virus), Arteriviridae (e.g., porcine respiratory and reproductive syndrome virus or equine arteritis virus), Bunyaviridae (e.g., phlebovirus or hantavirus), Caliciviridae (e.g., Norwalk virus), Coronaviridae (e.g., coronavirus or torovirus), Filoviridae (e.g., Ebola-like viruses), Flaviviridae (e.g., hepacivirus or flavivirus), Herpesviridae (e.g., simplexvirus, varicellovirus, cytomegalovirus, roseolovirus, or lymphocryptovirus), Orthomyxoviridae (e.g., influenza virus or thogotovirus), Parvoviridae (e.g., parvovirus), Picomaviridae (e.g., enterovirus or hepatovirus), Poxviridae (e.g., orthopoxvirus, avipoxvirus, or leporipoxvirus), Retroviridae (e.g., lentivirus or spumavirus), Reoviridae (e.g., rotavirus), Rhabdoviridae (e.g., lyssavirus, novirhabdovirus, or vesiculovirus), and Togaviridae (e.g., alphavirus or rubivirus). Specific examples of these viruses include human respiratory coronavirus, influenza viruses A-C, hepatitis viruses A to G, and herpes simplex viruses 1-9.

Exemplary viral pathogens are shown below in Table 4.

TABLE 4

| | Examples, by | |
|---|---|---|
| | Family (Human | |
| Baltimore classification | Host) | Species/Pathology example |
| dsDNA viruses | Adenoviridae | Example Respiratory infection |
| dsDNA viruses | Polyomaviridae | Example - progressive multifocal |
| | | leukoencephalopathy |
| dsDNA viruses | Papiliomaviradae | Example - betapapilloma virus (warts, malignant |
| | | tumors) |
| dsDNA viruses | Poxviridae | Example - Molluscum contagiosum (skin lesions) |
| dsDNA viruses | Herpesvirales | Example - Varicellovirus (Chickenpox) |
| ssDNA viruses (+strand | Anelloviridae | Asymptomatic, may be associate with hepatitis, |
| or “sense”) DNA | | pulmonary disease, hematological disorders, |
| | | myopathy, lupus |
| ssDNA viruses (+strand | Parvoviridae | Example - Fifth disease |
| or “sense”) DNA | | |
| dsRNA | Reoviridae | Example - Colorado tick fever |
| viruses (e.g., Reoviruses) | | |
| (+)ssRNA viruses | Coronaviridae | Example - Pneumonia, gastroenteritis |
| (+strand or sense) RNA | | |
| (+)ssRNA viruses | Picornaviridae | Example - Myocarditis |
| (+strand or sense) RNA | | |
| (+)ssRNA viruses | Astoviridae | Example - infantile gastroenteritis |
| (+strand or sense) RNA | | |
| (+)ssRNA viruses | Caliciviridae | Example - Norovirus - gastroenteritis |
| (+strand or sense) RNA | | |
| (+)ssRNA viruses | Flaviviridae | Example - Dengue, Zika |
| (+strand or sense) RNA | | |
| (+)ssRNA viruses | Hepeviridae | Example - Hepatits |
| (+strand or sense) RNA | | |
| (+)ssRNA viruses | Togaviridae | Example - Rubella |
| (+strand or sense) RNA | | |
| (−)ssRNA viruses | Rhabdoviridae | Example - Rabies |
| (−strand or antisense) RNA | | |
| (−)ssRNA viruses | Filoviridae | Example - Ebola, Marburg |
| (−strand or antisense) RNA | | |
| (−)ssRNA viruses | Paramyxoviridae | Example - Mumps |
| (−strand or antisense) RNA | | |
| (−)ssRNA viruses | Pneumovirinae | Example - Respiratory tract infection |
| (−strand or antisense) RNA | | |
| (−)ssRNA viruses | Arenaviridae | Example - Enchephalitis, Hemorrhagic fever |
| (−strand or antisense) RNA | | |
| (−)ssRNA viruses | Bunyaviridae | Example - Hantavirus pulmonary |
| (−strand or antisense) RNA | | syndrome/hemorrhagic fever |
| (−)ssRNA viruses | Deltavirus | Example - hepatitis, cirrhosis |
| (−strand or antisense) RNA | | |
| (−)ssRNA viruses | Orthomyxoviridae | Example - Influenza A, Influenza B |
| (−strand or antisense) RNA | | |
| ssRNA-RT viruses | Retroviridae | Example - lentivirus - HIV |
| (+strand or sense) RNA with | | |
| DNA intermediate in life- | | |
| cycle | | |
| dsDNA-RT viruses | Hepadnaviridae | Example disease - hepatitis, cirrhosis, hepatocellular |
| | | carcinoma |

According to some embodiments, the disclosure provides a ceDNA as described herein comprising a nucleic acid sequence that encodes one or more antigens, or immunogenic peptides, for treating COVID-19. According to some embodiments, the nucleic acid encodes the SARS-CoV-2 spike protein.

The spike protein contains an S1 subunit that facilitates binding of the coronavirus to cell surface proteins. Accordingly, the S1 subunit of the spike protein controls which cells are infected by the coronavirus. The spike protein also contains a S2 subunit, which is a transmembrane subunit that facilitates viral and cellular membrane fusion.

The complete genome of severe acute respiratory syndrome coronavirus 2 isolate Wuhan-Hu-1 is set forth as GenBank Accession No. MN908947.3. The amino acid sequence of the wild type spike glycoprotein (S), is set forth below as SEQ ID NO: ______:

MFVFLVLLPLVSSQCVNLTTRTQLPPAYTNSFTRGVYYPDKVFRSS
VLHSTQDLFLPFFSNVTWFHAIHVSGTNGTKRFDNPVLPFNDGVY
FASTEKSNIIRGWIFGTTLDSKTQSLLIVNNATNVVIKVCEFQFC
NDPFLGVYYHKNNKSWMESEFRVYSSANNCTFEYVSQPFLMDLEG
KQGNFKNLREFVFKNIDGYFKIYSKHTPINLVRDLPQGFSALEPL
VDLPIGINITRFQTLLALHRSYLTPGDSSSGWTAGAAAYYVGYLQ
PRTFLLKYNENGTITDAVDCALDPLSETKCTLKSFTVEKGIYQTS
NFRVQPTESIVRFPNITNLCPFGEVFNATRFASVYAWNRKRISNC
VADYSVLYNSASFSTFKCYGVSPTKLNDLCFTNVYADSFVIRGDE
VRQIAPGQTGKIADYNYKLPDDFTGCVIAWNSNNLDSKVGGNYNY
LYRLFRKSNLKPFERDISTEIYQAGSTPCNGVEGENCYFPLQSYG
FQPTNGVGYQPYRVVVLSFELLHAPATVCGPKKSTNLVKNKCVNF
NFNGLTGTGVLTESNKKFLPFQQFGRDIADTTDAVRDPQTLEILD
ITPCSFGGVSVITPGTNTSNQVAVLYQDVNCTEVPVAIHADQLTP
TWRVYSTGSNVFQTRAGCLIGAEHVNNSYECDIPIGAGICASYQT
QTNSPRRARSVASQSIIAYTMSLGAENSVAYSNNSIAIPTNFTIS
VTTEILPVSMTKTSVDCTMYICGDSTECSNLLLQYGSFCTQLNRA
LTGIAVEQDKNTQEVFAQVKQIYKTPPIKDFGGFNFSQILPDPSK
PSKRSFIEDLLFNKVTLADAGFIKQYGDCLGDIAARDLICAQKFN
GLTVLPPLLTDEMIAQYTSALLAGTITSGWTFGAGAALQIPFAMQ
MAYRENGIGVTQNVLYENQKLIANQFNSAIGKIQDSLSSTASALG
KLQDVVNQNAQALNTLVKQLSSNFGAISSVLNDILSRLDKVEAEV
QIDRLITGRLQSLQTYVTQQLIRAAEIRASANLAATKMSECVLGQ
SKRVDFCGKGYHLMSFPQSAPHGVVFLHVTYVPAQEKNFTTAPAI
CHDGKAHFPREGVFVSNGTHWFVTQRNFYEPQIITTDNTFVSGNC
DVVIGIVNNTVYDPLQPELDSFKEELDKYFKNHTSPDVDLGDISG
INASVVNIQKEIDRLNEVAKNLNESLIDLQELGKYEQYIKWPWYI
WLGFIAGLIAIVMVTIMLCCMTSCCSCLKGCCSCGSCCKFDEDDS
EPVLKGVKLHYT

According to some embodiments, the antigen, or immunogenic peptide, is the stabilized prefusion SARS-CoV-2 spike protein (SARS-CoV-2 S (2P)).

According to some embodiments, the antigen, or immunogenic peptide, is a bacterial antigen or immunogenic peptide. According to some embodiments, the disclosure provides a ceDNA as described herein comprising a nucleic acid sequence that encodes one or more bacterial antigens, or immunogenic peptides.

Bacterial infections include, but are not limited to, Mycobacteria, Rickettsia, Mycoplasma, Neisseria meningitides, Neisseria gonorrheoeae, Legionella, Vibrio cholerae, Streptococci, Staphylococcus aureus, Staphylococcus epidermidis, Pseudomonas aeruginosa, Corynobacteria diphtheriae, Clostridium spp., enterotoxigenic Eschericia coli, Bacillus anthracis, Rickettsia, Bartonella henselae, Bartonella quintana, Coxiella burnetii, chlamydia, Mycobacterium leprae, Salmonella; shigella; Yersinia enterocolitica; Yersinia pseudotuberculosis; Legionella pneumophila; Mycobacterium tuberculosis; Listeria monocytogenes; Mycoplasma spp.; Pseudomonas fluorescens; Vibrio cholerae; Haemophilus influenzae; Bacillus anthracis; Treponema pallidum; Leptospira; Borrelia; Corynebacterium diphtheriae; Francisella; Brucella melitensis; Campylobacter jejuni; Enterobacter; Proteus mirabilis; Proteus; and Klebsiella pneumoniae.

Exemplary bacterial infections are shown in Table 5 below.

TABLE 5

| Phylum | By Genus | Species/Pathology Example |
|---|---|---|
| Proteobacteria | Erlichia | Erlichosis (Pain, malaise, fever gastrointestinal disorder, |
| | | confusion rash. Can be fatal) |
| Proteobacteria | Rickettsia | Two groups of diseases from different subspecies - Spotted |
| | | fever; Typhus |
| Proteobacteria | Brucella | Brucellosis. 4 known species cause disease in humans. Can be |
| | | sporadic or chronic. Fever, malaise, lesions |
| Proteobacteria | Bartonella | Multiple species. Pathophysiology includes endocarditis, |
| | | neuroretinitis, cat scratch disease, peliosis hepititus, general |
| | | bactermia angiomatosis, Carrion's disease, Trench fever |
| Proteobacteria | Bordetella | Multiple species. Adherence to ciliated ephilium drives |
| | | respiratory tract infections. B. pertussis = subspecies example |
| Proteobacteria | Neisseria | Includes N meningitidis (bacterial meningitis and septicemia) |
| | | and N gonorrheae (Gonorrhea) - colonize mucosal surfaces |
| Proteobacteria | Francisella | Examples include f. tularensis (tularemia), F novicida, |
| | | F philomiragia (septicemia) |
| Proteobacteria | Legionella | Examples include L pneumophila - legionnaires disease and |
| | | pontiac fever |
| Proteobacteria | Coxiella | Coxiella burnetii - Q Fever |
| Proteobacteria | Moraxella | Examples include M. catarrhalis (lower respiratory tract |
| | | infection) and Moraxella lacunata (blepharoconjunctivitis) |
| Proteobacteria | Pseudomonas | Multiple groups and subspecies therein. Examples include |
| | | P. aeruginosa - opportunistic infection in cystic fobrosis, burn and |
| | | immunocompromised patients. Colonizes skin, lungs, kidney, |
| | | urinary tract. Found on most medical equipment - develops |
| | | enduring biofilms |
| Proteobacteria | Vibrio | Multiple species - foodborne disease generally implicated with |
| | | gastroenteritis - example - V. cholerae. Can also cause |
| | | septicemia in open wounds |
| Proteobacteria | Plesimonas | Example P. Shigelloides - causes gastrointestinal disease |
| Proteobacteria | Aeromonas | Cornucopia of human disease including and not limited to |
| | | gastrointestinal disease, wound and soft tissue infection, blood |
| | | borne dyscrasias |
| Proteobacteria | Citrobacter | Examples: C. freundii, C. koseri, C. amalonaticus. Infect |
| | | urinary tract, can cause infant meningitis and sepsis |
| Proteobacteria | Enterobacter | Multiple species - Urinary and respiratory tract infections are |
| | | most common. Example E. aerogenes are a common source of |
| | | opportunistic and/or nosocomial infections |
| Proteobacteria | Escerichia | Common cause of gastrointestinal infections. Example E. coli |
| Proteobacteria | Klebsiella | Multiple species and sub-species. Cause a variety of |
| | | opporuntistic infections: Pneumonia, UTI, septicemia, |
| | | meningitis, diarrhea, soft tissue infection |
| Proteobacteria | Proteus | Urinary tract infections (including kidney) - P. vulgaris, |
| | | P. mirabilis, P. penneri |
| Proteobacteria | Providencia | Multiple species cause. Urinary tract infections. Example: |
| | | P. stuartii |
| Proteobacteria | Morganella | Single species with 2 subspecies. M. morganii. Cause |
| | | opportunistic infections (wound, UTI) |
| Proteobacteria | Salmonella | Two species S. bongori and S. enterica, along with multiple |
| | | subspecies. Typical non-typhoidal salmonella causes |
| | | gastrointestinal disease. In developing countries it also causes |
| | | blood infections. Typhiidal disease causes typhoid fever, |
| | | hypovolemic shock, septic shock |
| Proteobacteria | Serratia | Multiple species: Opportunisite infection that often develops |
| | | biofilms. Colonizes respiratory and urinary tract. Responsible |
| | | for 2% of nosocomial infections of blood, LRT, UT, surgical |
| | | wounds, skin and soft tissue infection. Example s. marcescens |
| Proteobacteria | Shigella | 4 species. Causes shigellosis (leading bacterial cause of |
| | | diarrhea). Example : S. dysenteriae |
| Proteobacteria | Yersinia | Multiple species and sub-species. Example: Y. pestis causes the |
| | | plague |
| Proteobacteria | Pasteurella | Multiple species. P. multocida species is the most frequent |
| | | example of human infection ( - symptoms include swelling, |
| | | cellulitis, wound drainage and arthritis) |
| Proteobacteria | Hemophilus | Multiple species. Example H influenzae (Hib) causes |
| | | penumonia, sepsis and bacterial meningitis in young children |
| Proteobacteria | Campylobacter | Multiple species. Cause campylobacteriosis (gastrointestinal |
| | | disease - inflammatory diarrhea/dysentary). Example |
| | | C. rectus |
| Proteobacteria | Heliobacter | Example H pylori. Causes gastritis and gastric ulcers |
| Frimicutes | Clostridia | Multiple Species. Examples: C. Difficile, C. botulinum, |
| | | C. tetani. Cause a variety of serious conditions, from colitis to |
| | | paralysis |
| Frimicutes | Mycoplasmas | Multiple species. Examples: M. genitalium, M pneumoniae. |
| | | Ureaplasma species. Associated with sexually transmited |
| | | disease, infertility and infant respiratory distress and brain |
| | | hemorrhage. P1 antigen is primary virulence factor, which is |
| | | also expressed on erthyrocytes. This can lead to autoantibody |
| | | agglutination |
| Frimicutes | Bacillus | One of the most diverse genus from a speciation perspective. |
| | | Examples B. anthracis (anthrax), B. cereus (food poisoning) |
| Frimicutes | Listeria | 15 identified species. Example: L. monocytogenes food |
| | | poisining. Less frequently seen disease manifestation = |
| | | listeriosis (sepsis and meningitis with a 20% fatality rate) |
| Frimicutes | Staphylococcus | Multiple species and sub. Example: S. aureus. Disease can |
| | | range form folliculitis to necrotizing pneumonia and |
| | | endocarditis. Commonly drug resistant (MRSA) |
| Frimicutes | Enterococcus | Multiple species. Cause UTI, Bacteremia, endocarditis, |
| | | diverticulitis, meningitis, prostatitis. Example: E. faecium. |
| | | Increasingly drug resistant (VRE) |
| Frimicutes | Lactobacillus | Often considered beneficial, but can cause septicemiua, |
| | | endocarditis, rheumatic vascular disease and dental |
| | | caries(particularly in immuno-compromised patients). Example |
| | | L. rhamnosus |
| Frimicutes | Streptococcus | Multiple species. Cause strep throat, pink eye, meningitis, |
| | | bacterial pneumonia, sepsis, endocarditis, erysipelas, necrotizing |
| | | fasciitis. Examples: S. pyogenes, S. pneumoniae, S. sanguinis |
| Actinobacteria | Nocardia | Multiple species. Low virulence and generally infect the |
| | | immunocompromised only. Cause penumonia, endocarditis, |
| | | encephalitis, cellulitis. Example N. asteroids |
| Actinobacteria | Mycobacterium | Multiple species. Cause tuberculosis, leprosy. Examples |
| | | M. tuberculosis, M. leprae |
| Actinobacteria | Corynebacterium | Multiple species. Cause diptheria, colonizes prosthetics, and |
| | | can cause skin infections, endocarditis, pheumonitis. |
| | | Nosocomial |
| Actinobacteria | Actinomyces | Multiple species. Cause periodontal abscesses, |
| | | lympadenopathy, thoracic disease and abdominal abscess. |
| | | Example: Aggregatibacter actinomycetemcomitans |
| Chlamydia | Chlamydia | 4 species. Most common bacterial STD and can cause blindness. |
| | | Example Chlamydia trachomatis. |
| Spirochetes | Borrelia | 52 species. Cause Lyme disease and relapsing fever (severe |
| | | bacterimia). Example: Borrelia burgdorferi |
| Spirochetes | Leptospira | Multiple species (13 cause disease in humans). Cause |
| | | leptospirosis - symtpoms range from hedaches and fatigue to |
| | | meningitis, kidney failure and pulmonary hemorrhage. |
| Spirochetes | Treponema | Multiple species. Cuases syphilis Example: Treponema |
| | | pallidum. |
| Bacteroids | Bacteroides | Multiple species. Colonize the gut and cause infections |
| | | associated with surgery, appendicitis etc. Example: B. fragilis |

According to some embodiments, the antigen, or immunogenic peptide, is a fungal antigen or immunogenic peptide. According to some embodiments, the disclosure provides a ceDNA as described herein comprising a nucleic acid sequence that encodes one or more fungal antigens, or immunogenic peptides.

Exemplary fungal infections are shown in Table 6 below.

TABLE 6

| Fungal | | |
|---|---|---|
| Group | Pathogen Species | Pathology example |
| Yeast | Candida albicans | Oral thrush, onychomycosis |
| Yeast | Candida glabrata | Vaginitis, esophageal candidisis |
| Yeast | Candida krusei | Invasive candidiasis, neutropenia |
| Yeast | Candida | Catheter and central line infections, biofims, UTI, endocarditis, |
| | parapsilosis | meningitis |
| Yeast | Candida tropicalis | Candidemia, oral thrush |
| Yeast | Rhodotorula | Sepsis, catheter infections, fungemia, meningitis, peritonitis |
| | mucilaginosa | |
| Yeast | Sporothrix schenckii | Sporotrichosis (lympocutaneous tissue, skin ulcerations) |
| | complex | |
| Yeast | Cryptococcus | Cryptococcal meningitis, pulmonary infection, osteomyelitis |
| | neoformans | |
| Yeast | Cryptococcus gattii | Cryptococcal meningitis, pulmonary infection, osteomyelitis (more |
| | | common in immunocompromised patients thant C. neoformans, |
| | | which is more prevelant in immunocompetent patients) |
| Molds | Alternaria alternata | Allergic fungal rhinosinusitis, Keratitis, peritonitis, allergic |
| | | respiratory disease |
| Molds | Apophysomyces | Cutaneous and subcutaneous infection |
| | variabilis | |
| Molds | Aspergillus | Chronic pulmonary aspergillosis, Asthma exacerbation with fingal |
| | fumigatus | sensitization, chronic invasive sinusitis, invasive and disseminated |
| | | aspergillosis |
| Molds | Aspergillus flavus | Chronic cavity pulmonary aspergillosis, Cutaneous and wound |
| | | infection, endocarditis, pericarditis and CNS infection, UTI |
| Molds | Aspergillus niger | Otomycosis, SAFS, Allergic bronchopulmonary aspergillosis, |
| | | invasive pulmonary aspergillosis, disseminated aspergillosis |
| Molds | Aspergillus terreus | ABPA, aspergillus bronchitis, invasive aspergillosis, disseminated |
| | | aspergillosis |
| Molds | Cladosphialophora | Chromoblastomycosis, mycetoma, phaehyphomycosis |
| | spp. | |
| Molds | Exserohilum | Skin, corneal infection, invasive infection of sinus heart and lung |
| Molds | Fonsecaea pedrosoi | Chromoblastomycosis |
| Molds | Fusarium | Keratitis, Onychomycosis, endophthalmitis, skin infection, |
| | oxysporum | sinusitis, disseminated infection |
| Molds | Fusarium solani | Keratitis, Onychomycosis, endophthalmitis, skin infection, |
| | | sinusitis, disseminated infection |
| Molds | Lichtheimia | cutaneous, pulmonary, rhinocerebral CNS and disseminated |
| | corymbifera | infection (rare) |
| Molds | Lichtheimia ramosa | cutaneous, pulmonary, rhinocerebral CNS and disseminated |
| | | infection (rare) |
| Molds | Rhizopus | Mucormycosis |
| | microsporus | |
| Molds | Stachybotrys | idiopathic pulmonary hemorrhage (infants) |
| Molds | Trichophyton | Tiñea pedis, corporis, cruris, onychomycosis and occasionally |
| | interdigitale | Tinea capitis |
| Molds | Trichophyton | Tinea of the groin, glabrous skin, feet, hands, and the nails. Tinea |
| | rubrum | cruris, tinea corporis, tinea pedis, tinea manuum, and |
| | | onychomycosis |
| Dimorphic | Histoplasma | Pneumonia, multi-organ failure |
| Fungi | capsulatum | |
| Dimorphic | Pneumocystis | Pneumonia (mostly in immune supressed patients) |
| Fungi | jirovecii | |
| Dimorphic | Paracoccidioides | Disseminated systemic infection |
| Fungi | brasiliensis | |
| Dimorphic | Penicillium | Disseminated infection with prominent skin lesions |
| Fungi | marneffei | (immunocompromised patients) |
| Dimorphic | Blastomyces | Skin infections, rearely disseminated |
| Fungi | | |
| Dimorphic | Coccidioides | Meningitis, disseminated disease, lung nodule, chronic cavitary |
| Fungi | | pulmonary coccidioidmycosis |

According to some embodiments, the antigen, or immunogenic peptide, is a parasitic antigen or immunogenic peptide. According to some embodiments, the disclosure provides a ceDNA as described herein comprising a nucleic acid sequence that encodes one or more fungal antigens, or immunogenic peptides.

Exemplary parasitic infections are shown in Table 7 below.

TABLE 7

| Parasitic | | |
|---|---|---|
| Group | Pathogen Species | Disease/Pathology Example |
| Amoeba/ | Acanthamoeba | Acanthamoeba Infection |
| Protozoa | | |
| Amoeba/ | Acanthamoeba | Acanthamoeba Keratitis Infection |
| Protozoa | | |
| Amoeba/ | Trypanosoma brucei | African Sleeping Sickness (African trypanosomiasis) |
| Protozoa | | |
| Amoeba/ | Entamoeba histolytica | Amebiasis (Entamoeba histolytica Infection) |
| Protozoa | | |
| Amoeba/ | Trypanosoma cruzi | American Trypanosomiasis (Chagas Disease) |
| Protozoa | | |
| Amoeba/ | Balantidium coli | Balantidiasis (Balantidium Infection) |
| Protozoa | | |
| Amoeba/ | Balamuthia mandrillaris | Balamuthia (Granulomatous Amebic Encephalitis (GAE)) |
| Protozoa | | |
| Amoeba/ | Cryptosporidium | Cryptosporidiosis (Cryptosporidium Infection) |
| Protozoa | | |
| Amoeba/ | Cyclospora | Cyclosporiasis (Cyclospora Infection) |
| Protozoa | | |
| Amoeba/ | Taenia solium | Cysticercosis (Neurocysticercosis) |
| Protozoa | | |
| Amoeba/ | Cystoisospora belli | Cystoisospora Infection (Cystoisosporiasis) formerly Isospora |
| Protozoa | | Infection |
| Amoeba/ | Dientamoeba fragilis | Dientamoeba fragilis Infection |
| Protozoa | | |
| Amoeba/ | Entamoeba histolytica | Entamoeba histolytica Infection (Amebiasis) |
| Protozoa | | |
| Amoeba/ | Giardia | Giardiasis (Giardia Infection) |
| Protozoa | intestinalis, Giardia | |
| | lamblia, or Giardia | |
| | duodenalis | |
| Amoeba/ | Leishmania | Kala-azar (Leishmaniasis, Leishmania Infection) |
| Protozoa | | |
| Amoeba/ | Acanthamoeba | Keratitis (Acanthamoeba Infection) |
| Protozoa | | |
| Amoeba/ | Multiple species. | Malaria (Plasmodium Infection) |
| Protozoa | Examples: P. vivax and | |
| | P. ovale | |
| Amoeba/ | Naegleria fowleri | Naegleria Infection |
| Protozoa | “brain-eating amoeba” | |
| Amoeba/ | Sappinia ameoba | Sappinia (amebic encephalitis) |
| Protozoa | | |
| Amoeba/ | Sarcocystis | Sarcocystosis (Sarcocystosis Infection) |
| Protozoa | | |
| Amoeba/ | Toxoplasma gondii | Toxoplasmosis (Toxoplasma Infection) - protazoa |
| Protozoa | | |
| Amoeba/ | Trichomonas vaginalis | Trichomoniasis (Trichomonas Infection) - protozoa |
| Protozoa | | |
| Amoeba/ | Trypanosoma brucei | Trypanosomiasis, African (African Sleeping Sickness, Sleeping |
| Protozoa | | Sickness) - protozoa |
| Amoeba/ | Babesia (various | Babesiosis (Babesia Infection) - similar lifecycle to malaria, |
| Protozoa | species) | taken up by RBCs |
| Arthropod | Cimex lectulariusand C. | Bed Bugs |
| | hemipterus | |
| Arthropod | Pediculus humanus | Body Lice Infestation (Pediculosis) |
| | humanus | |
| Arthropod | Phthirus pubis | Crabs (Pubic Lice) |
| Arthropod | Pediculus humanus | Head Lice Infestation (Pediculosis) |
| | capitis | |
| Arthropod | Sarcoptes | Mite Infestation (Scabies) |
| | scabiei var. hominis | |
| Arthropod | infection of a fly larva. | Myiasis |
| | Examples: Dermatobia | |
| | hominis | |
| Other | group of obligate | Microsporidiosis (Microsporidia Infection) |
| | intracellular parasitic | |
| | fungi. Examples: M. | |
| | africanum, Nosema | |
| | ocularum | |
| Other | Pneumocystis jirovecii | Pneumocystis jirovecii Pneumonia |
| | (considered a parasitic | |
| | fungi) | |
| Protist | Blastocystis hominis | Blastocystis hominis Infection |
| Worm | Echinococcus | Alveolar Echinococcosis (Echinococcosis, Hydatid Disease) |
| | granulosus | |
| Worm | Hookworm (various | Ancylostomiasis (Hookworm) |
| | species) | |
| Worm | Angiostrongylus | Angiostrongyliasis (Angiostrongylus Infection) |
| | (various species) | |
| | nematode | |
| Worm | anisakid nematodes | Anisakiasis (Anisakis Infection, Pseudoterranova Infection) |
| Worm | Ascaris lumbricoides | Ascariasis (Ascaris Infection, Intestinal Roundworms) |
| Worm | B. procyonis | Baylisascariasis (Baylisascaris Infection, Raccoon Roundworm) |
| Worm | S. mansoni | Bilharzia (Schistosomiasis) |
| Worm | Capillaria | Capillariasis (Capillaria Infection) |
| | hepatica and Capillaria | |
| | philippinensis | |
| Worm | Various species, | Cercarial Dermatitis (Swimmer's Itch) |
| | example: | |
| | Austrobilharzia | |
| | variglandis | |
| Worm | Clonorchis liver fluke | Clonorchiasis (Clonorchis Infection) |
| Worm | Diphyllobothrium | Diphyllobothriasis (Diphyllobothrium Infection) |
| | latum (tapeworm) | |
| Worm | Dipylidiu tapeworm | Dipylidium caninum Infection (dog or cat tapeworm infection) |
| Worm | Dirofilaria roundworms | Dirofilariasis (Dirofilaria Infection) |
| Worm | Guinea worm | Dracunculiasis (Guinea Worm Disease) |
| Worm | Echinococcus | Echinococcosis (Cystic, Alveolar Hydatid Disease) |
| | granulosus & | |
| | Echinococcus | |
| | multilocularis | |
| Worm | Wuchereria | Elephantiasis (Filariasis, Lymphatic Filariasis) |
| | bancrofti, Brugia malayi | |
| | and Brugia timori | |
| Worm | Enterobius vermicularis | Enterobiasis (Pinworm Infection) |
| Worm | Fasciola hepatica | Fascioliasis (Fasciola(liver fluke) Infection) |
| Worm | Fasciolopsis buski | Fasciolopsiasis (Fasciolopsis Infection) |
| Worm | Gnathostoma: several | Gnathostomiasis (Gnathostoma Infection) |
| | species of parasitic | |
| | nematodes | |
| Worm | trematode Heterophyes | Heterophyiasis (Heterophyes Infection) |
| | heterophyes | |
| Worm | Multiple species. | Hookworm Infection, Human |
| | Example: L: Filariform | |
| Worm | Multiple species. | Hookworm Infection, Zoonotic (Ancylostomiasis, Cutaneous |
| | Example: Ancylostoma | Larva Migrans [CLM]) |
| | brazilense, A. caninum, | |
| | A. ceylanicum | |
| Worm | H. nana (dwarf | Hymenolepiasis (Hymenolepis Infection) |
| | tapeworm) | |
| Worm | Various species, | Intestinal Roundworms (Ascariasis, Ascaris Infection) |
| | example: A. | |
| | lumbricoides | |
| Worm | Loa loa: parasitic | Loiasis (Loa loa Infection) |
| | worm | |
| Worm | Taenia solium | Neurocysticercosis (Cysticercosis) |
| Worm | Toxocara canis and | Ocular Larva Migrans (Toxocariasis, Toxocara Infection, |
| | Toxocara cati | Visceral Larva Migrans) |
| Worm | Onchocerca volvulus | Onchocerciasis (River Blindness) |
| Worm | Opisthorchis (liver | Opisthorchiasis (Opisthorchis Infection) |
| | fluke). Example: O. | |
| | felineus | |
| Worm | Paragonimus(lung | Paragonimiasis (Paragonimus Infection) |
| | fluke) | |
| Worm | anisakid nematodes | Pseudoterranova Infection (Anisakiasis, Anisakis Infection) |
| Worm | Strongyloides | Strongyloidiasis (Strongyloides Infection) |
| | nematodes. Example: S. | |
| | stercoralis | |
| Worm | Taenia saginata | Taeniasis (Taenia Infection, Tapeworm Infection) |
| Worm | Trichinella | Trichinellosis (Trichinosis) |
| Worm | T. trichiura | Trichuriasis (Whipworm Infection, Trichuris Infection) |

Other diseases and disorders are contemplated for treatment by the ceDNA vectors of the present disclosure. Examples include, but are not limited to, cardiovascular diseases and immune diseases.

It is well within the abilities of one of skill in the art to take a known and/or publically available protein sequence of e.g., an antigen, or immunogenic peptide, and reverse engineer a cDNA sequence to encode such a protein.

### Multipeptide Vaccines

According to some embodiments, the ceDNA vectors described herein are comprised in a ceDNA vector composition comprising multiple antigens or immunogenic peptides. For example, in formulating a multipeptide vaccine for treating cancer, it is not only important to identify and characterize tumor-associated antigens expressed on the cancer of interest, but also the combinations of different epitopes from the tumor-associated antigens that increase the likelihood of a response to more than one epitope for the patient. To counter the tumor's ability to evade therapies directed against it, the present disclosure utilizes a variety of specific peptides in the vaccine.

According to some embodiments, more than one epitope from the same protein can be used in the multipeptide vaccine.

IV. ceDNA Vector for Use in Production of Antigens

Embodiments of the disclosure are based on methods and compositions comprising close ended linear duplexed (ceDNA) vectors that can express antigens, or immunogenic peptides. As described herein, antigens, or immunogenic peptides, may be selected from a variety of pathogens, including, e.g., bacterial, viral, fungal and parasitic infectious agents, or cancer or cancer-associated antigens, or the like. Still other targets may include an autoimmune condition such as rheumatoid arthritis (RA) or multiple sclerosis (MS).

According to some embodiments, the transgene is a nucleic acid sequence encoding an antigen or immunogenic peptide. The ceDNA vector is preferably duplex, e.g., self-complementary, over at least a portion of the molecule, such as the expression cassette (e.g., ceDNA is not a double stranded circular molecule). The ceDNA vector has covalently closed ends, and thus is resistant to exonuclease digestion (e.g., exonuclease I or exonuclease III), e.g., for over an hour at 37° C.

In general, a ceDNA vector for expression of antigens, or immunogenic peptides, as disclosed herein, comprises in the 5′ to 3′ direction: a first adeno-associated virus (AAV) inverted terminal repeat (ITR), a nucleic acid sequence of interest (for example an expression cassette as described herein) and a second AAV ITR. The ITR sequences selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization.

Encompassed herein are methods and compositions comprising the ceDNA vector for production of antigens, or immunogenic peptides, which may further include a delivery system, such as but not limited to, a liposome nanoparticle delivery system. Non-limiting exemplary liposome nanoparticle systems encompassed for use are disclosed herein. According to some aspects, the disclosure provides for a lipid nanoparticle comprising ceDNA and an ionizable lipid. For example, a lipid nanoparticle formulation that is made and loaded with a ceDNA vector obtained by the process is disclosed in International Application PCT/US2018/050042, filed on Sep. 7, 2018, which is incorporated herein.

The ceDNA vectors as disclosed herein have no packaging constraints imposed by the limiting space within the viral capsid. ceDNA vectors represent a viable eukaryotically-produced alternative to prokaryote-produced plasmid DNA vectors, as opposed to encapsulated AAV genomes. This permits the insertion of control elements, e.g., regulatory switches as disclosed herein, large transgenes, multiple transgenes etc.

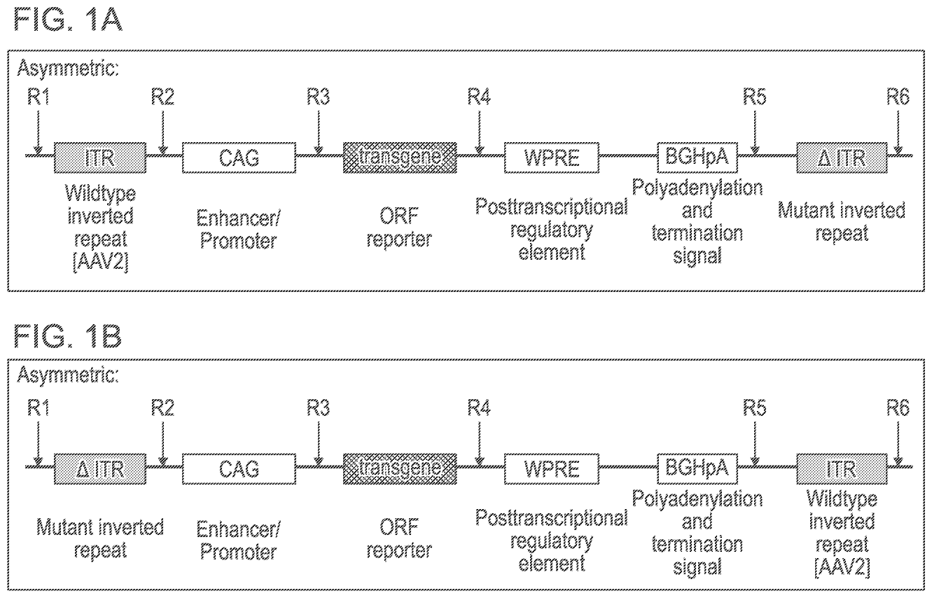

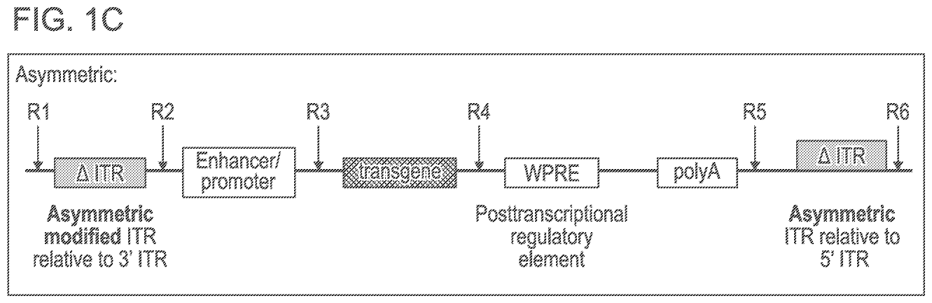

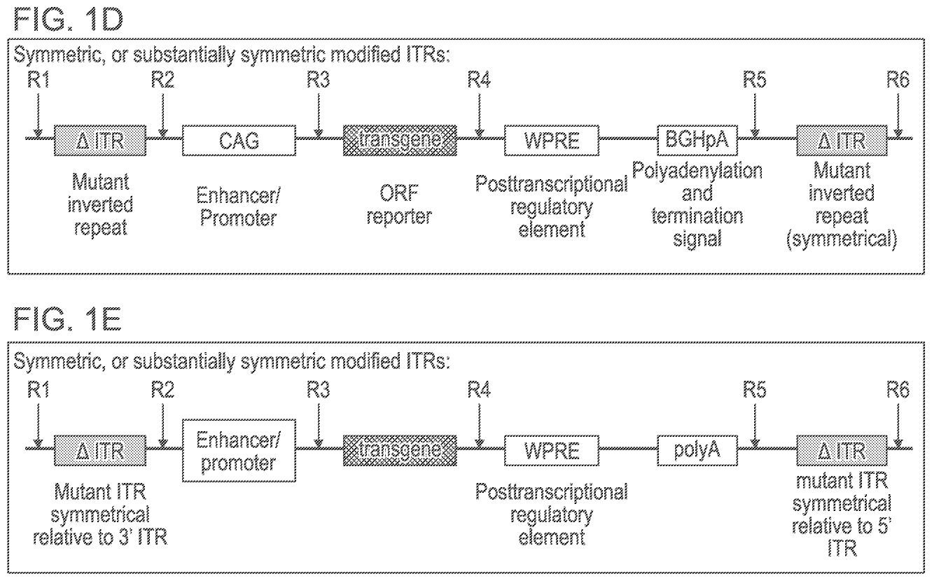

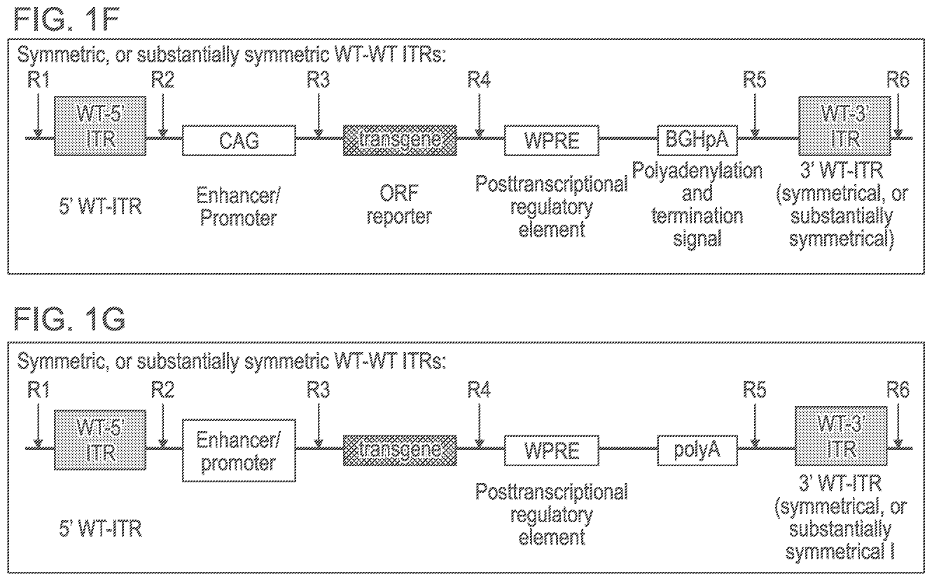

FIG. 1A-1E show schematics of non-limiting, exemplary ceDNA vectors for expression of antigens, or immunogenic peptides, or the corresponding sequence of ceDNA plasmids. ceDNA vectors for expression of antigens, or immunogenic peptides, are capsid-free and can be obtained from a plasmid encoding in this order: a first ITR, an expression cassette comprising a transgene and a second ITR. The expression cassette may include one or more regulatory sequences that allows and/or controls the expression of the transgene, e.g., where the expression cassette can comprise one or more of, in this order: an enhancer/promoter, an ORF reporter (transgene), a post-transcription regulatory element (e.g., WPRE), and a polyadenylation and termination signal (e.g., BGH polyA).

The expression cassette can also comprise an internal ribosome entry site (IRES) and/or a 2A element. The cis-regulatory elements include, but are not limited to, a promoter, a riboswitch, an insulator, a mir-regulatable element, a post-transcriptional regulatory element, a tissue- and cell type-specific promoter and an enhancer. According to some embodiments the ITR can act as the promoter for the transgene. According to some embodiments, the ceDNA vector comprises additional components to regulate expression of the transgene, for example, a regulatory switch, for controlling and regulating the expression of the antigens, or immunogenic peptides, and can include if desired, a regulatory switch which is a kill switch to enable controlled cell death of a cell comprising a ceDNA vector.

The expression cassette can comprise more than 4000 nucleotides, 5000 nucleotides, 10,000 nucleotides or 20,000 nucleotides, or 30,000 nucleotides, or 40,000 nucleotides or 50,000 nucleotides, or any range between about 4000-10,000 nucleotides or 10,000-50,000 nucleotides, or more than 50,000 nucleotides. According to some embodiments, the expression cassette can comprise a transgene in the range of 500 to 50,000 nucleotides in length. According to some embodiments, the expression cassette can comprise a transgene in the range of 500 to 75,000 nucleotides in length. According to some embodiments, the expression cassette can comprise a transgene which is in the range of 500 to 10,000 nucleotides in length. According to some embodiments, the expression cassette can comprise a transgene which is in the range of 1000 to 10,000 nucleotides in length. According to some embodiments, the expression cassette can comprise a transgene which is in the range of 500 to 5,000 nucleotides in length. The ceDNA vectors do not have the size limitations of encapsidated AAV vectors, thus enable delivery of a large-size expression cassette to provide efficient transgene expression. According to some embodiments, the ceDNA vector is devoid of prokaryote-specific methylation.

Sequences provided in the expression cassette, expression construct of a ceDNA vector for expression of antigens, or immunogenic peptides, described herein can be codon optimized for the target host cell. As used herein, the term “codon optimized” or “codon optimization” refers to the process of modifying a nucleic acid sequence for enhanced expression in the cells of the vertebrate of interest, e.g., mouse or human, by replacing at least one, more than one, or a significant number of codons of the native sequence (e.g., a prokaryotic sequence) with codons that are more frequently or most frequently used in the genes of that vertebrate. Various species exhibit particular bias for certain codons of a particular amino acid. Typically, codon optimization does not alter the amino acid sequence of the original translated protein. Optimized codons can be determined using e.g., Aptagen's GENE FORGE® codon optimization and custom gene synthesis platform (Aptagen, Inc., 2190 Fox Mill Rd. Suite 300, Herndon, Va. 20171) or another publicly available database. According to some embodiments, the nucleic acid is optimized for human expression.

A transgene expressed by the ceDNA vector for expression of antigens, or immunogenic peptides, as disclosed herein encodes antigens, or immunogenic peptides. There are many structural features of ceDNA vectors that differ from plasmid-based expression vectors. ceDNA vectors may possess one or more of the following features: the lack of original (i.e., not inserted) bacterial DNA, the lack of a prokaryotic origin of replication, being self-containing, i.e., they do not require any sequences other than the two ITRs, including the Rep binding and terminal resolution sites (RBS and TRS), and an exogenous sequence between the ITRs, the presence of ITR sequences that form hairpins, and the absence of bacterial-type DNA methylation or indeed any other methylation considered abnormal by a mammalian host. In general, it is preferred for the present vectors not to contain any prokaryotic DNA but it is contemplated that some prokaryotic DNA may be inserted as an exogenous sequence, as a non-limiting example in a promoter or enhancer region. Another important feature distinguishing ceDNA vectors from plasmid expression vectors is that ceDNA vectors are single-strand linear DNA having closed ends, while plasmids are always double-strand DNA.

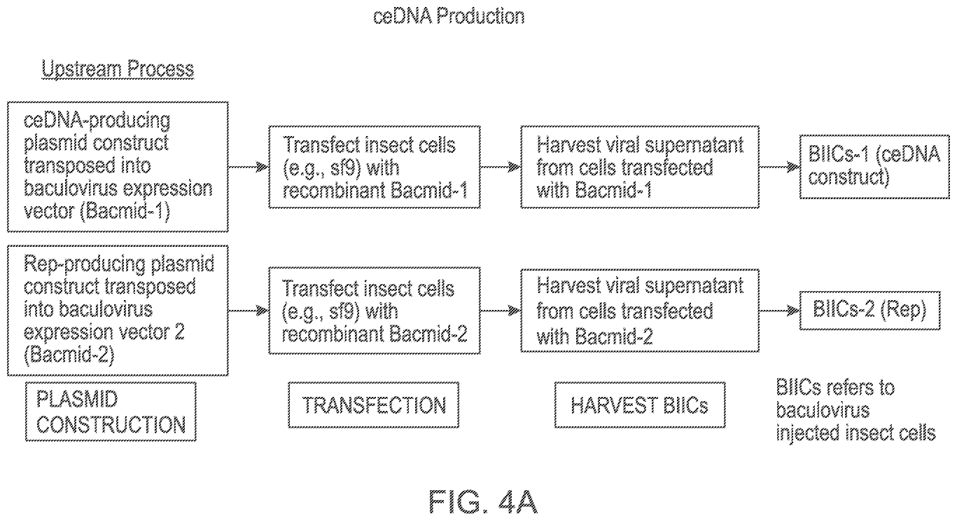

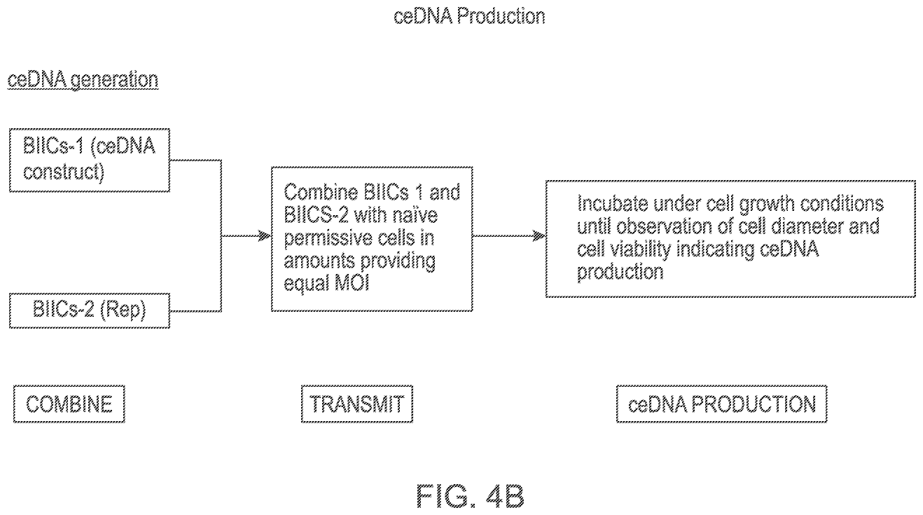

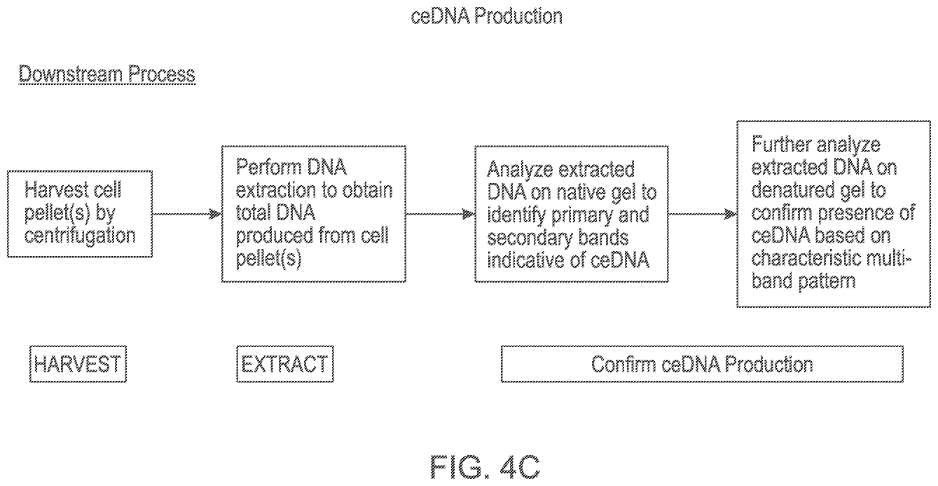

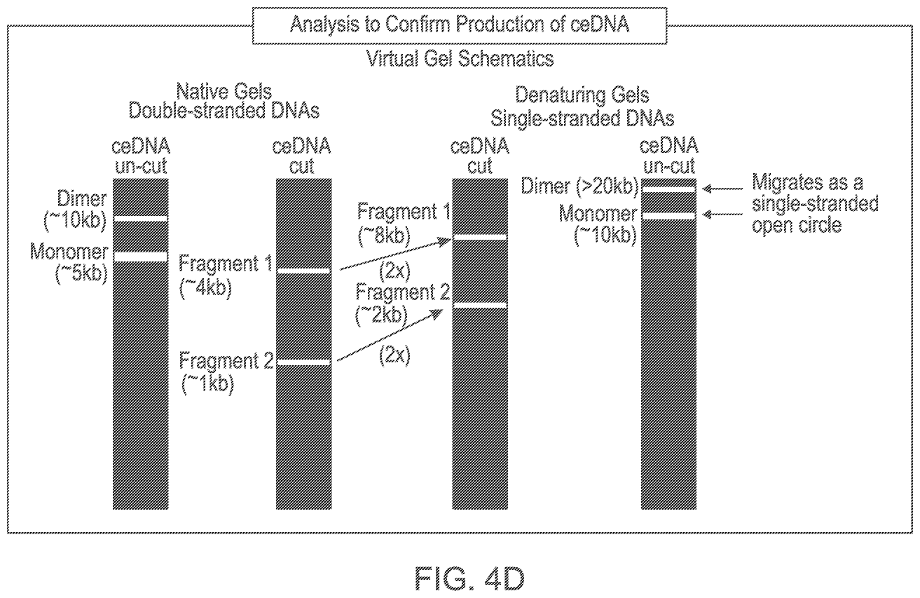

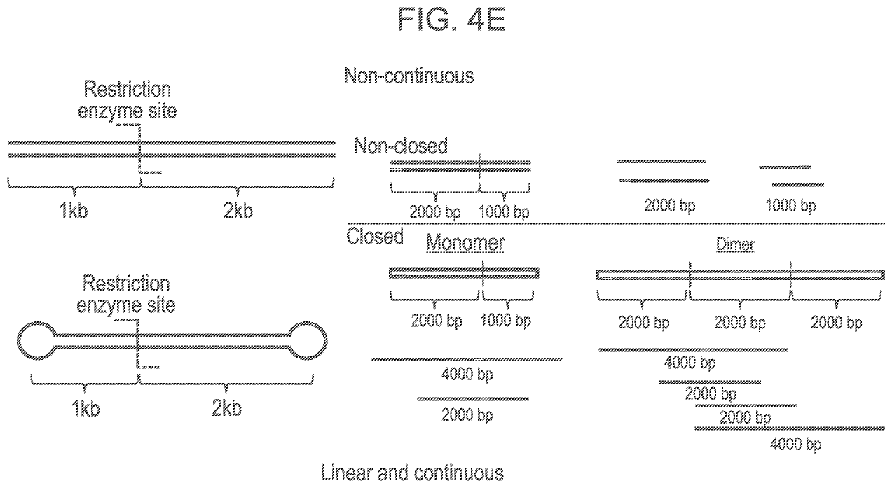

ceDNA vectors for expression of antigens, or immunogenic peptides, produced by the methods provided herein preferably have a linear and continuous structure rather than a non-continuous structure, as determined by restriction enzyme digestion assay (FIG. 4D). The linear and continuous structure is believed to be more stable from attack by cellular endonucleases, as well as less likely to be recombined and cause mutagenesis. Thus, a ceDNA vector in the linear and continuous structure is a preferred embodiment. The continuous, linear, single strand intramolecular duplex ceDNA vector can have covalently bound terminal ends, without sequences encoding AAV capsid proteins. These ceDNA vectors are structurally distinct from plasmids (including ceDNA plasmids described herein), which are circular duplex nucleic acid molecules of bacterial origin. The complimentary strands of plasmids may be separated following denaturation to produce two nucleic acid molecules, whereas in contrast, ceDNA vectors, while having complimentary strands, are a single DNA molecule and therefore even if denatured, remain a single molecule. According to some embodiments, ceDNA vectors as described herein can be produced without DNA base methylation of prokaryotic type, unlike plasmids. Therefore, the ceDNA vectors and ceDNA-plasmids are different both in term of structure (in particular, linear versus circular) and also in view of the methods used for producing and purifying these different objects (see below), and also in view of their DNA methylation which is of prokaryotic type for ceDNA-plasmids and of eukaryotic type for the ceDNA vector.

There are several advantages of using a ceDNA vector for expression of antigens, or immunogenic peptides, as described herein over plasmid-based expression vectors, such advantages include, but are not limited to: 1) plasmids contain bacterial DNA sequences and are subjected to prokaryotic-specific methylation, e.g., 6-methyl adenosine and 5-methyl cytosine methylation, whereas capsid-free AAV vector sequences are of eukaryotic origin and do not undergo prokaryotic-specific methylation; as a result, capsid-free AAV vectors are less likely to induce inflammatory and immune responses compared to plasmids; 2) while plasmids require the presence of a resistance gene during the production process, ceDNA vectors do not; 3) while a circular plasmid is not delivered to the nucleus upon introduction into a cell and requires overloading to bypass degradation by cellular nucleases, ceDNA vectors contain viral cis-elements, i.e., ITRs, that confer resistance to nucleases and can be designed to be targeted and delivered to the nucleus. It is hypothesized that the minimal defining elements indispensable for ITR function are a Rep-binding site (RBS; 5′-GCGCGCTCGCTCGCTC-3′ (SEQ ID NO: ______) for AAV2) and a terminal resolution site (TRS; 5′-AGTTGG-3′ (SEQ ID NO: ______) for AAV2) plus a variable palindromic sequence allowing for hairpin formation; and 4) ceDNA vectors do not have the over-representation of CpG dinucleotides often found in prokaryote-derived plasmids that reportedly binds a member of the Toll-like family of receptors, eliciting a T cell-mediated immune response. In contrast, transductions with capsid-free AAV vectors disclosed herein can efficiently target cell and tissue-types that are difficult to transduce with conventional AAV virions using various delivery reagent.

### Inverted Terminal Repeats (ITRs)

As disclosed herein, ceDNA vectors for expression of antigens, or immunogenic peptides, contain a transgene or nucleic acid sequence positioned between two inverted terminal repeat (ITR) sequences, where the ITR sequences can be an asymmetrical ITR pair or a symmetrical- or substantially symmetrical ITR pair, as these terms are defined herein. A ceDNA vector as disclosed herein can comprise ITR sequences that are selected from any of: (i) at least one WT ITR and at least one modified AAV inverted terminal repeat (mod-ITR) (e.g., asymmetric modified ITRs); (ii) two modified ITRs where the mod-ITR pair have a different three-dimensional spatial organization with respect to each other (e.g., asymmetric modified ITRs), or (iii) symmetrical or substantially symmetrical WT-WT ITR pair, where each WT-ITR has the same three-dimensional spatial organization, or (iv) symmetrical or substantially symmetrical modified ITR pair, where each mod-ITR has the same three-dimensional spatial organization, where the methods of the present disclosure may further include a delivery system, such as but not limited to a liposome nanoparticle delivery system.

According to some embodiments, the ITR sequence can be from viruses of the Parvoviridae family, which includes two subfamilies: Parvovirinae, which infect vertebrates, and Densovirinae, which infect insects. The subfamily Parvovirinae (referred to as the parvoviruses) includes the genus Dependovirus, the members of which, under most conditions, require coinfection with a helper virus such as adenovirus or herpes virus for productive infection. The genus Dependovirus includes adeno-associated virus (AAV), which normally infects humans (e.g., serotypes 2, 3A, 3B, 5, and 6) or primates (e.g., serotypes 1 and 4), and related viruses that infect other warm-blooded animals (e.g., bovine, canine, equine, and ovine adeno-associated viruses). The parvoviruses and other members of the Parvoviridae family are generally described in Kenneth I. Berns, “Parvoviridae: The Viruses and Their Replication,” Chapter 69 in FIELDS VIROLOGY (3d Ed. 1996).

While ITRs exemplified in the specification and Examples herein are AAV2 WT-ITRs, one of ordinary skill in the art is aware that one can as stated above use ITRs from any known parvovirus, for example a dependovirus such as AAV (e.g., AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261), chimeric ITRs, or ITRs from any synthetic AAV. According to some embodiments, the AAV can infect warm-blooded animals, e.g., avian (AAAV), bovine (BAAV), canine, equine, and ovine adeno-associated viruses. According to some embodiments the ITR is from B19 parvovirus (GenBank Accession No: NC 000883), Minute Virus from Mouse (MVM) (GenBank Accession No. NC 001510); goose parvovirus (GenBank Accession No. NC 001701); snake parvovirus 1 (GenBank Accession No. NC 006148). According to some embodiments, the 5′ WT-ITR can be from one serotype and the 3′ WT-ITR from a different serotype, as discussed herein.

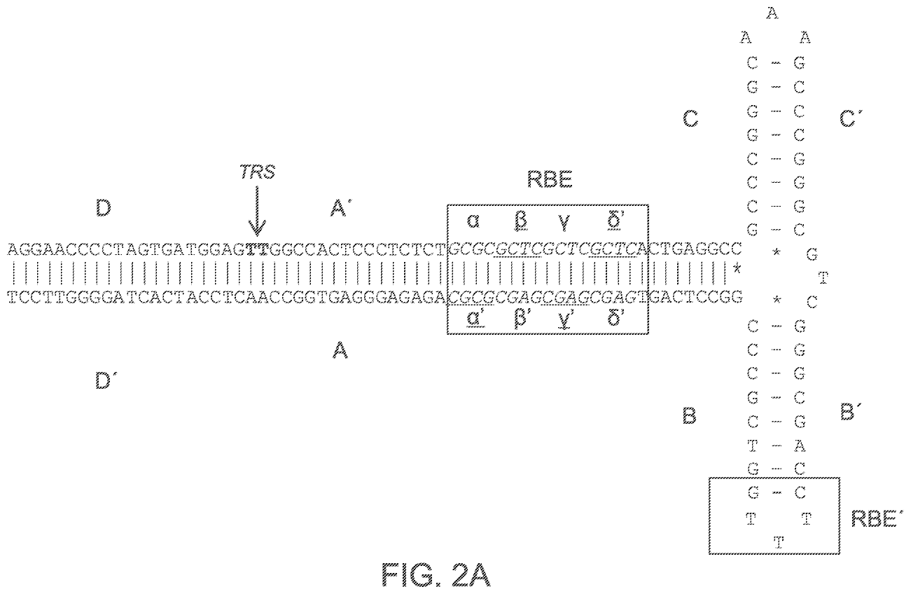

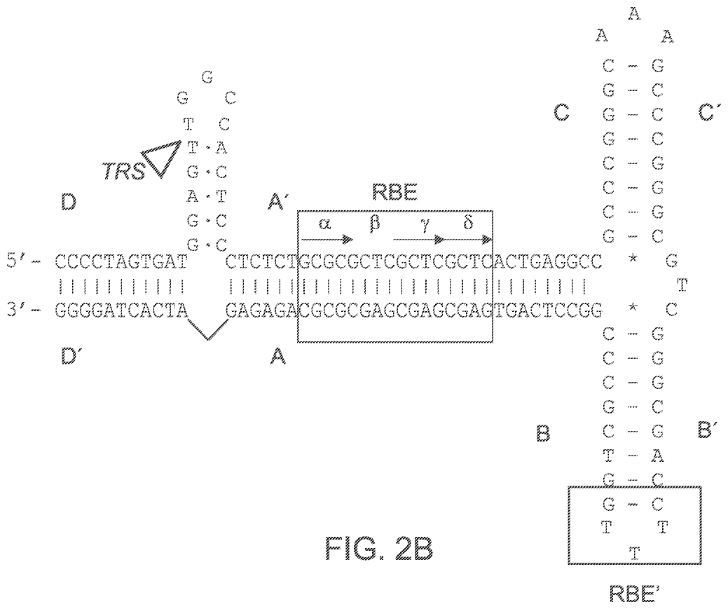

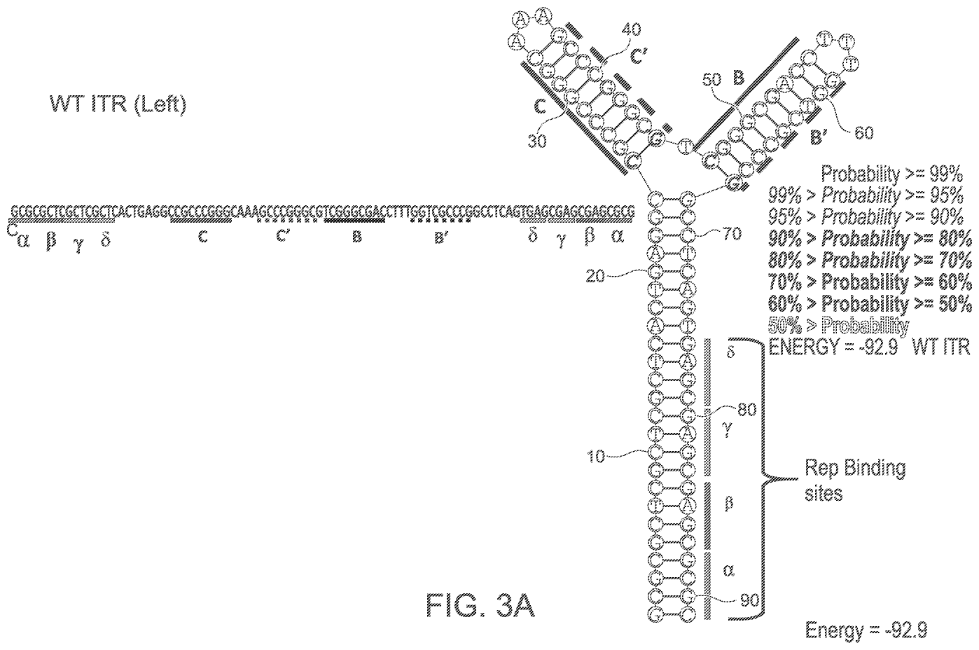

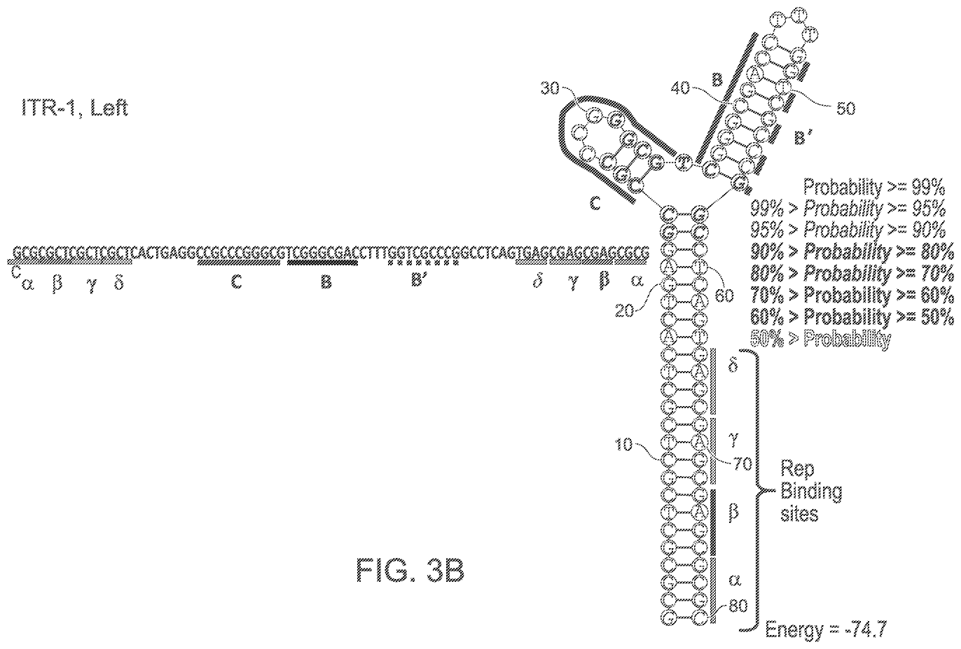

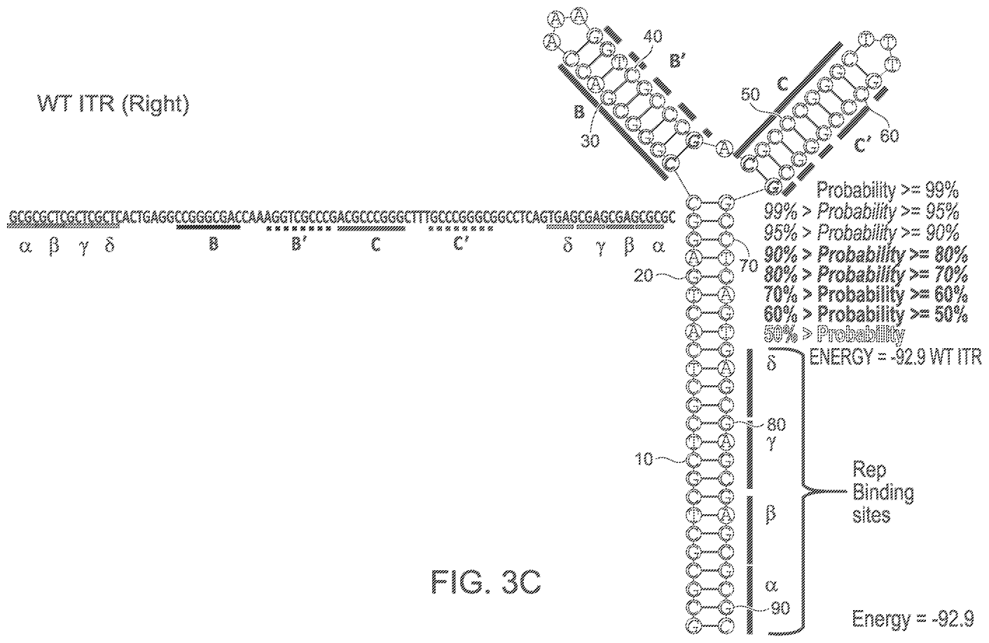

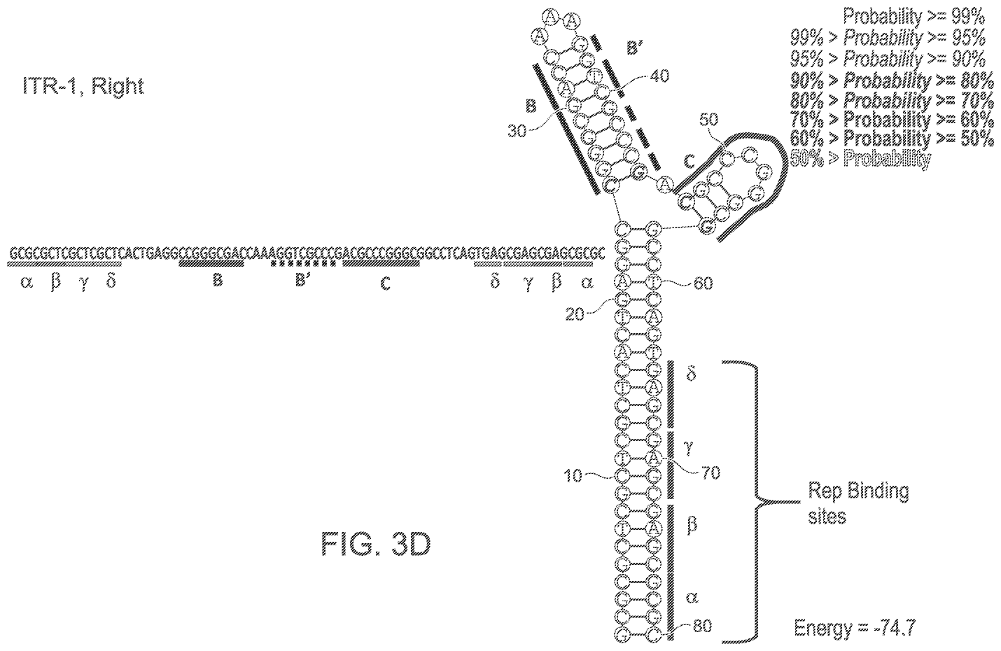

An ordinarily skilled artisan is aware that ITR sequences have a common structure of a double-stranded Holliday junction, which typically is a T-shaped or Y-shaped hairpin structure (see e.g., FIG. 2A and FIG. 3A), where each WT-ITR is formed by two palindromic arms or loops (B-B′ and C-C′) embedded in a larger palindromic arm (A-A′), and a single stranded D sequence, (where the order of these palindromic sequences defines the flip or flop orientation of the ITR). See, for example, structural analysis and sequence comparison of ITRs from different AAV serotypes (AAV1-AAV6) and described in Grimm et al., J. Virology, 2006; 80(1); 426-439; Yan et al., J. Virology, 2005; 364-379; Duan et al., Virology 1999; 261; 8-14. One of ordinary skill in the art can readily determine WT-ITR sequences from any AAV serotype for use in a ceDNA vector or ceDNA-plasmid based on the exemplary AAV2 ITR sequences provided herein. See, for example, the sequence comparison of ITRs from different AAV serotypes (AAV1-AAV6, and avian AAV (AAAV) and bovine AAV (BAAV)) described in Grimm et al., J. Virology, 2006; 80(1); 426-439; that show the % identity of the left ITR of AAV2 to the left ITR from other serotypes: AAV-1 (84%), AAV-3 (86%), AAV-4 (79%), AAV-5 (58%), AAV-6 (left ITR) (100%) and AAV-6 (right ITR) (82%).

### Symmetrical ITR Pairs

According to some embodiments, a ceDNA vector for expression of antigens, or immunogenic peptides, as described herein comprises, in the 5′ to 3′ direction: a first adeno-associated virus (AAV) inverted terminal repeat (ITR), a nucleic acid sequence of interest (for example an expression cassette as described herein) and a second AAV ITR, where the first ITR (5′ ITR) and the second ITR (3′ ITR) are symmetric, or substantially symmetrical with respect to each other—that is, a ceDNA vector can comprise ITR sequences that have a symmetrical three-dimensional spatial organization such that their structure is the same shape in geometrical space, or have the same A, C-C′ and B-B′ loops in 3D space. In such an embodiment, a symmetrical ITR pair, or substantially symmetrical ITR pair can be modified ITRs (e.g., mod-ITRs) that are not wild-type ITRs. A mod-ITR pair can have the same sequence which has one or more modifications from wild-type ITR and are reverse complements (inverted) of each other. In alternative embodiments, a modified ITR pair are substantially symmetrical as defined herein, that is, the modified ITR pair can have a different sequence but have corresponding or the same symmetrical three-dimensional shape.

### (i) Wildtype ITRs

According to some embodiments, the symmetrical ITRs, or substantially symmetrical ITRs are wild type (WT-ITRs) as described herein. That is, both ITRs have a wild-type sequence, but do not necessarily have to be WT-ITRs from the same AAV serotype. That is, according to some embodiments, one WT-ITR can be from one AAV serotype, and the other WT-ITR can be from a different AAV serotype. In such an embodiment, a WT-ITR pair are substantially symmetrical as defined herein, that is, they can have one or more conservative nucleotide modification while still retaining the symmetrical three-dimensional spatial organization.

Accordingly, as disclosed herein, ceDNA vectors contain a transgene or nucleic acid sequence positioned between two flanking wild-type inverted terminal repeat (WT-ITR) sequences, that are either the reverse complement (inverted) of each other, or alternatively, are substantially symmetrical relative to each other—that is a WT-ITR pair have symmetrical three-dimensional spatial organization. According to some embodiments, a wild-type ITR sequence (e.g., AAV WT-ITR) comprises a functional Rep binding site (RBS; e.g., 5′-GCGCGCTCGCTCGCTC-3′ for AAV2, SEQ ID NO: ______) and a functional terminal resolution site (TRS; e.g., 5′-AGTT-3′, SEQ ID NO: ______).

According to some aspect, ceDNA vectors for expression of antigens, or immunogenic peptides, are obtainable from a vector polynucleotide that encodes a nucleic acid operatively positioned between two WT inverted terminal repeat sequences (WT-ITRs) (e.g., AAV WT-ITRs). That is, both ITRs have a wild type sequence, but do not necessarily have to be WT-ITRs from the same AAV serotype. That is, according to some embodiments, one WT-ITR can be from one AAV serotype, and the other WT-ITR can be from a different AAV serotype. In such an embodiment, the WT-ITR pair are substantially symmetrical as defined herein, that is, they can have one or more conservative nucleotide modification while still retaining the symmetrical three-dimensional spatial organization. According to some embodiments, the 5′ WT-ITR is from one AAV serotype, and the 3′ WT-ITR is from the same or a different AAV serotype. According to some embodiments, the 5′ WT-ITR and the 3′WT-ITR are mirror images of each other, that is they are symmetrical. According to some embodiments, the 5′ WT-ITR and the 3′ WT-ITR are from the same AAV serotype.

WT ITRs are well known. According to some embodiment the two ITRs are from the same AAV2 serotype. In certain embodiments one can use WT from other serotypes. There are a number of serotypes that are homologous, e.g., AAV2, AAV4, AAV6, AAV8. According to some embodiments, closely homologous ITRs (e.g., ITRs with a similar loop structure) can be used. In another embodiment, one can use AAV WT ITRs that are more diverse, e.g., AAV2 and AAV5, and still another embodiment, one can use an ITR that is substantially WT—that is, it has the basic loop structure of the WT but some conservative nucleotide changes that do not alter or affect the properties. When using WT-ITRs from the same viral serotype, one or more regulatory sequences may further be used. In certain embodiments, the regulatory sequence is a regulatory switch that permits modulation of the activity of the ceDNA, e.g., the expression of the encoded antigens, or immunogenic peptides.

According to some embodiments, one aspect of the technology described herein relates to a ceDNA vector for expression of antigens, or immunogenic peptides, wherein the ceDNA vector comprises at least one nucleic acid sequence encoding, e.g., a HC and/or a LC, operably positioned between two wild-type inverted terminal repeat sequences (WT-ITRs), wherein the WT-ITRs can be from the same serotype, different serotypes or substantially symmetrical with respect to each other (i.e., have the symmetrical three-dimensional spatial organization such that their structure is the same shape in geometrical space, or have the same A, C-C′ and B-B′ loops in 3D space). According to some embodiments, the symmetric WT-ITRs comprises a functional terminal resolution site and a Rep binding site. According to some embodiments, the nucleic acid sequence encodes a transgene, and wherein the vector is not in a viral capsid.

According to some embodiments, the WT-ITRs are the same but the reverse complement of each other. For example, the sequence AACG in the 5′ ITR may be CGTT (i.e., the reverse complement) in the 3′ ITR at the corresponding site. According to some example, the 5′ WT-ITR sense strand comprises the sequence of ATCGATCG and the corresponding 3′ WT-ITR sense strand comprises CGATCGAT (i.e., the reverse complement of ATCGATCG). According to some embodiments, the WT-ITRs ceDNA further comprises a terminal resolution site and a replication protein binding site (RPS) (sometimes referred to as a replicative protein binding site), e.g., a Rep binding site.

Exemplary WT-ITR sequences for use in the ceDNA vectors for expression of antigens, or immunogenic peptides, comprising WT-ITRs are shown in Table 2 herein, which shows pairs of WT-ITRs (5′ WT-ITR and the 3′ WT-ITR).

As an exemplary example, the present disclosure provides a ceDNA vector for expression of antigens, or immunogenic peptides, comprising a promoter operably linked to a transgene (e.g., nucleic acid sequence), with or without the regulatory switch, where the ceDNA is devoid of capsid proteins and is: (a) produced from a ceDNA-plasmid (e.g., see FIGS. 1F-1G) that encodes WT-ITRs, where each WT-ITR has the same number of intramolecularly duplexed base pairs in its hairpin secondary configuration (preferably excluding deletion of any AAA or TTT terminal loop in this configuration compared to these reference sequences), and (b) is identified as ceDNA using the assay for the identification of ceDNA by agarose gel electrophoresis under native gel and denaturing conditions in Example 1.

According to some embodiments, the flanking WT-ITRs are substantially symmetrical to each other. In this embodiment the 5′ WT-ITR can be from one serotype of AAV, and the 3′ WT-ITR from a different serotype of AAV, such that the WT-ITRs are not identical reverse complements. For example, the 5′ WT-ITR can be from AAV2, and the 3′ WT-ITR from a different serotype (e.g., AAV1, 3, 4, 5, 6, 7, 8, 9, 10, 11, and 12. According to some embodiments, WT-ITRs can be selected from two different parvoviruses selected from any to of: AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. According to some embodiments, such a combination of WT ITRs is the combination of WT-ITRs from AAV2 and AAV6. According to some embodiments, the substantially symmetrical WT-ITRs are when one is inverted relative to the other ITR at least 90% identical, at least 95% identical, at least 96% . . . 97% . . . 98% . . . 99% . . . 99.5% and all points in between, and has the same symmetrical three-dimensional spatial organization.

According to some embodiments, a WT-ITR pair are substantially symmetrical as they have symmetrical three-dimensional spatial organization, e.g., have the same 3D organization of the A, C-C′. B-B′ and D arms. According to some embodiments, a substantially symmetrical WT-ITR pair are inverted relative to the other, and are at least 95% identical, at least 96% . . . 97% . . . 98% . . . 99% . . . 99.5% and all points in between, to each other, and one WT-ITR retains the Rep-binding site (RBS) of 5′-GCGCGCTCGCTCGCTC-3′ (SEQ ID NO: 60) and a terminal resolution site (trs). According to some embodiments, a substantially symmetrical WT-ITR pair are inverted relative to each other, and are at least 95% identical, at least 96% . . . 97% . . . 98% . . . 99% . . . 99.5% and all points in between, to each other, and one WT-ITR retains the Rep-binding site (RBS) of 5′-GCGCGCTCGCTCGCTC-3′ (SEQ ID NO: ______) and a terminal resolution site (trs) and in addition to a variable palindromic sequence allowing for hairpin secondary structure formation. Homology can be determined by standard means well known in the art such as BLAST (Basic Local Alignment Search Tool), BLASTN at default setting.

According to some embodiments, the structural element of the ITR can be any structural element that is involved in the functional interaction of the ITR with a large Rep protein (e.g., Rep 78 or Rep 68). In certain embodiments, the structural element provides selectivity to the interaction of an ITR with a large Rep protein, i.e., determines at least in part which Rep protein functionally interacts with the ITR. In other embodiments, the structural element physically interacts with a large Rep protein when the Rep protein is bound to the ITR. Each structural element can be, e.g., a secondary structure of the ITR, a nucleic acid sequence of the ITR, a spacing between two or more elements, or a combination of any of the above. According to some embodiments, the structural elements are selected from the group consisting of an A and an A′ arm, a B and a B′ arm, a C and a C′ arm, a D arm, a Rep binding site (RBE) and an RBE′ (i.e., complementary RBE sequence), and a terminal resolution sire (trs).

By way of example only, Table 8 indicates exemplary combinations of WT-ITRs.

Table 8: Exemplary combinations of WT-ITRs from the same serotype or different serotypes, or different parvoviruses. The order shown is not indicative of the ITR position, for example, “AAV1, AAV2” demonstrates that the ceDNA can comprise a WT-AAV1 ITR in the 5′ position, and a WT-AAV2 ITR in the 3′ position, or vice versa, a WT-AAV2 ITR the 5′ position, and a WT-AAV1 ITR in the 3′ position. Abbreviations: AAV serotype 1 (AAV1), AAV serotype 2 (AAV2), AAV serotype 3 (AAV3), AAV serotype 4 (AAV4), AAV serotype 5 (AAV), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12); AAVrh8, AAVrh1, AAV-DJ, and AAV-DJ8 genome (E.g., NCB: NC 002077; NC 001401; NC001729; NC001829; NC006152; NC 006260; NC 006261), ITRs from warm-blooded animals (avian AAV (AAAV), bovine AAV (BAAV), canine, equine, and ovine AAV), ITRs from B19 Parvovirus (GenBank Accession No: NC 000883), Minute Virus from Mouse (MVM) (GenBank Accession No. NC 001510); Goose: goose parvovirus (GenBank Accession No. NC 001701); snake: snake parvovirus 1 (GenBank Accession No. NC 006148).

TABLE 8
Exemplary combinations of WT-ITRs

| | | | | |
|---|---|---|---|---|
| AAV1, AAV1 | AAV2, AAV2 | AAV3, AAV3 | AAV4, AAV4 | AAV5, AAV5 |
| AAV1, AAV2 | AAV2, AAV3 | AAV3, AAV4 | AAV4, AAV5 | AAV5, AAV6 |
| AAV1, AAV3 | AAV2, AAV4 | AAV3, AAV5 | AAV4, AAV6 | AAV5, AAV7 |
| AAV1, AAV4 | AAV2, AAV5 | AAV3, AAV6 | AAV4, AAV7 | AAV5, AAV8 |
| AAV1, AAV5 | AAV2, AAV6 | AAV3, AAV7 | AAV4, AAV8 | AAV5, AAV9 |
| AAV1, AAV6 | AAV2, AAV7 | AAV3, AAV8 | AAV4, AAV9 | AAV5, AAV10 |
| AAV1, AAV7 | AAV2, AAV8 | AAV3, AAV9 | AAV4, AAV10 | AAV5, AAV11 |
| AAV1, AAV8 | AAV2, AAV9 | AAV3, AAV10 | AAV4, AAV11 | AAV5, AAV12 |
| AAV1, AAV9 | AAV2, AAV10 | AAV3, AAV11 | AAV4, AAV12 | AAV5, AAVRH8 |
| AAV1, AAV10 | AAV2, AAV11 | AAV3, AAV12 | AAV4, AAVRH8 | AAV5, AAVRH10 |
| AAV1, AAV11 | AAV2, AAV12 | AAV3, AAVRH8 | AAV4, AAVRH10 | AAV5, AAV13 |
| AAV1, AAV12 | AAV2, AAVRH8 | AAV3, AAVRH10 | AAV4, AAV13 | AAV5, AAVDJ |
| AAV1, AAVRH8 | AAV2, AAVRH10 | AAV3, AAV13 | AAV4, AAVDJ | AAV5, AAVDJ8 |
| AAV1, AAVRH10 | AAV2, AAV13 | AAV3, AAVDJ | AAV4, AAVDJ8 | AAV5, AVIAN |
| AAV1, AAV13 | AAV2, AAVDJ | AAV3, AAVDJ8 | AAV4, AVIAN | AAV5, BOVINE |
| AAV1, AAVDJ | AAV2, AAVDJ8 | AAV3, AVIAN | AAV4, BOVINE | AAV5, CANINE |
| AAV1, AAVDJ8 | AAV2, AVIAN | AAV3, BOVINE | AAV4, CANINE | AAV5, EQUINE |
| AAV1, AVIAN | AAV2, BOVINE | AAV3, CANINE | AAV4, EQUINE | AAV5, GOAT |
| AAV1, BOVINE | AAV2, CANINE | AAV3, EQUINE | AAV4, GOAT | AAV5, SHRIMP |
| AAV1, CANINE | AAV2, EQUINE | AAV3, GOAT | AAV4, SHRIMP | AAV5, PORCINE |
| AAV1, EQUINE | AAV2, GOAT | AAV3, SHRIMP | AAV4, PORCINE | AAV5, INSECT |
| AAV1, GOAT | AAV2, SHRIMP | AAV3, PORCINE | AAV4, INSECT | AAV5, OVINE |
| AAV1, SHRIMP | AAV2, PORCINE | AAV3, INSECT | AAV4, OVINE | AAV5, B19 |
| AAV1, PORCINE | AAV2, INSECT | AAV3, OVINE | AAV4, B19 | AAV5, MVM |
| AAV1, INSECT | AAV2, OVINE | AAV3, B19 | AAV4, MVM | AAV5, GOOSE |
| AAV1, OVINE | AAV2, B19 | AAV3, MVM | AAV4, GOOSE | AAV5, SNAKE |
| AAV1, B19 | AAV2, MVM | AAV3, GOOSE | AAV4, SNAKE | |
| AAV1, MVM | AAV2, GOOSE | AAV3, SNAKE | | |
| AAV1, GOOSE | AAV2, SNAKE | | | |
| AAV1, SNAKE | | | | |
| AAV6, AAV6 | AAV7, AAV7 | AAV8, AAV8 | AAV9, AAV9 | AAV10, AAV10 |
| AAV6, AAV7 | AAV7, AAV8 | AAV8, AAV9 | AAV9, AAV10 | AAV10, AAV11 |
| AAV6, AAV8 | AAV7, AAV9 | AAV8, AAV10 | AAV9, AAV11 | AAV10, AAV12 |
| AAV6, AAV9 | AAV7, AAV10 | AAV8, AAV11 | AAV9, AAV12 | AAV10, AAVRH8 |
| AAV6, AAV10 | AAV7, AAV11 | AAV8, AAV12 | AAV9, AAVRH8 | AAV10, AAVRH10 |
| AAV6, AAV11 | AAV7, AAV12 | AAV8, AAVRH8 | AAV9, AAVRH10 | AAV10, AAV13 |
| AAV6, AAV12 | AAV7, AAVRH8 | AAV8, AAVRH10 | AAV9, AAV13 | AAV10, AAVDJ |
| AAV6, AAVRH8 | AAV7, AAVRH10 | AAV8, AAV13 | AAV9, AAVDJ | AAV10, AAVDJ8 |
| AAV6, AAVRH10 | AAV7, AAV13 | AAV8, AAVDJ | AAV9, AAVDJ8 | AAV10, AVIAN |
| AAV6, AAV13 | AAV7, AAVDJ | AAV8, AAVDJ8 | AAV9, AVIAN | AAV10, BOVINE |
| AAV6, AAVDJ | AAV7, AAVDJ8 | AAV8, AVIAN | AAV9, BOVINE | AAV10, CANINE |
| AAV6, AAVDJ8 | AAV7, AVIAN | AAV8, BOVINE | AAV9, CANINE | AAV10, EQUINE |
| AAV6, AVIAN | AAV7, BOVINE | AAV8, CANINE | AAV9, EQUINE | AAV10, GOAT |
| AAV6, BOVINE | AAV7, CANINE | AAV8, EQUINE | AAV9, GOAT | AAV10, SHRIMP |
| AAV6, CANINE | AAV7, EQUINE | AAV8, GOAT | AAV9, SHRIMP | AAV10, PORCINE |
| AAV6, EQUINE | AAV7, GOAT | AAV8, SHRIMP | AAV9, PORCINE | AAV10, INSECT |
| AAV6, GOAT | AAV7, SHRIMP | AAV8, PORCINE | AAV9, INSECT | AAV10, OVINE |
| AAV6, SHRIMP | AAV7, PORCINE | AAV8, INSECT | AAV9, OVINE | AAV10, B19 |
| AAV6, PORCINE | AAV7, INSECT | AAV8, OVINE | AAV9, B19 | AAV10, MVM |
| AAV6, INSECT | AAV7, OVINE | AAV8, B19 | AAV9, MVM | AAV10, GOOSE |
| AAV6, OVINE | AAV7, B19 | AAV8, MVM | AAV9, GOOSE | AAV10, SNAKE |
| AAV6, B19 | AAV7, MVM | AAV8, GOOSE | AAV9, SNAKE | |
| AAV6, MVM | AAV7, GOOSE | AAV8, SNAKE | | |
| AAV6, GOOSE | AAV7, SNAKE | | | |
| AAV6, SNAKE | | | | |
| AAV11, AAV11 | AAV12, AAV12 | AAVRH8, AAVRH8 | AAVRH10, AAVRH10 | AAV13, AAV13 |
| AAV11, AAV12 | AAV12, AAVRH8 | AAVRH8, AAVRH10 | AAVRH10, AAV13 | AAV13, AAVDJ |
| AAV11, AAVRH8 | AAV12, AAVRH10 | AAVRH8, AAV13 | AAVRH10, AAVDJ | AAV13, AAVDJ8 |
| AAV11, AAVRH10 | AAV12, AAV13 | AAVRH8, AAVDJ | AAVRH10, AAVDJ8 | AAV13, AVIAN |
| AAV11, AAV13 | AAV12, AAVDJ | AAVRH8, AAVDJ8 | AAVRH10, AVIAN | AAV13, BOVINE |
| AAV11, AAVDJ | AAV12, AAVDJ8 | AAVRH8, AVIAN | AAVRH10, BOVINE | AAV13, CANINE |
| AAV11, AAVDJ8 | AAV12, AVIAN | AAVRH8, BOVINE | AAVRH10, CANINE | AAV13, EQUINE |
| AAV11, AVIAN | AAV12, BOVINE | AAVRH8, CANINE | AAVRH10, EQUINE | AAV13, GOAT |
| AAV11, BOVINE | AAV12, CANINE | AAVRH8, EQUINE | AAVRH10, GOAT | AAV13, SHRIMP |
| AAV11, CANINE | AAV12, EQUINE | AAVRH8, GOAT | AAVRH10, SHRIMP | AAV13, PORCINE |
| AAV11, EQUINE | AAV12, GOAT | AAVRH8, SHRIMP | AAVRH10, PORCINE | AAV13, INSECT |
| AAV11, GOAT | AAV12, SHRIMP | AAVRH8, PORCINE | AAVRH10, INSECT | AAV13, OVINE |
| AAV11, SHRIMP | AAV12, PORCINE | AAVRH8, INSECT | AAVRH10, OVINE | AAV13, B19 |
| AAV11, PORCINE | AAV12, INSECT | AAVRH8, OVINE | AAVRH10, B19 | AAV13, MVM |
| AAV11, INSECT | AAV12, OVINE | AAVRH8, B19 | AAVRH10, MVM | AAV13, GOOSE |
| AAV11, OVINE | AAV12, B19 | AAVRH8, MVM | AAVRH10, GOOSE | AAV13, SNAKE |
| AAV11, B19 | AAV12, MVM | AAVRH8, GOOSE | AAVRH10, SNAKE | |
| AAV11, MVM | AAV12, GOOSE | AAVRH8, SNAKE | | |
| AAV11, GOOSE | AAV12, SNAKE | | | |
| AAV11, SNAKE | | | | |
| AAVDJ, AAVDJ | AAVDJ8, AVVDJ8 | AVIAN, AVIAN | BOVINE, BOVINE | CANINE, |
| | | | | CANINE |
| AAVDJ, AAVDJ8 | AAVDJ8, AVIAN | AVIAN, BOVINE | BOVINE, CANINE | CANINE, EQUINE |
| AAVDJ, AVIAN | AAVDJ8, BOVINE | AVIAN, CANINE | BOVINE, EQUINE | CANINE, GOAT |
| AAVDJ, BOVINE | AAVDJ8, CANINE | AVIAN, EQUINE | BOVINE, GOAT | CANINE, SHRIMP |
| AAVDJ, CANINE | AAVDJ8, EQUINE | AVIAN, GOAT | BOVINE, SHRIMP | CANINE, PORCINE |
| AAVDJ, EQUINE | AAVDJ8, GOAT | AVIAN, SHRIMP | BOVINE, PORCINE | CANINE, INSECT |
| AAVDJ, GOAT | AAVDJ8, SHRIMP | AVIAN, PORCINE | BOVINE, INSECT | CANINE, OVINE |
| AAVDJ, SHRIMP | AAVDJ8, PORCINE | AVIAN, INSECT | BOVINE, OVINE | CANINE, B19 |
| AAVDJ, PORCINE | AAVDJ8, INSECT | AVIAN, OVINE | BOVINE, B19 | CANINE, MVM |
| AAVDJ, INSECT | AAVDJ8, OVINE | AVIAN, B19 | BOVINE, MVM | CANINE, GOOSE |
| AAVDJ, OVINE | AAVDJ8, B19 | AVIAN, MVM | BOVINE, GOOSE | CANINE, SNAKE |
| AAVDJ, B19 | AAVDJ8, MVM | AVIAN, GOOSE | BOVINE, SNAKE | |
| AAVDJ, MVM | AAVDJ8, GOOSE | AVIAN, SNAKE | | |
| AAVDJ, GOOSE | AAVDJ8, SNAKE | | | |
| AAVDJ, SNAKE | | | | |
| EQUINE, EQUINE | GOAT, GOAT | SHRIMP, SHRIMP | PORCINE, PORCINE | INSECT, INSECT |
| EQUINE, GOAT | GOAT, SHRIMP | SHRIMP, PORCINE | PORCINE, INSECT | INSECT, OVINE |
| EQUINE, SHRIMP | GOAT, PORCINE | SHRIMP, INSECT | PORCINE, OVINE | INSECT, B19 |
| EQUINE, PORCINE | GOAT, INSECT | SHRIMP, OVINE | PORCINE, B19 | INSECT, MVM |
| EQUINE, INSECT | GOAT, OVINE | SHRIMP, B19 | PORCINE, MVM | INSECT, GOOSE |
| EQUINE, OVINE | GOAT, B19 | SHRIMP, MVM | PORCINE, GOOSE | INSECT, SNAKE |
| EQUINE, B19 | GOAT, MVM | SHRIMP, GOOSE | PORCINE, SNAKE | |
| EQUINE, MVM | GOAT, GOOSE | SHRIMP, SNAKE | | |
| EQUINE, GOOSE | GOAT, SNAKE | | | |
| EQUINE, SNAKE | | | | |
| OVINE, OVINE | B19, B19 | MVM, MVM | GOOSE, GOOSE | SNAKE, SNAKE |
| OVINE, B19 | B19, MVM | MVM, GOOSE | GOOSE, SNAKE | |
| OVINE, MVM | B19, GOOSE | MVM, SNAKE | | |
| OVINE, GOOSE | B19, SNAKE | | | |
| OVINE, SNAKE | | | | |

By way of example only, Table 9 shows the sequences of exemplary WT-ITRs from some different AAV serotypes.

TABLE 9
Exemplary WT-ITRs

| AAV | | |
|---|---|---|
| serotype | 5′ WT-ITR (LEFT) | 3′ WT-ITR (RIGHT) |
| AAV1 | 5′- | 5′- |
| | TTGCCCACTCCCTCTCTGCGCGCTCGCT | TTACCCTAGTGATGGAGTTGCCCACTCCC |
| | CGCTCGGTGGGGCCTGCGGACCAAAGG | TCTCTGCGCGCGTCGCTCGCTCGGTGGG |
| | TCCGCAGACGGCAGAGGTCTCCTCTGCC | GCCGGCAGAGGAGACCTCTGCCGTCTGC |
| | GGCCCCACCGAGCGAGCGACGCGCGCA | GGACCTTTGGTCCGCAGGCCCCACCGAG |
| | GAGAGGGAGTGGGCAACTCCATCACTA | CGAGCGAGCGCGCAGAGAGGGAGTGGG |
| | GGGTAA-3′ | CAA-3′ (SEQ ID NO: 10) |
| | (SEQ ID NO: 5) | |
| AAV2 | CCTGCAGGCAGCTGCGCGCTCGCTCGCT | AGGAACCCCTAGTGATGGAGTTGGCCAC |
| | CACTGAGGCCGCCCGGGCAAAGCCCGG | TCCCTCTCTGCGCGCTCGCTCGCTCACTG |
| | GCGTCGGGCGACCTTTGGTCGCCCGGCC | AGGCCGGGCGACCAAAGGTCGCCCGAC |
| | TCAGTGAGCGAGCGAGCGCGCAGAGAG | GCCCGGGCTTTGCCCGGGCGGCCTCAGT |
| | GGAGTGGCCAACTCCATCACTAGGGGT | GAGCGAGCGAGCGCGCAGCTGCCTGCAG |
| | TCCT (SEQ ID NO: 2) | G (SEQ ID NO: 1) |
| AAV3 | 5′- | 5′- |
| | TTGGCCACTCCCTCTATGCGCACTCGCT | ATACCTCTAGTGATGGAGTTGGCCACTC |
| | CGCTCGGTGGGGCCTGGCGACCAAAGG | CCTCTATGCGCACTCGCTCGCTCGGTGG |
| | TCGCCAGACGGACGTGGGTTTCCACGTC | GGCCGGACGTGGAAACCCACGTCCGTCT |
| | CGGCCCCACCGAGCGAGCGAGTGCGCA | GGCGACCTTTGGTCGCCAGGCCCCACCG |
| | TAGAGGGAGTGGCCAACTCCATCACTA | AGCGAGCGAGTGCGCATAGAGGGAGTG |
| | GAGGTAT-3′ (SEQ ID NO: 6) | GCCAA-3′ (SEQ ID NO: 11) |
| AAV4 | 5′- | 5′- |
| | TTGGCCACTCCCTCTATGCGCGCTCGCT | AGTTGGCCACATTAGCTATGCGCGCTCG |
| | CACTCACTCGGCCCTGGAGACCAAAGG | CTCACTCACTCGGCCCTGGAGACCAAAG |
| | TCTCCAGACTGCCGGCCTCTGGCCGGCA | GTCTCCAGACTGCCGGCCTCTGGCCGGC |
| | GGGCCGAGTGAGTGAGCGAGCGCGCAT | AGGGCCGAGTGAGTGAGCGAGCGCGCA |
| | AGAGGGAGTGGCCAACT-3′ | TAGAGGGAGTGGCCAA-3′ |
| | (SEQ ID NO: 7) | (SEQ ID NO: 12) |
| AAV5 | 5′- | 5′- |
| | TCCCCCCTGTCGCGTTCGCTCGCTCGCT | CTTACAAAACCCCCTTGCTTGAGAGTGT |
| | GGCTCGTTTGGGGGGGCGACGGCCAGA | GGCACTCTCCCCCCTGTCGCGTTCGCTCG |
| | GGGCCGTCGTCTGGCAGCTCTTTGAGCT | CTCGCTGGCTCGTTTGGGGGGGTGGCAG |
| | GCCACCCCCCCAAACGAGCCAGCGAGC | CTCAAAGAGCTGCCAGACGACGGCCCTC |
| | GAGCGAACGCGACAGGGGGGAGAGTGC | TGGCCGTCGCCCCCCCAAACGAGCCAGC |
| | CACACTCTCAAGCAAGGGGGTTTTGTAA | GAGCGAGCGAACGCGACAGGGGGGA-3′ |
| | G -3′ (SEQ ID NO: 8) | (SEQ ID NO: 13) |
| AAV6 | 5′- | 5′- |
| | TTGCCCACTCCCTCTAATGCGCGCTCGC | ATACCCCTAGTGATGGAGTTGCCCACTC |
| | TCGCTCGGTGGGGCCTGCGGACCAAAG | CCTCTATGCGCGCTCGCTCGCTCGGTGG |
| | GTCCGCAGACGGCAGAGGTCTCCTCTGC | GGCCGGCAGAGGAGACCTCTGCCGTCTG |
| | CGGCCCCACCGAGCGAGCGAGCGCGCA | CGGACCTTTGGTCCGCAGGCCCCACCGA |
| | TAGAGGGAGTGGGCAACTCCATCACTA | GCGAGCGAGCGCGCATTAGAGGGAGTG |
| | GGGGTAT-3′ (SEQ ID NO: 9) | GGCAA (SEQ ID NO: 14) |

According to some embodiments, the nucleic acid sequence of the WT-ITR sequence can be modified (e.g., by modifying 1, 2, 3, 4 or 5, or more nucleotides or any range therein), whereby the modification is a substitution for a complementary nucleotide, e.g., G for a C, and vice versa, and T for an A, and vice versa.

In certain embodiments of the present disclosure, the ceDNA vector for expression of antigens, or immunogenic peptides, does not have a WT-ITR consisting of the nucleic acid sequence selected from any of: SEQ ID NOs: 1, 2, 5-14. In alternative embodiments of the present disclosure, if a ceDNA vector has a WT-ITR comprising the nucleic acid sequence selected from any of: SEQ ID NOs: 1, 2, 5-14, then the flanking ITR is also WT and the ceDNA vector comprises a regulatory switch, e.g., as disclosed herein and in International application PCT/US18/49996 (e.g., see Table 11 of PCT/US18/49996, incorporated by reference in its entirety herein). According to some embodiments, the ceDNA vector for expression of antigens, or immunogenic peptides, comprises a regulatory switch as disclosed herein and a WT-ITR selected having the nucleic acid sequence selected from any of the group consisting of: SEQ ID NO: 1, 2, 5-14.

The ceDNA vector for expression of antigens, or immunogenic peptides, as described herein can include WT-ITR structures that retains an operable RBE, trs and RBE′ portion. FIG. 2A and FIG. 2B, using wild-type ITRs for exemplary purposes, show one possible mechanism for the operation of a trs site within a wild type ITR structure portion of a ceDNA vector. According to some embodiments, the ceDNA vector for expression of antigens, or immunogenic peptides, contains one or more functional WT-ITR polynucleotide sequences that comprise a Rep-binding site (RBS; 5′-GCGCGCTCGCTCGCTC-3′ (SEQ ID NO: ______) for AAV2) and a terminal resolution site (TRS; 5′-AGTT (SEQ ID NO: ______)). According to some embodiments, at least one WT-ITR is functional. In alternative embodiments, where a ceDNA vector for expression of antigens, or immunogenic peptides, comprises two WT-ITRs that are substantially symmetrical to each other, at least one WT-ITR is functional and at least one WT-ITR is non-functional.

Modified ITRs (Mod-ITRs) in General for ceDNA Vectors Comprising Asymmetric ITR Pairs or Symmetric ITR Pairs

As discussed herein, a ceDNA vector for expression of antigens, or immunogenic peptides, can comprise a symmetrical ITR pair or an asymmetrical ITR pair. In both instances, one or both of the ITRs can be modified ITRs—the difference being that in the first instance (i.e., symmetric mod-ITRs), the mod-ITRs have the same three-dimensional spatial organization (i.e., have the same A-A′, C-C′ and B-B′ arm configurations), whereas in the second instance (i.e., asymmetric mod-ITRs), the mod-ITRs have a different three-dimensional spatial organization (i.e., have a different configuration of A-A′, C-C′ and B-B′ arms).

According to some embodiments, a modified ITR is an ITRs that is modified by deletion, insertion, and/or substitution as compared to a wild-type ITR sequence (e.g., AAV ITR). According to some embodiments, at least one of the ITRs in the ceDNA vector comprises a functional Rep binding site (RBS; e.g., 5′-GCGCGCTCGCTCGCTC-3′ for AAV2) and a functional terminal resolution site (TRS; e.g., 5′-AGTT-3′) According to some embodiments, at least one of the ITRs is a non-functional ITR. According to some embodiments, the different or modified ITRs are not each wild type ITRs from different serotypes.

Specific alterations and mutations in the ITRs are described in detail herein, but in the context of ITRs, “altered” or “mutated” or “modified”, it indicates that nucleotides have been inserted, deleted, and/or substituted relative to the wild-type, reference, or original ITR sequence. The altered or mutated ITR can be an engineered ITR. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature.

According to some embodiments, a mod-ITR may be synthetic. According to some embodiments, a synthetic ITR is based on ITR sequences from more than one AAV serotype. In another embodiment, a synthetic ITR includes no AAV-based sequence. In yet another embodiment, a synthetic ITR preserves the ITR structure described above although having only some or no AAV-sourced sequence. According to some aspects, a synthetic ITR may interact preferentially with a wild type Rep or a Rep of a specific serotype, or According to some instances will not be recognized by a wild-type Rep and be recognized only by a mutated Rep.

The skilled artisan can determine the corresponding sequence in other serotypes by known means. For example, determining if the change is in the A, A′, B, B′, C, C′ or D region and determine the corresponding region in another serotype. One can use BLAST® (Basic Local Alignment Search Tool) or other homology alignment programs at default status to determine the corresponding sequence. The disclosure further provides populations and pluralities of ceDNA vectors comprising mod-ITRs from a combination of different AAV serotypes—that is, one mod-ITR can be from one AAV serotype and the other mod-ITR can be from a different serotype. Without wishing to be bound by theory, according to some embodiment one ITR can be from or based on an AAV2 ITR sequence and the other ITR of the ceDNA vector can be from or be based on any one or more ITR sequence of AAV serotype 1 (AAV1), AAV serotype 4 (AAV4), AAV serotype 5 (AAV5), AAV serotype 6 (AAV6), AAV serotype 7 (AAV7), AAV serotype 8 (AAV8), AAV serotype 9 (AAV9), AAV serotype 10 (AAV10), AAV serotype 11 (AAV11), or AAV serotype 12 (AAV12).

Any parvovirus ITR can be used as an ITR or as a base ITR for modification. Preferably, the parvovirus is a dependovirus. More preferably AAV. The serotype chosen can be based upon the tissue tropism of the serotype. AAV2 has a broad tissue tropism, AAV1 preferentially targets to neuronal and skeletal muscle, and AAV5 preferentially targets neuronal, retinal pigmented epithelia, and photoreceptors. AAV6 preferentially targets skeletal muscle and lung. AAV8 preferentially targets liver, skeletal muscle, heart, and pancreatic tissues. AAV9 preferentially targets liver, skeletal and lung tissue. According to some embodiments, the modified ITR is based on an AAV2 ITR.

More specifically, the ability of a structural element to functionally interact with a particular large Rep protein can be altered by modifying the structural element. For example, the nucleic acid sequence of the structural element can be modified as compared to the wild-type sequence of the ITR. According to some embodiments, the structural element (e.g., A arm, A′ arm, B arm, B′ arm, C arm, C′ arm, D arm, RBE, RBE′, and trs) of an ITR can be removed and replaced with a wild-type structural element from a different parvovirus. For example, the replacement structure can be from AAV1, AAV2, AAV3, AAV4, AAV5, AAV6, AAV7, AAV8, AAV9, AAV10, AAV11, AAV12, AAV13, snake parvovirus (e.g., royal python parvovirus), bovine parvovirus, goat parvovirus, avian parvovirus, canine parvovirus, equine parvovirus, shrimp parvovirus, porcine parvovirus, or insect AAV. For example, the ITR can be an AAV2 ITR and the A or A′ arm or RBE can be replaced with a structural element from AAV5. In another example, the ITR can be an AAV5 ITR and the C or C′ arms, the RBE, and the trs can be replaced with a structural element from AAV2. In another example, the AAV ITR can be an AAV5 ITR with the B and B′ arms replaced with the AAV2 ITR B and B′ arms.

By way of example only, Table 10 indicates exemplary modifications of at least one nucleotide (e.g., a deletion, insertion and/or substitution) in regions of a modified ITR, where X is indicative of a modification of at least one nucleic acid (e.g., a deletion, insertion and/or substitution) in that section relative to the corresponding wild-type ITR. According to some embodiments, any modification of at least one nucleotide (e.g., a deletion, insertion and/or substitution) in any of the regions of C and/or C′ and/or B and/or B′ retains three sequential T nucleotides (i.e., TTT) in at least one terminal loop. For example, if the modification results in any of: a single arm ITR (e.g., single C-C′ arm, or a single B-B′ arm), or a modified C-B′ arm or C′-B arm, or a two arm ITR with at least one truncated arm (e.g., a truncated C-C′ arm and/or truncated B-B′ arm), at least the single arm, or at least one of the arms of a two arm ITR (where one arm can be truncated) retains three sequential T nucleotides (i.e., TTT) in at least one terminal loop. According to some embodiments, a truncated C-C′ arm and/or a truncated B-B′ arm has three sequential T nucleotides (i.e., TTT) in the terminal loop.

TABLE 10
Exemplary combinations of modifications of at least one nucleotide
(e.g., a deletion, insertion and/or substitution) to different
B-B′ and C-C′ regions or arms of ITRs (X indicates
a nucleotide modification, e.g., addition, deletion or substitution
of at least one nucleotide in the region).

| B region | B′ region | C region | C′ region |
|---|---|---|---|
| X | | | |
| | X | | |
| X | X | | |
| | | X | |
| | | | X |
| | | X | X |
| X | | X | |
| X | | | X |
| | X | X | |
| | X | | X |
| X | X | X | |
| X | X | | X |
| X | | X | X |
| | X | X | X |
| X | X | X | X |

According to some embodiments, mod-ITR for use in a ceDNA vector for expression of Antigens, or immunogenic peptides, comprises an asymmetric ITR pair, or a symmetric mod-ITR pair as disclosed herein, can comprise any one of the combinations of modifications shown in Table 10, and also a modification of at least one nucleotide in any one or more of the regions selected from: between A′ and C, between C and C′, between C′ and B, between B and B′ and between B′ and A. According to some embodiments, any modification of at least one nucleotide (e.g., a deletion, insertion and/or substitution) in the C or C′ or B or B′ regions, still preserves the terminal loop of the stem-loop. According to some embodiments, any modification of at least one nucleotide (e.g., a deletion, insertion and/or substitution) between C and C′ and/or B and B′ retains three sequential T nucleotides (i.e., TTT) in at least one terminal loop. In alternative embodiments, any modification of at least one nucleotide (e.g., a deletion, insertion and/or substitution) between C and C′ and/or B and B′ retains three sequential A nucleotides (i.e., AAA) in at least one terminal loop. According to some embodiments, a modified ITR for use herein can comprise any one of the combinations of modifications shown in Table 10, and also a modification of at least one nucleotide (e.g., a deletion, insertion and/or substitution) in any one or more of the regions selected from: A′, A and/or D. For example, a modified ITR for use herein can comprise any one of the combinations of modifications shown in Table 10, and also a modification of at least one nucleotide (e.g., a deletion, insertion and/or substitution) in the A region. According to some embodiments, a modified ITR for use herein can comprise any one of the combinations of modifications shown in Table 10, and also a modification of at least one nucleotide (e.g., a deletion, insertion and/or substitution) in the A′ region. According to some embodiments, a modified ITR for use herein can comprise any one of the combinations of modifications shown in Table 10, and also a modification of at least one nucleotide (e.g., a deletion, insertion and/or substitution) in the A and/or A′ region. According to some embodiments, a modified ITR for use herein can comprise any one of the combinations of modifications shown in Table 10, and also a modification of at least one nucleotide (e.g., a deletion, insertion and/or substitution) in the D region.

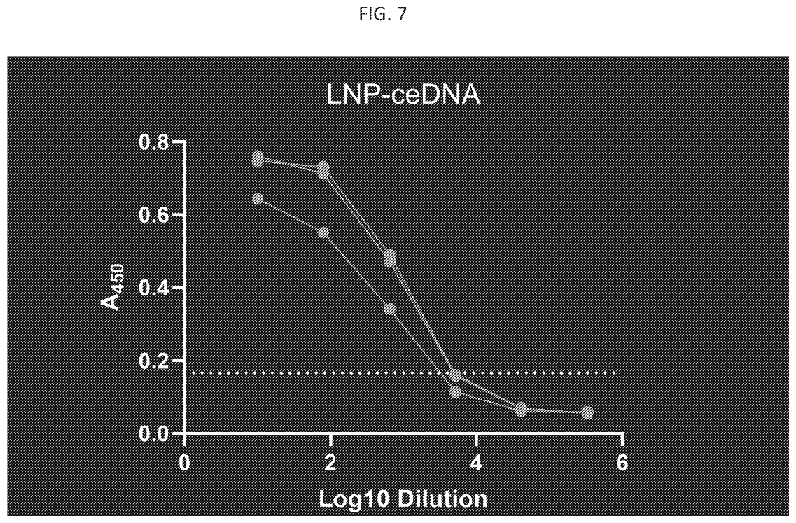

According to some embodiments, the nucleotide sequence of the structural element can be modified (e.g., by modifying 1, 2, 3, 4, 5, 6, 7, 8, 9, 10, 11, 12, 13, 14, 15, 16, 17, 18, 19, or 20 or more nucleotides or any range therein) to produce a modified structural element. According to some embodiments, the specific modifications to the ITRs are exemplified herein (e.g., SEQ ID NOS: 3, 4, 15-47, 101-116 or 165-187, or shown in FIG. 7A-7B of International Patent Application No. PCT/US2018/064242, filed on Dec. 6, 2018 (e.g., SEQ ID Nos 97-98, 101-103, 105-108, 111-112, 117-134, 545-54 in PCT/US2018/064242). According to some embodiments, an ITR can be modified (e.g., by modifying 1, 2, 3, 4, 5, 6, 7, 8, 9, 10, 11, 12, 13, 14, 15, 16, 17, 18, 19, or 20 or more nucleotides or any range therein). In other embodiments, the ITR can have at least 80%, at least 85%, at least 90%, at least 95%, at least 96%, at least 97%, at least 98%, at least 99%, or more sequence identity with one of the modified ITRs of SEQ ID NOS: 3, 4, 15-47, 101-116 or 165-187, or the RBE-containing section of the A-A′ arm and C-C′ and B-B′ arms of SEQ ID NO: 3, 4, 15-47, 101-116 or 165-187, or shown in Tables 2-9 (i.e., SEQ ID NO: 110-112, 115-190, 200-468) of International Patent Application No. PCT/US18/49996, which is incorporated herein in its entirety by reference.

According to some embodiments, a modified ITR can for example, comprise removal or deletion of all of a particular arm, e.g., all or part of the A-A′ arm, or all or part of the B-B′ arm or all or part of the C-C′ arm, or alternatively, the removal of 1, 2, 3, 4, 5, 6, 7, 8, 9 or more base pairs forming the stem of the loop so long as the final loop capping the stem (e.g., single arm) is still present (e.g., see ITR-21 in FIG. 7A of PCT/US2018/064242, filed Dec. 6, 2018). According to some embodiments, a modified ITR can comprise the removal of 1, 2, 3, 4, 5, 6, 7, 8, 9 or more base pairs from the B-B′ arm. According to some embodiments, a modified ITR can comprise the removal of 1, 2, 3, 4, 5, 6, 7, 8, 9 or more base pairs from the C-C′ arm (see, e.g., ITR-1 in FIG. 3B, or ITR-45 in FIG. 7A of International Patent Application No. PCT/US2018/064242, filed Dec. 6, 2018). According to some embodiments, a modified ITR can comprise the removal of 1, 2, 3, 4, 5, 6, 7, 8, 9 or more base pairs from the C-C′ arm and the removal of 1, 2, 3, 4, 5, 6, 7, 8, 9 or more base pairs from the B-B′ arm. Any combination of removal of base pairs is envisioned, for example, 6 base pairs can be removed in the C-C′ arm and 2 base pairs in the B-B′ arm. As an illustrative example, FIG. 3B shows an exemplary modified ITR with at least 7 base pairs deleted from each of the C portion and the C′ portion, a substitution of a nucleotide in the loop between C and C′ region, and at least one base pair deletion from each of the B region and B′ regions such that the modified ITR comprises two arms where at least one arm (e.g., C-C′) is truncated. According to some embodiments, the modified ITR also comprises at least one base pair deletion from each of the B region and B′ regions, such that the B-B′ arm is also truncated relative to WT ITR.

According to some embodiments, a modified ITR can have between 1 and 50 (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50) nucleotide deletions relative to a full-length wild-type ITR sequence. According to some embodiments, a modified ITR can have between 1 and 30 nucleotide deletions relative to a full-length WT ITR sequence. According to some embodiments, a modified ITR has between 2 and 20 nucleotide deletions relative to a full-length wild-type ITR sequence.

According to some embodiments, a modified ITR does not contain any nucleotide deletions in the RBE-containing portion of the A or A′ regions, so as not to interfere with DNA replication (e.g., binding to an RBE by Rep protein, or nicking at a terminal resolution site). According to some embodiments, a modified ITR encompassed for use herein has one or more deletions in the B, B′, C, and/or C region as described herein.

According to some embodiments, a ceDNA vector for expression of antigens, or immunogenic peptides, comprising a symmetric ITR pair or asymmetric ITR pair comprises a regulatory switch as disclosed herein and at least one modified ITR selected having the nucleotide sequence selected from any of the group consisting of: SEQ ID NO: 3, 4, 15-47, 101-116 or 165-187.

In another embodiment, the structure of the structural element can be modified. For example, the structural element a change in the height of the stem and/or the number of nucleotides in the loop. For example, the height of the stem can be about 2, 3, 4, 5, 6, 7, 8, or 9 nucleotides or more or any range therein. According to some embodiments, the stem height can be about 5 nucleotides to about 9 nucleotides and functionally interacts with Rep. In another embodiment, the stem height can be about 7 nucleotides and functionally interacts with Rep. In another example, the loop can have 3, 4, 5, 6, 7, 8, 9, or 10 nucleotides or more or any range therein.

In another embodiment, the number of GAGY binding sites or GAGY-related binding sites within the RBE or extended RBE can be increased or decreased. According to some example, the RBE or extended RBE, can comprise 1, 2, 3, 4, 5, or 6 or more GAGY binding sites or any range therein. Each GAGY binding site can independently be an exact GAGY sequence or a sequence similar to GAGY as long as the sequence is sufficient to bind a Rep protein.

In another embodiment, the spacing between two elements (such as but not limited to the RBE and a hairpin) can be altered (e.g., increased or decreased) to alter functional interaction with a large Rep protein. For example, the spacing can be about 1, 2, 3, 4, 5, 6, 7, 8, 9, 10, 11, 12, 13, 14, 15, 16, 17, 18, 19, 20, or 21 nucleotides or more or any range therein.

The ceDNA vector for expression of antigens, or immunogenic peptides, as described herein can include an ITR structure that is modified with respect to the wild type AAV2 ITR structure disclosed herein, but still retains an operable RBE, trs and RBE′ portion. FIG. 2A and FIG. 2B show one possible mechanism for the operation of a trs site within a wild type ITR structure portion of a ceDNA vector for expression of antigens, or immunogenic peptides. According to some embodiments, the ceDNA vector for expression of antigens, or immunogenic peptides, contains one or more functional ITR polynucleotide sequences that comprise a Rep-binding site (RBS; 5′-GCGCGCTCGCTCGCTC-3′ for AAV2) and a terminal resolution site (TRS; 5′-AGTT). According to some embodiments, at least one ITR (wt or modified ITR) is functional. In alternative embodiments, where a ceDNA vector for expression of antigens, or immunogenic peptides, comprises two modified ITRs that are different or asymmetrical to each other, at least one modified ITR is functional and at least one modified ITR is non-functional.

According to some embodiments, the modified ITR (e.g., the left or right ITR) of a ceDNA vector for expression of antigens, or immunogenic peptides, as described herein has modifications within the loop arm, the truncated arm, or the spacer. Exemplary sequences of ITRs having modifications within the loop arm, the truncated arm, or the spacer are listed in Table 2 (i.e., SEQ ID NOS: 135-190, 200-233); Table 3 (e.g., SEQ ID Nos: 234-263); Table 4 (e.g., SEQ ID NOs: 264-293); Table 5 (e.g., SEQ ID Nos: 294-318 herein); Table 6 (e.g., SEQ ID NO: 319-468; and Tables 7-9 (e.g., SEQ ID Nos: 101-110, 111-112, 115-134) or Table 10A or 10B (e.g., SEQ ID Nos: 9, 100, 469-483, 484-499) of International Patent Application No. PCT/US18/49996, which is incorporated herein in its entirety by reference.

According to some embodiments, the modified ITR for use in a ceDNA vector for expression of antigens, or immunogenic peptides, comprising an asymmetric ITR pair, or symmetric mod-ITR pair is selected from any or a combination of those shown in Tables 2, 3, 4, 5, 6, 7, 8, 9 and 10A-10B of International Patent Application No. PCT/US18/49996 which is incorporated herein in its entirety by reference.

Additional exemplary modified ITRs for use in a ceDNA vector for expression of antigens, or immunogenic peptides, comprising an asymmetric ITR pair, or symmetric mod-ITR pair in each of the above classes are provided in Tables 11A and 11B. The predicted secondary structure of the Right modified ITRs in Table 11A are shown in FIG. 7A of International Patent Application No. PCT/US2018/064242, filed Dec. 6, 2018, and the predicted secondary structure of the Left modified ITRs in Table 11B are shown in FIG. 7B of International Patent Application No. PCT/US2018/064242, filed Dec. 6, 2018, which is incorporated herein in its entirety by reference.

Table 11A and Table 11B list the SEQ ID NOs of exemplary right and left modified ITRs.

TABLE 11A
Exemplary modified right ITRs. These exemplary modified right ITRs can comprise
the RBE of GCGCGCTCGCTCGCTC-3′ (spacer of ACTGAGGC), the spacer complement
GCCTCAGT and RBE′ (i.e., complement to RBE) of GAGCGAGCGAGCGCGC.

| ITR | | SEQ ID |
|---|---|---|
| Construct | Sequence | NO: |
| ITR-18 | AGGAACCCCTAGTGATGGAGTTGGCCACTCCCTCTCTGCGCGCTCG | 15 |
| Right | CTCGCTCACTGAGGCGCACGCCCGGGTTTCCCGGGCGGCCTCAGTG | |
| | AGCGAGCGAGCGCGCAGCTGCCTGCAGG | |
| ITR-19 | AGGAACCCCTAGTGATGGAGTTGGCCACTCCCTCTCTGCGCGCTCG | 16 |
| Right | CTCGCTCACTGAGGCCGACGCCCGGGCTTTGCCCGGGCGGCCTCA | |
| | GTGAGCGAGCGAGCGCGCAGCTGCCTGCAGG | |
| ITR-20 | AGGAACCCCTAGTGATGGAGTTGGCCACTCCCTCTCTGCGCGCTCG | 17 |
| Right | CTCGCTCACTGAGGCCGGGCGACCAAAGGTCGCCCGACGCCCGGG | |
| | CGCCTCAGTGAGCGAGCGAGCGCGCAGCTGCCTGCAGG | |
| ITR-21 | AGGAACCCCTAGTGATGGAGTTGGCCACTCCCTCTCTGCGCGCTCG | 18 |
| Right | CTCGCTCACTGAGGCTTTGCCTCAGTGAGCGAGCGAGCGCGCAGC | |
| | TGCCTGCAGG | |
| ITR-22 | AGGAACCCCTAGTGATGGAGTTGGCCACTCCCTCTCTGCGCGCTCG | 19 |
| Right | CTCGCTCACTGAGGCCGGGCGACAAAGTCGCCCGACGCCCGGGCT | |
| | TTGCCCGGGCGGCCTCAGTGAGCGAGCGAGCGCGCAGCTGCCTGC | |
| | AGG | |
| ITR-23 | AGGAACCCCTAGTGATGGAGTTGGCCACTCCCTCTCTGCGCGCTCG | 20 |
| Right | CTCGCTCACTGAGGCCGGGCGAAAATCGCCCGACGCCCGGGCTTT | |
| | GCCCGGGCGGCCTCAGTGAGCGAGCGAGCGCGCAGCTGCCTGCAG | |
| | G | |
| ITR-24 | AGGAACCCCTAGTGATGGAGTTGGCCACTCCCTCTCTGCGCGCTCG | 21 |
| Right | CTCGCTCACTGAGGCCGGGCGAAACGCCCGACGCCCGGGCTTTGC | |
| | CCGGGCGGCCTCAGTGAGCGAGCGAGCGCGCAGCTGCCTGCAGG | |
| ITR-25 | AGGAACCCCTAGTGATGGAGTTGGCCACTCCCTCTCTGCGCGCTCG | 22 |
| Right | CTCGCTCACTGAGGCCGGGCAAAGCCCGACGCCCGGGCTTTGCCC | |
| | GGGCGGCCTCAGTGAGCGAGCGAGCGCGCAGCTGCCTGCAGG | |
| ITR-26 | AGGAACCCCTAGTGATGGAGTTGGCCACTCCCTCTCTGCGCGCTCG | 23 |
| Right | CTCGCTCACTGAGGCCGGGCGACCAAAGGTCGCCCGACGCCCGGG | |
| | TTTCCCGGGCGGCCTCAGTGAGCGAGCGAGCGCGCAGCTGCCTGC | |
| | AGG | |
| ITR-27 | AGGAACCCCTAGTGATGGAGTTGGCCACTCCCTCTCTGCGCGCTCG | 24 |
| Right | CTCGCTCACTGAGGCCGGGCGACCAAAGGTCGCCCGACGCCCGGT | |
| | TTCCGGGCGGCCTCAGTGAGCGAGCGAGCGCGCAGCTGCCTGCAG | |
| | G | |
| ITR-28 | AGGAACCCCTAGTGATGGAGTTGGCCACTCCCTCTCTGCGCGCTCG | 25 |
| Right | CTCGCTCACTGAGGCCGGGCGACCAAAGGTCGCCCGACGCCCGTT | |
| | TCGGGCGGCCTCAGTGAGCGAGCGAGCGCGCAGCTGCCTGCAGG | |
| ITR-29 | AGGAACCCCTAGTGATGGAGTTGGCCACTCCCTCTCTGCGCGCTCG | 26 |
| Right | CTCGCTCACTGAGGCCGGGCGACCAAAGGTCGCCCGACGCCCTTT | |
| | GGGCGGCCTCAGTGAGCGAGCGAGCGCGCAGCTGCCTGCAGG | |
| ITR-30 | AGGAACCCCTAGTGATGGAGTTGGCCACTCCCTCTCTGCGCGCTCG | 27 |
| Right | CTCGCTCACTGAGGCCGGGCGACCAAAGGTCGCCCGACGCCTTTG | |
| | GCGGCCTCAGTGAGCGAGCGAGCGCGCAGCTGCCTGCAGG | |
| ITR-31 | AGGAACCCCTAGTGATGGAGTTGGCCACTCCCTCTCTGCGCGCTCG | 28 |
| Right | CTCGCTCACTGAGGCCGGGCGACCAAAGGTCGCCCGACGCTTTGC | |
| | GGCCTCAGTGAGCGAGCGAGCGCGCAGCTGCCTGCAGG | |
| ITR-32 | AGGAACCCCTAGTGATGGAGTTGGCCACTCCCTCTCTGCGCGCTCG | 29 |
| Right | CTCGCTCACTGAGGCCGGGCGACCAAAGGTCGCCCGACGTTTCGG | |
| | CCTCAGTGAGCGAGCGAGCGCGCAGCTGCCTGCAGG | |
| ITR-49 | AGGAACCCCTAGTGATGGAGTTGGCCACTCCCTCTCTGCGCGCTCG | 30 |
| Right | CTCGCTCACTGAGGCCGGGCGACCAAAGGTCGCCCGACGGCCTCA | |
| | GTGAGCGAGCGAGCGCGCAGCTGCCTGCAGG | |
| ITR-50 | AGGAACCCCTAGTGATGGAGTTGGCCACTCCCTCTCTGCGCGCTCG | 31 |
| right | CTCGCTCACTGAGGCCGGGCGACCAAAGGTCGCCCGACGCCCGGG | |
| | CGGCCTCAGTGAGCGAGCGAGCGCGCAGCTGCCTGCAGG | |

TABLE 11B
Exemplary modified left ITRs. These exemplary modified left ITRs can comprise the
RBE of GCGCGCTCGCTCGCTC-3′, spacer of ACTGAGGC, the spacer complement GCCTCAGT
and RBE complement (RBE′) of GAGCGAGCGAGCGCGC.

| | | |
|---|---|---|
| ITR-33 Left | CCTGCAGGCAGCTGCGCGCTCGCTCGCTCACTGAGGCCGCCCGGGAA | 32 |
| | ACCCGGGCGTGCGCCTCAGTGAGCGAGCGAGCGCGCAGAGAGGGAGT | |
| | GGCCAACTCCATCACTAGGGGTTCCT | |
| ITR-34 Left | CCTGCAGGCAGCTGCGCGCTCGCTCGCTCACTGAGGCCGTCGGGCGA | 33 |
| | CCTTTGGTCGCCCGGCCTCAGTGAGCGAGCGAGCGCGCAGAGAGGGA | |
| | GTGGCCAACTCCATCACTAGGGGTTCCT | |
| ITR-35 Left | CCTGCAGGCAGCTGCGCGCTCGCTCGCTCACTGAGGCCGCCCGGGCA | 34 |
| | AAGCCCGGGCGTCGGCCTCAGTGAGCGAGCGAGCGCGCAGAGAGGG | |
| | AGTGGCCAACTCCATCACTAGGGGTTCCT | |
| ITR-36 Left | CCTGCAGGCAGCTGCGCGCTCGCTCGCTCACTGAGGCGCCCGGGCGTC | 35 |
| | GGGCGACCTTTGGTCGCCCGGCCTCAGTGAGCGAGCGAGCGCGCAGA | |
| | GAGGGAGTGGCCAACTCCATCACTAGGGGTTCCT | |
| ITR-37 Left | CCTGCAGGCAGCTGCGCGCTCGCTCGCTCACTGAGGCAAAGCCTCAGT | 36 |
| | GAGCGAGCGAGCGCGCAGAGAGGGAGTGGCCAACTCCATCACTAGG | |
| | GGTTCCT | |
| ITR-38 Left | CCTGCAGGCAGCTGCGCGCTCGCTCGCTCACTGAGGCCGCCCGGGCA | 37 |
| | AAGCCCGGGCGTCGGGCGACTTTGTCGCCCGGCCTCAGTGAGCGAGC | |
| | GAGCGCGCAGAGAGGGAGTGGCCAACTCCATCACTAGGGGTTCCT | |
| ITR-39 Left | CCTGCAGGCAGCTGCGCGCTCGCTCGCTCACTGAGGCCGCCCGGGCA | 38 |
| | AAGCCCGGGCGTCGGGCGATTTTCGCCCGGCCTCAGTGAGCGAGCGA | |
| | GCGCGCAGAGAGGGAGTGGCCAACTCCATCACTAGGGGTTCCT | |
| ITR-40 Left | CCTGCAGGCAGCTGCGCGCTCGCTCGCTCACTGAGGCCGCCCGGGCA | 39 |
| | AAGCCCGGGCGTCGGGCGTTTCGCCCGGCCTCAGTGAGCGAGCGAGC | |
| | GCGCAGAGAGGGAGTGGCCAACTCCATCACTAGGGGTTCCT | |
| ITR-41 Left | CCTGCAGGCAGCTGCGCGCTCGCTCGCTCACTGAGGCCGCCCGGGCA | 40 |
| | AAGCCCGGGCGTCGGGCTTTGCCCGGCCTCAGTGAGCGAGCGAGCGC | |
| | GCAGAGAGGGAGTGGCCAACTCCATCACTAGGGGTTCCT | |
| ITR-42 Left | CCTGCAGGCAGCTGCGCGCTCGCTCGCTCACTGAGGCCGCCCGGGAA | 41 |
| | ACCCGGGCGTCGGGCGACCTTTGGTCGCCCGGCCTCAGTGAGCGAGC | |
| | GAGCGCGCAGAGAGGGAGTGGCCAACTCCATCACTAGGGGTTCCT | |
| ITR-43 Left | CCTGCAGGCAGCTGCGCGCTCGCTCGCTCACTGAGGCCGCCCGGAAA | 42 |
| | CCGGGCGTCGGGCGACCTTTGGTCGCCCGGCCTCAGTGAGCGAGCGA | |
| | GCGCGCAGAGAGGGAGTGGCCAACTCCATCACTAGGGGTTCCT | |
| ITR-44 Left | CCTGCAGGCAGCTGCGCGCTCGCTCGCTCACTGAGGCCGCCCGAAAC | 43 |
| | GGGCGTCGGGCGACCTTTGGTCGCCCGGCCTCAGTGAGCGAGCGAGC | |
| | GCGCAGAGAGGGAGTGGCCAACTCCATCACTAGGGGTTCCT | |
| ITR-45 Left | CCTGCAGGCAGCTGCGCGCTCGCTCGCTCACTGAGGCCGCCCAAAGG | 44 |
| | GCGTCGGGCGACCTTTGGTCGCCCGGCCTCAGTGAGCGAGCGAGCGC | |
| | GCAGAGAGGGAGTGGCCAACTCCATCACTAGGGGTTCCT | |
| ITR-46 Left | CCTGCAGGCAGCTGCGCGCTCGCTCGCTCACTGAGGCCGCCAAAGGC | 45 |
| | GTCGGGCGACCTTTGGTCGCCCGGCCTCAGTGAGCGAGCGAGCGCGC | |
| | AGAGAGGGAGTGGCCAACTCCATCACTAGGGGTTCCT | |
| ITR-47 Left | CCTGCAGGCAGCTGCGCGCTCGCTCGCTCACTGAGGCCGCAAAGCGT | 46 |
| | CGGGCGACCTTTGGTCGCCCGGCCTCAGTGAGCGAGCGAGCGCGCAG | |
| | AGAGGGAGTGGCCAACTCCATCACTAGGGGTTCCT | |
| ITR-48 Left | CCTGCAGGCAGCTGCGCGCTCGCTCGCTCACTGAGGCCGAAACGTCG | 47 |
| | GGCGACCTTTGGTCGCCCGGCCTCAGTGAGCGAGCGAGCGCGCAGAG | |
| | AGGGAGTGGCCAACTCCATCACTAGGGGTTCCT | |

According to some embodiments, a ceDNA vector for expression of antigens, or immunogenic peptides, comprises, in the 5′ to 3′ direction: a first adeno-associated virus (AAV) inverted terminal repeat (ITR), a nucleic acid sequence of interest (for example an expression cassette as described herein) and a second AAV ITR, where the first ITR (5′ ITR) and the second ITR (3′ ITR) are asymmetric with respect to each other—that is, they have a different 3D-spatial configuration from one another. As an exemplary embodiment, the first ITR can be a wild-type ITR and the second ITR can be a mutated or modified ITR, or vice versa, where the first ITR can be a mutated or modified ITR and the second ITR a wild-type ITR. According to some embodiments, the first ITR and the second ITR are both mod-ITRs, but have different sequences, or have different modifications, and thus are not the same modified ITRs, and have different 3D spatial configurations. Stated differently, a ceDNA vector with asymmetric ITRs comprises ITRs where any changes According to some ITR relative to the WT-ITR are not reflected in the other ITR; or alternatively, where the asymmetric ITRs have a modified asymmetric ITR pair can have a different sequence and different three-dimensional shape with respect to each other. Exemplary asymmetric ITRs in the ceDNA vector for expression of antigens, or immunogenic peptides, and for use to generate a ceDNA-plasmid are shown in Table 11A and 11B.

In an alternative embodiment, a ceDNA vector for expression of antigens, or immunogenic peptides, comprises two symmetrical mod-ITRs—that is, both ITRs have the same sequence, but are reverse complements (inverted) of each other. According to some embodiments, a symmetrical mod-ITR pair comprises at least one or any combination of a deletion, insertion, or substitution relative to wild type ITR sequence from the same AAV serotype. The additions, deletions, or substitutions in the symmetrical ITR are the same but the reverse complement of each other. For example, an insertion of 3 nucleotides in the C region of the 5′ ITR would be reflected in the insertion of 3 reverse complement nucleotides in the corresponding section in the C′ region of the 3′ ITR. Solely for illustration purposes only, if the addition is AACG in the 5′ ITR, the addition is CGTT in the 3′ ITR at the corresponding site. For example, if the 5′ ITR sense strand is ATCGATCG with an addition of AACG between the G and A to result in the sequence ATCGAACGATCG. The corresponding 3′ ITR sense strand is CGATCGAT (the reverse complement of ATCGATCG) with an addition of CGTT (i.e. the reverse complement of AACG) between the T and C to result in the sequence CGATCGTTCGAT (the reverse complement of ATCGAACGATCG).

In alternative embodiments, the modified ITR pair are substantially symmetrical as defined herein—that is, the modified ITR pair can have a different sequence but have corresponding or the same symmetrical three-dimensional shape. For example, one modified ITR can be from one serotype and the other modified ITR be from a different serotype, but they have the same mutation (e.g., nucleotide insertion, deletion or substitution) in the same region. Stated differently, for illustrative purposes only, a 5′ mod-ITR can be from AAV2 and have a deletion in the C region, and the 3′ mod-ITR can be from AAV5 and have the corresponding deletion in the C′ region, and provided the 5′ mod-ITR and the 3′ mod-ITR have the same or symmetrical three-dimensional spatial organization, they are encompassed for use herein as a modified ITR pair.

According to some embodiments, a substantially symmetrical mod-ITR pair has the same A, C-C′ and B-B′ loops in 3D space, e.g., if a modified ITR in a substantially symmetrical mod-ITR pair has a deletion of a C-C′ arm, then the cognate mod-ITR has the corresponding deletion of the C-C′ loop and also has a similar 3D structure of the remaining A and B-B′ loops in the same shape in geometric space of its cognate mod-ITR. By way of example only, substantially symmetrical ITRs can have a symmetrical spatial organization such that their structure is the same shape in geometrical space. This can occur, e.g., when a G-C pair is modified, for example, to a C-G pair or vice versa, or A-T pair is modified to a T-A pair, or vice versa. Therefore, using the exemplary example above of modified 5′ ITR as a ATCGAACGATCG, and modified 3′ ITR as CGATCGTTCGAT (i.e., the reverse complement of ATCGAACGATCG), these modified ITRs would still be symmetrical if, for example, the 5′ ITR had the sequence of ATCGAACCATCG, where G in the addition is modified to C, and the substantially symmetrical 3′ ITR has the sequence of CGATCGTTCGAT, without the corresponding modification of the T in the addition to a. According to some embodiments, such a modified ITR pair are substantially symmetrical as the modified ITR pair has symmetrical stereochemistry.

Table 12 shows exemplary symmetric modified ITR pairs (i.e., a left modified ITRs and the symmetric right modified ITR) for use in a ceDNA vector for expression of antigens, or immunogenic peptides. The bold (red) portion of the sequences identify partial ITR sequences (i.e., sequences of A-A′, C-C′ and B-B′ loops), also shown in FIGS. 31A-46B. These exemplary modified ITRs can comprise the RBE of GCGCGCTCGCTCGCTC-3′, spacer of ACTGAGGC, the spacer complement and RBE′ (i.e., complement to RBE) of GAGCGAGCGAGCGCGC.

TABLE 12
Exemplary symmetric modified ITR pairs in a ceDNA vector for expression of antigens, or
immunogenic peptides.
Exemplary symmetric modified ITR pairs in a ceDNA vector for expression of FIX protein

| LEFT modified ITR | | Symmetric RIGHT modified ITR | |
|---|---|---|---|
| (modified 5′ ITR) | | (modified 3′ ITR) | |
| SEQ ID | CCTGCAGGCAGCTGCGCGCTCG | SEQ ID NO: 15 | AGGAACCCCTAGTGATGG |
| NO:32 | CTCGCTCACTGAGGCCGCCCG | (ITR-18, right) | AGTTGGCCACTCCCTCTCT |
| (ITR-33 | GGAAACCCGGGCGTGCGCCTC | | GCGCGCTCGCTCGCTCA |
| left) | AGTGAGCGAGCGAGCGCGCAG | | CTGAGGCGCACGCCCGG |
| | AGAGGGAGTGGCCAACTCCATC | | GTTTCCCGGGCGGCCTC |
| | ACTAGGGGTTCCT | | AGTGAGCGAGCGAGCGC |
| | | | GCAGCTGCCTGCAGG |
| SEQ ID | CCTGCAGGCAGCTGCGCGCTCG | SEQ ID NO: 48 | AGGAACCCCTAGTGATGG |
| NO: 33 | CTCGCTCACTGAGGCCGTCGG | (ITR-51, right) | AGTTGGCCACTCCCTCTCT |
| (ITR-34 | GCGACCTTTGGTCGCCCGGCC | | GCGCGCTCGCTCGCTCA |
| left) | TCAGTGAGCGAGCGAGCGCGC | | CTGAGGCCGGGCGACCA |
| | AGAGAGGGAGTGGCCAACTCCA | | AAGGTCGCCCGACGGCC |
| | TCACTAGGGGTTCCT | | TCAGTGAGCGAGCGAGC |
| | | | GCGCAGCTGCCTGCAGG |
| SEQ ID | CCTGCAGGCAGCTGCGCGCTCG | SEQ ID NO: 16 | AGGAACCCCTAGTGATGG |
| NO: 34 | CTCGCTCACTGAGGCCGCCCG | (ITR-19, right) | AGTTGGCCACTCCCTCTCT |
| (ITR-35 | GGCAAAGCCCGGGCGTCGGCC | | GCGCGCTCGCTCGCTCA |
| left) | TCAGTGAGCGAGCGAGCGCGC | | CTGAGGCCGACGCCCGG |
| | AGAGAGGGAGTGGCCAACTCCA | | GCTTTGCCCGGGCGGCC |
| | TCACTAGGGGTTCCT | | TCAGTGAGCGAGCGAGC |
| | | | GCGCAGCTGCCTGCAGG |
| SEQ ID | CCTGCAGGCAGCTGCGCGCTCG | SEQ ID NO: 17 | AGGAACCCCTAGTGATGG |
| NO: 35 | CTCGCTCACTGAGGCGCCCGG | (ITR-20, right) | AGTTGGCCACTCCCTCTCT |
| (ITR-36 | GCGTCGGGCGACCTTTGGTCG | | GCGCGCTCGCTCGCTCA |
| left) | CCCGGCCTCAGTGAGCGAGCG | | CTGAGGCCGGGCGACCA |
| | AGCGCGCAGAGAGGGAGTGGC | | AAGGTCGCCCGACGCCC |
| | CAACTCCATCACTAGGGGTTCCT | | GGGCGCCTCAGTGAGCG |
| | | | AGCGAGCGCGCAGCTGC |
| | | | CTGCAGG |
| SEQ ID | CCTGCAGGCAGCTGCGCGCTCG | SEQ ID NO: 18 | AGGAACCCCTAGTGATGG |
| NO: 36 | CTCGCTCACTGAGGCAAAGCC | (ITR-21, right) | AGTTGGCCACTCCCTCTCT |
| (ITR-37 | TCAGTGAGCGAGCGAGCGCGC | | GCGCGCTCGCTCGCTCA |
| left) | AGAGAGGGAGTGGCCAACTCCA | | CTGAGGCTTTGCCTCAG |
| | TCACTAGGGGTTCCT | | TGAGCGAGCGAGCGCGC |
| | | | AGCTGCCTGCAGG |
| SEQ ID | CCTGCAGGCAGCTGCGCGCTCG | SEQ ID NO: 19 | AGGAACCCCTAGTGATGG |
| NO: 37 | CTCGCTCACTGAGGCCGCCCG | (ITR-22 right) | AGTTGGCCACTCCCTCTCT |
| (ITR-38 | GGCAAAGCCCGGGCGTCGGGC | | GCGCGCTCGCTCGCTCA |
| left) | GACTTTGTCGCCCGGCCTCAG | | CTGAGGCCGGGCGACAA |
| | TGAGCGAGCGAGCGCGCAGAG | | AGTCGCCCGACGCCCGG |
| | AGGGAGTGGCCAACTCCATCAC | | GCTTTGCCCGGGCGGCC |
| | TAGGGGTTCCT | | TCAGTGAGCGAGCGAGC |
| | | | GCGCAGCTGCCTGCAGG |
| SEQ ID | CCTGCAGGCAGCTGCGCGCTCG | SEQ ID NO: 20 | AGGAACCCCTAGTGATGG |
| NO: 38 | CTCGCTCACTGAGGCCGCCCG | (ITR-23, right) | AGTTGGCCACTCCCTCTC |
| (ITR-39 | GGCAAAGCCCGGGCGTCGGGC | | TGCGCGCTCGCTCGCTCA |
| left) | GATTTTCGCCCGGCCTCAGTG | | CTGAGGCCGGGCGAAAA |
| | AGCGAGCGAGCGCGCAGAGAG | | TCGCCCGACGCCCGGGC |
| | GGAGTGGCCAACTCCATCACTA | | TTTGCCCGGGCGGCCTC |
| | GGGGTTCCT | | AGTGAGCGAGCGAGCGC |
| | | | GCAGCTGCCTGCAGG |
| SEQ ID | CCTGCAGGCAGCTGCGCGCTCG | SEQ ID NO: 21 | AGGAACCCCTAGTGATGG |
| NO: 39 | CTCGCTCACTGAGGCCGCCCG | (ITR-24, right) | AGTTGGCCACTCCCTCTC |
| (ITR-40 | GGCAAAGCCCGGGCGTCGGGC | | TGCGCGCTCGCTCGCTCA |
| left) | GTTTCGCCCGGCCTCAGTGAG | | CTGAGGCCGGGCGAAAC |
| | CGAGCGAGCGCGCAGAGAGGG | | GCCCGACGCCCGGGCTT |
| | AGTGGCCAACTCCATCACTAGG | | TGCCCGGGCGGCCTCAG |
| | GGTTCCT | | TGAGCGAGCGAGCGCGC |
| | | | AGCTGCCTGCAGG |
| SEQ ID | CCTGCAGGCAGCTGCGCGCTCG | SEQ ID NO: 22 | AGGAACCCCTAGTGATGG |
| NO: 40 | CTCGCTCACTGAGGCCGCCCG | (ITR-25 right) | AGTTGGCCACTCCCTCTCT |
| (ITR-41 | GGCAAAGCCCGGGCGTCGGGC | | GCGCGCTCGCTCGCTCA |
| left) | TTTGCCCGGCCTCAGTGAGCG | | CTGAGGCCGGGCAAAGC |
| | AGCGAGCGCGCAGAGAGGGAG | | CCGACGCCCGGGCTTTG |
| | TGGCCAACTCCATCACTAGGGGT | | CCCGGGCGGCCTCAGTG |
| | TCCT | | AGCGAGCGAGCGCGCAG |
| | | | CTGCCTGCAGG |
| SEQ ID | CCTGCAGGCAGCTGCGCGCTCG | SEQ ID NO: 23 | AGGAACCCCTAGTGATGG |
| NO: 41 | CTCGCTCACTGAGGCCGCCCG | (ITR-26 right) | AGTTGGCCACTCCCTCTC |
| (ITR-42 | GGAAACCCGGGCGTCGGGCGA | | TGCGCGCTCGCTCGCTCA |
| left) | CCTTTGGTCGCCCGGCCTCAG | | CTGAGGCCGGGCGACCA |
| | TGAGCGAGCGAGCGCGCAGAG | | AAGGTCGCCCGACGCCC |
| | AGGGAGTGGCCAACTCCATCAC | | GGGTTTCCCGGGCGGCC |
| | TAGGGGTTCCT | | TCAGTGAGCGAGCGAGC |
| | | | GCGCAGCTGCCTGCAGG |
| SEQ ID | CCTGCAGGCAGCTGCGCGCTCG | SEQ ID NO: 24 | AGGAACCCCTAGTGATGG |
| NO: | CTCGCTCACTGAGGCCGCCCG | (ITR-27 right) | AGTTGGCCACTCCCTCTCT |
| 42(ITR-43 | GAAACCGGGCGTCGGGCGACC | | GCGCGCTCGCTCGCTCA |
| left) | TTTGGTCGCCCGGCCTCAGTG | | CTGAGGCCGGGCGACCA |
| | AGCGAGCGAGCGCGCAGAGAG | | AAGGTCGCCCGACGCCC |
| | GGAGTGGCCAACTCCATCACTA | | GGTTTCCGGGCGGCCTC |
| | GGGGTTCCT | | AGTGAGCGAGCGAGCGC |
| | | | GCAGCTGCCTGCAGG |
| SEQ ID | CCTGCAGGCAGCTGCGCGCTCG | SEQ ID NO: 25 | AGGAACCCCTAGTGATGG |
| NO: 43 | CTCGCTCACTGAGGCCGCCCG | (ITR-28 right) | AGTTGGCCACTCCCTCTC |
| (ITR-44 | AAACGGGCGTCGGGCGACCTT | | TGCGCGCTCGCTCGCTCA |
| left) | TGGTCGCCCGGCCTCAGTGAG | | CTGAGGCCGGGCGACCA |
| | CGAGCGAGCGCGCAGAGAGGG | | AAGGTCGCCCGACGCCC |
| | AGTGGCCAACTCCATCACTAGG | | GTTTCGGGCGGCCTCAG |
| | GGTTCCT | | TGAGCGAGCGAGCGCGC |
| | | | AGCTGCCTGCAGG |
| SEQ ID | CCTGCAGGCAGCTGCGCGCTCG | SEQ ID NO:26 | AGGAACCCCTAGTGATGG |
| NO:44 | CTCGCTCACTGAGGCCGCCCA | (ITR-29, right) | AGTTGGCCACTCCCTCTC |
| (ITR-45 | AAGGGCGTCGGGCGACCTTTG | | TGCGCGCTCGCTCGCTCA |
| left) | GTCGCCCGGCCTCAGTGAGCG | | CTGAGGCCGGGCGACCA |
| | AGCGAGCGCGCAGAGAGGGAG | | AAGGTCGCCCGACGCCC |
| | TGGCCAACTCCATCACTAGGGGT | | TTTGGGCGGCCTCAGTG |
| | TCCT | | AGCGAGCGAGCGCGCAG |
| | | | CTGCCTGCAGG |
| SEQ ID | CCTGCAGGCAGCTGCGCGCTCG | SEQ ID NO: | AGGAACCCCTAGTGATGG |
| NO:45 | CTCGCTCACTGAGGCCGCCAA | 27(ITR-30, right) | AGTTGGCCACTCCCTCTCT |
| (ITR-46 | AGGCGTCGGGCGACCTTTGGT | | GCGCGCTCGCTCGCTCA |
| left) | CGCCCGGCCTCAGTGAGCGAG | | CTGAGGCCGGGCGACCA |
| | CGAGCGCGCAGAGAGGGAGTG | | AAGGTCGCCCGACGCCT |
| | GCCAACTCCATCACTAGGGGTTC | | TTGGCGGCCTCAGTGAG |
| | CT | | CGAGCGAGCGCGCAGCT |
| | | | GCCTGCAGG |
| SEQ ID | CCTGCAGGCAGCTGCGCGCTCG | SEQ ID NO: 28 | AGGAACCCCTAGTGATGG |
| NO: 46 | CTCGCTCACTGAGGCCGCAAA | (ITR-31, right) | AGTTGGCCACTCCCTCTCT |
| (ITR-47, | GCGTCGGGCGACCTTTGGTCG | | GCGCGCTCGCTCGCTCA |
| left) | CCCGGCCTCAGTGAGCGAGCG | | CTGAGGCCGGGCGACCA |
| | AGCGCGCAGAGAGGGAGTGGC | | AAGGTCGCCCGACGCTT |
| | CAACTCCATCACTAGGGGTTCCT | | TGCGGCCTCAGTGAGCG |
| | | | AGCGAGCGCGCAGCTGC |
| | | | CTGCAGG |
| SEQ ID | CCTGCAGGCAGCTGCGCGCTCG | SEQ ID NO: 29 | AGGAACCCCTAGTGATGG |
| NO: 47 | CTCGCTCACTGAGGCCGAAAC | (ITR-32 right) | AGTTGGCCACTCCCTCTCT |
| (ITR-48, | GTCGGGCGACCTTTGGTCGCC | | GCGCGCTCGCTCGCTCA |
| left) | CGGCCTCAGTGAGCGAGCGAG | | CTGAGGCCGGGCGACCA |
| | CGCGCAGAGAGGGAGTGGCCA | | AAGGTCGCCCGACGTTT |
| | ACTCCATCACTAGGGGTTCCT | | CGGCCTCAGTGAGCGAG |
| | | | CGAGCGCGCAGCTGCCT |
| | | | GCAGG |

According to some embodiments, a ceDNA vector for expression of antigens, or immunogenic peptides, comprising an asymmetric ITR pair can comprise an ITR with a modification corresponding to any of the modifications in ITR sequences or ITR partial sequences shown in any one or more of Tables 11A-11B herein, or the sequences shown in FIG. 7A-7B of International Patent Application No. PCT/US2018/064242, filed Dec. 6, 2018, which is incorporated herein in its entirety, or disclosed in Tables 2, 3, 4, 5, 6, 7, 8, 9 or 10A-10B of International Patent Application No. PCT/US18/49996 filed Sep. 7, 2018 which is incorporated herein in its entirety by reference.

Exemplary ceDNA Vectors

As described above, the present disclosure relates to recombinant ceDNA expression vectors and ceDNA vectors that encode antigens, or immunogenic peptides, comprising any one of: an asymmetrical ITR pair, a symmetrical ITR pair, or substantially symmetrical ITR pair as described above. In certain embodiments, the disclosure relates to recombinant ceDNA vectors for expression of antigens, or immunogenic peptides, having flanking ITR sequences and a transgene, where the ITR sequences are asymmetrical, symmetrical or substantially symmetrical relative to each other as defined herein, and the ceDNA further comprises a nucleic acid sequence of interest (for example an expression cassette comprising the nucleic acid of a transgene) located between the flanking ITRs, wherein said nucleic acid molecule is devoid of viral capsid protein coding sequences.

The ceDNA expression vector for expression of antigens, or immunogenic peptides, may be any ceDNA vector that can be conveniently subjected to recombinant DNA procedures including nucleic acid sequence(s) as described herein, provided at least one ITR is altered. The ceDNA vectors for expression of antigens, or immunogenic peptides, of the present disclosure are compatible with the host cell into which the ceDNA vector is to be introduced. In certain embodiments, the ceDNA vectors may be linear. In certain embodiments, the ceDNA vectors may exist as an extrachromosomal entity. In certain embodiments, the ceDNA vectors of the present disclosure may contain an element(s) that permits integration of a donor sequence into the host cell's genome. As used herein “transgene”, “nucleic acid sequence” and “heterologous nucleic acid sequence” are synonymous, and encode antigens, or immunogenic peptides, as described herein.

Referring now to FIGS. 1A-1G, schematics of the functional components of two non-limiting plasmids useful in making a ceDNA vector for expression of antigens, or immunogenic peptides, are shown. FIG. 1A, 1B, 1D, 1F show the construct of ceDNA vectors or the corresponding sequences of ceDNA plasmids for expression of antigens, or immunogenic peptides. ceDNA vectors are capsid-free and can be obtained from a plasmid encoding in this order: a first ITR, an expressible transgene cassette and a second ITR, where the first and second ITR sequences are asymmetrical, symmetrical or substantially symmetrical relative to each other as defined herein. ceDNA vectors for expression of antigens, or immunogenic peptides, are capsid-free and can be obtained from a plasmid encoding in this order: a first ITR, an expressible transgene (protein or nucleic acid) and a second ITR, where the first and second ITR sequences are asymmetrical, symmetrical or substantially symmetrical relative to each other as defined herein. According to some embodiments, the expressible transgene cassette includes, as needed: an enhancer/promoter, one or more homology arms, a donor sequence, a post-transcription regulatory element (e.g., WPRE, e.g., SEQ ID NO: 67)), and a polyadenylation and termination signal (e.g., BGH polyA, e.g., SEQ ID NO: 68).

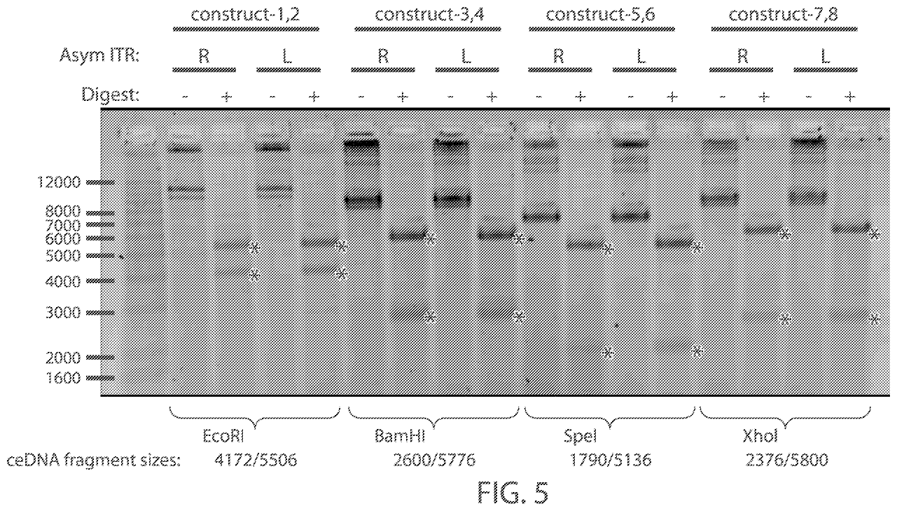

FIG. 5 is a gel confirming the production of ceDNA from multiple plasmid constructs using the method described in the Examples. The ceDNA is confirmed by a characteristic band pattern in the gel, as discussed with respect to FIG. 4A above and in the Examples.

### Regulatory Elements

The ceDNA vectors for expression of antigens, or immunogenic peptides, as described herein comprising an asymmetric ITR pair or symmetric ITR pair as defined herein, can further comprise a specific combination of cis-regulatory elements. The cis-regulatory elements include, but are not limited to, a promoter, a riboswitch, an insulator, a mir-regulatable element, a post-transcriptional regulatory element, a tissue- and cell type-specific promoter and an enhancer.

According to some embodiments, sequences of various cis-regulatory elements can be selected from any of those disclosed in International Application No. PCT/US2021/023891, filed on Mar. 24, 2021, the contents of which are incorporated by reference in its entirety herein.

In embodiments, the second nucleic acid sequence includes a regulatory sequence, and a nucleic acid sequence encoding a nuclease. In certain embodiments the gene regulatory sequence is operably linked to the nucleic acid sequence encoding the nuclease. In certain embodiments, the regulatory sequence is suitable for controlling the expression of the nuclease in a host cell. In certain embodiments, the regulatory sequence includes a suitable promoter sequence, being able to direct transcription of a gene operably linked to the promoter sequence, such as a nucleic acid sequence encoding the nuclease(s) of the present disclosure. In certain embodiments, the second nucleic acid sequence includes an intron sequence linked to the 5′ terminus of the nucleic acid sequence encoding the nuclease. In certain embodiments, an enhancer sequence is provided upstream of the promoter to increase the efficacy of the promoter. In certain embodiments, the regulatory sequence includes an enhancer and a promoter, wherein the second nucleic acid sequence includes an intron sequence upstream of the nucleic acid sequence encoding a nuclease, wherein the intron includes one or more nuclease cleavage site(s), and wherein the promoter is operably linked to the nucleic acid sequence encoding the nuclease.

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6, e.g., SEQ ID NO: 80) (Miyagishi et al., Nature Biotechnology 20, 497-500 (2002)), an enhanced U6 promoter (e.g., Xia et al., Nucleic Acids Res. 2003 Sep. 1; 31(17)), a human H1 promoter (H1) (e.g., SEQ ID NO: 81 or SEQ ID NO: 155), a CAG promoter, a human alpha 1-antitypsin (HAAT) promoter (e.g., SEQ ID NO: 82), and the like. In certain embodiments, these promoters are altered at their downstream intron containing end to include one or more nuclease cleavage sites. In certain embodiments, the DNA containing the nuclease cleavage site(s) is foreign to the promoter DNA.

According to some embodiments, a promoter may also be a promoter from a human gene such as human ubiquitin C (hUbC), human actin, human myosin, human hemoglobin, human muscle creatine, or human metallothionein.

According to some embodiments, the promoter is a tissue-specific promoter. According to further embodiments, the tissue-specific promoter is a liver specific promoter. According to some embodiments, the antigen, or immunogenic protein, is targeted to the liver and/or produced in the liver by the liver specific promoter.

Any liver specific promoter known in the art is contemplated for use in the present disclosure. According to some embodiments, the liver specific promoter is selected from, but not limited to, human alpha 1-antitypsin (HAAT), natural or synthetic. According to some embodiments, delivery to the liver can be achieved using endogenous ApoE specific targeting of the composition comprising a ceDNA vector to hepatocytes via the low density lipoprotein (LDL) receptor present on the surface of the hepatocyte.

Non-limiting examples of suitable promoters for use in accordance with the present disclosure include, but are not limited to, any of the following: the CAG promoter, the EF1a promoter, IE2 promoter and the rat EF1-α promoter, mEFI promoter, or 1E1 promoter fragment.

According to some embodiments, a promoter can be selected from any promoter sequence disclosed in International Application No. PCT/US2021/023891, filed on Mar. 24, 2021, the contents of which are incorporated by reference in its entirety herein.

### Polyadenylation Sequences:

A sequence encoding a polyadenylation sequence can be included in the ceDNA vector for expression of antigens, or immunogenic peptides, to stabilize an mRNA expressed from the ceDNA vector, and to aid in nuclear export and translation. According to some embodiments, the ceDNA vector does not include a polyadenylation sequence. In other embodiments, the ceDNA vector for expression of antigens, or immunogenic peptides, includes at least 1, at least 2, at least 3, at least 4, at least 5, at least 10, at least 15, at least 20, at least 25, at least 30, at least 40, least 45, at least 50 or more adenine dinucleotides. According to some embodiments, the polyadenylation sequence comprises about 43 nucleotides, about 40-50 nucleotides, about 40-55 nucleotides, about 45-50 nucleotides, about 35-50 nucleotides, or any range there between.

The expression cassettes can include any poly-adenylation sequence known in the art or a variation thereof. Some expression cassettes can also include SV40 late polyA signal upstream enhancer (USE) sequence. According to some embodiments, a USE sequence can be used in combination with SV40 pA or heterologous poly-A signal. PolyA sequences are located 3′ of the transgene encoding the antigens, or immunogenic peptides.

According to some embodiments, a polyadenylation sequence can be selected from any polyadenylation sequence disclosed in International Application No. PCT/US2021/023891, filed on Mar. 24, 2021, the contents of which are incorporated by reference in its entirety herein.

The expression cassettes can also include a post-transcriptional element to increase the expression of a transgene. According to some embodiments, Woodchuck Hepatitis Virus (WHP) posttranscriptional regulatory element (WPRE) is used to increase the expression of a transgene. Other posttranscriptional processing elements such as the post-transcriptional element from the thymidine kinase gene of herpes simplex virus, or hepatitis B virus (HBV) can be used.

According to some embodiments, a posttranscritptional regulatory element can be selected from any posttranscriptional regulatory element sequence disclosed in International Application No. PCT/US2021/023891, filed on Mar. 24, 2021, the contents of which are incorporated by reference in its entirety herein.

According to some embodiments, one or more nucleic acid sequences that encode an antigen, or immunogenic protein, can also encode a secretory sequence so that the protein is directed to the Golgi Apparatus and Endoplasmic Reticulum and folded into the correct conformation by chaperone molecules as it passes through the ER and out of the cell. Exemplary secretory sequences include, but are not limited to VH-02 and VK-A26) and IgK signal sequence, as well as a Glue secretory signal that allows the tagged protein to be secreted out of the cytosol, TMD-ST secretory sequence, that directs the tagged protein to the golgi.

According to some embodiments, a secretory sequence can be selected from any secretory sequence disclosed in International Application No. PCT/US2021/023891, filed on Mar. 24, 2021, the contents of which are incorporated by reference in its entirety herein.

### Nuclear Localization Sequences

According to some embodiments, the ceDNA vector for expression of antigens, or immunogenic peptides, comprises one or more nuclear localization sequences (NLSs), for example, 1, 2, 3, 4, 5, 6, 7, 8, 9, 10, or more NLSs. According to some embodiments, the one or more NLSs are located at or near the amino-terminus, at or near the carboxy-terminus, or a combination of these (e.g., one or more NLS at the amino-terminus and/or one or more NLS at the carboxy terminus). When more than one NLS is present, each can be selected independently of the others, such that a single NLS is present in more than one copy and/or in combination with one or more other NLSs present According to some or more copies.

According to some embodiments, a NLS can be selected from any NLS disclosed in International Application No. PCT/US2021/023891, filed on Mar. 24, 2021, the contents of which are incorporated by reference in its entirety herein.

V. Method of Production of a ceDNA Vector

### Production in General

Certain methods for the production of a ceDNA vector for expression of antigens, or immunogenic peptides, comprising an asymmetrical ITR pair or symmetrical ITR pair as defined herein is described in section IV of International application PCT/US18/49996 filed Sep. 7, 2018, which is incorporated herein in its entirety by reference. According to some embodiments, a ceDNA vector for expression of antigens, or immunogenic peptides, as disclosed herein can be produced using insect cells, as described herein. In alternative embodiments, a ceDNA vector for expression of antigens, or immunogenic peptides, as disclosed herein can be produced synthetically and according to some embodiments, in a cell-free method, as disclosed in International Application PCT/US19/14122, filed Jan. 18, 2019, which is incorporated herein in its entirety by reference.

As described herein, according to some embodiments, a ceDNA vector for expression of antigens, or immunogenic peptides, can be obtained, for example, by the process comprising the steps of: a) incubating a population of host cells (e.g., insect cells) harboring the polynucleotide expression construct template (e.g., a ceDNA-plasmid, a ceDNA-Bacmid, and/or a ceDNA-baculovirus), which is devoid of viral capsid coding sequences, in the presence of a Rep protein under conditions effective and for a time sufficient to induce production of the ceDNA vector within the host cells, and wherein the host cells do not comprise viral capsid coding sequences; and b) harvesting and isolating the ceDNA vector from the host cells. The presence of Rep protein induces replication of the vector polynucleotide with a modified ITR to produce the ceDNA vector in a host cell. However, no viral particles (e.g., AAV virions) are expressed. Thus, there is no size limitation such as that naturally imposed in AAV or other viral-based vectors.

The presence of the ceDNA vector isolated from the host cells can be confirmed by digesting DNA isolated from the host cell with a restriction enzyme having a single recognition site on the ceDNA vector and analyzing the digested DNA material on a non-denaturing gel to confirm the presence of characteristic bands of linear and continuous DNA as compared to linear and non-continuous DNA.

In yet another aspect, the disclosure provides for use of host cell lines that have stably integrated the DNA vector polynucleotide expression template (ceDNA template) into their own genome in production of the non-viral DNA vector, e.g., as described in Lee, L. et al. (2013) Plos One 8(8): e69879. Preferably, Rep is added to host cells at an MOI of about 3. When the host cell line is a mammalian cell line, e.g., HEK293 cells, the cell lines can have polynucleotide vector template stably integrated, and a second vector such as herpes virus can be used to introduce Rep protein into cells, allowing for the excision and amplification of ceDNA in the presence of Rep and helper virus.

According to some embodiments, the host cells used to make the ceDNA vectors for expression of antigens, or immunogenic peptides, as described herein are insect cells, and baculovirus is used to deliver both the polynucleotide that encodes Rep protein and the non-viral DNA vector polynucleotide expression construct template for ceDNA, e.g., as described in FIGS. 4A-4C and Example 1. According to some embodiments, the host cell is engineered to express Rep protein.

The ceDNA vector is then harvested and isolated from the host cells. The time for harvesting and collecting ceDNA vectors described herein from the cells can be selected and optimized to achieve a high-yield production of the ceDNA vectors. For example, the harvest time can be selected in view of cell viability, cell morphology, cell growth, etc. According to some embodiments, cells are grown and harvested a sufficient time after baculoviral infection to produce ceDNA vectors but before most cells start to die due to the baculoviral toxicity. The DNA vectors can be isolated using plasmid purification kits such as Qiagen Endo-Free Plasmid kits. Other methods developed for plasmid isolation can be also adapted for DNA vectors. Generally, any nucleic acid purification methods can be adopted.

The DNA vectors can be purified by any means known to those of skill in the art for purification of DNA. According to some embodiments, ceDNA vectors are purified as DNA molecules. In another embodiment, the ceDNA vectors are purified as exosomes or microparticles.

The presence of the ceDNA vector for expression of antigens, or immunogenic peptides, can be confirmed by digesting the vector DNA isolated from the cells with a restriction enzyme having a single recognition site on the DNA vector and analyzing both digested and undigested DNA material using gel electrophoresis to confirm the presence of characteristic bands of linear and continuous DNA as compared to linear and non-continuous DNA. FIG. 4C and FIG. 4D illustrate one embodiment for identifying the presence of the closed ended ceDNA vectors produced by the processes herein.

According to some embodiments, the ceDNA is synthetically produced in a cell-free environment.

ceDNA Plasmid

A ceDNA-plasmid is a plasmid used for later production of a ceDNA vector for expression of antigens, or immunogenic peptides, as described herein. According to some embodiments, a ceDNA-plasmid can be constructed using known techniques to provide at least the following as operatively linked components in the direction of transcription: (1) a modified 5′ ITR sequence; (2) an expression cassette containing a cis-regulatory element, for example, a promoter, inducible promoter, regulatory switch, enhancers and the like; and (3) a modified 3′ ITR sequence, where the 3′ ITR sequence is symmetric relative to the 5′ ITR sequence. According to some embodiments, the expression cassette flanked by the ITRs comprises a cloning site for introducing an exogenous sequence. The expression cassette replaces the rep and cap coding regions of the AAV genomes.

According to some aspects, a ceDNA vector for expression of antigens, or immunogenic peptides, is obtained from a plasmid, referred to herein as a “ceDNA-plasmid” encoding in this order: a first adeno-associated virus (AAV) inverted terminal repeat (ITR), an expression cassette comprising a transgene, and a mutated or modified AAV ITR, wherein said ceDNA-plasmid is devoid of AAV capsid protein coding sequences. In alternative embodiments, the ceDNA-plasmid encodes in this order: a first (or 5′) modified or mutated AAV ITR, an expression cassette comprising a transgene, and a second (or 3′) modified AAV ITR, wherein said ceDNA-plasmid is devoid of AAV capsid protein coding sequences, and wherein the 5′ and 3′ ITRs are symmetric relative to each other. In alternative embodiments, the ceDNA-plasmid encodes in this order: a first (or 5′) modified or mutated AAV ITR, an expression cassette comprising a transgene, and a second (or 3′) mutated or modified AAV ITR, wherein said ceDNA-plasmid is devoid of AAV capsid protein coding sequences, and wherein the 5′ and 3′ modified ITRs have the same modifications (i.e., they are inverse complement or symmetric relative to each other).

In a further embodiment, the ceDNA-plasmid system is devoid of viral capsid protein coding sequences (i.e., it is devoid of AAV capsid genes but also of capsid genes of other viruses). In addition, in a particular embodiment, the ceDNA-plasmid is also devoid of AAV Rep protein coding sequences. Accordingly, in a preferred embodiment, ceDNA-plasmid is devoid of functional AAV cap and AAV rep genes GG-3′ for AAV2) plus a variable palindromic sequence allowing for hairpin formation.

A ceDNA-plasmid of the present disclosure can be generated using natural nucleic acid sequences of the genomes of any AAV serotypes well known in the art. According to some embodiments, the ceDNA-plasmid backbone is derived from the AAV1, AAV2, AAV3, AAV4, AAV5, AAV 5, AAV7, AAV8, AAV9, AAV10, AAV 11, AAV12, AAVrh8, AAVrh10, AAV-DJ, and AAV-DJ8 genome. E.g., NCBI: NC 002077; NC 001401; NC001729; NC001829; NC006152; NC 006260; NC 006261; Kotin and Smith, The Springer Index of Viruses, available at the URL maintained by Springer (at the address oesys.springer.de/viruses/database/mkchapter.asp?virID=42.04) (note—references to a URL or database refer to the contents of the URL or database as of the effective filing date of this application). In a particular embodiment, the ceDNA-plasmid backbone is derived from the AAV2 genome. In another particular embodiment, the ceDNA-plasmid backbone is a synthetic backbone genetically engineered to include at its 5′ and 3′ ITRs derived from one of these AAV genomes.

A ceDNA-plasmid can optionally include a selectable or selection marker for use in the establishment of a ceDNA vector-producing cell line. According to some embodiments, the selection marker can be inserted downstream (i.e., 3′) of the 3′ ITR sequence. In another embodiment, the selection marker can be inserted upstream (i.e., 5′) of the 5′ ITR sequence. Appropriate selection markers include, for example, those that confer drug resistance. Selection markers can be, for example, a blasticidin S-resistance gene, kanamycin, geneticin, and the like. In a preferred embodiment, the drug selection marker is a blasticidin S-resistance gene.

An exemplary ceDNA (e.g., rAAV0) vector for expression of antigens, or immunogenic peptides, is produced from an rAAV plasmid. A method for the production of a rAAV vector, can comprise: (a) providing a host cell with a rAAV plasmid as described above, wherein both the host cell and the plasmid are devoid of capsid protein encoding genes, (b) culturing the host cell under conditions allowing production of an ceDNA genome, and (c) harvesting the cells and isolating the AAV genome produced from said cells.

Exemplary Method of Making the ceDNA Vectors from ceDNA Plasmids

Methods for making capsid-less ceDNA vectors for expression of antigens, or immunogenic peptides, are also provided herein, notably a method with a sufficiently high yield to provide sufficient vector for in vivo experiments.

According to some embodiments, a method for the production of a ceDNA vector for expression of antigens, or immunogenic peptides, comprises the steps of: (1) introducing the nucleic acid construct comprising an expression cassette and two symmetric ITR sequences into a host cell (e.g., Sf9 cells), (2) optionally, establishing a clonal cell line, for example, by using a selection marker present on the plasmid, (3) introducing a Rep coding gene (either by transfection or infection with a baculovirus carrying said gene) into said insect cell, and (4) harvesting the cell and purifying the ceDNA vector. The nucleic acid construct comprising an expression cassette and two ITR sequences described above for the production of ceDNA vector can be in the form of a ceDNA plasmid, or Bacmid or Baculovirus generated with the ceDNA plasmid as described below. The nucleic acid construct can be introduced into a host cell by transfection, viral transduction, stable integration, or other methods known in the art.

### Cell Lines

Host cell lines used in the production of a ceDNA vector for expression of antigens, or immunogenic peptides, can include insect cell lines derived from Spodoptera frugiperda, such as Sf9 Sf21, or Trichoplusia ni cell, or other invertebrate, vertebrate, or other eukaryotic cell lines including mammalian cells. Other cell lines known to an ordinarily skilled artisan can also be used, such as HEK293, Huh-7, HeLa, HepG2, HeplA, 911, CHO, COS, MeWo, NIH3T3, A549, HT1 180, monocytes, and mature and immature dendritic cells. Host cell lines can be transfected for stable expression of the ceDNA-plasmid for high yield ceDNA vector production.

CeDNA-plasmids can be introduced into Sf9 cells by transient transfection using reagents (e.g., liposomal, calcium phosphate) or physical means (e.g., electroporation) known in the art. Alternatively, stable Sf9 cell lines which have stably integrated the ceDNA-plasmid into their genomes can be established. Such stable cell lines can be established by incorporating a selection marker into the ceDNA-plasmid as described above. If the ceDNA-plasmid used to transfect the cell line includes a selection marker, such as an antibiotic, cells that have been transfected with the ceDNA-plasmid and integrated the ceDNA-plasmid DNA into their genome can be selected for by addition of the antibiotic to the cell growth media. Resistant clones of the cells can then be isolated by single-cell dilution or colony transfer techniques and propagated.

Isolating and Purifying ceDNA Vectors

Examples of the process for obtaining and isolating ceDNA vectors are described in FIGS. 4A-4E and the specific examples below. ceDNA-vectors for expression of antigens, or immunogenic peptides, disclosed herein can be obtained from a producer cell expressing AAV Rep protein(s), further transformed with a ceDNA-plasmid, ceDNA-bacmid, or ceDNA-baculovirus. Plasmids useful for the production of ceDNA vectors include plasmids that encode antigens, or immunogenic peptides, or plasmids encoding one or more REP proteins.

According to some aspect, a polynucleotide encodes the AAV Rep protein (Rep 78 or 68) delivered to a producer cell in a plasmid (Rep-plasmid), a bacmid (Rep-bacmid), or a baculovirus (Rep-baculovirus). The Rep-plasmid, Rep-bacmid, and Rep-baculovirus can be generated by methods described above.

Methods to produce a ceDNA vector for expression of antigens, or immunogenic peptides, are described herein. Expression constructs used for generating a ceDNA vector for expression of antigens, or immunogenic peptides, as described herein can be a plasmid (e.g., ceDNA-plasmids), a Bacmid (e.g., ceDNA-bacmid), and/or a baculovirus (e.g., ceDNA-baculovirus). By way of an example only, a ceDNA-vector can be generated from the cells co-infected with ceDNA-baculovirus and Rep-baculovirus. Rep proteins produced from the Rep-baculovirus can replicate the ceDNA-baculovirus to generate ceDNA-vectors. Alternatively, ceDNA vectors for expression of antigens, or immunogenic peptides, can be generated from the cells stably transfected with a construct comprising a sequence encoding the AAV Rep protein (Rep78/52) delivered in Rep-plasmids, Rep-bacmids, or Rep-baculovirus. CeDNA-Baculovirus can be transiently transfected to the cells, be replicated by Rep protein and produce ceDNA vectors.

The bacmid (e.g., ceDNA-bacmid) can be transfected into permissive insect cells such as Sf9, Sf21, Tni (Trichoplusia ni) cell, High Five cell, and generate ceDNA-baculovirus, which is a recombinant baculovirus including the sequences comprising the symmetric ITRs and the expression cassette. ceDNA-baculovirus can be again infected into the insect cells to obtain a next generation of the recombinant baculovirus. Optionally, the step can be repeated once or multiple times to produce the recombinant baculovirus in a larger quantity.

The time for harvesting and collecting ceDNA vectors for expression of antigens, or immunogenic peptides, as described herein from the cells can be selected and optimized to achieve a high-yield production of the ceDNA vectors. For example, the harvest time can be selected in view of cell viability, cell morphology, cell growth, etc. Usually, cells can be harvested after sufficient time after baculoviral infection to produce ceDNA vectors (e.g., ceDNA vectors) but before majority of cells start to die because of the viral toxicity. The ceDNA-vectors can be isolated from the Sf9 cells using plasmid purification kits such as Qiagen ENDO-FREE PLASMID® kits. Other methods developed for plasmid isolation can be also adapted for ceDNA vectors. Generally, any art-known nucleic acid purification methods can be adopted, as well as commercially available DNA extraction kits.

Alternatively, purification can be implemented by subjecting a cell pellet to an alkaline lysis process, centrifuging the resulting lysate and performing chromatographic separation. As one non-limiting example, the process can be performed by loading the supernatant on an ion exchange column (e.g., SARTOBIND Q®) which retains nucleic acids, and then eluting (e.g., with a 1.2 M NaCl solution) and performing a further chromatographic purification on a gel filtration column (e.g., 6 fast flow GE). The capsid-free AAV vector is then recovered by, e.g., precipitation.

According to some embodiments, ceDNA vectors for expression of antigens, or immunogenic peptides, can also be purified in the form of exosomes, or microparticles. It is known in the art that many cell types release not only soluble proteins, but also complex protein/nucleic acid cargoes via membrane microvesicle shedding (Cocucci et al, 2009; EP 10306226.1) Such vesicles include microvesicles (also referred to as microparticles) and exosomes (also referred to as nanovesicles), both of which comprise proteins and RNA as cargo. Microvesicles are generated from the direct budding of the plasma membrane, and exosomes are released into the extracellular environment upon fusion of multivesicular endosomes with the plasma membrane. Thus, ceDNA vector-containing microvesicles and/or exosomes can be isolated from cells that have been transduced with the ceDNA-plasmid or a bacmid or baculovirus generated with the ceDNA-plasmid.

Microvesicles can be isolated by subjecting culture medium to filtration or ultracentrifugation at 20,000×g, and exosomes at 100,000×g. The optimal duration of ultracentrifugation can be experimentally-determined and will depend on the particular cell type from which the vesicles are isolated. Preferably, the culture medium is first cleared by low-speed centrifugation (e.g., at 2000×g for 5-20 minutes) and subjected to spin concentration using, e.g., an AMICON® spin column (Millipore, Watford, UK). Microvesicles and exosomes can be further purified via FACS or MACS by using specific antibodies that recognize specific surface antigens present on the microvesicles and exosomes. Other microvesicle and exosome purification methods include, but are not limited to, immunoprecipitation, affinity chromatography, filtration, and magnetic beads coated with specific antibodies or aptamers. Upon purification, vesicles are washed with, e.g., phosphate-buffered saline. One advantage of using microvesicles or exosome to deliver ceDNA-containing vesicles is that these vesicles can be targeted to various cell types by including on their membranes proteins recognized by specific receptors on the respective cell types. (See also EP 10306226)

Another aspect of the disclosure herein relates to methods of purifying ceDNA vectors from host cell lines that have stably integrated a ceDNA construct into their own genome. According to some embodiments, ceDNA vectors are purified as DNA molecules. In another embodiment, the ceDNA vectors are purified as exosomes or microparticles.

FIG. 5 of International application PCT/US18/49996 shows a gel confirming the production of ceDNA from multiple ceDNA-plasmid constructs using the method described in the Examples. The ceDNA is confirmed by a characteristic band pattern in the gel, as discussed with respect to FIG. 4D in the Examples.

### VI. Pharmaceutical Compositions

In another aspect, pharmaceutical compositions are provided. The pharmaceutical composition comprises a ceDNA vector for expression of antigens, or immunogenic peptides, as described herein and a pharmaceutically acceptable carrier or diluent.

The ceDNA vectors for expression of antigens, or immunogenic peptides, as disclosed herein can be incorporated into pharmaceutical compositions suitable for administration to a subject for in vivo delivery to cells, tissues, or organs of the subject. Typically, the pharmaceutical composition comprises a ceDNA-vector as disclosed herein and a pharmaceutically acceptable carrier.

Pharmaceutical formulations disclosed herein include liquid, e.g., aqueous, solutions that may be directly administered, and lyophilized powders which may be reconstituted into solutions by adding a diluent before administration. in certain embodiments, a formulation comprising a ceDNA vector as disclosed herein, with or without at least one additional therapeutic agent, can be formulated as a lyophilizate using appropriate excipients. Lyophilization can be performed using a generic Lyophilization cycle on a commercially available lyophilizer (e.g., a VirTis Lab Scale Lyophilizer).

According to some embodiments, the ceDNA vectors for expression of antigens, or immunogenic peptides, as described herein can be incorporated into a pharmaceutical composition suitable for a desired route of therapeutic administration (e.g., parenteral administration). Passive tissue transduction via high pressure intravenous or intra-arterial infusion, as well as intracellular injection, such as intranuclear microinjection or intracytoplasmic injection, are also contemplated. Pharmaceutical compositions for therapeutic purposes can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization including a ceDNA vector can be formulated to deliver a transgene in the nucleic acid to the cells of a recipient, resulting in the therapeutic expression of the transgene or donor sequence therein. The composition can also include a pharmaceutically acceptable carrier.

Pharmaceutically active compositions comprising a ceDNA vector for expression of antigens, or immunogenic peptides, can be formulated to deliver a transgene for various purposes to the cell, e.g., cells of a subject.

Pharmaceutical compositions for therapeutic purposes typically must be sterile and stable under the conditions of manufacture and storage. The composition can be formulated as a solution, microemulsion, dispersion, liposomes, or other ordered structure suitable to high ceDNA vector concentration. Sterile injectable solutions can be prepared by incorporating the ceDNA vector compound in the required amount in an appropriate buffer with one or a combination of ingredients enumerated above, as required, followed by filtered sterilization.

In certain embodiments, the formulation for parenteral administration can be stored in lyophilized form or in a solution. In certain embodiments, parenteral formulations generally are placed into a container having a sterile access port, for example, an intravenous solution bag or vial having a stopper pierceable by a hypodermic injection needle.

In certain embodiments, once the pharmaceutical formulation has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

A ceDNA vector for expression of antigens, or immunogenic peptides, as disclosed herein can be incorporated into a pharmaceutical composition suitable for topical, systemic, intra-amniotic, intrathecal, intracranial, intra-arterial, intravenous, intralymphatic, intraperitoneal, subcutaneous, tracheal, intra-tissue (e.g., intramuscular, intracardiac, intrahepatic, intrarenal, intracerebral), intrathecal, intravesical, conjunctival (e.g., extra-orbital, intraorbital, retroorbital, intraretinal, subretinal, choroidal, sub-choroidal, intrastromal, intracameral and intravitreal), intracochlear, and mucosal (e.g., oral, rectal, nasal) administration. Passive tissue transduction via high pressure intravenous or intraarterial infusion, as well as intracellular injection, such as intranuclear microinjection or intracytoplasmic injection, are also contemplated.

According to some aspects, the methods provided herein comprise delivering one or more ceDNA vectors for expression of antigens, or immunogenic peptides, as disclosed herein to a host cell. Also provided herein are cells produced by such methods, and organisms (such as animals, plants, or fungi) comprising or produced from such cells. Methods of delivery of nucleic acids can include lipofection, nucleofection, microinjection, biolistics, liposomes, immunoliposomes, polycation or lipid:nucleic acid conjugates, naked DNA, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Delivery can be to cells (e.g., in vitro or ex vivo administration) or target tissues (e.g., in vivo administration).

Various techniques and methods are known in the art for delivering nucleic acids to cells. For example, nucleic acids, such as ceDNA for expression of antigens, or immunogenic peptides, can be formulated into lipid nanoparticles (LNPs), lipidoids, liposomes, lipid nanoparticles, lipoplexes, or core-shell nanoparticles. Typically, LNPs are composed of nucleic acid (e.g., ceDNA) molecules, one or more ionizable or cationic lipids (or salts thereof), one or more non-ionic or neutral lipids (e.g., a phospholipid), a molecule that prevents aggregation (e.g., PEG or a PEG-lipid conjugate), and optionally a sterol (e.g., cholesterol).

Another method for delivering nucleic acids, such as ceDNA for expression of antigens, or immunogenic peptides, to a cell is by conjugating the nucleic acid with a ligand that is internalized by the cell. For example, the ligand can bind a receptor on the cell surface and internalized via endocytosis. The ligand can be covalently linked to a nucleotide in the nucleic acid. Exemplary conjugates for delivering nucleic acids into a cell are described, example, in WO2015/006740, WO2014/025805, WO2012/037254, WO2009/082606, WO2009/073809, WO2009/018332, WO2006/112872, WO2004/090108, WO2004/091515 and WO2017/177326.

Nucleic acids, such as ceDNA vectors for expression of antigens, or immunogenic peptides, can also be delivered to a cell by transfection. Useful transfection methods include, but are not limited to, lipid-mediated transfection, cationic polymer-mediated transfection, or calcium phosphate precipitation. Transfection reagents are well known in the art and include, but are not limited to, TurboFect Transfection Reagent (Thermo Fisher Scientific), Pro-Ject Reagent (Thermo Fisher Scientific), TRANSPASS™ P Protein Transfection Reagent (New England Biolabs), CHARIOT™ Protein Delivery Reagent (Active Motif), PROTEOJUICE™ Protein Transfection Reagent (EMD Millipore), 293fectin, LIPOFECTAMINE™ 2000, LIPOFECTAMINE™ 3000 (Thermo Fisher Scientific), LIPOFECTAMINE™ (Thermo Fisher Scientific), LIPOFECTIN™ (Thermo Fisher Scientific), DMRIE-C, CELLFECTIN™ (Thermo Fisher Scientific), OLIGOFECTAMINE™ (Thermo Fisher Scientific), LIPOFECTACE™, FUGENE™ (Roche, Basel, Switzerland), FUGENE™ HD (Roche), TRANSFECTAM™ (Transfectam, Promega, Madison, Wis.), TFX-10™ (Promega), TFX-20™ (Promega), TFX-50™ (Promega), TRANSFECTIN™ (BioRad, Hercules, Calif.), SILENTFECT™ (Bio-Rad), Effectene™ (Qiagen, Valencia, Calif.), DC-chol (Avanti Polar Lipids), GENEPORTER™ (Gene Therapy Systems, San Diego, Calif.), DHARMAFECT 1™ (Dharmacon, Lafayette, Colo.), DHARMAFECT 2™ (Dharmacon), DHARMAFECT 3™ (Dharmacon), DHARMAFECT 4™ (Dharmacon), ESCORT™ III (Sigma, St. Louis, Mo.), and ESCORT™ IV (Sigma Chemical Co.). Nucleic acids, such as ceDNA, can also be delivered to a cell via microfluidics methods known to those of skill in the art.

ceDNA vectors for expression of antigens, or immunogenic peptides, as described herein can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

The ceDNA vectors for expression of antigens, or immunogenic peptides, in accordance with the present disclosure can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids. Exemplary liposomes and liposome formulations, including but not limited to polyethylene glycol (PEG)-functional group containing compounds are disclosed in International Application PCT/US2018/050042, filed on Sep. 7, 2018 and in International application PCT/US2018/064242, filed on Dec. 6, 2018, e.g., see the section entitled “Pharmaceutical Formulations”.

Various delivery methods known in the art or modification thereof can be used to deliver ceDNA vectors in vitro or in vivo. According to some embodiments, ceDNA vectors for expression of antigens, or immunogenic peptides, are delivered by making transient penetration in cell membrane by mechanical, electrical, ultrasonic, hydrodynamic, or laser-based energy so that DNA entrance into the targeted cells is facilitated. For example, a ceDNA vector can be delivered by transiently disrupting cell membrane by squeezing the cell through a size-restricted channel or by other means known in the art. According to some embodiments, a ceDNA vector alone is directly injected as naked DNA into any one of: any one or more tissues selected from: lung, liver, kidneys, gallbladder, prostate, adrenal gland, heart, intestine, stomach, skin, thymus, cardiac muscle or skeletal muscle. According to some embodiments, a ceDNA vector is delivered by gene gun. Gold or tungsten spherical particles (1-3 m diameter) coated with capsid-free AAV vectors can be accelerated to high speed by pressurized gas to penetrate into target tissue cells.

Compositions comprising a ceDNA vector for expression of antigens, or immunogenic peptides, and a pharmaceutically acceptable carrier are specifically contemplated herein. According to some embodiments, the ceDNA vector is formulated with a lipid delivery system, for example, liposomes as described herein. According to some embodiments, such compositions are administered by any route desired by a skilled practitioner. The compositions may be administered to a subject by different routes including orally, parenterally, sublingually, transdermally, rectally, transmucosally, topically, via inhalation, via buccal administration, intrapleurally, intravenous, intra-arterial, intraperitoneal, subcutaneous, intramuscular, intranasal intrathecal, and intraarticular or combinations thereof. For veterinary use, the composition may be administered as a suitably acceptable formulation in accordance with normal veterinary practice. The veterinarian may readily determine the dosing regimen and route of administration that is most appropriate for a particular animal. The compositions may be administered by traditional syringes, needleless injection devices, “microprojectile bombardment gene guns”, or other physical methods such as electroporation (“EP”), hydrodynamic methods, or ultrasound.

According to some cases, a ceDNA vector for expression of antigens, or immunogenic peptides, is delivered by hydrodynamic injection, which is a simple and highly efficient method for direct intracellular delivery of any water-soluble compounds and particles into internal organs and skeletal muscle in an entire limb.

According to some embodiments, ceDNA vectors for expression of antigens, or immunogenic peptides, are delivered by ultrasound by making nanoscopic pores in membrane to facilitate intracellular delivery of DNA particles into cells of internal organs or tumors, so the size and concentration of plasmid DNA have great role in efficiency of the system. According to some embodiments, ceDNA vectors are delivered by magnetofection by using magnetic fields to concentrate particles containing nucleic acid into the target cells.

According to some embodiments, chemical delivery systems can be used, for example, by using nanomeric complexes, which include compaction of negatively charged nucleic acid by polycationic nanomeric particles, belonging to cationic liposome/micelle or cationic polymers.

Cationic lipids used for the delivery method includes, but not limited to monovalent cationic lipids, polyvalent cationic lipids, guanidine containing compounds, cholesterol derivative compounds, cationic polymers, (e.g., poly(ethylenimine), poly-L-lysine, protamine, other cationic polymers), and lipid-polymer hybrid.

### A. Exosomes:

According to some embodiments, a ceDNA vector for expression of antigens, or immunogenic peptides, as disclosed herein is delivered by being packaged in an exosome. Exosomes are small membrane vesicles of endocytic origin that are released into the extracellular environment following fusion of multivesicular bodies with the plasma membrane. Their surface consists of a lipid bilayer from the donor cell's cell membrane, they contain cytosol from the cell that produced the exosome, and exhibit membrane proteins from the parental cell on the surface. Exosomes are produced by various cell types including epithelial cells, B and T lymphocytes, mast cells (MC) as well as dendritic cells (DC). According to some embodiments, exosomes with a diameter between 10 nm and 1 μm, between 20 nm and 500 nm, between 30 nm and 250 nm, between 50 nm and 100 nm are envisioned for use. Exosomes can be isolated for a delivery to target cells using either their donor cells or by introducing specific nucleic acids into them. Various approaches known in the art can be used to produce exosomes containing capsid-free AAV vectors of the present disclosure.

### Microparticle/Nanoparticles

According to some embodiments, a ceDNA vector for expression of antigens, or immunogenic peptides, as disclosed herein is delivered by a lipid nanoparticle. Generally, lipid nanoparticles comprise an ionizable amino lipid (e.g., heptatriaconta-6,9,28,31-tetraen-19-yl 4-(dimethylamino)butanoate, DLin-MC3-DMA, a phosphatidylcholine (1,2-distearoyl-sn-glycero-3-phosphocholine, DSPC), cholesterol and a coat lipid (polyethylene glycol-dimyristolglycerol, PEG-DMG), for example as disclosed by Tam et al. (2013). Advances in Lipid Nanoparticles for siRNA delivery. Pharmaceuticals 5(3): 498-507.

According to some embodiments, a lipid nanoparticle has a mean diameter between about 10 and about 1000 nm. According to some embodiments, a lipid nanoparticle has a diameter that is less than 300 nm. According to some embodiments, a lipid nanoparticle has a diameter between about 10 and about 300 nm. According to some embodiments, a lipid nanoparticle has a diameter that is less than 200 nm. According to some embodiments, a lipid nanoparticle has a diameter between about 25 and about 200 nm. According to some embodiments, a lipid nanoparticle preparation (e.g., composition comprising a plurality of lipid nanoparticles) has a size distribution in which the mean size (e.g., diameter) is about 70 nm to about 200 nm, and more typically the mean size is about 100 nm or less.

Various lipid nanoparticles known in the art can be used to deliver ceDNA vector for expression of antigens, or immunogenic peptides, as disclosed herein. For example, various delivery methods using lipid nanoparticles are described in U.S. Pat. Nos. 9,404,127, 9,006,417 and 9,518,272.

### Conjugates

According to some embodiments, a ceDNA vector for expression of antigens, or immunogenic peptides, as disclosed herein is conjugated (e.g., covalently bound to an agent that increases cellular uptake. An “agent that increases cellular uptake” is a molecule that facilitates transport of a nucleic acid across a lipid membrane. For example, a nucleic acid can be conjugated to a lipophilic compound (e.g., cholesterol, tocopherol, etc.), a cell penetrating peptide (CPP) (e.g., penetratin, TAT, Syn1B, etc.), and polyamines (e.g., spermine). Further examples of agents that increase cellular uptake are disclosed, for example, in Winkler (2013). Oligonucleotide conjugates for therapeutic applications. Ther. Deliv. 4(7); 791-809.

According to some embodiments, a ceDNA vector for expression of antigens, or immunogenic peptides, as disclosed herein is conjugated to a polymer (e.g., a polymeric molecule) or a folate molecule (e.g., folic acid molecule). Generally, delivery of nucleic acids conjugated to polymers is known in the art, for example as described in WO2000/34343 and WO2008/022309. According to some embodiments, a ceDNA vector for expression of antigens, or immunogenic peptides, as disclosed herein is conjugated to a poly(amide) polymer, for example as described by U.S. Pat. No. 8,987,377. According to some embodiments, a nucleic acid described by the disclosure is conjugated to a folic acid molecule as described in U.S. Pat. No. 8,507,455.

According to some embodiments, a ceDNA vector for expression of antigens, or immunogenic peptides, as disclosed herein is conjugated to a carbohydrate, for example as described in U.S. Pat. No. 8,450,467.

### Nanocapsule

Alternatively, nanocapsule formulations of a ceDNA vector for expression of antigens, or immunogenic peptides, as disclosed herein can be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

### Liposomes

The ceDNA vectors for expression of antigens, or immunogenic peptides, in accordance with the present disclosure can be added to liposomes for delivery to a cell or target organ in a subject. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

The formation and use of liposomes is generally known to those of skill in the art. Liposomes have been developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

### Exemplary Liposome and Lipid Nanoparticle (LNP) Compositions

The ceDNA vectors for expression of antigens, or immunogenic peptides, in accordance with the present disclosure can be added to liposomes for delivery to a cell, e.g., a cell in need of expression of the transgene. Liposomes are vesicles that possess at least one lipid bilayer. Liposomes are typical used as carriers for drug/therapeutic delivery in the context of pharmaceutical development. They work by fusing with a cellular membrane and repositioning its lipid structure to deliver a drug or active pharmaceutical ingredient (API). Liposome compositions for such delivery are composed of phospholipids, especially compounds having a phosphatidylcholine group, however these compositions may also include other lipids.

Lipid nanoparticles (LNPs) comprising ceDNA vectors are disclosed in International Application PCT/US2018/050042, filed on Sep. 7, 2018; International Application PCT/US2018/064242, filed on Dec. 6, 2018; and International Application PCT/US2022/025455, filed on Apr. 20, 2022, which are incorporated herein in their entirety and envisioned for use in the methods and compositions for ceDNA vectors for expression of antigens, or immunogenic peptides, as disclosed herein.

According to some aspects, the disclosure provides for a liposome formulation that includes one or more compounds with a polyethylene glycol (PEG) functional group (so-called “PEG-ylated compounds”) which can reduce the immunogenicity/antigenicity of, provide hydrophilicity and hydrophobicity to the compound(s) and reduce dosage frequency. Or the liposome formulation simply includes polyethylene glycol (PEG) polymer as an additional component. In such aspects, the molecular weight of the PEG or PEG functional group can be from 62 Da to about 5,000 Da.

According to some aspects, the disclosure provides for a liposome formulation that will deliver an API with extended release or controlled release profile over a period of hours to weeks. According to some related aspects, the liposome formulation may comprise aqueous chambers that are bound by lipid bilayers. In other related aspects, the liposome formulation encapsulates an API with components that undergo a physical transition at elevated temperature which releases the API over a period of hours to weeks.

According to some aspects, the liposome formulation comprises sphingomyelin and one or more lipids disclosed herein. According to some aspects, the liposome formulation comprises optisomes.

According to some aspects, the disclosure provides for a liposome formulation that includes one or more lipids selected from: N-(carbonyl-methoxypolyethylene glycol 2000)-1,2-distearoyl-sn-glycero-3-phosphoethanolamine sodium salt, (distearoyl-sn-glycero-phosphoethanolamine), MPEG (methoxy polyethylene glycol)-conjugated lipid, HSPC (hydrogenated soy phosphatidylcholine); PEG (polyethylene glycol); DSPE (distearoyl-sn-glycero-phosphoethanolamine); DSPC (distearoylphosphatidylcholine); DOPC (dioleoylphosphatidylcholine); DPPG (dipalmitoylphosphatidylglycerol); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine). cholesteryl sulphate (CS), dipalmitoylphosphatidylglycerol (DPPG), DOPC (dioleoly-sn-glycero-phosphatidylcholine) or any combination thereof.

According to some aspects, the disclosure provides for a liposome formulation comprising phospholipid, cholesterol and a PEG-ylated lipid in a molar ratio of 56:38:5. According to some aspects, the liposome formulation's overall lipid content is from 2-16 mg/mL. According to some aspects, the disclosure provides for a liposome formulation comprising a lipid containing a phosphatidylcholine functional group, a lipid containing an ethanolamine functional group and a PEG-ylated lipid. According to some aspects, the disclosure provides for a liposome formulation comprising a lipid containing a phosphatidylcholine functional group, a lipid containing an ethanolamine functional group and a PEG-ylated lipid in a molar ratio of 3:0.015:2 respectively. According to some aspects, the disclosure provides for a liposome formulation comprising a lipid containing a phosphatidylcholine functional group, cholesterol and a PEG-ylated lipid. According to some aspects, the disclosure provides for a liposome formulation comprising a lipid containing a phosphatidylcholine functional group and cholesterol. According to some aspects, the PEG-ylated lipid is PEG-2000-DSPE. According to some aspects, the disclosure provides for a liposome formulation comprising DPPG, soy PC, MPEG-DSPE lipid conjugate and cholesterol.

According to some aspects, the disclosure provides for a liposome formulation comprising one or more lipids containing a phosphatidylcholine functional group and one or more lipids containing an ethanolamine functional group. According to some aspects, the disclosure provides for a liposome formulation comprising one or more: lipids containing a phosphatidylcholine functional group, lipids containing an ethanolamine functional group, and sterols, e.g., cholesterol. According to some aspects, the liposome formulation comprises DOPC/DEPC; and DOPE.

According to some aspects, the disclosure provides for a liposome formulation further comprising one or more pharmaceutical excipients, e.g., sucrose and/or glycine.

According to some aspects, the disclosure provides for a liposome formulation that is either unilamellar or multilamellar in structure. According to some aspects, the disclosure provides for a liposome formulation that comprises multi-vesicular particles and/or foam-based particles. According to some aspects, the disclosure provides for a liposome formulation that are larger in relative size to common nanoparticles and about 150 to 250 nm in size. According to some aspects, the liposome formulation is a lyophilized powder.

According to some aspects, the disclosure provides for a liposome formulation that is made and loaded with ceDNA vectors disclosed or described herein, by adding a weak base to a mixture having the isolated ceDNA outside the liposome. This addition increases the pH outside the liposomes to approximately 7.3 and drives the API into the liposome. According to some aspects, the disclosure provides for a liposome formulation having a pH that is acidic on the inside of the liposome. In such cases the inside of the liposome can be at pH 4-6.9, and more preferably pH 6.5. In other aspects, the disclosure provides for a liposome formulation made by using intra-liposomal drug stabilization technology. In such cases, polymeric or non-polymeric highly charged anions and intra-liposomal trapping agents are utilized, e.g., polyphosphate or sucrose octasulfate.

According to some aspects, the disclosure provides for a lipid nanoparticle comprising ceDNA and an ionizable lipid. For example, a lipid nanoparticle formulation that is made and loaded with ceDNA obtained by the process as disclosed in International Application PCT/US2018/050042, filed on Sep. 7, 2018, which is incorporated herein. This can be accomplished by high energy mixing of ethanolic lipids with aqueous ceDNA at low pH which protonates the ionizable lipid and provides favorable energetics for ceDNA/lipid association and nucleation of particles. The particles can be further stabilized through aqueous dilution and removal of the organic solvent. The particles can be concentrated to the desired level.

Generally, the lipid nanoparticles are prepared at a total lipid to ceDNA (mass or weight) ratio of from about 10:1 to 60:1. According to some embodiments, the lipid to ceDNA ratio (mass/mass ratio; w/w ratio) can be in the range of from about 1:1 to about 60:1, from about 1:1 to about 55:1, from about 1:1 to about 50:1, from about 1:1 to about 45:1, from about 1:1 to about 40:1, from about 1:1 to about 35:1, from about 1:1 to about 30:1, from about 1:1 to about 25:1, from about 10:1 to about 14:1, from about 3:1 to about 15:1, from about 4:1 to about 10:1, from about 5:1 to about 9:1, about 6:1 to about 9:1; from about 30:1 to about 60:1. According to some embodiments, the lipid particles (e.g., lipid nanoparticles) are prepared at a ceDNA (mass or weight) to total lipid ratio of about 60:1. According to some embodiments, the lipid particles are prepared at a total lipid to ceDNA (mass or weight) ratio of from about 10:1 to 30:1. According to some embodiments, the lipid to ceDNA ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and ceDNA can be adjusted to provide a desired N/P ratio, for example, N/P ratio of 3, 4, 5, 6, 7, 8, 9, 10 or higher. Generally, the lipid particle formulation's overall lipid content can range from about 5 mg/ml to about 30 mg/mL.

The ionizable lipid is typically employed to condense the nucleic acid cargo, e.g., ceDNA at low pH and to drive membrane association and fusogenicity. Generally, ionizable lipids are lipids comprising at least one amino group that is positively charged or becomes protonated under acidic conditions, for example at pH of 6.5 or lower. Ionizable lipids are also referred to as cationic lipids herein.

Exemplary ionizable lipids are described in International PCT patent publications WO2015/095340, WO2015/199952, WO2018/011633, WO2017/049245, WO2015/061467, WO2012/040184, WO2012/000104, WO2015/074085, WO2016/081029, WO2017/004143, WO2017/075531, WO2017/117528, WO2011/022460, WO2013/148541, WO2013/116126, WO2011/153120, WO2012/044638, WO2012/054365, WO2011/090965, WO2013/016058, WO2012/162210, WO2008/042973, WO2010/129709, WO2010/144740, WO2012/099755, WO2013/049328, WO2013/086322, WO2013/086373, WO2011/071860, WO2009/132131, WO2010/048536, WO2010/088537, WO2010/054401, WO2010/054406, WO2010/054405, WO2010/054384, WO2012/016184, WO2009/086558, WO2010/042877, WO2011/000106, WO2011/000107, WO2005/120152, WO2011/141705, WO2013/126803, WO2006/007712, WO2011/038160, WO2005/121348, WO2011/066651, WO2009/127060, WO2011/141704, WO2006/069782, WO2012/031043, WO2013/006825, WO2013/033563, WO2013/089151, WO2017/099823, WO2015/095346, and WO2013/086354, and US patent publications US2016/0311759, US2015/0376115, US2016/0151284, US2017/0210697, US2015/0140070, US2013/0178541, US2013/0303587, US2015/0141678, US2015/0239926, US2016/0376224, US2017/0119904, US2012/0149894, US2015/0057373, US2013/0090372, US2013/0274523, US2013/0274504, US2013/0274504, US2009/0023673, US2012/0128760, US2010/0324120, US2014/0200257, US2015/0203446, US2018/0005363, US2014/0308304, US2013/0338210, US2012/0101148, US2012/0027796, US2012/0058144, US2013/0323269, US2011/0117125, US2011/0256175, US2012/0202871, US2011/0076335, US2006/0083780, US2013/0123338, US2015/0064242, US2006/0051405, US2013/0065939, US2006/0008910, US2003/0022649, US2010/0130588, US2013/0116307, US2010/0062967, US2013/0202684, US2014/0141070, US2014/0255472, US2014/0039032, US2018/0028664, US2016/0317458, and US2013/0195920, the contents of all of which are incorporated herein by reference in their entirety.

According to some embodiments, the ionizable lipid is MC3 (6Z,9Z,28Z,31Z)-heptatriaconta-6,9,28,31-tetraen-19-yl-4-(dimethylamino) butanoate (DLin-MC3-DMA or MC3) having the following structure:

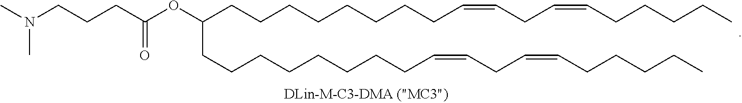 [SMILES: CCCCC/C=C\C/C=C\CCCCCCCCC(CCCCCCCC/C=C\C/C=C\CCCCC)OC(=O)CCCN(C)C ; "."]

The lipid DLin-MC3-DMA is described in Jayaraman et al., Angew. Chem. Int. Ed Engl. (2012), 51(34): 8529-8533, content of which is incorporated herein by reference in its entirety.

According to some embodiments, the ionizable lipid is the lipid ATX-002 as described in WO2015/074085, content of which is incorporated herein by reference in its entirety.

According to some embodiments, the ionizable lipid is (13Z,16Z)—N,N-dimethyl-3-nonyldocosa-13,16-dien-1-amine (Compound 32), as described in WO2012/040184, content of which is incorporated herein by reference in its entirety.

According to some embodiments, the ionizable lipid is Compound 6 or Compound 22 as described in WO2015/199952, content of which is incorporated herein by reference in its entirety.

According to some embodiments, the ionizable lipid is selected from Lipid 1 through Lipid 25 as described in PCT/US2022/025455, content of which is incorporated herein by reference in its entirety.

According to some embodiments, the ionizable lipid is selected from the group consisting of the following:

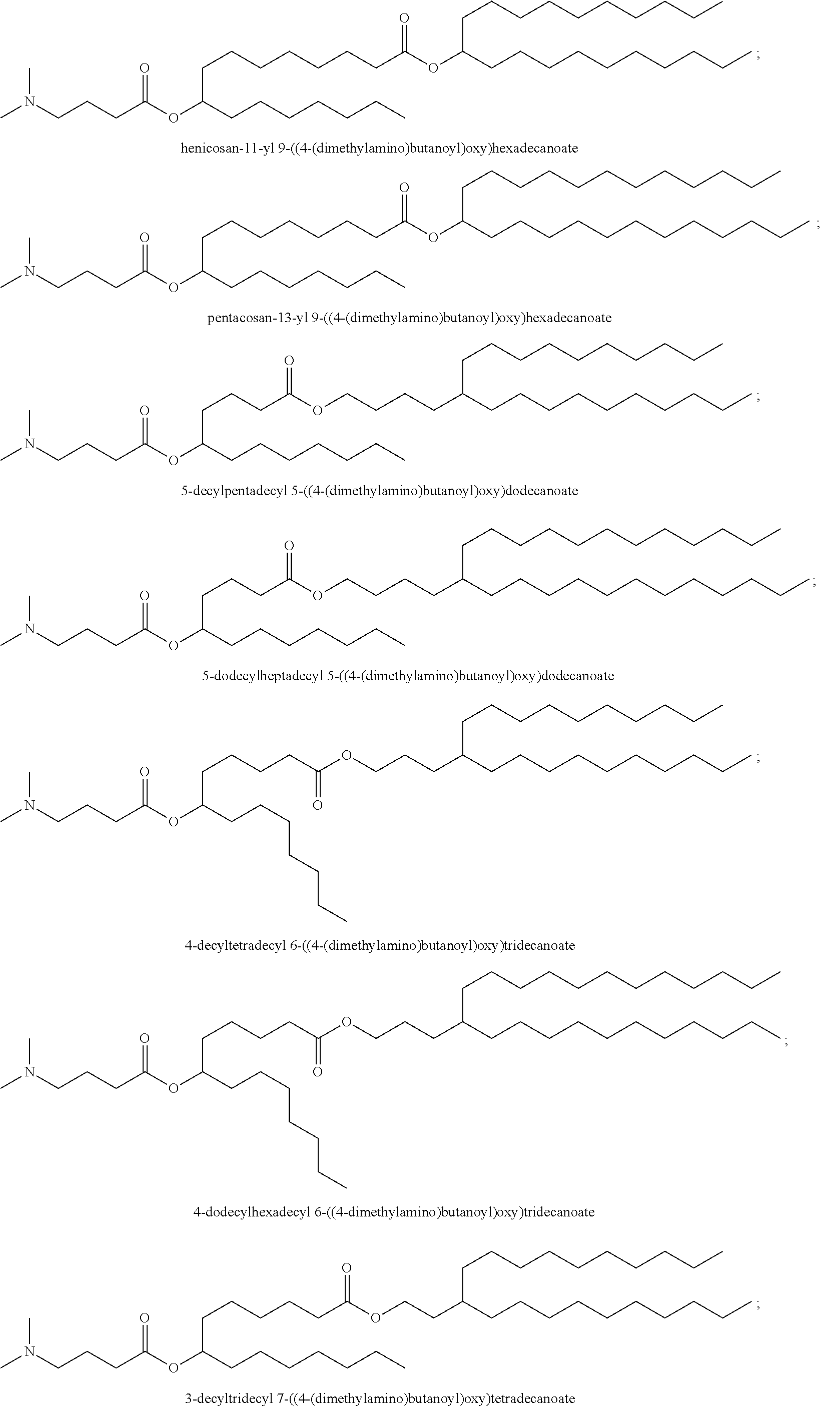

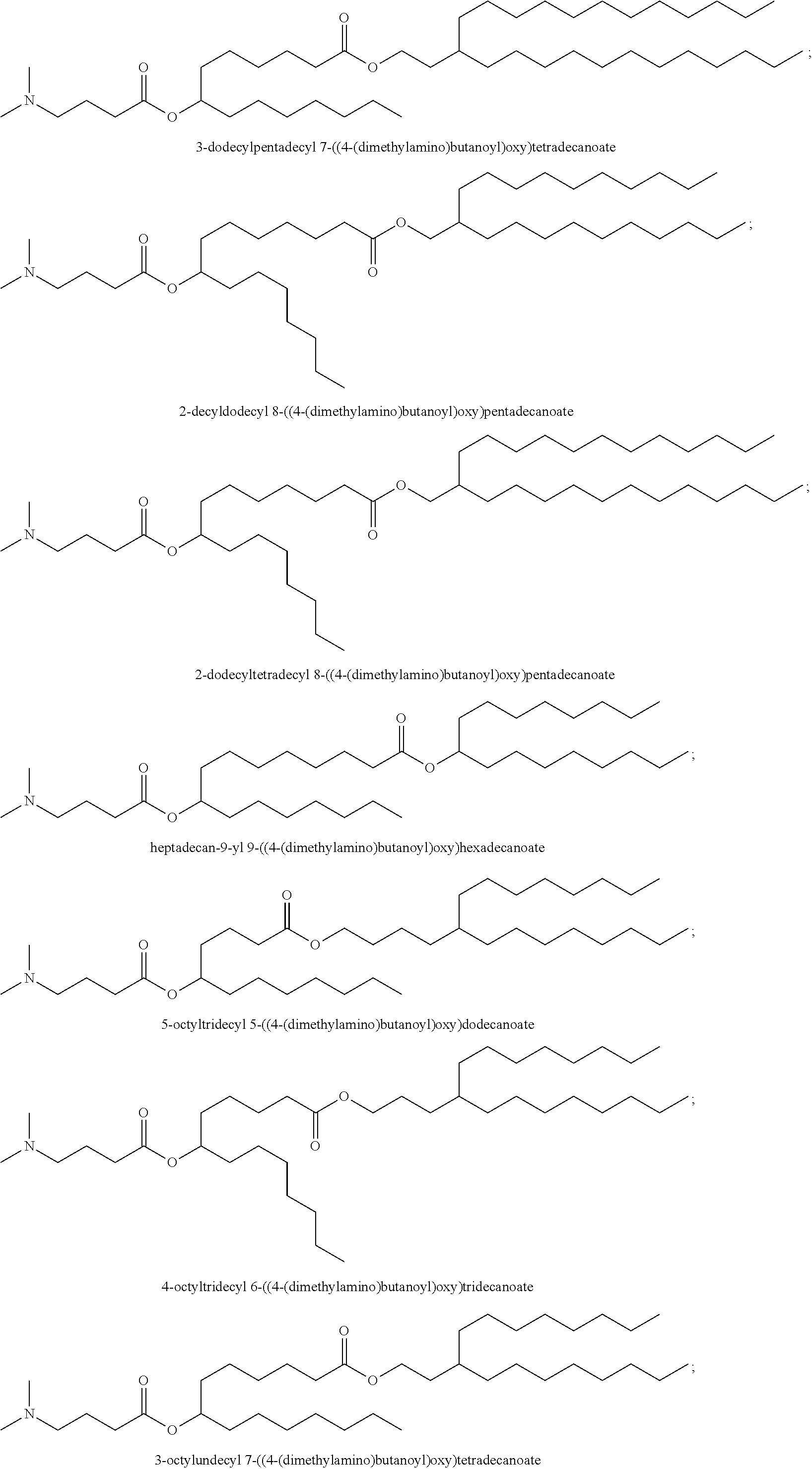

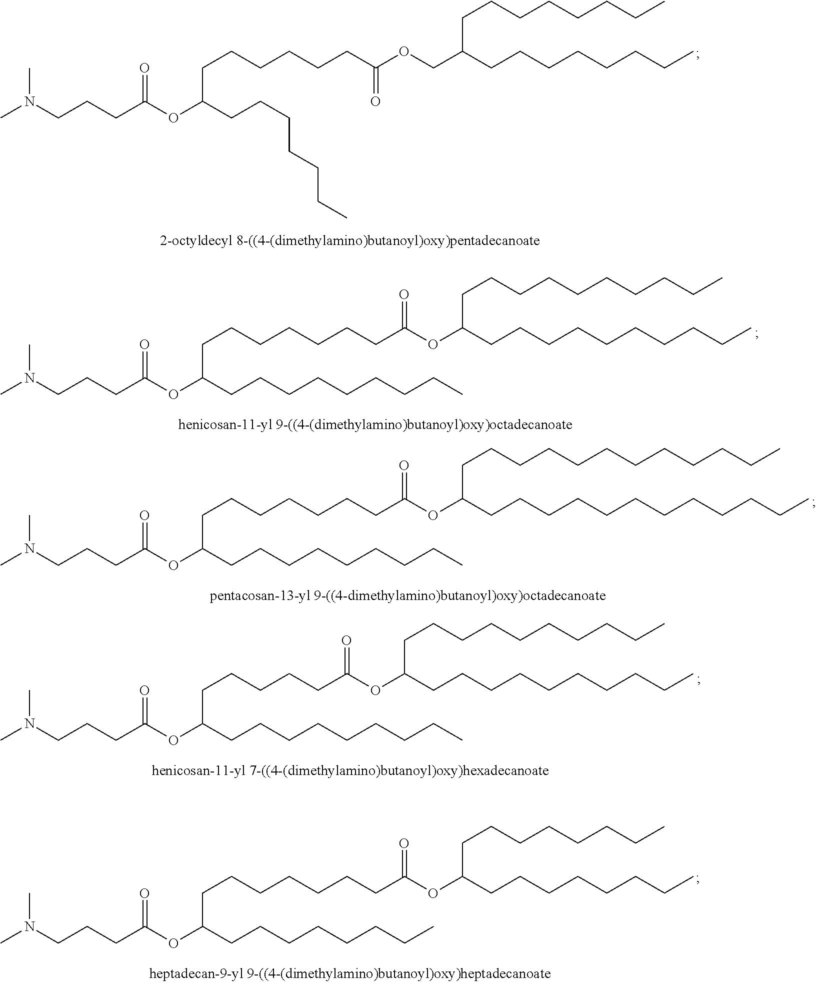

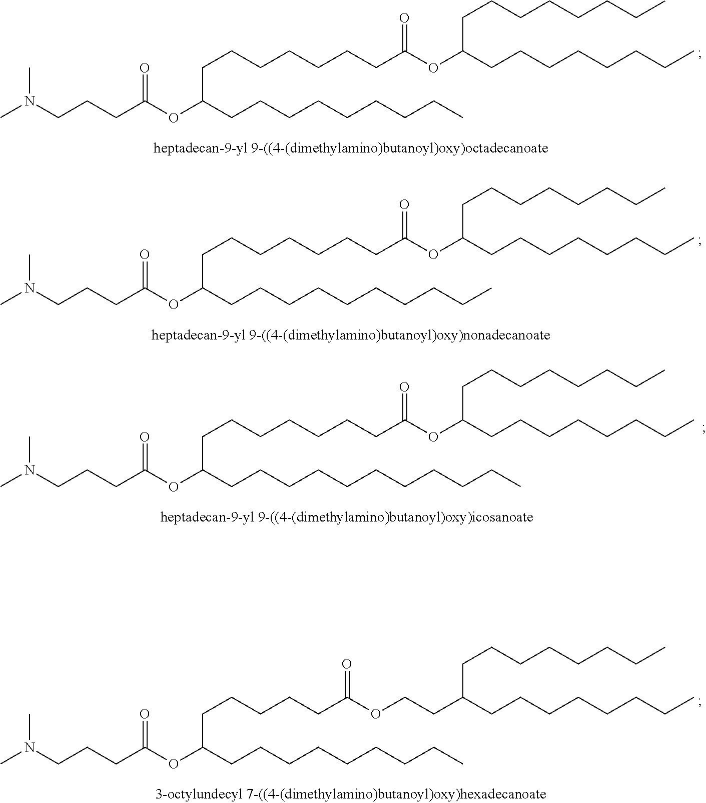

Without limitations, ionizable lipid can comprise 20-90% (mol) of the total lipid present in the lipid nanoparticle. For example, ionizable lipid molar content can be 20-70% (mol), 30-60% (mol) or 40-50% (mol) of the total lipid present in the lipid nanoparticle. According to some embodiments, ionizable lipid comprises from about 50 mol % to about 90 mol % of the total lipid present in the lipid nanoparticle.

According to some aspects, the lipid nanoparticle can further comprise a non-cationic lipid. Non-ionic lipids include amphipathic lipids, neutral lipids and anionic lipids. Accordingly, the non-cationic lipid can be a neutral uncharged, zwitterionic, or anionic lipid. Non-cationic lipids are typically employed to enhance fusogenicity.

Exemplary non-cationic lipids envisioned for use in the methods and compositions as disclosed herein are described in International Application PCT/US2018/050042, filed on Sep. 7, 2018, and PCT/US2018/064242, filed on Dec. 6, 2018 which is incorporated herein in its entirety. Exemplary non-cationic lipids are described in International Application Publication WO2017/099823 and US patent publication US2018/0028664, the contents of both of which are incorporated herein by reference in their entirety.

The non-cationic lipid can comprise 0-30% (mol) of the total lipid present in the lipid nanoparticle. For example, the non-cationic lipid content is 5-20% (mol) or 10-15% (mol) of the total lipid present in the lipid nanoparticle. In various embodiments, the molar ratio of ionizable lipid to the neutral lipid ranges from about 2:1 to about 8:1.

According to some embodiments, the lipid nanoparticles do not comprise any phospholipids. According to some aspects, the lipid nanoparticle can further comprise a component, such as a sterol, to provide membrane integrity.

One exemplary sterol that can be used in the lipid nanoparticle is cholesterol and derivatives thereof. Exemplary cholesterol derivatives are described in International application WO2009/127060 and US patent publication US2010/0130588, contents of both of which are incorporated herein by reference in their entirety.

The component providing membrane integrity, such as a sterol, can comprise 0-50% (mol) of the total lipid present in the lipid nanoparticle. According to some embodiments, such a component is 20-50% (mol) 30-40% (mol) of the total lipid content of the lipid nanoparticle.

According to some aspects, the lipid nanoparticle can further comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. According to some embodiments, the conjugated lipid molecule is a PEG-lipid conjugate, for example, a (methoxy polyethylene glycol)-conjugated lipid. Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, and US2017/0119904, the contents of all of which are incorporated herein by reference in their entirety.

According to some embodiments, a PEG-lipid is a compound as defined in US2018/0028664, the content of which is incorporated herein by reference in its entirety. According to some embodiments, a PEG-lipid is disclosed in US20150376115 or in US2016/0376224, the content of both of which is incorporated herein by reference in its entirety.

The PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. According to some examples, the PEG-lipid can be selected from the group consisting of PEG-DMG, 1,2-dimyristoyl-sn-glycero-3-phosphoethanolamine-N-[methoxy(polyethylene glycol)-2000],

Lipids conjugated with a molecule other than a PEG can also be used in place of PEG-lipid. For example, polyoxazoline (POZ)-lipid conjugates, polyamide-lipid conjugates (such as ATTA-lipid conjugates), and cationic-polymer lipid (CPL) conjugates can be used in place of or in addition to the PEG-lipid. Exemplary conjugated lipids, i.e., PEG-lipids, (POZ)-lipid conjugates, ATTA-lipid conjugates and cationic polymer-lipids are described in the International patent application publications WO1996/010392, WO1998/051278, WO2002/087541, WO2005/026372, WO2008/147438, WO2009/086558, WO2012/000104, WO2017/117528, WO2017/099823, WO2015/199952, WO2017/004143, WO2015/095346, WO2012/000104, WO2012/000104, and WO2010/006282, US patent application publications US2003/0077829, US2005/0175682, US2008/0020058, US2011/0117125, US2013/0303587, US2018/0028664, US2015/0376115, US2016/0376224, US2016/0317458, US2013/0303587, US2013/0303587, and US20110123453, and US patents U.S. Pat. Nos. 5,885,613, 6,287,591, 6,320,017, and 6,586,559, the contents of all of which are incorporated herein by reference in their entirety.

### Combinations

According to some embodiments, the ceDNA vector is administered in combination with one or more additional therapeutic agents, e.g., an anti-cancer therapeutic, an autoimmune therapeutic, an infectious disease therapeutic. According to some embodiments, the agent is a second antigen or immunogenic peptide, as described herein. In some embodiments, the effect of the ceDNA and the additional agent is synergistic. The term “synergistic” or “synergy” means a more than additive effect of a combination of two or more agents compared to their individual effects. In some embodiments, synergistic activity is present when a first agent produces a detectable level of an output X, a second agent produces a detectable level of the output X, and the first and second agents together produce a more-than-additive level of the output X.

Some human tumors can be eliminated by a patient's immune system. For example, administration of a monoclonal antibody targeted to an immune “checkpoint” molecule can lead to complete response and tumor remission. A mode of action of such antibodies is through inhibition of an immune regulatory molecule that the tumors have co-opted as protection from an anti-tumor immune response. By inhibiting these “checkpoint” molecules (e.g., with an antagonistic antibody), a patient's CD8+ T cells may be allowed to proliferate and destroy tumor cells. For example, administration of a monoclonal antibody targeted to by way of example, without limitation, CTLA-4 or PD-1 can lead to complete response and tumor remission. The mode of action of such antibodies is through inhibition of CTLA-4 or PD-1 that the tumors have co-opted as protection from an anti-tumor immune response. By inhibiting these “checkpoint” molecules (e.g., with an antagonistic antibody), a patient's CD8+ T cells may be allowed to proliferate and destroy tumor cells.

Thus, the ceDNA vectors comprising a nucleic acid sequence encoding one or more tumor associated antigens provided herein can be used in combination with one or more blocking antibodies targeted to an immune “checkpoint” molecule. For instance, in some embodiments, the compositions provided herein can be used in combination with one or more blocking antibodies targeted to a molecule such as CTLA-4 or PD-1.

According to some embodiments, a ceDNA composition is administered with an adjuvant. Adjuvants include, but are not limited to, Freund's adjuvant, GM-CSF, Montanide (e.g., Montanide IMS 1312, Montanide ISA 206, Montanide ISA 50V, and Montanide ISA-51), 1018 ISS, aluminium salts, Amplivax®, AS15, BCG, CP-870,893, CpG7909, CyaA, dSLIM, flagellin or TLRS ligands derived from flagellin, FLT3 ligand, IC30, IC31, Imiquimod (ALDARA®), resiquimod, ImuFact IMP321, Interleukins such as IL-2, IL-4, IL-7, IL-12, IL-13, IL-15, IL-21, IL-23, Interferon-α or -β, or pegylated derivatives thereof, IS Patch, ISS, ISCOMATRIX, ISCOMs, JuvImmune, LipoVac, MALP2, MF59, monophosphoryl lipid A, water-in-oil and oil-in-water emulsions, OK-432, OM-174, OM-197-MP-EC, ONTAK, OspA, PepTel® vector system, poly(lactid co-glycolid) [PLG]-based and dextran microparticles, talactoferrin SRL172, virosomes and other virus-like particles, YF-17D, VEGF trap, R848, beta-glucan, Pam3Cys, Aquila's QS21 stimulon, mycobacterial extracts and synthetic bacterial cell wall mimics, Ribi's Detox, Quil, Superfos, cyclophosphamide, sunitinib, bevacizumab, celebrex, NCX-4016, sildenafil, tadalafil, vardenafil, sorafenib, temozolomide, temsirolimus, XL-999, CP-547632, pazopanib, VEGF Trap, ZD2171, AZD2171, and anti-CTLA4 antibodies. CpG immunostimulatory oligonucleotides can be used to enhance the effects of adjuvants in a vaccine setting.

According to some embodiments, the nucleic acid sequence of the ceDNA vector further comprises a sequence that encodes an adjuvant.

Also provided herein is a pharmaceutical composition comprising the lipid nanoparticle-encapsulated insect-cell produced, or a synthetically produced ceDNA vector for expression of antigens, or immunogenic peptides, as described herein and a pharmaceutically acceptable carrier or excipient.

According to some aspects, the disclosure provides for a lipid nanoparticle formulation further comprising one or more pharmaceutical excipients. According to some embodiments, the lipid nanoparticle formulation further comprises sucrose, tris, trehalose and/or glycine.

The ceDNA vector can be complexed with the lipid portion of the particle or encapsulated in the lipid position of the lipid nanoparticle. According to some embodiments, the ceDNA can be fully encapsulated in the lipid position of the lipid nanoparticle, thereby protecting it from degradation by a nuclease, e.g., in an aqueous solution. According to some embodiments, the ceDNA in the lipid nanoparticle is not substantially degraded after exposure of the lipid nanoparticle to a nuclease at 37° C. for at least about 20, 30, 45, or 60 minutes. According to some embodiments, the ceDNA in the lipid nanoparticle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours.

In certain embodiments, the lipid nanoparticles are substantially non-toxic to a subject, e.g., to a mammal such as a human. According to some aspects, the lipid nanoparticle formulation is a lyophilized powder.

According to some embodiments, lipid nanoparticles are solid core particles that possess at least one lipid bilayer. In other embodiments, the lipid nanoparticles have a non-bilayer structure, i.e., a non-lamellar (i.e., non-bilayer) morphology. Without limitations, the non-bilayer morphology can include, for example, three dimensional tubes, rods, cubic symmetries, etc. For example, the morphology of the lipid nanoparticles (lamellar vs. non-lamellar) can readily be assessed and characterized using, e.g., Cryo-TEM analysis as described in US2010/0130588, the content of which is incorporated herein by reference in its entirety.

According to some further embodiments, the lipid nanoparticles having a non-lamellar morphology are electron dense. According to some aspects, the disclosure provides for a lipid nanoparticle that is either unilamellar or multilamellar in structure. According to some aspects, the disclosure provides for a lipid nanoparticle formulation that comprises multi-vesicular particles and/or foam-based particles.

By controlling the composition and concentration of the lipid components, one can control the rate at which the lipid conjugate exchanges out of the lipid particle and, in turn, the rate at which the lipid nanoparticle becomes fusogenic. In addition, other variables including, e.g., pH, temperature, or ionic strength, can be used to vary and/or control the rate at which the lipid nanoparticle becomes fusogenic. Other methods which can be used to control the rate at which the lipid nanoparticle becomes fusogenic will be apparent to those of ordinary skill in the art based on this disclosure. It will also be apparent that by controlling the composition and concentration of the lipid conjugate, one can control the lipid particle size.

The pKa of formulated cationic lipids can be correlated with the effectiveness of the LNPs for delivery of nucleic acids (see Jayaraman et al, Angewandte Chemie, International Edition (2012), 51(34), 8529-8533; Semple et al, Nature Biotechnology 28, 172-176 (2010), both of which are incorporated by reference in their entirety). The preferred range of pKa is ˜5 to ˜7. The pKa of the cationic lipid can be determined in lipid nanoparticles using an assay based on fluorescence of 2-(p-toluidino)-6-napthalene sulfonic acid (TNS).

### VII. Methods of Treatment

A ceDNA vector for expression of antigens, or immunogenic peptides, as disclosed herein can also be used in a method for the delivery of a nucleic acid sequence of interest to a target cell (e.g., a host cell). The method may in particular be a method for delivering antigens and immunogenic peptides to a cell of a subject in need thereof and treating a disease or disorder.

Targets for the antibodies, or antigen-binding fragments described herein, (i.e., antigens) may be selected from a variety of pathogens, including, e.g., bacterial, viral, fungal and parasitic infectious agents. Suitable targets may further include cancer or cancer-associated antigens, or the like. Still other targets may include an autoimmune condition such as rheumatoid arthritis (RA) or multiple sclerosis (MS).

In addition, the disclosure provides a method for the delivery of antigens and immunogenic peptides to a cell of a subject in need thereof, comprising multiple administrations of the ceDNA vector of the disclosure encoding said antigens and immunogenic peptides. Since the ceDNA vector of the disclosure does not induce an immune response like that typically observed against encapsidated viral vectors, such a multiple administration strategy will likely have greater success in a ceDNA-based system. The ceDNA vector are administered in sufficient amounts to transfect the cells of a desired tissue and to provide sufficient levels of gene transfer and expression of the antigens and immunogenic peptides without undue adverse effects.

Delivery of a ceDNA vector for expression of antigens and immunogenic peptides as described herein is not limited to delivery of the expressed antigens, or immunogenic peptides. For example, conventionally produced (e.g., using a cell-based production method (e.g., insect-cell production methods) or synthetically produced ceDNA vectors as described herein may be used with other delivery systems provided to provide a portion of the gene therapy. One non-limiting example of a system that may be combined with the ceDNA vectors in accordance with the present disclosure includes systems which separately deliver one or more co-factors or immune suppressors for effective gene expression of the ceDNA vector expressing the antigens, or immunogenic peptides.

Targets for the immunoglobulin constructs described herein may be selected from a variety of pathogens, including, e.g., bacterial, viral, fungal and parasitic infectious agents. Suitable targets may further include cancer or cancer-associated antigens, or the like. Still other targets may include an autoimmune condition such as rheumatoid arthritis (RA) or multiple sclerosis (MS).

Examples of viral targets include influenza virus from the orthomyxovirudae family, which includes: Influenza A, Influenza B, and Influenza C. The type A viruses are the most virulent human pathogens. The serotypes of influenza A which have been associated with pandemics include, H1N1, which caused Spanish Flu in 1918, and Swine Flu in 2009; H2N2, which caused Asian Flu in 1957; H3N2, which caused Hong Kong Flu in 1968; H5N1, which caused Bird Flu in 2004; H7N7; H1N2; H9N2; H7N2; H7N3; and H10N7.

Broadly neutralizing antibodies against influenza A have been described. As used herein, a “broadly neutralizing antibody” refers to a neutralizing antibody which can neutralize multiple strains from multiple subtypes. For example, CR6261 [The Scripps Institute/Crucell] has been described as a monoclonal antibody that binds to a broad range of the influenza virus including the 1918 “Spanish flu” (SC1918/H1) and to a virus of the H5N1 class of avian influenza that jumped from chickens to a human in Vietnam in 2004 (Viet04/H5). CR6261 recognizes a highly conserved helical region in the membrane-proximal stem of hemagglutinin, the predominant protein on the surface of the influenza virus. This antibody is described in WO 2010/130636, incorporated by reference herein. Another neutralizing antibody, F10 [XOMA Ltd] has been described as being useful against H1N1 and H5N1. [Sui et al, Nature Structural and Molecular Biology (Sui, et al. 2009, 16(3):265-73)] Other antibodies against influenza, e.g., Fab28 and Fab49, may be selected. See, e.g., WO 2010/140114 and WO 2009/115972, which are incorporated by reference. Still other antibodies, such as those described in WO 2010/010466, US Published Patent Publication US/2011/076265, and WO 2008/156763, may be readily selected.

Other target pathogenic viruses include, arenaviruses (including funin, machupo, and Lassa), filoviruses (including Marburg and Ebola), hantaviruses, picornaviridae (including rhinoviruses, echovirus), coronaviruses, paramyxovirus, morbillivirus, respiratory syncytial virus, togavirus, coxsackievirus, parvovirus B19, parainfluenza, adenoviruses, reoviruses, variola (Variola major (Smallpox)) and Vaccinia (Cowpox) from the poxvirus family, and varicella-zoster (pseudorabies).

Viral hemorrhagic fevers are caused by members of the arenavirus family (Lassa fever) (which family is also associated with Lymphocytic choriomeningitis (LCM)), filovirus (ebola virus), and hantavirus (puremala). The members of picornavirus (a subfamily of rhinoviruses), are associated with the common cold in humans. The coronavirus family includes a number of non-human viruses such as infectious bronchitis virus (poultry), porcine transmissible gastroenteric virus (pig), porcine hemagglutinatin encephalomyelitis virus (pig), feline infectious peritonitis virus (cat), feline enteric coronavirus (cat), canine coronavirus (dog). The human respiratory coronaviruses, have been putatively associated with the common cold, non-A, B or C hepatitis, and sudden acute respiratory syndrome (SARS). The paramyxovirus family includes parainfluenza Virus Type 1, parainfluenza Virus Type 3, bovine parainfluenza Virus Type 3, rubulavirus (mumps virus), parainfluenza Virus Type 2, parainfluenza virus Type 4, Newcastle disease virus (chickens), rinderpest, morbillivirus, which includes measles and canine distemper, and pneumovirus, which includes respiratory syncytial virus (RSV). The parvovirus family includes feline parvovirus (feline enteritis), feline panleucopeniavirus, canine parvovirus, and porcine parvovirus. The adenovirus family includes viruses (EX, AD7, ARD, O.B.) which cause respiratory disease.

A neutralizing antibody construct against a bacterial pathogen may also be selected for use in the present disclosure. In one embodiment, the neutralizing antibody construct is directed against the bacteria itself. In another embodiment, the neutralizing antibody construct is directed against a toxin produced by the bacteria. Examples of airborne bacterial pathogens include, e.g., Neisseria meningitidis (meningitis), Klebsiella pneumonia (pneumonia), Pseudomonas aeruginosa (pneumonia), Pseudomonas pseudomallei (pneumonia), Pseudomonas mallei (pneumonia), Acinetobacter (pneumonia), Moraxella catarrhalis, Moraxella lacunata, Alkaligenes, Cardiobacterium, Haemophilus influenzae (flu), Haemophilus parainfluenzae, Bordetella pertussis (whooping cough), Francisella tularensis (pneumonia/fever), Legionella pneumonia (Legionnaires disease), Chlamydia psittaci (pneumonia), Chlamydia pneumoniae (pneumonia), Mycobacterium tuberculosis (tuberculosis (TB)), Mycobacterium kansasii (TB), Mycobacterium avium (pneumonia), Nocardia asteroides (pneumonia), Bacillus anthracis (anthrax), Staphylococcus aureus (pneumonia), Streptococcus pyogenes (scarlet fever), Streptococcus pneumoniae (pneumonia), Corynebacteria diphtheria (diphtheria), Mycoplasma pneumoniae (pneumonia).

The causative agent of anthrax is a toxin produced by Bacillus anthracis. Neutralizing antibodies against protective agent (PA), one of the three peptides which form the toxoid, have been described. The other two polypeptides consist of lethal factor (LF) and edema factor (EF). Anti-PA neutralizing antibodies have been described as being effective in passively immunization against anthrax. See, e.g., U.S. Pat. No. 7,442,373; R. Sawada-Hirai et al, J Immune Based Ther Vaccines. 2004; 2: 5. (on-line 2004 May 12). Still other anti-anthrax toxin neutralizing antibodies have been described and/or may be generated. Similarly, neutralizing antibodies against other bacteria and/or bacterial toxins may be used to generate an AAV-delivered anti-pathogen construct as described herein.

Other infectious diseases may be caused by airborne fungi including, e.g., Aspergillus species, Absidia corymbifera, Rhixpus stolonifer, Mucor plumbeaus, Cryptococcus neoformans, Histoplasm capsulatum, Blastomyces dermatitidis, Coccidioides immitis, Penicillium species, Micropolyspora faeni, Thermoactinomyces vulgaris, Alternaria alternate, Cladosporium species, Helminthosporium, and Stachybotrys species.

In addition, passive immunization may be used to prevent fungal infections (e.g., athlete's foot), ringworm, or viruses, bacteria, parasites, fungi, and other pathogens which can be transmitted by direct contact. In addition, a variety of conditions which affect household pets, cattle and other livestock, and other animals. For example, in dogs, infection of the upper respiratory tract by canine sinonasal aspergillosis causes significant disease. In cats, upper respiratory disease or feline respiratory disease complex originating in the nose causes morbidity and mortality if left untreated. Cattle are prone to infections by the infectious bovine rhinotracheitis (commonly called IBR or red nose) is an acute, contagious virus disease of cattle. In addition, cattle are prone to Bovine Respiratory Syncytial Virus (BRSV) which causes mild to severe respiratory disease and can impair resistance to other diseases. Still other pathogens and diseases will be apparent to one of skill in the art. See, e.g., U.S. Pat. No. 5,811,524, which describes generation of anti-respiratory syncytial virus (RSV) neutralizing antibodies. The techniques described therein are applicable to other pathogens. Such an antibody may be used intact or its sequences (scaffold) modified to generate an artificial or recombinant neutralizing antibody construct. Such methods have been described [see, e.g., WO 2010/13036; WO 2009/115972; WO 2010/140114].

Anti-neoplastic immunoglobulins as described herein may target a human epidermal growth factor receptor (HER), such as HER2. For example, trastuzumab is a recombinant IgGI kappa, humanized monoclonal antibody that selectively binds with high affinity in a cell-based assay (Kd=5 nM) to the extracellular domain of the human epidermal growth factor receptor protein. The commercially available product is produced in CHO cell culture. See, e.g., www.drugbank.ca/drugs/DB00072. The amino acid sequences of the trastuzumab light chains 1 and 2 and heavy chains 1 and 2, as well as sequences obtained from a study of the x-ray structure of trastuzumab, are provided on this database at accession number DB00072, which sequences are incorporated herein by reference. See, also, 212-Pb-TCMC-trastuzumab [Areva Med, Bethesda, Md.].

Another antibody of interest includes, e.g., pertuzumab, a recombinant humanized monoclonal antibody that targets the extracellular dimerization domain (Subdomain II) of the human epidermal growth factor receptor 2 protein (HER2). It consists of two heavy chains and two lights chains that have 448 and 214 residues respectively. FDA approved Jun. 8, 2012. The amino acid sequences of its heavy chain and light chain are provided, e.g., in www.drugbank.ca/drugs/DB06366 (synonyms include 2C4, MOAB 2C4, monoclonal antibody 2C4, and rhuMAb-2C4) on this database at accession number DB06366. In addition to HER2, other HER targets may be selected.

For example, MM-121/SAR256212 is a fully human monoclonal antibody that targets the HER3 receptor [Merrimack's Network Biology] and which has been reported to be useful in the treatment of non-small cell lung cancer (NSCLC), breast cancer and ovarian cancer. SAR256212 is an investigational fully human monoclonal antibody that targets the HER3 (ErbB3) receptor [Sanofi Oncology]. Another anti-Her3/EGFR antibody is RG7597 [Genentech], described as being useful in head and neck cancers. Another antibody, margetuximab (or MGAH22), a next-generation, Fc-optimized monoclonal antibody (mAb) that targets HER [MacroGenics], may also be utilized.

Alternatively, other human epithelial cell surface markers and/or other tumor receptors or antigens may be targeted. Examples of other cell surface marker targets include, e.g., 5T4, CA-125, CEA (e.g., targeted by labetuzumab), CD3, CD19, CD20 (e.g., targeted by rituximab), CD22 (e.g., targeted by epratuzumab or veltuzumab), CD30, CD33, CD40, CD44, CD51 (also integrin avP3), CD133 (e.g., glioblastoma cells), CTLA-4 (e.g., Ipilimumab used in treatment of, e.g., neuroblastoma)), Chemokine (C-X-C Motif) Receptor 2 (CXCR2) (expressed in different regions in brain; e.g., Anti-CXCR2 (extracellular) antibody #ACR-012 (Alomene Labs)); EpCAM, fibroblast activation protein (FAP) [see, e.g., WO 2012020006 A2, brain cancers], folate receptor alpha (e.g., pediatric ependymal brain tumors, head and neck cancers), fibroblast growth factor receptor 1 (FGFR1) (see, et al, WO2012125124A1 for discussion treatment of cancers with anti-FGFR1 antibodies), FGFR2 (see, e.g., antibodies described in WO2013076186A and WO2011143318A2), FGFR3 (see, e.g., antibodies described in U.S. Pat. No. 8,187,601 and WO2010111367A1), FGFR4 (see, e.g., anti-FGFR4 antibodies described in WO2012138975A1), hepatocyte growth factor (HGF) (see, e.g., antibodies in WO2010119991A3), integrin a51, IGF-1 receptor, gangioloside GD2 (see, e.g., antibodies described in WO2011160119A2), ganglioside GD3, transmembrane glycoprotein NMB (GPNMB) (associated with gliomas, among others and target of the antibody glembatumumab (CR011), mucin, MUC1, phosphatidylserine (e.g., targeted by bavituximab, Peregrine Pharmaceuticals, Inc], prostatic carcinoma cells, PD-L1 (e.g., nivolumab (BMS-936558, MDX-1106, ONO-4538), a fully human gG4, e.g., metastatic melanoma], platelet-derived growth factor receptor, alpha (PDGFR a) or CD140, tumor associated glycoprotein 72 (TAG-72), tenascin C, tumor necrosis factor (TNF) receptor (TRAIL-R2), vascular endothelial growth factor (VEGF)-A (e.g., targeted by bevacizumab) and VEGFR2 (e.g., targeted by ramucirumab).

Other antibodies and their targets include, e.g., APN301 (hu14.19-IL2), a monoclonal antibody [malignant melanoma and neuroblastoma in children, Apeiron Biolgics, Vienna, Austria]. See, also, e.g., monoclonal antibody, 8H9, which has been described as being useful for the treatment of solid tumors, including metastatic brain cancer. The monoclonal antibody 8H9 is a mouse IgGI antibody with specificity for the B7H3 antigen [United Therapeutics Corporation]. This mouse antibody can be humanized Still other immunoglobulin constructs targeting the B7-H3 and/or the B7-H4 antigen may be used herein. Another antibody is S58 (anti-GD2, neuroblastoma). Cotara™ [Perregrince Pharmaceuticals] is a monoclonal antibody described for treatment of recurrent glioblastoma. Other antibodies may include, e.g., avastin, ficlatuzumab, medi-575, and olaratumab. Still other immunoglobulin constructs or monoclonal antibodies may be selected for use herein. See, e.g., Medicines in Development Biologics, 2013 Report, pp. 1-87, a publication of PhRMA's Communications & Public Affairs Department. (202) 835-3460, which is incorporated by reference herein.

For example, immunogens may be selected from a variety of viral families. Example of viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Another viral family includes the calcivirus family, which encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinating encephalomyelitis virus (pig), feline infectious peritonitis virus (cats), feline enteric coronavirus (cat), canine coronavirus (dog), and human respiratory coronaviruses, which may cause the common cold and/or non-A, B or C hepatitis. Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the rhabdovirus family, which includes the genera vesiculovirus (e.g., Vesicular Stomatitis Virus), and the general lyssavirus (e.g., rabies).

Within the rhabdovirus family, suitable antigens may be derived from the G protein or the N protein. The family filoviridae, which includes hemorrhagic fever viruses such as Marburg and Ebola virus, may be a suitable source of antigens. The paramyxovirus family includes parainfluenza Virus Type 1, parainfluenza Virus Type 3, bovine parainfluenza Virus Type 3, rubulavirus (mumps virus), parainfluenza Virus Type 2, parainfluenza virus Type 4, Newcastle disease virus (chickens), rinderpest, morbillivirus, which includes measles and canine distemper, and pneumovirus, which includes respiratory syncytial virus. The influenza virus is classified within the family orthomyxovirus and is a suitable source of antigen (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bunyaviruses. The arenavirus family provides a source of antigens against LCM and Lassa fever virus. The reovirus family includes the genera reovirus, rotavirus (which causes acute gastroenteritis in children), orbiviruses, and cultivirus (Colorado Tick fever, Lebombo (humans), equine encephalosis, blue tongue).

The retrovirus family includes the sub-family oncorivirinal which encompasses such human and veterinary diseases as feline leukemia virus, HTLVI and HTLVII, lentivirinal (which includes human immunodeficiency virus (HIV), simian immunodeficiency virus (SIV), feline immunodeficiency virus (FIV), equine infectious anemia virus, and spumavirinal). Among the lentiviruses, many suitable antigens have been described and can readily be selected as targets. Examples of suitable HIV and SIV antigens include, without limitation the gag, pol, Vif, Vpx, VPR, Env, Tat, Nef, and Rev proteins, as well as various fragments thereof. For example, suitable fragments of the Env protein may include any of its subunits such as the gp120, gp160, gp41, or smaller fragments thereof, e.g., of at least about 8 amino acids in length. Similarly, fragments of the tat protein may be selected. [See, U.S. Pat. Nos. 5,891,994 and 6,193,981.] See, also, the HIV and SIV proteins described in D. H. Barouch et al, J. Virol., 75(5):2462-2467 (March 2001), and R. R. Amara, et al, Science, 292:69-74 (6 Apr. 2001). In another example, the HIV and/or SIV immunogenic proteins or peptides may be used to form fusion proteins or other immunogenic molecules. See, e.g., the HIV-1 Tat and/or Nef fusion proteins and immunization regimens described in WO 01/54719, published Aug. 2, 2001, and WO 99/16884, published Apr. 8, 1999. The invention is not limited to the HIV and/or SIV immunogenic proteins or peptides described herein. In addition, a variety of modifications to these proteins has been described or could readily be made by one of skill in the art. See, e.g., the modified gag protein that is described in U.S. Pat. No. 5,972,596.

The papovavirus family includes the sub-family polyomaviruses (BKU and JCU viruses) and the sub-family papillomavirus (associated with cancers or malignant progression of papilloma). The adenovirus family includes viruses (EX, AD7, ARD, O.B.) which cause respiratory disease and/or enteritis. The parvovirus family includes feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (HCMV, muromegalovirus) and the sub-family gammaherpesvirinae, which includes the genera lymphocryptovirus, EBV (Burkitts lymphoma), infectious rhinotracheitis, Marek's disease virus, and rhadinovirus. The poxvirus family includes the sub-family chordopoxvirinae, which encompasses the genera orthopoxvirus (Variola (Smallpox) and Vaccinia (Cowpox)), parapoxvirus, avipoxvirus, capripoxvirus, leporipoxvirus, suipoxvirus, and the sub-family entomopoxvirinae. The hepadnavirus family includes the Hepatitis B virus. One unclassified virus which may be suitable source of antigens is the Hepatitis delta virus. Still other viral sources may include avian infectious bursal disease virus and porcine respiratory and reproductive syndrome virus. The alphavirus family includes equine arteritis virus and various Encephalitis viruses.

Other pathogenic targets for antibodies may include, e.g., bacteria, fungi, parasitic microorganisms or multicellular parasites which infect human and non-human vertebrates, or from a cancer cell or tumor cell. Examples of bacterial pathogens include pathogenic gram-positive cocci include pneumococci; staphylococci; and streptococci. Pathogenic gram-negative cocci include meningococcus; gonococcus. Pathogenic enteric gram-negative bacilli include enterobacteriaceae; pseudomonas, acinetobacteria and eikenella; melioidosis; salmonella; shigella; haemophilus; moraxella; H. ducreyi (which causes chancroid); brucella; Franisella tularensis (which causes tularemia); yersinia (pasteurella); streptobacillus moniliformis and spirillum; Gram-positive bacilli include Listeria monocytogenes; Erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism; other clostridia; tuberculosis; leprosy; and other mycobacteria. Pathogenic spirochetal diseases include syphilis; treponematoses: yaws, pinta and endemic syphilis; and leptospirosis. Other infections caused by higher pathogen bacteria and pathogenic fungi include actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever, and Rickettsialpox. Examples of mycoplasma and chlamydial infections include: Mycoplasma pneumoniae; lymphogranuloma venereum; psittacosis; and perinatal chlamydial infections. Pathogenic eukaryotes encompass pathogenic protozoa and helminthes and infections produced thereby include: amebiasis; malaria; leishmaniasis; trypanosomiasis; toxoplasmosis; Pneumocystis carinii; Trichans; Toxoplasma gondii; babesiosis; giardiasis; trichinosis; filariasis; schistosomiasis; nematodes; trematodes or flukes; and cestode (tapeworm) infections.

Many of these organisms and/or toxins produced thereby have been identified by the Centers for Disease Control [(CDC), Department of Health and Human Services, USA], as agents which have potential for use in biological attacks. For example, some of these biological agents, include, Bacillus anthracis (anthrax), Clostridium botulinum and its toxin (botulism), Yersinia pestis (plague), variola major (smallpox), Francisella tularensis (tularemia), and viral hemorrhagic fevers [filoviruses (e.g., Ebola, Marburg], and arenaviruses [e.g., Lassa, Machupo]), all of which are currently classified as Category A agents; Coxiella burnetti (Q fever); Brucella species (brucellosis), Burkholderia mallei (glanders), Burkholderia pseudomallei (meloidosis), Ricinus communis and its toxin (ricin toxin), Clostridium perfringens and its toxin (epsilon toxin), Staphylococcus species and their toxins (enterotoxin B), Chlamydia psittaci (psittacosis), water safety threats (e.g., Vibrio cholerae, Crytosporidium parvum), Typhus fever (Richettsia powazekii), and viral encephalitis (alphaviruses, e.g., Venezuelan equine encephalitis; eastern equine encephalitis; western equine encephalitis); all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vectors and other constructs described herein are useful to target antigens from these organisms, viruses, their toxins or other by-products, which will prevent and/or treat infection or other adverse reactions with these biological agents.

An effective or therapeutically effective dose of a ceDNA vector for expression of antigens and immunogenic peptides as described herein, for treating or preventing a viral infection refers to the amount of the ceDNA vector for expression of antigen, or immunogenic peptide, as described herein, antigens and immunogenic peptides that is sufficient to alleviate one or more signs and/or symptoms of the infection in the treated subject, whether by inducing the regression or elimination of such signs and/or symptoms or by inhibiting the progression of such signs and/or symptoms. The dose amount may vary depending upon the age and the size of a subject to be administered, target disease, conditions, route of administration, and the like. In an embodiment of the disclosure, an effective or therapeutically effective dose of antibody or antigen-binding fragment thereof of the present disclosure, for treating or preventing viral infection, e.g., in an adult human subject, is about 0.01 to about 200 mg/kg, e.g., up to about 150 mg/kg. In an embodiment of the disclosure, the dosage is up to about 10.8 or 11 grams (e.g., about 1, 2, 3, 4, 5, 6, 7, 8, 9, 10 or 11 grams). Depending on the severity of the disease or infection, the frequency and the duration of the treatment can be adjusted. In certain embodiments, the ceDNA vector for expression of the antigens and immunogenic peptides as described herein can be administered at an initial dose, followed by one or more secondary doses. In certain embodiments, the initial dose may be followed by administration of a second or a plurality of subsequent doses of antigen in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

The subject that is administered the ceDNA vector may have a viral infection, e.g., an influenza infection, or be predisposed to developing an infection. Subjects predisposed to developing an infection, or subjects who may be at elevated risk for contracting an infection (e.g., of coronavirus or influenza virus), include subjects with compromised immune systems because of autoimmune disease, subjects receiving immunosuppressive therapy (for example, following organ transplant), subjects afflicted with human immunodeficiency syndrome (HIV) or acquired immune deficiency syndrome (AIDS), subjects with forms of anemia that deplete or destroy white blood cells, subjects receiving radiation or chemotherapy, or subjects afflicted with an inflammatory disorder. Additionally, subjects of very young (e.g., 5 years of age or younger) or old age (e.g., 65 years of age or older) are at increased risk. Moreover, a subject may be at risk of contracting a viral infection due to proximity to an outbreak of the disease, e.g., subject resides in a densely-populated city or in close proximity to subjects having confirmed or suspected infections of a virus, or choice of employment, e.g., hospital worker, pharmaceutical researcher, traveler to infected area, or frequent flier.

The present disclosure also encompasses prophylactically administering a ceDNA vector for expression of antigen, or immunogenic peptide, as described herein, to a subject who is at risk of a disease or disorder, e.g., a viral infection so as to prevent such infection. “Prevent” or “preventing” means to administer a ceDNA vector for expression of antigen, or immunogenic peptide, as described herein, to a subject to inhibit the manifestation of a disease or infection (e.g., viral infection) in the body of a subject, for which the ceDNA vector for expression of antigens, or immunogenic peptides, as described herein is effective when administered to the subject at an effective or therapeutically effective amount or dose.

According to some embodiments, a sign or symptom of a viral infection in a subject is survival or proliferation of virus in the body of the subject, e.g., as determined by viral titer assay (e.g., coronavirus propagation in embryonated chicken eggs or coronavirus spike protein assay). Other signs and symptoms of viral infection are discussed herein.

As noted above, according to some embodiments the subject may be a non-human animal, and the antibodies and antigen-binding fragments discussed herein may be used in a veterinary context to treat and/or prevent disease in the non-human animals (e.g., cats, dogs, pigs, cows, horses, goats, rabbits, sheep, and the like).

The present disclosure provides a method for treating or preventing viral infection (e.g., coronavirus infection) or for inducing the regression or elimination or inhibiting the progression of at least one sign or symptom of viral infection such as: fever or feeling feverish/chills; cough; sore throat; runny or stuffy nose; sneezing; muscle or body aches; headaches; fatigue (tiredness); vomiting; diarrhea; respiratory tract infection; chest discomfort; shortness of breath; bronchitis; and/or pneumonia, which sign or symptom is secondary to viral infection, in a subject in need thereof (e.g., a human), by administering a therapeutically effective amount of a ceDNA vector for expression of antigens, or immunogenic peptides, as described herein to the subject.

### Ex Vivo Treatment

According to some embodiments, cells are removed from a subject, a ceDNA vector for expression of antigens, or immunogenic peptides, as disclosed herein is introduced therein, and the cells are then replaced back into the subject. Methods of removing cells from subject for treatment ex vivo, followed by introduction back into the subject are known in the art (see, e.g., U.S. Pat. No. 5,399,346; the disclosure of which is incorporated herein in its entirety). Alternatively, a ceDNA vector is introduced into cells from another subject, into cultured cells, or into cells from any other suitable source, and the cells are administered to a subject in need thereof.

Cells transduced with a ceDNA vector for expression of antigens, or immunogenic peptides, as disclosed herein are preferably administered to the subject in a “therapeutically-effective amount” in combination with a pharmaceutical carrier. Those skilled in the art will appreciate that the therapeutic effects need not be complete or curative, as long as some benefit is provided to the subject.

According to some embodiments, a ceDNA vector for expression of antigens, or immunogenic peptides, as disclosed herein can encode an antibody, and antigen-binding fragment thereof, as described herein that is to be produced in a cell in vitro, ex vivo, or in vivo. For example, in contrast to the use of the ceDNA vectors described herein in a method of treatment as discussed herein, according to some embodiments a ceDNA vector for expression of antigens, or immunogenic peptides, may be introduced into cultured cells and the expressed antigens, or immunogenic peptides, isolated from the cells after a period of time, e.g., for the production of antibodies and fusion proteins. According to some embodiments, the cultured cells comprising a ceDNA vector for expression of antigens, or immunogenic peptides, as disclosed herein can be used for commercial production of antibodies or fusion proteins, e.g., serving as a cell source for small or large scale biomanufacturing of antibodies or fusion proteins. In alternative embodiments, a ceDNA vector for expression of antigens, or immunogenic peptides, as disclosed herein is introduced into cells in a host non-human subject, for in vivo production of antibodies or fusion proteins, including small scale production as well as for commercial large scale antigens, or immunogenic peptides, production.

The ceDNA vectors for expression of antigens and immunogenic peptides as disclosed herein can be used in both veterinary and medical applications. Suitable subjects for ex vivo gene delivery methods as described above include both avians (e.g., chickens, ducks, geese, quail, turkeys and pheasants) and mammals (e.g., humans, bovines, ovines, caprines, equines, felines, canines, and lagomorphs), with mammals being preferred. Human subjects are most preferred. Human subjects include neonates, infants, juveniles, and adults.

### Dose Ranges

Provided herein are methods of treatment comprising administering to the subject an effective amount of a composition comprising a ceDNA vector encoding antigens, or immunogenic peptides, as described herein.

In vivo and/or in vitro assays can optionally be employed to help identify optimal dosage ranges for use. The precise dose to be employed in the formulation will also depend on the route of administration, and the seriousness of the condition, and should be decided according to the judgment of the person of ordinary skill in the art and each subject's circumstances. Effective doses can be extrapolated from dose-response curves derived from in vitro or animal model test systems

A ceDNA vector for expression of antigens, or immunogenic peptides, as disclosed herein is administered in sufficient amounts to transfect the cells of a desired tissue and to provide sufficient levels of gene transfer and expression without undue adverse effects. Conventional and pharmaceutically acceptable routes of administration include, but are not limited to, those described above in the “Administration” section, such as direct delivery to the selected organ (e.g., intraportal delivery to the liver), oral, inhalation (including intranasal and intratracheal delivery), intraocular, intravenous, intramuscular, subcutaneous, intradermal, intratumoral, and other parental routes of administration. Routes of administration can be combined, if desired.

The dose of the amount of a ceDNA vectors for expression of antigens, or immunogenic peptides, as disclosed herein required to achieve a particular “therapeutic effect,” will vary based on several factors including, but not limited to: the route of nucleic acid administration, the level of gene or RNA expression required to achieve a therapeutic effect, the specific disease or disorder being treated, and the stability of the gene(s), RNA product(s), or resulting expressed protein(s). One of skill in the art can readily determine a ceDNA vector dose range to treat a patient having a particular disease or disorder based on the aforementioned factors, as well as other factors that are well known in the art.

Dosage regime can be adjusted to provide the optimum therapeutic response. For example, the oligonucleotide can be repeatedly administered, e.g., several doses can be administered daily or the dose can be proportionally reduced as indicated by the exigencies of the therapeutic situation. One of ordinary skill in the art will readily be able to determine appropriate doses and schedules of administration of the subject oligonucleotides, whether the oligonucleotides are to be administered to cells or to subjects.

A “therapeutically effective dose” for clinical use will fall in a relatively broad range that can be determined through clinical trials and will depend on the particular application (e.g., neural cells will require very small amounts, while systemic injection would require large amounts). For example, for direct in vivo injection into skeletal or cardiac muscle of a human subject, a therapeutically effective dose will be on the order of from about 1 μg to 100 g of the ceDNA vector. If exosomes or microparticles are used to deliver the ceDNA vector, then a therapeutically effective dose can be determined experimentally, but is expected to deliver from 1 μg to about 100 g of vector. Moreover, a therapeutically effective dose is an amount ceDNA vector that expresses a sufficient amount of the transgene to have an effect on the subject that results in a reduction According to some or more symptoms of the disease, but does not result in significant off-target or significant adverse side effects. According to some embodiments, a “therapeutically effective amount” is an amount of an expressed antigens, or immunogenic peptides, that is sufficient to produce a statistically significant, measurable change in reduction of a given disease symptom. Such effective amounts can be gauged in clinical trials as well as animal studies for a given ceDNA vector composition.

For in vitro transfection, an effective amount of a ceDNA vectors for expression of antigens, or immunogenic peptides, as disclosed herein to be delivered to cells (1×10⁶ cells) will be on the order of 0.1 to 100 μg ceDNA vector, preferably 1 to 20 μg, and more preferably 1 to 15 μg or 8 to 10 μg. Larger ceDNA vectors will require higher doses. If exosomes or microparticles are used, an effective in vitro dose can be determined experimentally but would be intended to deliver generally the same amount of the ceDNA vector.

Treatment can involve administration of a single dose or multiple doses. According to some embodiments, more than one dose can be administered to a subject; in fact, multiple doses can be administered as needed, because the ceDNA vector does not elicit an anti-capsid host immune response due to the absence of a viral capsid. As such, one of skill in the art can readily determine an appropriate number of doses. According to some embodiments, the doses are administered in a prime-dose dosing regime.

Without wishing to be bound by any particular theory, the lack of typical anti-viral immune response elicited by administration of a ceDNA vector as described by the disclosure (i.e., the absence of capsid components) allows the ceDNA vector for expression of antigens, or immunogenic peptides, to be administered to a host on multiple occasions. According to some embodiments, the number of occasions in which a nucleic acid is delivered to a subject is in a range of 2 to 10 times (e.g., 2, 3, 4, 5, 6, 7, 8, 9, or 10 times). According to some embodiments, a ceDNA vector is delivered to a subject more than 5 times. According to some embodiments, a ceDNA vector is delivered to a subject more than 3 times. According to some embodiments, a ceDNA vector is delivered to a subject more than 2 times.

According to some embodiments, a dose of a ceDNA vector for expression of antigens, or immunogenic peptides, as disclosed herein is administered to a subject no more than once per calendar day (e.g., a 24-hour period). According to some embodiments, a dose of a ceDNA vector is administered to a subject no more than once per 2, 3, 4, 5, 6, or 7 calendar days. According to some embodiments, a dose of a ceDNA vector for expression of antigens, or immunogenic peptides, as disclosed herein is administered to a subject no more than once per calendar week (e.g., 7 calendar days). According to some embodiments, a dose of a ceDNA vector is administered to a subject no more than bi-weekly (e.g., once in a two calendar week period). According to some embodiments, a dose of a ceDNA vector is administered to a subject no more than once per calendar month (e.g., once in 30 calendar days). According to some embodiments, a dose of a ceDNA vector is administered to a subject no more than once per six calendar months. According to some embodiments, a dose of a ceDNA vector is administered to a subject no more than once per calendar year (e.g., 365 days or 366 days in a leap year).

According to some embodiments, a dose of a ceDNA vector is administered on day 0. Following the initial treatment at day 0, a second dosing (re-dose) can be performed in about 1 week, about 2 weeks, about 3 weeks, about 4 weeks, about 5 weeks, about 6 weeks, about 7 weeks, about 8 weeks, or about 3 months, about 4 months, about 5 months, about 6 months, about 7 months, about 8 months, about 9 months, about 10 months, about 11 months, or about 1 year, about 2 years, about 3 years, about 4 years, about 5 years, about 6 years, about 7 years, about 8 years, about 9 years, about 10 years, about 11 years, about 12 years, about 13 years, about 14 years, about 15 years, about 16 years, about 17 years, about 18 years, about 19 years, about 20 years, about 21 years, about 22 years, about 23 years, about 24 years, about 25 years, about 26 years, about 27 years, about 28 years, about 29 years, about 30 years, about 31 years, about 32 years, about 33 years, about 34 years, about 35 years, about 36 years, about 37 years, about 38 years, about 39 years, about 40 years, about 41 years, about 42 years, about 43 years, about 44 years, about 45 years, about 46 years, about 47 years, about 48 years, about 49 years or about 50 years after the initial treatment with the ceDNA vector.

According to some embodiments, re-dosing of the therapeutic nucleic acid results in an increase in expression of the therapeutic nucleic acid. According to some embodiments, the increase of expression of the therapeutic nucleic acid after re-dosing, compared to the expression of the therapeutic nucleic acid after the first dose is about 0.5-fold to about 10-fold, about 1-fold to about 5-fold, about 1-fold to about 2-fold, or about 0.5-fold, about 1-fold, about 2-fold, about 3-fold, about 4-fold, about 5-fold, about 6-fold, about 7-fold, about 8-fold, about 9-fold or about 10-fold higher after re-dosing of the therapeutic nucleic acid.

In particular embodiments, more than one administration (e.g., two, three, four or more administrations) of a ceDNA vector for expression of antigens, or immunogenic peptides, as disclosed herein may be employed to achieve the desired level of antibody expression over a period of various intervals, e.g., daily, weekly, monthly, yearly, etc.

According to some embodiments, a therapeutic antigens, or immunogenic peptides, encoded by a ceDNA vector as disclosed herein can be regulated by a regulatory switch, inducible or repressible promotor so that it is expressed in a subject for at least 1 hour, at least 2 hours, at least 5 hours, at least 10 hours, at least 12 hours, at least 18 hours, at least 24 hours, at least 36 hours, at least 48 hours, at least 72 hours, at least 1 week, at least 2 weeks, at least 1 month, at least 2 months, at least 6 months, at least 12 months/one year, at least 2 years, at least 5 years, at least 10 years, at least 15 years, at least 20 years, at least 30 years, at least 40 years, at least 50 years or more. According to some embodiments, the expression can be achieved by repeated administration of the ceDNA vectors described herein at predetermined or desired intervals.

As described herein, according to some embodiments, a ceDNA vector expressing a Antigens, or immunogenic peptides, can be administered in combination with an additional compound.

### Unit Dosage Forms

According to some embodiments, the pharmaceutical compositions comprising a ceDNA vector for expression of antigens, or immunogenic peptides, as disclosed herein can conveniently be presented in unit dosage form. A unit dosage form will typically be adapted to one or more specific routes of administration of the pharmaceutical composition. According to some embodiments, the unit dosage form is adapted for intravenous, intramuscular, or subcutaneous administration. According to some embodiments, the unit dosage form is adapted for administration by inhalation. According to some embodiments, the unit dosage form is adapted for administration by a vaporizer. According to some embodiments, the unit dosage form is adapted for administration by a nebulizer. According to some embodiments, the unit dosage form is adapted for administration by an aerosolizer. According to some embodiments, the unit dosage form is adapted for oral administration, for buccal administration, or for sublingual administration.

Testing for Successful Gene Expression Using a ceDNA Vector

Assays well known in the art can be used to test the efficiency of gene delivery of antigens, or immunogenic peptides, by a ceDNA vector can be performed in both in vitro and in vivo models. Levels of the expression of the Antigens, or immunogenic peptides, by ceDNA can be assessed by one skilled in the art by measuring mRNA and protein levels of the Antigens, or immunogenic peptides, (e.g., reverse transcription PCR, western blot analysis, and enzyme-linked immunosorbent assay (ELISA)). According to some embodiments, ceDNA comprises a reporter protein that can be used to assess the expression of the antibodies, and the antigen-binding fragments thereof, for example by examining the expression of the reporter protein by fluorescence microscopy or a luminescence plate reader. For in vivo applications, protein function assays can be used to test the functionality of a given Antigens, or immunogenic peptides, to determine if gene expression has successfully occurred. One skilled will be able to determine the best test for measuring functionality of antigens, or immunogenic peptides, expressed by the ceDNA vector in vitro or in vivo.

It is contemplated herein that the effects of gene expression of antigens, or immunogenic peptides, from the ceDNA vector in a cell or subject can last for at least 1 month, at least 2 months, at least 3 months, at least four months, at least 5 months, at least six months, at least 10 months, at least 12 months, at least 18 months, at least 2 years, at least 5 years, at least 10 years, at least 20 years, or can be permanent.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents is based on the information available to the applicants and does not constitute any admission as to the correctness of the dates or contents of these documents.

### EXAMPLES

The following examples are provided by way of illustration not limitation. It will be appreciated by one of ordinary skill in the art that ceDNA vectors can be constructed from any of the wild-type or modified ITRs described herein, and that the following exemplary methods can be used to construct and assess the activity of such ceDNA vectors. While the methods are exemplified with certain ceDNA vectors, they are applicable to any ceDNA vector in keeping with the description.

### Example 1: Constructing ceDNA Vectors Using an Insect Cell-Based Method

Production of the ceDNA vectors using a polynucleotide construct template is described in Example 1 of PCT/US18/49996, which is incorporated herein in its entirety by reference. For example, a polynucleotide construct template used for generating the ceDNA vectors of the present disclosure can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g., ceDNA-plasmid, ceDNA-bacmid, ceDNA-baculovirus genome etc.) via Rep proteins, and second, Rep mediated replication of the excised ceDNA vector.

An exemplary method to produce ceDNA vectors is from a ceDNA-plasmid as described herein. Referring to FIGS. 1A and 1B, the polynucleotide construct template of each of the ceDNA-plasmids includes both a left modified ITR and a right modified ITR with the following between the ITR sequences: (i) an enhancer/promoter; (ii) a cloning site for a transgene; (iii) a posttranscriptional response element (e.g., the woodchuck hepatitis virus posttranscriptional regulatory element (WPRE)); and (iv) a poly-adenylation signal (e.g., from bovine growth hormone gene (BGHpA). Unique restriction endonuclease recognition sites (R1-R6) (shown in FIG. 1A and FIG. 1B) were also introduced between each component to facilitate the introduction of new genetic components into the specific sites in the construct. R3 (PmeI) GTTTAAAC (SEQ ID NO: 123) and R4 (Pacd) TTAATTAA (SEQ ID NO: 124) enzyme sites are engineered into the cloning site to introduce an open reading frame of a transgene. These sequences were cloned into a pFastBac HT B plasmid obtained from ThermoFisher Scientific.

Production of ceDNA-Bacmids:

DH10Bac competent cells (MAX EFFICIENCY® DH10Bac™ Competent Cells, Thermo Fisher) were transformed with either test or control plasmids following a protocol according to the manufacturer's instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (Φ80dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test constructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest and change in cell viability every day for 4 to 5 days.

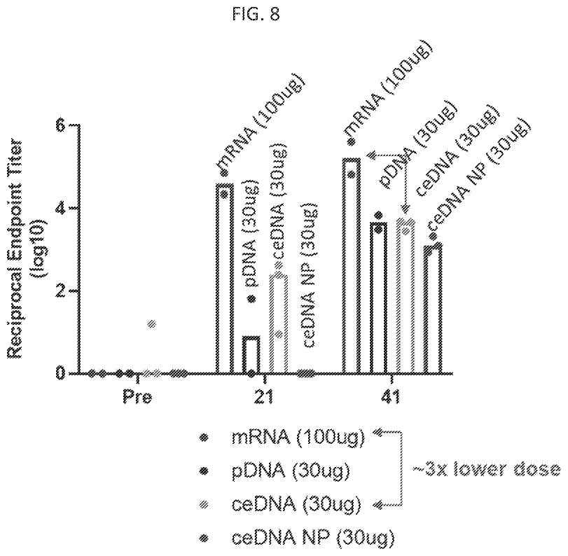

A “Rep-plasmid” as disclosed in FIG. 8A of PCT/US18/49996, which is incorporated herein in its entirety by reference, was produced in a pFASTBAC™-Dual expression vector (ThermoFisher) comprising both the Rep78 (SEQ ID NO: 131 or 133) and Rep52 (SEQ ID NO: 132) or Rep68 (SEQ ID NO: 130) and Rep40 (SEQ ID NO: 129). The Rep-plasmid was transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (Φ80dlacZΔM15 marker provides α-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

With reference to FIG. 4B, Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm.

ceDNA vectors can be assessed by identified by agarose gel electrophoresis under native or denaturing conditions as illustrated in FIG. 4D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the DNA obtained from co-infected Sf9 cells (as described herein) with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIGS. 4D and 4E, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a non-encapsidated vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 4D).

As used herein, the phrase “assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately ⅓× and ⅔× of the DNA vector length. This resolves the bands on both native and denaturing gels. Before denaturation, it is important to remove the buffer from the sample. The Qiagen PCR clean-up kit or desalting “spin columns,” e.g., GE HEALTHCARE ILUSTRA™ MICROSPIN™ G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1×TBE or TAE and transferred to distilled water or 1×TBE/TAE with 1×SYBR Gold. Bands can then be visualized with e.g., Thermo Fisher, SYBR® Gold Nucleic Acid Gel Stain (10,000× Concentrate in DMSO) and epifluorescent light (blue) or UV (312 nm).

The purity of the generated ceDNA vector can be assessed using any art-known method. As one exemplary and non-limiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg. then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

For comparative purposes, Example 1 describes the production of ceDNA vectors using an insect cell-based method and a polynucleotide construct template, and is also described in Example 1 of PCT/US18/49996, which is incorporated herein in its entirety by reference. For example, a polynucleotide construct template used for generating the ceDNA vectors of the present disclosure according to Example 1 can be a ceDNA-plasmid, a ceDNA-Bacmid, and/or a ceDNA-baculovirus. Without being limited to theory, in a permissive host cell, in the presence of e.g., Rep, the polynucleotide construct template having two symmetric ITRs and an expression construct, where at least one of the ITRs is modified relative to a wild-type ITR sequence, replicates to produce ceDNA vectors. ceDNA vector production undergoes two steps: first, excision (“rescue”) of template from the template backbone (e.g., ceDNA-plasmid, ceDNA-bacmid, ceDNA-baculovirus genome etc.) via Rep proteins, and second, Rep mediated replication of the excised ceDNA vector.

An exemplary method to produce ceDNA vectors in a method using insect cell is from a ceDNA-plasmid as described herein. Referring to FIGS. 1A and 1B, the polynucleotide construct template of each of the ceDNA-plasmids includes both a left modified ITR and a right modified ITR with the following between the ITR sequences: (i) an enhancer/promoter; (ii) a cloning site for a transgene; (iii) a posttranscriptional response element (e.g., the woodchuck hepatitis virus posttranscriptional regulatory element (WPRE)); and (iv) a poly-adenylation signal (e.g., from bovine growth hormone gene (BGHpA). Unique restriction endonuclease recognition sites (R1-R6) (shown in FIG. 1A and FIG. 1B) were also introduced between each component to facilitate the introduction of new genetic components into the specific sites in the construct. R3 (PmeI) GTTTAAAC (SEQ ID NO: 123) and R4 (PacI) TTAATTAA (SEQ ID NO: 124) enzyme sites are engineered into the cloning site to introduce an open reading frame of a transgene. These sequences were cloned into a pFastBac HT B plasmid obtained from ThermoFisher Scientific.

Production of ceDNA-Bacmids:

DH10Bac competent cells (MAX EFFICIENCY® DH10Bac™ Competent Cells, Thermo Fisher) were transformed with either test or control plasmids following a protocol according to the manufacturer's instructions. Recombination between the plasmid and a baculovirus shuttle vector in the DH10Bac cells were induced to generate recombinant ceDNA-bacmids. The recombinant bacmids were selected by screening a positive selection based on blue-white screening in E. coli (Φ80dlacZΔM15 marker provides α-complementation of the β-galactosidase gene from the bacmid vector) on a bacterial agar plate containing X-gal and IPTG with antibiotics to select for transformants and maintenance of the bacmid and transposase plasmids. White colonies caused by transposition that disrupts the β-galactoside indicator gene were picked and cultured in 10 ml of media.

The recombinant ceDNA-bacmids were isolated from the E. coli and transfected into Sf9 or Sf21 insect cells using FugeneHD to produce infectious baculovirus. The adherent Sf9 or Sf21 insect cells were cultured in 50 ml of media in T25 flasks at 25° C. Four days later, culture medium (containing the P0 virus) was removed from the cells, filtered through a 0.45 μm filter, separating the infectious baculovirus particles from cells or cell debris.

Optionally, the first generation of the baculovirus (P0) was amplified by infecting naïve Sf9 or Sf21 insect cells in 50 to 500 ml of media. Cells were maintained in suspension cultures in an orbital shaker incubator at 130 rpm at 25° C., monitoring cell diameter and viability, until cells reach a diameter of 18-19 nm (from a naïve diameter of 14-15 nm), and a density of ˜4.0E+6 cells/mL. Between 3 and 8 days post-infection, the P1 baculovirus particles in the medium were collected following centrifugation to remove cells and debris then filtration through a 0.45 μm filter.

The ceDNA-baculovirus comprising the test constructs were collected and the infectious activity, or titer, of the baculovirus was determined. Specifically, four×20 ml Sf9 cell cultures at 2.5E+6 cells/ml were treated with P1 baculovirus at the following dilutions: 1/1000, 1/10,000, 1/50,000, 1/100,000, and incubated at 25-27° C. Infectivity was determined by the rate of cell diameter increase and cell cycle arrest, and change in cell viability every day for 4 to 5 days.

A “Rep-plasmid” was produced in a pFASTBAC™-Dual expression vector (ThermoFisher) comprising both the Rep78 (SEQ ID NO: 131 or 133) or Rep68 (SEQ ID NO: 130) and Rep52 (SEQ ID NO: 132) or Rep40 (SEQ ID NO: 129). The Rep-plasmid was transformed into the DH10Bac competent cells (MAX EFFICIENCY® DH10Bac™ Competent Cells (Thermo Fisher) following a protocol provided by the manufacturer. Recombination between the Rep-plasmid and a baculovirus shuttle vector in the DH10Bac cells were induced to generate recombinant bacmids (“Rep-bacmids”). The recombinant bacmids were selected by a positive selection that included-blue-white screening in E. coli (Φ80dlacZΔM15 marker provides α-complementation of the β-galactosidase gene from the bacmid vector) on a bacterial agar plate containing X-gal and IPTG. Isolated white colonies were picked and inoculated in 10 ml of selection media (kanamycin, gentamicin, tetracycline in LB broth). The recombinant bacmids (Rep-bacmids) were isolated from the E. coli and the Rep-bacmids were transfected into Sf9 or Sf21 insect cells to produce infectious baculovirus.

The Sf9 or Sf21 insect cells were cultured in 50 ml of media for 4 days, and infectious recombinant baculovirus (“Rep-baculovirus”) were isolated from the culture. Optionally, the first generation Rep-baculovirus (P0) were amplified by infecting naïve Sf9 or Sf21 insect cells and cultured in 50 to 500 ml of media. Between 3 and 8 days post-infection, the P1 baculovirus particles in the medium were collected either by separating cells by centrifugation or filtration or another fractionation process. The Rep-baculovirus were collected and the infectious activity of the baculovirus was determined. Specifically, four×20 mL Sf9 cell cultures at 2.5×10⁶ cells/mL were treated with P1 baculovirus at the following dilutions, 1/1000, 1/10,000, 1/50,000, 1/100,000, and incubated. Infectivity was determined by the rate of cell diameter increase and cell cycle arrest, and change in cell viability every day for 4 to 5 days.

ceDNA Vector Generation and Characterization

Sf9 insect cell culture media containing either (1) a sample-containing a ceDNA-bacmid or a ceDNA-baculovirus, and (2) Rep-baculovirus described above were then added to a fresh culture of Sf9 cells (2.5E+6 cells/ml, 20 ml) at a ratio of 1:1000 and 1:10,000, respectively. The cells were then cultured at 130 rpm at 25° C. 4-5 days after the co-infection, cell diameter and viability are detected. When cell diameters reached 18-20 nm with a viability of ˜70-80%, the cell cultures were centrifuged, the medium was removed, and the cell pellets were collected. The cell pellets are first resuspended in an adequate volume of aqueous medium, either water or buffer. The ceDNA vector was isolated and purified from the cells using Qiagen MIDI PLUS™ purification protocol (Qiagen, 0.2 mg of cell pellet mass processed per column).

Yields of ceDNA vectors produced and purified from the Sf9 insect cells were initially determined based on UV absorbance at 260 nm. The purified ceDNA vectors can be assessed for proper closed-ended configuration using the electrophoretic methodology described in Example 5.

### Example 2: Synthetic ceDNA Production Via Excision from a Double-Stranded DNA Molecule

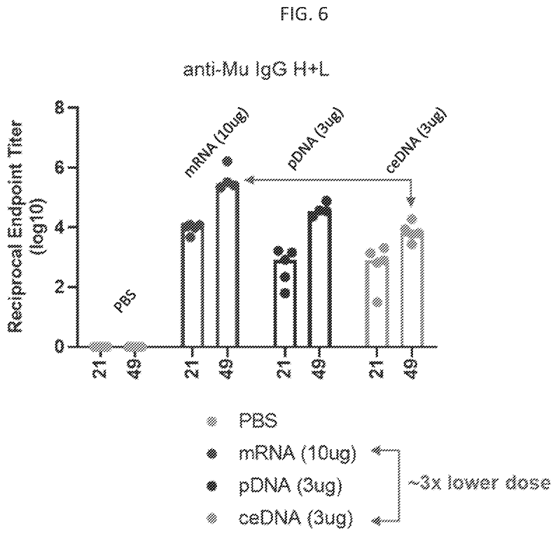

Synthetic production of the ceDNA vectors is described in Examples 2-6 of International Application PCT/US19/14122, filed Jan. 18, 2019, which is incorporated herein in its entirety by reference. One exemplary method of producing a ceDNA vector using a synthetic method that involves the excision of a double-stranded DNA molecule. In brief, a ceDNA vector can be generated using a double stranded DNA construct, e.g., see FIGS. 7A-8E of PCT/US19/14122. According to some embodiments, the double stranded DNA construct is a ceDNA plasmid, e.g., see, e.g., FIG. 6 in International patent application PCT/US2018/064242, filed Dec. 6, 2018).

According to some embodiments, a construct to make a ceDNA vector comprises a regulatory switch as described herein.

For illustrative purposes, Example 2 describes producing ceDNA vectors as exemplary closed-ended DNA vectors generated using this method. However, while ceDNA vectors are exemplified in this Example to illustrate in vitro synthetic production methods to generate a closed-ended DNA vector by excision of a double-stranded polynucleotide comprising the ITRs and expression cassette (e.g., nucleic acid sequence) followed by ligation of the free 3′ and 5′ ends as described herein, one of ordinary skill in the art is aware that one can, as illustrated above, modify the double stranded DNA polynucleotide molecule such that any desired closed-ended DNA vector is generated, including but not limited to, doggybone DNA, dumbbell DNA and the like. Exemplary ceDNA vectors for production of antibodies or fusion proteins that can be produced by the synthetic production method described in Example 2 are discussed in the sections entitled “III ceDNA vectors in general”. Exemplary antibodies and fusion proteins expressed by the ceDNA vectors are described in the section entitled “IIC Exemplary antibodies and fusion proteins expressed by the ceDNA vectors”.

The method involves (i) excising a sequence encoding the expression cassette from a double-stranded DNA construct and (ii) forming hairpin structures at one or more of the ITRs and (iii) joining the free 5′ and 3′ ends by ligation, e.g., by T4 DNA ligase.

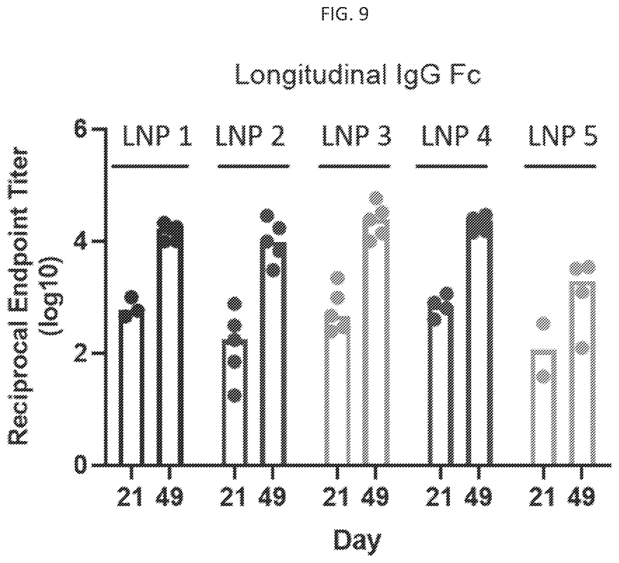

The double-stranded DNA construct comprises, in 5′ to 3′ order: a first restriction endonuclease site; an upstream ITR; an expression cassette; a downstream ITR; and a second restriction endonuclease site. The double-stranded DNA construct is then contacted with one or more restriction endonucleases to generate double-stranded breaks at both of the restriction endonuclease sites. One endonuclease can target both sites, or each site can be targeted by a different endonuclease as long as the restriction sites are not present in the ceDNA vector template. This excises the sequence between the restriction endonuclease sites from the rest of the double-stranded DNA construct (see FIG. 9 of PCT/US19/14122). Upon ligation a closed-ended DNA vector is formed.

One or both of the ITRs used in the method may be wild-type ITRs. Modified ITRs may also be used, where the modification can include deletion, insertion, or substitution of one or more nucleotides from the wild-type ITR in the sequences forming B and B′ arm and/or C and C′ arm (see, e.g., FIGS. 6-8 and 10 FIG. 11B of PCT/US19/14122), and may have two or more hairpin loops (see, e.g., FIGS. 6-8 FIG. 11B of PCT/US19/14122) or a single hairpin loop (see, e.g., FIG. 10A-10B FIG. 11B of PCT/US19/14122). The hairpin loop modified ITR can be generated by genetic modification of an existing oligo or by de novo biological and/or chemical synthesis.

In a non-limiting example, ITR-6 Left and Right (SEQ ID NOS: 111 and 112), include 40 nucleotide deletions in the B-B′ and C-C′ arms from the wild-type ITR of AAV2. Nucleotides remaining in the modified ITR are predicted to form a single hairpin structure. Gibbs free energy of unfolding the structure is about −54.4 kcal/mol. Other modifications to the ITR may also be made, including optional deletion of a functional Rep binding site or a Trs site.

### Example 3: ceDNA Production Via Oligonucleotide Construction

Another exemplary method of producing a ceDNA vector using a synthetic method that involves assembly of various oligonucleotides, is provided in Example 3 of PCT/US19/14122, where a ceDNA vector is produced by synthesizing a 5′ oligonucleotide and a 3′ ITR oligonucleotide and ligating the ITR oligonucleotides to a double-stranded polynucleotide comprising an expression cassette. FIG. 11B of PCT/US19/14122 shows an exemplary method of ligating a 5′ ITR oligonucleotide and a 3′ ITR oligonucleotide to a double stranded polynucleotide comprising an expression cassette.

As disclosed herein, the ITR oligonucleotides can comprise WT-ITRs (e.g., see FIG. 3A, FIG. 3C), or modified ITRs (e.g., see, FIG. 3B and FIG. 3D). (See also, e.g., FIGS. 6A, 6B, 7A and 7B of PCT/US19/14122, which is incorporated herein in its entirety). Exemplary ITR oligonucleotides include, but are not limited to SEQ ID NOS: 134-145 (e.g., see Table 7 in of PCT/US19/14122). Modified ITRs can include deletion, insertion, or substitution of one or more nucleotides from the wild-type ITR in the sequences forming B and B′ arm and/or C and C′ arm. ITR oligonucleotides, comprising WT-ITRs or mod-ITRs as described herein, to be used in the cell-free synthesis, can be generated by genetic modification or biological and/or chemical synthesis. As discussed herein, the ITR oligonucleotides in Examples 2 and 3 can comprise WT-ITRs, or modified ITRs (mod-ITRs) in symmetrical or asymmetrical configurations, as discussed herein.

### Example 4: ceDNA Production Via a Single-Stranded DNA Molecule

Another exemplary method of producing a ceDNA vector using a synthetic method is provided in Example 4 of PCT/US19/14122, and uses a single-stranded linear DNA comprising two sense ITRs which flank a sense expression cassette sequence and are attached covalently to two antisense ITRs which flank an antisense expression cassette, the ends of which single stranded linear DNA are then ligated to form a closed-ended single-stranded molecule. One non-limiting example comprises synthesizing and/or producing a single-stranded DNA molecule, annealing portions of the molecule to form a single linear DNA molecule which has one or more base-paired regions of secondary structure, and then ligating the free 5′ and 3′ ends to each other to form a closed single-stranded molecule.

An exemplary single-stranded DNA molecule for production of a ceDNA vector comprises, from 5′ to 3′:
a sense first ITR;
a sense expression cassette sequence;
a sense second ITR;
an antisense second ITR;
an antisense expression cassette sequence; and
an antisense first ITR.

A single-stranded DNA molecule for use in the exemplary method of Example 4 can be formed by any DNA synthesis methodology described herein, e.g., in vitro DNA synthesis, or provided by cleaving a DNA construct (e.g., a plasmid) with nucleases and melting the resulting dsDNA fragments to provide ssDNA fragments.

Annealing can be accomplished by lowering the temperature below the calculated melting temperatures of the sense and antisense sequence pairs. The melting temperature is dependent upon the specific nucleotide base content and the characteristics of the solution being used, e.g., the salt concentration. Melting temperatures for any given sequence and solution combination are readily calculated by one of ordinary skill in the art.

The free 5′ and 3′ ends of the annealed molecule can be ligated to each other, or ligated to a hairpin molecule to form the ceDNA vector. Suitable exemplary ligation methodologies and hairpin molecules are described in Examples 2 and 3.

### Example 5: Purifying and/or Confirming Production of ceDNA

Any of the DNA vector products produced by the methods described herein, e.g., including the insect cell based production methods described in Example 1, or synthetic production methods described in Examples 2-4 can be purified, e.g., to remove impurities, unused components, or byproducts using methods commonly known by a skilled artisan; and/or can be analyzed to confirm that DNA vector produced, (in this instance, a ceDNA vector) is the desired molecule. An exemplary method for purification of the DNA vector, e.g., ceDNA is using Qiagen Midi Plus purification protocol (Qiagen) and/or by gel purification,

The following is an exemplary method for confirming the identity of ceDNA vectors.

ceDNA vectors can be assessed by identified by agarose gel electrophoresis under native or denaturing conditions as illustrated in FIG. 4D, where (a) the presence of characteristic bands migrating at twice the size on denaturing gels versus native gels after restriction endonuclease cleavage and gel electrophoretic analysis and (b) the presence of monomer and dimer (2×) bands on denaturing gels for uncleaved material is characteristic of the presence of ceDNA vector.

Structures of the isolated ceDNA vectors were further analyzed by digesting the purified DNA with restriction endonucleases selected for a) the presence of only a single cut site within the ceDNA vectors, and b) resulting fragments that were large enough to be seen clearly when fractionated on a 0.8% denaturing agarose gel (>800 bp). As illustrated in FIGS. 4C and 4D, linear DNA vectors with a non-continuous structure and ceDNA vector with the linear and continuous structure can be distinguished by sizes of their reaction products—for example, a DNA vector with a non-continuous structure is expected to produce 1 kb and 2 kb fragments, while a ceDNA vector with the continuous structure is expected to produce 2 kb and 4 kb fragments.

Therefore, to demonstrate in a qualitative fashion that isolated ceDNA vectors are covalently closed-ended as is required by definition, the samples were digested with a restriction endonuclease identified in the context of the specific DNA vector sequence as having a single restriction site, preferably resulting in two cleavage products of unequal size (e.g., 1000 bp and 2000 bp). Following digestion and electrophoresis on a denaturing gel (which separates the two complementary DNA strands), a linear, non-covalently closed DNA will resolve at sizes 1000 bp and 2000 bp, while a covalently closed DNA (i.e., a ceDNA vector) will resolve at 2× sizes (2000 bp and 4000 bp), as the two DNA strands are linked and are now unfolded and twice the length (though single stranded). Furthermore, digestion of monomeric, dimeric, and n-meric forms of the DNA vectors will all resolve as the same size fragments due to the end-to-end linking of the multimeric DNA vectors (see FIG. 4E).

As used herein, the phrase “assay for the Identification of DNA vectors by agarose gel electrophoresis under native gel and denaturing conditions” refers to an assay to assess the close-endedness of the ceDNA by performing restriction endonuclease digestion followed by electrophoretic assessment of the digest products. One such exemplary assay follows, though one of ordinary skill in the art will appreciate that many art-known variations on this example are possible. The restriction endonuclease is selected to be a single cut enzyme for the ceDNA vector of interest that will generate products of approximately ⅓× and ⅔× of the DNA vector length. This resolves the bands on both native and denaturing gels. Before denaturation, it is important to remove the buffer from the sample. The Qiagen PCR clean-up kit or desalting “spin columns,” e.g., GE HEALTHCARE ILUSTRA™ MICROSPIN™ G-25 columns are some art-known options for the endonuclease digestion. The assay includes for example, i) digest DNA with appropriate restriction endonuclease(s), 2) apply to e.g., a Qiagen PCR clean-up kit, elute with distilled water, iii) adding 10× denaturing solution (10×=0.5 M NaOH, 10 mM EDTA), add 10× dye, not buffered, and analyzing, together with DNA ladders prepared by adding 10× denaturing solution to 4×, on a 0.8-1.0% gel previously incubated with 1 mM EDTA and 200 mM NaOH to ensure that the NaOH concentration is uniform in the gel and gel box, and running the gel in the presence of 1× denaturing solution (50 mM NaOH, 1 mM EDTA). One of ordinary skill in the art will appreciate what voltage to use to run the electrophoresis based on size and desired timing of results. After electrophoresis, the gels are drained and neutralized in 1×TBE or TAE and transferred to distilled water or 1×TBE/TAE with 1×SYBR Gold. Bands can then be visualized with e.g., Thermo Fisher, SYBR® Gold Nucleic Acid Gel Stain (10,000× Concentrate in DMSO) and epifluorescent light (blue) or UV (312 nm). The foregoing gel-based method can be adapted to purification purposes by isolating the ceDNA vector from the gel band and permitting it to renature.

The purity of the generated ceDNA vector can be assessed using any art-known method. As one exemplary and non-limiting method, contribution of ceDNA-plasmid to the overall UV absorbance of a sample can be estimated by comparing the fluorescent intensity of ceDNA vector to a standard. For example, if based on UV absorbance 4 μg of ceDNA vector was loaded on the gel, and the ceDNA vector fluorescent intensity is equivalent to a 2 kb band which is known to be 1 μg, then there is 1 μg of ceDNA vector, and the ceDNA vector is 25% of the total UV absorbing material. Band intensity on the gel is then plotted against the calculated input that band represents—for example, if the total ceDNA vector is 8 kb, and the excised comparative band is 2 kb, then the band intensity would be plotted as 25% of the total input, which in this case would be 0.25 μg for 1.0 μg input. Using the ceDNA vector plasmid titration to plot a standard curve, a regression line equation is then used to calculate the quantity of the ceDNA vector band, which can then be used to determine the percent of total input represented by the ceDNA vector, or percent purity.

### Example 6: A Study to Evaluate Anti-Spike Antibody Responses after Intramuscular (IM) Administration of LNP:DNA Formulations in Female BALB/c Mice

The objective of the study was to evaluate the anti-spike protein antibody response after intramuscular (IM) injection of LNP:DNA formulations. The study design and details were carried out as set forth below.

### Study Design

Table 13 sets forth the design of the study. SARS-CoV-2 spike protein antigen was delivered as either ceDNA vector, plasmid DNA or mRNA. Test material was dosed in 10 groups of mice (n=5) at a dose level of 3 μg or 10 μg in a dose volume of 30 μl/animal. Groups 1 and 12 served as the controls. Dosing was performed by IM injection as set forth in the Table below. Day 49 was the terminal time point of the study.

TABLE 13

| Group | No. of | | Dose | | Dose | Terminal |
|---|---|---|---|---|---|---|
| No. | Animals | Test Material | Levels (μg) | Dosing Regimen ROA | Volume (μL/an) | Time Point |
| 1 | 5 | PBS | NA | Once on Days 0 & 28 by IM | 30 | Day 49 |
| 2 | 5 | pDNA:LNP | 30 | Once on Day 28 by IM | | |
| 3 | 5 | mRNA:LNP 1 | 10 | Once on Days 0 & 28 by IM | | |
| 4 | 5 | pDNA:LNP 1 | 3 | | | |
| 5 | 5 | ceDNA:LNP 1 | 3 | | | |
| 6 | 5 | ceDNA:LNP 2 | 3 | Once on Day 28 by IM | | |
| 7 | 5 | ceDNA:LNP 2 | 10 | | | |
| 8 | 5 | pDNA:LNP 1 | 3 | Once on Days 0 & 28 by IM | | |
| 9 | 5 | pDNA:LNP | 3 | | | |
| 10 | 5 | ceDNA:LNP 2 | 3 | | | |
| 11 | 5 | ceDNA:LNP 1 | 3 | | | |
| 12 | 5 | PBS | N/A | | | |

No. = Number;
an = animal;
IM = intramuscular;
ROA = route of administration

### Test System

The test system was as follows:
Species: Mus musculus
Strain: Balb/c mouse
Number of females: 60, plus 3 spares
Age: 7 weeks of age at arrival
Source: Charles River Laboratories

Housing: Animals were group housed in clear polycarbonate cages with contact bedding in a procedure room.

Food and Water: Animals were provided ad libitum Mouse Diet 5058 and filtered tap water acidified with 1N HCl to a targeted pH of 2.5-3.0.

### Test Material

Class of Compound: Recombinant DNA Vector: ceDNA; Nucleic acid based biologic (synthetic): pDNA & mRNA.

Dose Formulation: Test articles were supplied in a concentrated stock. Test article concentration was recorded at time of receipt.

Stock was warmed to room temperature and diluted with the provided PBS immediately, as necessary, prior to use. Prepared materials were stored at ˜4° C. if dosing was not performed immediately.

Test Material Administration: Test and control articles were dosed at 30p L per animal on Days 0 & 28 for Groups 1, 3, 4, 5, 8-12 and on Day 28 only for Groups 2, 6, & 7 by intramuscular administration into the LEFT gastrocnemius. Animals were anesthetized with inhalant isoflurane, to effect, for dose procedures.

Residual Materials: All residual open stock was retained for future dosing, refrigerated. Diluted dose materials were discarded after the completion of the dose administration.

### In-Life Observations and Measurements

Cage Side Observations (Animal Health Checks): Cage side animal health checks were performed at least once daily to check for general health, mortality and morbidity.

Clinical Observations: Clinical observations as well as injection site observations were performed ˜1, ˜5-6 and ˜24 hours, 2 days and 3 days post the Day 0 & 28 Test Material doses.

Body Weights: Body weights for all animals were recorded on Days 0, 1, 2, 3, 7, 11, 14, 21, 28, 29, 30, 31, 35, 39, 42, & 49 (prior to euthanasia). Additional body weights were recorded as requested.

In-life Imaging: On Days 4, 11, 21, 39, and 49 animals in Groups 10-12 were dosed with luciferin at 150 mg/kg (60 mg/mL) via intraperitoneal (IP) injection at 2.5 mL/kg. ≤15 minutes post each luciferin administration; all animals had an IVIS imaging session as set forth herein. Animals were placed in dorsal recumbency for the image as the liver was the target organ.

Anesthesia and Recovery: Animals were monitored continuously while under anesthesia, during recovery and until mobile, per testing facility SOPs.

### Blood Collection

All animals in Groups 1, 3, 4, 5, 8-12 had interim blood collected on Days 11, 21 & 39, and animals in Groups 2, 6, & 7 had interim blood collected on Day 39 as shown in Table 14 below.

All animals had whole blood for serum collection.

Whole blood for serum was collected by orbital or tail collection. Whole blood was collected into a serum separator with clot activator tube and processed into one (1) aliquot of serum per facility SOPs.

All samples were stored at nominally −70° C. until shipped to on dry ice.

TABLE 14
Blood Collection (Interim)

| | Whole Blood Sample Collection Times |
|---|---|
| | Orbital or Tail Collection Only |
| Group | (no saphenous) |
| Number | Serum^{a} |
| 1, 3, 4, 5, 8, | Days 11, 21, 39 |
| & 9 | |
| 2, 6, & 7 | Day 39 |
| Volume/ | ~100 μL whole blood |
| Portion | |
| Processing | One (1) aliquot |
| Storage | Frozen at nominally −70° C. |

^{a}Whole blood was collected into serum separator tubes, with clot activator

After each collection animals received 0.5-1.0 mL lactated Ringer's; subcutaneously.

Whole blood for serum was collected by saphenous vein. Whole blood was collected into a serum separator with clot activator tube and processed into one aliquot of serum.

All samples were stored at nominally −70° C. until shipped to on dry ice.

Anesthesia Recovery: As applicable, animals were monitored continuously while under anesthesia, during recovery and until mobile.

### Terminal Procedures and Collections

TABLE 15
Blood Collection- Terminal

| | Sample Collection Times |
|---|---|
| | Terminal Whole Blood |
| Group | (cardiac) |
| Number | Serum^{a} |
| 1-9 | Day 49 |
| Portion | MOV |
| Processing/ | Four (4) aliquots frozen at nominally −70° C. |
| Storage | |

MOV = maximum obtainable volume
^{a}Whole blood was collected into serum separator tubes, with clot activator

TABLE 16
Tissue Collection- Terminal

| | Sample Collection Times |
|---|---|
| Group | Spleen |
| Number | Splenocytes |
| 1-9 | Day 49 |
| Portion | MOV |
| Processing/ | Processed by Testing Facility and Frozen in Freezing |
| Storage | Medium |

Terminal Blood: Groups 1-12, on Day 49, terminal blood was collected from moribund animals that were euthanized prior to their scheduled time point. For all animals, whole blood from exsanguination was collected into a serum separator with clot activator tube and processed into four (4) aliquots of serum per facility SOPs. All samples were stored at nominally −70° C. until shipped to on dry ice.

Terminal Tissues: For Groups 1-9, spleens were harvested and weighed. Spleens were processed into splenocytes using Miltenyi Dissociation Kit per Testing Facility Protocol. After processing, spleens were counted, pelleted, and resuspended. Yield and dissociated cell viability was recorded. Up to 60 million cells were frozen as a suspension in Cell Culture Freezing medium (Gibco #12648010), at up to 10 million cells per mL. Cells were stored at nominally −70° C. until shipped on dry ice.

### Results

FIG. 6 shows spike protein antibody titer as determined on interim Day 21 and Day 49 endpoint of the study. The mRNA construct, representing the commercially available COVID-19 vaccine, was used as the benchmark for comparison of spike protein antibody titer. As shown in FIG. 6, the spike protein Ab titer when ceDNA was employed was within about 10 times of the mRNA benchmark in mice, even when a dose of ceDNA was 3 times lower than the dose of mRNA. FIG. 7 depicts detectable neutralization kinetics seen in the Balb/c mice group treated with ceDNA encoding SARS-CoV-2 spike protein antigen.

### In-Vivo Imaging Protocol (IVIS)

### Materials:

Appropriate syringe for luciferin administration
Appropriate device and/or syringe for luciferin administration
Firefly Luciferin
PBS
pH meter or equivalent
5-M NaOH
5-M HCl
K/X anesthetics or Isoflurane

### Procedure:

### Luciferin Preparation

Luciferin stock powder is stored at nominally −20° C. Store formulated luciferin in 1 mL aliquots at 2-8° C. protect from light. Formulated luciferin is stable for up to 3 weeks at 2-8° C., protected from light and stable for about 12 hrs at room temperature (RT).

Dissolve luciferin in PBS to a target concentration of 60 mg/mL at a sufficient volume and adjusted to pH=7.4 with 5-M NaOH (˜0.5 μl/mg luciferin) and HCl (˜0.5 L/mg luciferin) as needed.

Prepare the appropriate amount according to protocol including at least a ˜50% overage.

### Injection and Imaging (Note: Up to 5 Animals May be Imaged at One Time)

Shave animal's hair coat (as needed). Per protocol, inject 150 mg/kg of luciferin in PBS at 60 mg/mL via IP. Imaging can be performed immediately or up to 15 minutes post dose. Set isoflurane vaporizer to 1-3% (usually @2.5%) to anesthetize the animals during imaging sessions. Isoflurane anesthesia for imaging session: Place the Animal into the isoflurane chamber and wait for the isoflurane to take effect, about 2-3 minutes. Ensure that the anesthesia level on the side of the IVIS machine is positioned to the “on” position. Place animal(s) into the IVIS machine and shut the door. Log into the IVIS computer and open the desired Acquisition Protocol. Recommended acquisition settings for highest sensitivity are: camera height at D level, F/Stop at f1, binning at medium resolution, and exposure time to auto. Press the “ACQUIRE” in the camera control panel interface. Insert labels onto all acquired images. Images will be saved.

### Example 7: A 28-Day Immunogenicity and Tolerability Study by Intramuscular Injection in Cynomolgus Monkeys

The objectives of this study were to determine the immunogenicity profile of two COVID-19 vaccine formulations (LNP 1 and LNP 2), following repeat-dose intramuscular injection to cynomolgus monkeys, and to evaluate the potential reversibility of any findings.

### Test Material Identification

The test articles used were as follows:

Test Article Identification 1

| | Test Article 1 |
|---|---|
| Identification | ceDNA spike:LNP1 |
| Retest Date | Concomitant |
| Physical Description | Clear, colorless liquid |
| Concentration | 0.5 mg/mL |
| Density | Assume 1 g/mL |
| Storage Conditions | Temperature set to maintain |
| | 4° C. |

Test Article Identification 2

| | Test Article 2 |
|---|---|
| Identification | ceDNA spike:LNP 2 |
| Retest Date | Concomitant |
| Physical Description | Clear, colorless liquid |
| Concentration | 0.5 mg/mL |
| Density | Assume 1 g/mL |
| Storage Conditions | Temperature set to maintain |
| | 4° C. |

pDNA Positive Control Identification

| | Positive Control 1 |
|---|---|
| Identification | Plasmid spike:LNP 1 |
| Retest Date | Concomitant |
| Physical Description | Clear, colorless liquid |
| Concentration | 0.5 mg/mL |
| Density | Assume 1 g/mL |
| Storage Conditions | Temperature set to maintain |
| | 4° C. |

mRNA Positive Control Identification

| | Positive Control 2 |
|---|---|
| Identification | mRNA spike:LNP 1 |
| Retest Date | Concomitant |
| Physical Description | Clear, colorless liquid |
| Concentration | 0.5 mg/mL |
| Density | Assume 1 g/mL |
| Storage Conditions | Temperature set to maintain |
| | 4° C. |

Vehicle Identification

| | Vehicle 1 |
|---|---|
| Identification | Phosphate Buffered Saline, pH |
| | 7.4 |
| Alternate | PBS |
| Identification | |
| Storage Conditions | Temperature set to |
| | maintain 23° C. |

### Dose Formulation

Preparation of Formulations: Dose formulation analysis was performed. Dose formulations were divided into aliquots where required in order to allow them to be dispensed on each dosing occasion.

Dose Formulation Preparation

| | | Storage Conditions for |
|---|---|---|
| | | Dose Formulations |
| | | Between Dose |
| | | Preparation and Dose |
| | Frequency of | Administration Set |
| Dose Formulation | Preparation | to Maintain |
| Plasmid spike:LNP 1 | On Days 1 and 28 | Temperature set to |
| (Positive Control 1) | | maintain 4° C. |
| mRNA spike:LNP 1 | On Days 1 and 28 | Temperature set to |
| (Positive Control 2) | | maintain 4° C. |
| ceDNA spike:LNP 1 | On Days 1 and 28 | Temperature set to |
| | | maintain 4° C. |
| ceDNA spike:LNP 2 | On Days 1 and 28 | Temperature set to |
| | | maintain 4° C. |

All residual volumes, a minimum of 0.5 mL, including all unused volumes of dosing formulation from each dosing occasion were transferred to a freezer set to maintain −80° C. and later shipped to the Sponsor.

### Preparation Details

On the day of dose administration, the bulk test articles and positive control materials were transferred from refrigeration to ambient temperature for a minimum of 15 minutes (but no longer than 60 minutes) prior to the start of dose preparation. Bulk test articles and positive control materials were be gently swirled or gently inverted prior to use, i.e., no vortexing and vigorous shaking.

Dosing formulations were prepared in a biosafety cabinet at appropriate concentrations to meet dose level requirements by dilution of the bulk test article and positive control test material with vehicle, if required, in sterile polypropylene containers. Dosing formulations were not filtered. The physical appearance of the dosing formulations was recorded following dilution. Residual dosing formulations, including all unused volumes of dosing formulation, were retained following each day of dosing, and stored in a freezer set to maintain −80° C., where they were maintained for possible future concentration analysis.

Dosing was completed within 4 hours from end of preparation. All remaining bulk test article, positive control test material, and vehicle(s) was returned to −80° C. storage after completion of dosing until shipment to the Sponsor.

### Test System

Species: Cynomolgus monkey (Macaca fascicularis)

### Animal Screening

Method: All animals used on study had documentation to confirm one negative tuberculosis (TB) test. Additional TB testing was performed as required.

### Animal Identification

Method: Tattoo and/or a subcutaneously implanted electronic identification chip

### Environmental Acclimation

Method: The animals were acclimated to laboratory housing for at least 2 weeks before the initiation of dosing.

### Selection, Assignment, Replacement, and Disposition of Animals

Selection and Assignment: Animals were randomized and assigned to groups using a computer based procedure prior to transfer to study. Animals in poor health or at extremes of body weight range were not be assigned to groups.

Replacement: Before the initiation of dosing, any assigned animals considered unsuitable for use in the study were replaced by alternate animals. After initiation of dosing, study animals may be replaced during the replacement period with alternate animals in the event of accidental injury, non-test article-related health issues, or similar circumstances. The alternate animals may be used as replacements on the study within 3 days. General in-life assessments included alternate animals until released from study.

Disposition: The disposition of all animals was documented in the study records.

### Husbandry

### Housing

Housing: Group housed (up to 3 animals of the same dosing group together).

Caging: Stainless steel cages with mesh floors.

Cage Identification: Marked indicating study, group, animal/tattoo number(s), and sex.

Housing set-up is as specified in the USDA Animal Welfare Act (Code of Federal Regulations, Title 9) and as described in the Guide for the Care and Use of Laboratory Animals (NRC, Current Edition). The animals were separated during designated procedures/activities or as required for monitoring and/or health purposes as deemed appropriate by the Study Director and/or Clinical Veterinarian. The room(s) in which the animals were kept was documented in the study records.

### Animal Enrichment

Socially-housed animals were provided with psychological enrichments such as a device (e.g., toy). Also, small bits of fruit, cereal, or other treats may be occasionally given to the animals.

As a reward and means to promote operant conditioning and desired behavior, each animal may be offered a food treat following study-related procedures (e.g., dosing or sample collection) as deemed necessary throughout the duration of the study.

### Environmental Conditions

The targeted conditions for animal room environment were as follows:

Temperature: 64° F. to 84° F. (18° C. to 29° C.)

Humidity: 30% to 70%

Light Cycle: 12 hours light and 12 hours dark (except during designated procedures)

Ventilation: Ten or more air changes per hour with 100% fresh air (no recirculation)

Any deviations to temperature or humidity that exceed the alarm delay time frame that could affect the integrity of the study were reported.

### Food

Diet: PMI Nutrition International Certified Primate Chow No. 5048.

The diet was supplemented with fruit or vegetables at least 2-3 times weekly.

Frequency/Ration: The chow was provided in amounts appropriate for the size and age of the animals.

Analysis: Results of analysis for nutritional components and environmental contaminants are provided by the supplier and are on file at the Testing Facility. It is considered that there are no known contaminants in the feed that would interfere with the objectives of the study.

### Water

Type: Municipal tap water, treated by reverse osmosis and ultraviolet irradiation.

Frequency/Ration: Freely available to each animal via an automatic watering system (except during designated procedures).

Analysis: Periodic analysis of the water is performed, and results of these analyses are on file at the Testing Facility. It is considered that there are no known contaminants in the water that would interfere with the outcome of the study.

### Veterinary Care

Veterinary care was available throughout the course of the study and animals were examined by the veterinary staff as warranted by clinical signs or other changes.

### Experimental Design

| | | | Dose | Dose | No. of |
|---|---|---|---|---|---|
| Group | | Dose | Volume | Concentration | Male |
| No. | Test Material | (mg/dose) | (mL) | (mg/mL) | Animals |
| 1 | Plasmid spike:LNP 1 | 30 μg | 0.5 mL | 0.06 | 2 |
| | (Positive Control 1) | | | | |
| 2 | mRNA spike:LNP 1(Positive | 100 μg | 0.5 mL | 0.2 | 2 |
| | Control 2) | | | | |
| 3 | ceDNA spike:LNP 1 | 30 μg | 0.5 mL | 0.06 | 3 |
| 4 | ceDNA spike:LNP 2 (NP) | 30 μg | 0.5 mL | 0.06 | 3 |

^{a}All study animals were released to the Testing Facility no earlier than the day after the last in-life collection was completed (i.e., no earlier than the day after study Day 41).

### Administration of Test Materials

Dose Route: Intramuscular injection to anterior thigh
Frequency: Once daily
Duration: Days 1 and 28
Method: The first day of dosing was designated as Day 1. The animals were temporarily restrained for dose administration and were not sedated. Each injection site was marked with tattoo or indelible ink and re-marked as often as needed.

### In-Life Procedures, Observations and Measurements

General In-Life Assessments

| | | Frequency | |
|---|---|---|---|
| Parameter | Population^{a} | (Minimum Required) | Comments |
| Mortality | All study | At least once daily (afternoon) | The animals were |
| | animals | beginning by Week 3 | observed within their |
| | | | cage unless necessary for |
| | | | identification or |
| | | | confirmation of possible |
| | | | findings. |
| Cage Side | All study | At least once daily in the morning | The animals were |
| Observations | animals | beginning by Week 3 | observed within their |
| | | | cage unless necessary for |
| | | | identification or |
| | | | confirmation of possible |
| | | | findings.^{b} |
| Individual | All study | Weekly; from at least Week 3 | Not collected from |
| Body Weights | animals | | animals found dead or |
| | | | preterminally euthanized. |
| Food | All study | Once daily; from at least Week 3 | Qualitatively evaluated^{c} |
| Consumption | animals | | |
| Body | All study | At least twice during prestudy; | Was collected using a |
| Temperature | animals | on Day 1: 0.5, 1, 3, and 6 hours | digital infrared |
| | | postdose; and on Day 28: 0.5, 1, 3, and | thermometer. |
| | | 6 hours postdose | |
| Injection Site | All study | Predose on dosing days and daily | Injection sites were |
| Observations/ | animals | thereafter for 4 days or until any | examined for erythema, |
| Dermal | | reaction was resolved. In the event that | edema and other findings. |
| Scoring | | any injection site score other than “0” | Erythema and edema |
| | | was recorded for erythema, the injection | were evaluated in |
| | | site was monitored once daily until | accordance with the |
| | | resolution (i.e. “0”). | method of modified |
| | | | Draize. |

^{a}To include alternate animals until released from study.
^{b}For observations that cannot be attributed to an individual animal due to social housing (e.g., watery feces), the observation was attributed to each animal in the socialized group.
^{c}For observations of reduced appetite that cannot be attributed to an individual animal due to social housing, the observations wase attributed to each animal in the socialized group.

### Laboratory Evaluations

### Clinical Pathology

Sample Collection

| | | | Clinical |
|---|---|---|---|
| Group Nos. | Time Point | Hematology | Chemistry |
| All animals | Week 3 | X | X |
| Groups 1 to 4 | Day 21 | X | X |
| Groups 1 to 4 | Day 41 | X | X |
| Unscheduled euthanasia (when possible) | X | X | X |

| | | |
|---|---|---|
| Fasting | — | Yes |
| Method/Comments | Venipuncture^{a} | Venipuncture^{a} |
| Target Volume (mL)^{b} | 1 | 1.2 |
| Anticoagulant | K₃EDTA | None |
| Special Requirements | — | — |
| Processing | None | Serum |

X = sample to be collected; — = not applicable.
^{a}On days of unscheduled necropsy, if the attempts to collect blood by venipuncture were unsuccessful, blood was collected (as possible) from the vena cava immediately prior to
necropsy while the animals were under deep anesthesia.
^{b}Additional collection of protocol-specified blood samples was permitted (e.g., due to unacceptable sample quality) since the total blood collected was below the Testing Facility IACUC recommended maximum blood volume limit.

### Hematology

Hematology Parameters

| | |
|---|---|
| Red blood cell count | White blood cell count |
| Hemoglobin concentration | Neutrophil count (absolute) |
| Hematocrit | Lymphocyte count (absolute) |
| Mean corpuscular volume | Monocyte count (absolute) |
| Red blood cell distribution width | Eosinophil count (absolute) |
| Mean corpuscular hemoglobin | Basophil count (absolute) |
| concentration | Large unstained cells (absolute) |
| Mean corpuscular hemoglobin | Other cells (as appropriate) |
| Reticulocyte count (absolute) | |
| Platelet count | |

A blood smear was prepared from each hematology sample. Smears were examined if required to assess an animal's health following approval by the Study Director, or for confirmation of the hematology analyzer results. If additional examination of blood smears was deemed necessary, the smears were subsequently evaluated.

### Clinical Chemistry

Clinical Chemistry Parameters

| | |
|---|---|
| Alanine aminotransferase | Total protein |
| Aspartate aminotransferase | Albumin |
| Alkaline phosphatase | Globulin (calculated) |
| Gamma-glutamyltransferase | Albumin/globulin ratio |
| Lactate dehydrogenase | Glucose |
| Total bilirubin^{a} | Cholesterol |
| Urea nitrogen | Triglycerides |
| Creatinine | Sodium |
| Calcium | Potassium |
| Phosphorus | Chloride |
| Creatine kinase | Sample quality |

^{a}When total bilirubin is >1.0 mg/dL, direct bilirubin was measured and indirect bilirubin was calculated.

### Neutralizing Antibody Collection, Processing, and Analysis

Sample Collection Time Points

| Group Nos. | Study Day | Time Points (Relative to Dosing) |
|---|---|---|
| All Animals | Prestudy | — |
| 1 to 4 | Day 21 | Day 1, 20 days postdose |
| 1 to 4 | Day 41 | Day 21, 20 days postdose |

Unscheduled euthanasia (when possible)

| | |
|---|---|
| Method/Comments: | Venipuncture |
| | venipuncture were unsuccessful, blood was collected (as possible) from |
| | On days of unscheduled necropsy, if the attempts to collect blood by |
| | the vena cava immediately prior to necropsy while the animals were |
| | under deep anesthesia. |
| Target Volume (mL)^{a}: | 3 mL |
| Anticoagulant: | None |
| Special | None |
| Requirements: | |

— = not applicable; hr = hours; post = postdose; pre = predose.
^{a}Additional collection of protocol-specified blood samples was permitted (e.g., due to unacceptable sample quality) since the total blood collected is below the Testing Facility IACUC recommended maximum blood volume limit.

The samples were centrifuged and the resultant serum was separated, split into 5 approximately equal aliquots in uniquely labeled polypropylene tubes, and frozen immediately over dry ice or in a freezer set to maintain −80° C.

The samples were shipped and stored in a freezer set to maintain −70° C. or colder until analysis. The samples were be analyzed for neutralizing antibodies.

PMBC Sample Collection
Sample Collection Time Points

| Group Nos. | Study Day | Time Points (Relative to Dosing) |
|---|---|---|
| All Animals | Prestudy | — |
| 1 to 4 | Day 21 | Day 1, 20 days postdose |
| 1 to 4 | Day 41 | Day 21, 20 days postdose |

Unscheduled euthanasia (when possible)

| | |
|---|---|
| Method/Comments: | Venipuncture |
| | On days of unscheduled necropsy, if the attempts to collect blood by |
| | venipuncture are unsuccessful, blood will be collected (as possible) |
| | from the vena cava immediately prior to necropsy while the animals are |
| | under deep anesthesia. |
| Target Volume (mL)^{a}: | 4 mL |
| Anticoagulant: | Sodium Heparin |
| Special | The whole blood samples will be checked for clots at the animal room |
| Requirements: | level (i.e., visual check) and transferred at ambient temperature to the |
| | analytical laboratory at the Testing Facility. Upon receipt, the samples |
| | will be stored at ambient temperature until processing. In the event that |
| | clotting is discovered during processing the remaining unclotted |
| | sample will be processed to PBMC. |
| Processing: | To PBMC |

— = not applicable; hr = hours; post = postdose.
^{a}Additional collection of protocol-specified blood samples were permitted (e.g., due to unacceptable sample quality) since the total blood collected is below the Testing Facility IACUC recommended maximum blood volume limit.

### Peripheral Blood Mononuclear Cells (PBMCs) Isolation and Cryopreservation

Whole blood samples were transferred at ambient temperature to the appropriate laboratory at the Testing Facility within 1 hour of collection. The target concentration of PBMCs at the time of freezing was 4×10⁶ cells/mL.

PBMCs were isolated from blood samples per Testing Facility SOP. The resultant PBMCs were split into two approximately equal aliquots in uniquely labeled cryovials. Aliquots were stored frozen in a freezer set to maintain −80° C. for at least 24 hours. Cryovials were transferred within 72 hours from −80° C. freezer to liquid nitrogen (−140° C.).

The samples were shipped and then stored in liquid nitrogen (−140° C.) until analysis.

Cytokine Sample Collection, Processing, and Analysis

| Group Nos. | Study Day/Week | Time Points (Relative to Dosing) |
|---|---|---|
| All Animals | Prestudy | — |
| 1 to 4 | Day 1 | Day 1, pre; Day 1, 6 hr post |
| 1 to 4 | Day 28 | Day 28, pre; Day 28, 6 hr post |

Unscheduled euthanasia (when possible)

| Method: | Venipuncture |
|---|---|
| | On days of unscheduled necropsy, if the attempts to collect blood by |
| | venipuncture are unsuccessful, blood was collected (as possible) from |
| | the vena cava immediately prior to necropsy while the animals are under |
| | deep anesthesia. |
| Target Volume | 1.0 mL |
| (mL)^{a}: | |
| Anticoagulant: | None |
| Special | None |
| Requirements: | |

— = not applicable; hr = hours; post = postdose; pre = predose.
^{a}Additional collection of protocol-specified blood samples were permitted (e.g., due to unacceptable sample quality) since the total blood collected is below the Testing Facility IACUC recommended maximum blood volume limit.

The samples were centrifuged and the resultant serum was separated, split into two approximately equal aliquots in uniquely labeled polypropylene tubes, and frozen immediately over dry ice or in a freezer set to maintain −80° C.

The samples were shipped and stored in a freezer set to maintain −70° C. or colder until analysis.

The samples were analyzed in duplicate for IFN-α, IFN-γ, IL-1β, IL-6, IL-18, and TNF-α by multiplex Luminex assay using a qualified commercial kit supplied by Invitrogen.

### Results

FIG. 8 shows spike protein antibody titer as determined day 21 and the 41 day endpoint of the study in non-human primate (Cynomolgus monkey). The mRNA construct, representing the commercially available COVID-19 vaccine, was used (100 ug/dose) as the benchmark for comparison of spike protein antibody titer. As shown in FIG. 8, the spike protein Ab titer when ceDNA was employed (30 ug/dose) was within about 10 times of the mRNA benchmark, even when a dose of ceDNA was 3 times lower than the dose of mRNA. Moreover, the absolute binding titer of benchmark as determined in this experiment tracked closely to published clinical mRNA candidates. All doses were well-tolerated, with minimal injection site reactions and cytokines (not shown). Further, FIG. 8 demonstrates that the relative binding Ab titer (ceDNA vs. mRNA) translates to non-human primates.

Detectable viral neutralization was confirmed in ceDNA and mRNA constructs. In the ceDNA group, detectable neutralization at day 41, two weeks after the boost dose, appeared correlated with greater binding titer at day 21, 3 weeks after the first dose.

### Example 8: A Study to Evaluate Anti-Spike Antibody Responses after Intramuscular Administration of LNP:DNA Formulations in Female BALB/c Mice

ceDNA vectors were produced according to the methods described in Example 1 above.

The objective of the study was to evaluate the anti-spike protein antibody response after intramuscular (IM) injection of five (5) different LNP:ceDNA formulations (LNP1-5). The study design and details were carried out as set forth below.

### Study Design

Table 17 sets forth the design of the study. As shown in Table 17, a ceDNA comprising a nucleic acid encoding SARS-CoV-2 spike protein antigen was dosed in 7 groups (Groups 2-8) of mice (n=5) at a dose level of 3 μg or 10 μg in a dose volume of 30 μl/animal. Group 1 served as the control. Dosing was performed on Day 0 and Day 28 by intramuscular (IM) injection. Day 49 was the terminal time point of the study.

TABLE 17

| Group | No. of | | Dose | Dose | | Terminal |
|---|---|---|---|---|---|---|
| No. | Animals | Test Material | Levels (μg) | Volume (μL/an) | Dosing Regimen ROA | Time Point |
| 1 | 5 | PBS | NA | 30 | Once on Days 0 & 28 by IM | Day 49 |
| 2 | 5 | LNP 1:ceDNA | 3 | | | |
| 3 | 5 | LNP 2:ceDNA | 3 | | | |
| 4 | 5 | LNP 3:ceDNA | 3 | | | |
| 5 | 5 | LNP 4:ceDNA | 3 | | | |
| 6 | 5 | LNP 5:ceDNA | 3 | | | |
| 7 | 5 | LNP 3:ceDNA | 10 | | | |
| 8 | 5 | LNP 5:ceDNA | 10 | | | |

No. = Number;
an = animal;
IM = intramuscular;
ROA = route of administration

### Test System

The test system was as follows:
Species: Mus musculus
Strain: Balb/c mouse
Number of females: 40, plus 3 spares
Age: 6 weeks of age at arrival
Source: Charles River Laboratories

Housing: Animals were group housed in clear polycarbonate cages with contact bedding in a procedure room.

Food and Water: Animals were provided ad libitum Mouse Diet 5058 and filtered tap water acidified with 1N HCl to a targeted pH of 2.5-3.0.

### Test Material

Class of Compound: Recombinant DNA Vector: ceDNA

Dose Formulation: Test articles were supplied in a concentrated stock. Test article concentration was recorded at time of receipt.

Stock was warmed to room temperature and diluted with the provided PBS immediately, as necessary, prior to use. Prepared materials were stored at −4° C. if dosing was not performed immediately.

Test Material Administration: Test and control articles were dosed at 30 μL per animal on Days 0 & 28 for all Groups 1-8. Dosing was performed by intramuscular administration into the LEFT gastrocnemius. Animals were anesthetized with inhalant isoflurane, to effect, per facility SOPS for dose procedures.

Residual Materials: All residual open stock was retained for future dosing, refrigerated. Diluted dose materials were discarded after the completion of the dose administration.

### In-Life Observations and Measurements

Cage Side Observations (Animal Health Checks): Cage side animal health checks were performed at least once daily to check for general health, mortality and morbidity.

Clinical Observations: Clinical observations were performed on Days 0 and 28: 60-120 minutes post each dose and at the end of the work day (3-6 hours post) and on Days 1 and 29: 22-26 hours post the Day 0 and 28 Test Material dose.

Body Weights: Body weights for all animals (as applicable for remaining animals), were recorded on Days 0, 1, 2, 3, 7, 14, 21, 28, 29, 30, 31, 35, 42 & 49. Additional body weights were recorded as requested.

Anesthesia and Recovery: Animals were monitored continuously while under anesthesia, during recovery and until mobile, per testing facility SOPs.

### Blood Collection

All animals in Groups 1-8, had interim blood for serum collected on Day 0; 4-6 hours post Test Material dose as shown in Table 18 and Table 19 below.

Animals in Groups 1-8 had interim blood for serum collected on Day 21.

Whole blood for serum was collected by saphenous vein. Whole blood was collected into a serum separator with clot activator tube and processed into one (1) aliquot of serum per facility SOPs.

All samples were stored at nominally −70° C. until shipped to on dry ice.

TABLE 18
Blood Collection (Interim) for Cytokines

| | Sample Collection Times |
|---|---|
| | Whole Blood |
| Group | (Tail, saphenous or orbital) |
| Number | Serum^{a} |
| 1-8 | Day 0 |
| | 4-6 hours post Test Material dose (±5%) |
| Volume/ | ~150 μL whole blood |
| Portion | |
| Processing/ | 1 aliquot frozen at nominally −70° C. |
| Storage | (Charles River) |

^{a}Whole blood was collected into serum separator tubes, with clot activator

TABLE 19
Blood Collection (Interim)

| | Whole Blood Sample Collection Times |
|---|---|
| | Whole Blood |
| Group | (Tail, saphenous or orbital) |
| Number | Serum^{a} |
| 1-8 | Day 21 |
| Volume/ | ~100 μL whole blood |
| Portion | |
| Processing | One (1) aliquot |
| Storage | Frozen at nominally −70° C. |
| | (Gen Bio) |

^{a}Whole blood was be collected into serum separator tubes, with clot activator

After each collection animals received 0.5-1.0 mL lactated Ringer's; subcutaneously.

Whole blood for serum was collected by tail-vein nick, saphenous vein or orbital sinus puncture, under inhalant isoflurane. Whole blood was collected into a serum separator with clot activator tube and processed into two (2) aliquots of 25 μL of serum.

All samples were stored at nominally −70° C. until shipped to on dry ice.

Anesthesia Recovery: As applicable, animals were monitored continuously while under anesthesia, during recovery and until mobile.

### Results

FIG. 9 shows spike protein antibody titer as determined day 21 and day 49 of the study. Various ionizable lipid containing ceDNA formulations (LNP1-5) were tested to determine if certain lipids were preferable to others in the vaccine formulations. As shown in FIG. 9, LNP formulations (e.g., LNP 3 and 4) were more immunogenic than the other lipid formulations tested (e.g., LNP 5), indicating some lipids may be preferred over others in the ceDNA vaccine formulations. Non-limiting examples of ionizable lipids and LNP formulations that can be implemented in ceDNA vaccine formulations were exemplified in International Application PCT/US2022/025455, the entire content of which is incorporated herein in its entirety by reference.

### REFERENCES

All publications and references, including but not limited to patents and patent applications, cited in this specification and Examples herein are incorporated by reference in their entirety as if each individual publication or reference were specifically and individually indicated to be incorporated by reference herein as being fully set forth. Any patent application to which this application claims priority is also incorporated by reference herein in the manner described above for publications and references.

1. A capsid-free closed ended DNA (ceDNA) vector comprising at least one nucleic acid sequence between flanking inverted terminal (ITRs), wherein the at least one nucleic acid sequence encodes an antigen, or an immunogenic peptide.

2. The ceDNA vector of claim 1, wherein the antigen, or the immunogenic peptide, is derived from a bacterial, a viral, a fungal or a parasitic infectious agent.

3. The ceDNA vector of claim 1, wherein the antigen, or the immunogenic peptide, is a tumor associated antigen.

4. The ceDNA vector of claim 1, wherein the antigen, or the immunogenic peptide, is associated with an autoimmune condition.

5. The ceDNA vector any one of claims 1-4, wherein the antigen, or the immunogenic peptide, is selected from one or more of those set forth in Tables 1-8.

6. The ceDNA vector of any one of claims 1-5, comprising a promoter sequence operatively linked to the at least one nucleic acid sequence.

7. The ceDNA vector of any one of claims 1-6, wherein the ceDNA vector comprises at least one poly A sequence.

8. The ceDNA vector of any one of claims 1-7, wherein the ceDNA vector comprises a 5′ UTR and/or intron sequence.

9. The ceDNA vector of any one of claims 1-8, wherein the ceDNA vector comprises a 3′ UTR sequence.

10. The ceDNA vector of any one of claims 1-9, wherein the ceDNA vector comprises an enhancer sequence.

11. The ceDNA vector of any one of claims 1-10, wherein at least one ITR comprises a functional terminal resolution site and a Rep binding site.

12. The ceDNA vector of any one of claims 1-11, wherein one or both of the ITRs are from a virus selected from a Parvovirus, a Dependovirus, and an adeno-associated virus (AAV).

13. The ceDNA vector of any one of claims 1-12, wherein the flanking ITRs are symmetric or asymmetric with respect to one another.

14. The ceDNA vector of claim 13, wherein the flanking ITRs are symmetrical or substantially symmetrical.

15. The ceDNA vector of claim 13, wherein the flanking ITRs are asymmetric.

16. The ceDNA vector of any one of claims 1-15, wherein one or both of the ITRs are wild type, or wherein both of the ITRs are wild-type ITRs.

17. The ceDNA vector of any one of claims 1-16, wherein the flanking ITRs are from different viral serotypes.

18. The ceDNA vector of any one of claims 1-17, wherein the flanking ITRs are selected from any pair of viral serotypes shown in Table 8.

19. The ceDNA vector of any one of claims 1-18, wherein one or both of the ITRs comprises a sequence selected from one or more of the sequences in Table 9.

20. The ceDNA vector of any one of claims 1-19, wherein at least one of the ITRs is altered from a wild-type AAV ITR sequence by a deletion, addition, or substitution that affects the overall three-dimensional conformation of the ITR.

21. The ceDNA vector of any one of claims 1-20, wherein one or both of the ITRs are derived from an AAV serotype selected from AAV1, AAV2, AAV3, AAV4, AAV5, AAV6, AAV7, AAV8, AAV9, AAV10, AAV11, and AAV12.

22. The ceDNA vector of any one of claims 1-21, wherein one or both of the ITRs are synthetic.

23. The ceDNA vector of any one of claims 1-15, wherein one or both of the ITRs are not a wild type ITR, or wherein both of the ITRs are not wild-type ITRs.

24. The ceDNA vector of any one of claims 1-23, wherein one or both of the ITRs are modified by a deletion, insertion, and/or substitution in at least one of the ITR regions selected from A, A′, B, B′, C, C′, D, and D′.

25. The ceDNA vector of claim 24, wherein the deletion, insertion, and/or substitution results in the deletion of all or part of a stem-loop structure normally formed by the A, A′, B, B′, C, or C′ regions.

26. The ceDNA vector of any one of claims 1-25, wherein one or both of the ITRs are modified by a deletion, insertion, and/or substitution that results in the deletion of all or part of a stem-loop structure normally formed by the B and B′ regions.

27. The ceDNA vector of any one of claims 1-26, wherein one or both of the ITRs are modified by a deletion, insertion, and/or substitution that results in the deletion of all or part of a stem-loop structure normally formed by the C and C′ regions.

28. The ceDNA vector of any one of claims 1-27, wherein one or both of the ITRs are modified by a deletion, insertion, and/or substitution that results in the deletion of part of a stem-loop structure normally formed by the B and B′ regions and/or part of a stem-loop structure normally formed by the C and C′ regions.

29. The ceDNA vector of any one of claims 1-28, wherein one or both of the ITRs comprise a single stem-loop structure in the region that normally comprises a first stem-loop structure formed by the B and B′ regions and a second stem-loop structure formed by the C and C′ regions.

30. The ceDNA vector of any one of claims 1-29, wherein one or both of the ITRs comprise a single stem and two loops in the region that normally comprises a first stem-loop structure formed by the B and B′ regions and a second stem-loop structure formed by the C and C′ regions.

31. The ceDNA vector of any one of claims 1-30, wherein one or both of the ITRs comprise a single stem and a single loop in the region that normally comprises a first stem-loop structure formed by the B and B′ regions and a second stem-loop structure formed by the C and C′ regions.

32. The ceDNA vector of any one of claims 1-31, wherein both ITRs are altered in a manner that results in an overall three-dimensional symmetry when the ITRs are inverted relative to each other.

33. The ceDNA vector of any one of claims 1-32, wherein the ceDNA vector is encapsulated in a lipid nanoparticle (LNP).

34. The ceDNA vector of any one of claims 1-33, for use in a vaccine.

35. A method of expressing an antigen, or an immunogenic peptide, in a cell comprising contacting the cell with the ceDNA vector of any one of claims 1-33.

36. The method of claim 35, wherein the cell is in vitro or in vivo.

37. The method of claim 35 or claim 36, wherein the at least one nucleic acid sequence is codon optimized for expression in the cell.

38. A method of treating a subject with a bacterial, a viral, a parasitic or a fungal infection, comprising administering to the subject the ceDNA vector of any one of claims 1-33.

39. A method of treating a subject with a cancer, comprising administering to the subject the ceDNA vector of any one of claims 1-33.

40. A method of treating a subject with an autoimmune disease or disorder, comprising administering to the subject the ceDNA vector of any one of claims 1-33.

41. A method of preventing a bacterial, a viral, a parasitic or a fungal infection in a subject, comprising administering to the subject the ceDNA vector of any one of claims 1-33.

42. A method of preventing cancer in a subject, comprising administering to the subject the ceDNA vector of any one of claims 1-33.

43. A method of preventing an autoimmune disease in a subject, comprising administering to the subject the ceDNA vector of any one of claims 1-33.

44. The method of any one of claims 38-43, further comprising administering to the subject one or more additional therapeutic agents.

45. The method of any one of claims 38-43, wherein the ceDNA vector is administered by intravenous, subcutaneous, intratumoral or intramuscular injection.

46. A pharmaceutical composition comprising the ceDNA vector of any one of claims 1-33.

47. The pharmaceutical composition of claim 46, further comprising one or more additional therapeutic agents.

48. A vaccine composition comprising the ceDNA vector of any one of claims 1-33.

49. A composition comprising the ceDNA vector of any one of claims 1-33 and a lipid.

50. The composition of claim 49, wherein the lipid is a lipid nanoparticle (LNP).

51. The composition of claim 50, wherein the lipid nanoparticle comprises an ionizable lipid.

52. The composition of claim 51, wherein the ionizable lipid is selected from the group consisting of the following:

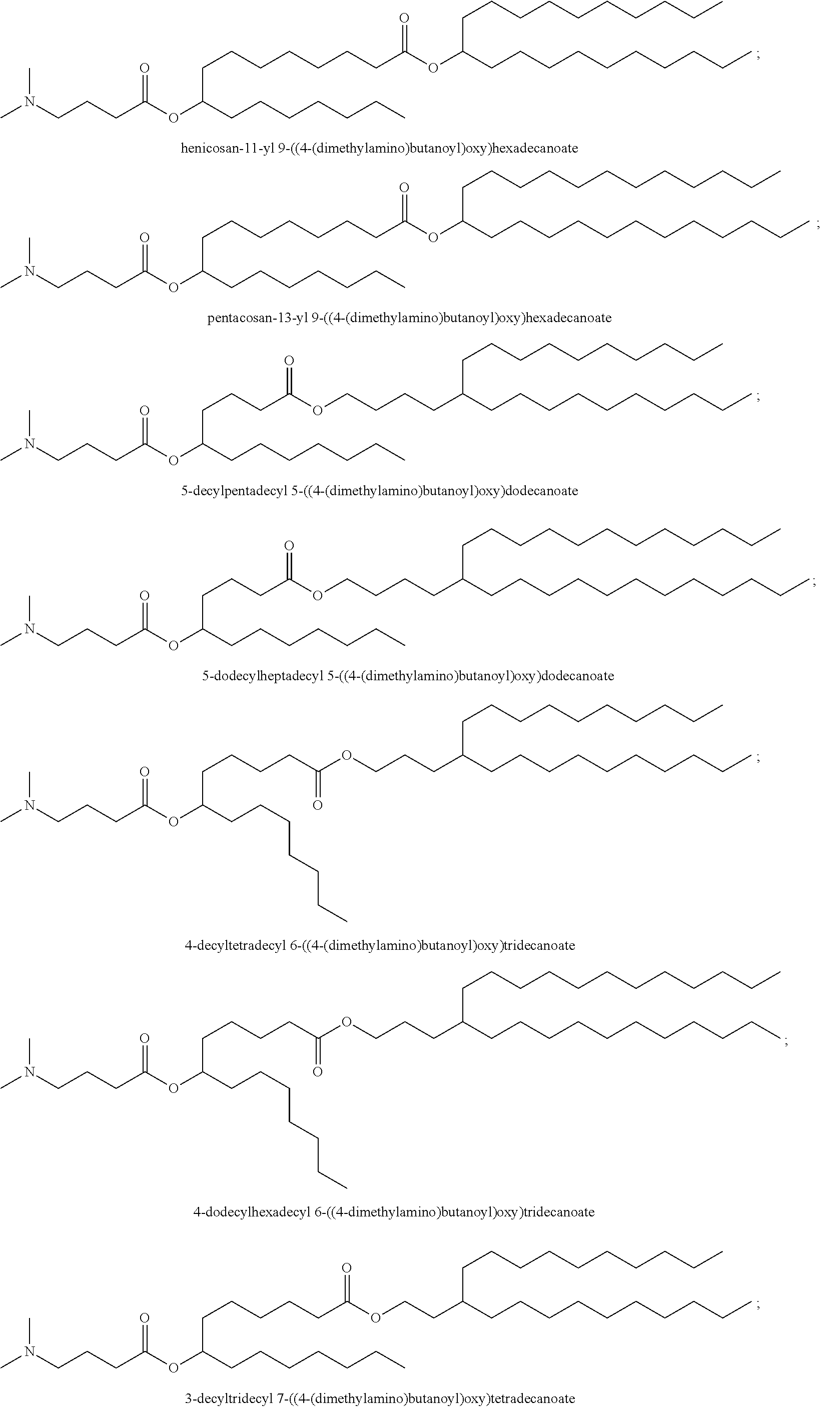

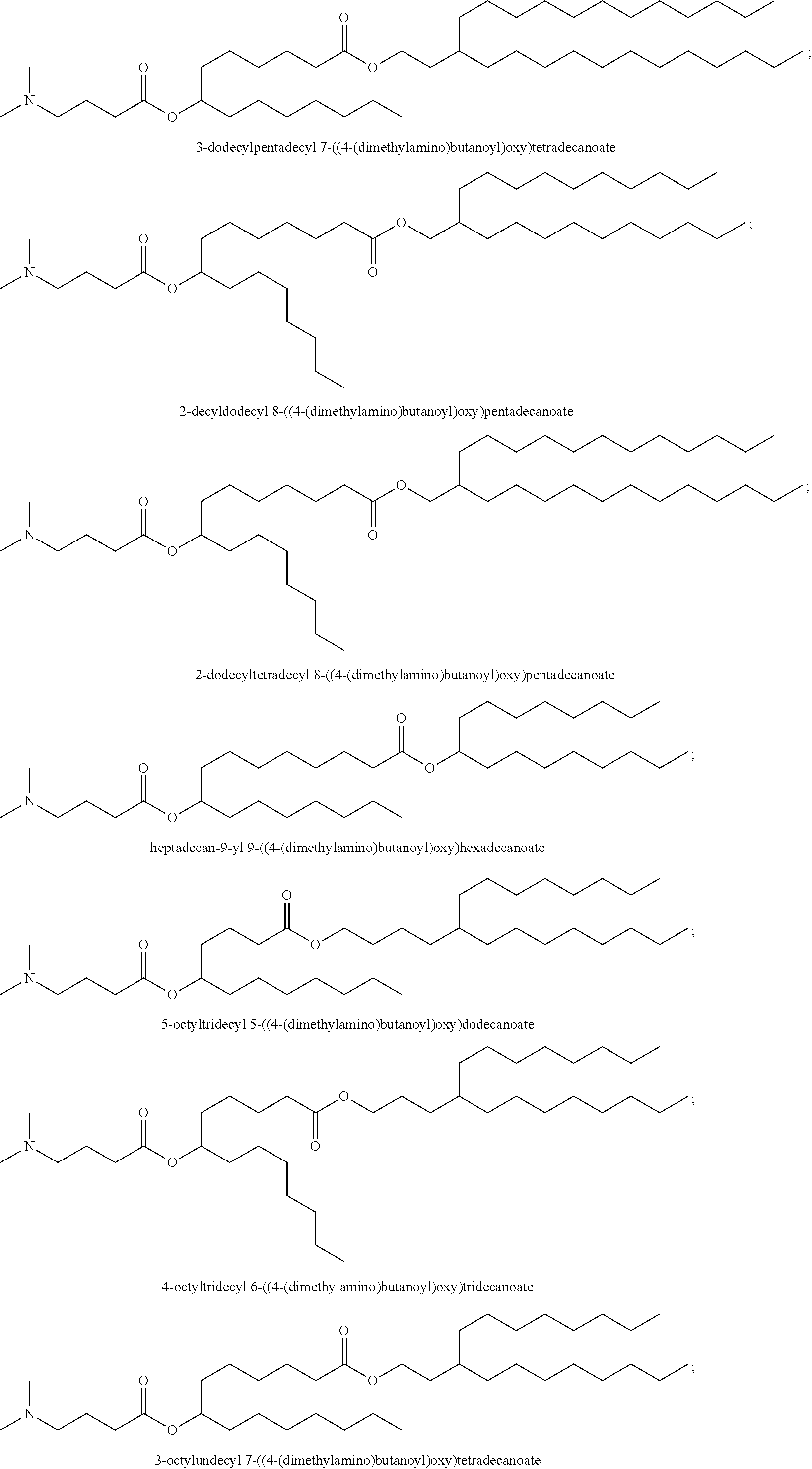

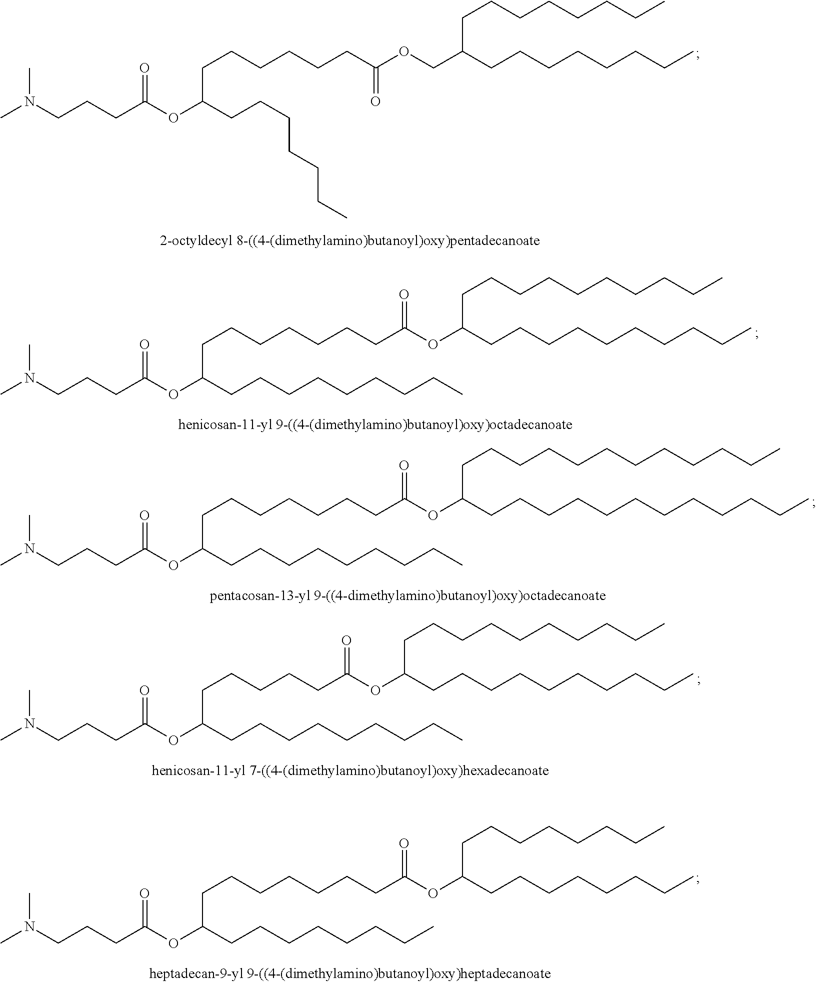

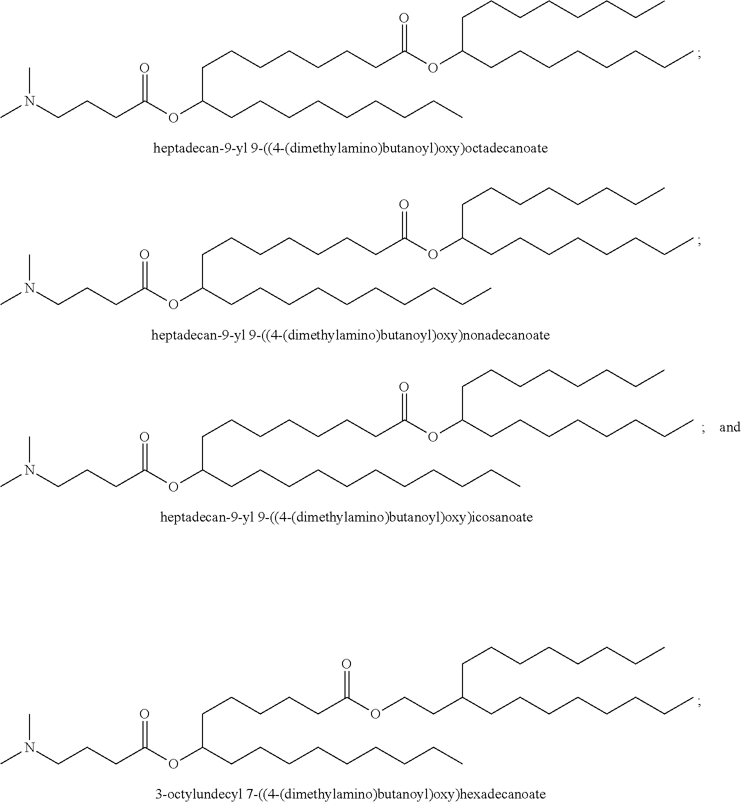

53. The composition of any one of claims 48-52, wherein the composition is lyophilized.

54. A kit comprising the ceDNA vector of any one of claims 1-33, the pharmaceutical composition of claim 46 or claim 47 or the composition of any one of claims 48-53.


SEQUENCE LISTING (135 sequences)

SEQ ID NO: 1 - DNA, 141 bp, Adeno-associated virus 2
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 2 - DNA, 141 bp, Adeno-associated virus 2
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 3-4 - skipped (2 entries)

SEQ ID NO: 5 - DNA, 143 bp, Adeno-associated virus
ttgcccactccctctctgcgcgctcgctcgctcggtggggcctgcggaccaaaggtccgcagacggcagaggtctcctctgccggccccaccgagcgagcgacgcgcgcagagagggagtgggcaactccatcactagggtaa

SEQ ID NO: 6 - DNA, 144 bp, Adeno-associated virus 3
ttggccactccctctatgcgcactcgctcgctcggtggggcctggcgaccaaaggtcgccagacggacgtgggtttccacgtccggccccaccgagcgagcgagtgcgcatagagggagtggccaactccatcactagaggtat

SEQ ID NO: 7 - DNA, 127 bp, Adeno-associated virus 4
ttggccactccctctatgcgcgctcgctcactcactcggccctggagaccaaaggtctccagactgccggcctctggccggcagggccgagtgagtgagcgagcgcgcatagagggagtggccaact

SEQ ID NO: 8 - DNA, 166 bp, Adeno-associated virus 5
tcccccctgtcgcgttcgctcgctcgctggctcgtttgggggggcgacggccagagggccgtcgtctggcagctctttgagctgccacccccccaaacgagccagcgagcgagcgaacgcgacaggggggagagtgccacactctcaagcaagggggttttgtaag

SEQ ID NO: 9 - DNA, 144 bp, Adeno-associated virus 6
ttgcccactccctctaatgcgcgctcgctcgctcggtggggcctgcggaccaaaggtccgcagacggcagaggtctcctctgccggccccaccgagcgagcgagcgcgcatagagggagtgggcaactccatcactaggggtat

SEQ ID NO: 10 - DNA, 143 bp, Adeno-associated virus
ttaccctagtgatggagttgcccactccctctctgcgcgcgtcgctcgctcggtggggccggcagaggagacctctgccgtctgcggacctttggtccgcaggccccaccgagcgagcgagcgcgcagagagggagtgggcaa

SEQ ID NO: 11 - DNA, 144 bp, Adeno-associated virus 3
atacctctagtgatggagttggccactccctctatgcgcactcgctcgctcggtggggccggacgtggaaacccacgtccgtctggcgacctttggtcgccaggccccaccgagcgagcgagtgcgcatagagggagtggccaa

SEQ ID NO: 12 - DNA, 127 bp, Adeno-associated virus 4
agttggccacattagctatgcgcgctcgctcactcactcggccctggagaccaaaggtctccagactgccggcctctggccggcagggccgagtgagtgagcgagcgcgcatagagggagtggccaa

SEQ ID NO: 13 - DNA, 166 bp, Adeno-associated virus 5
cttacaaaacccccttgcttgagagtgtggcactctcccccctgtcgcgttcgctcgctcgctggctcgtttgggggggtggcagctcaaagagctgccagacgacggccctctggccgtcgcccccccaaacgagccagcgagcgagcgaacgcgacagggggga

SEQ ID NO: 14 - DNA, 144 bp, Adeno-associated virus 6
atacccctagtgatggagttgcccactccctctatgcgcgctcgctcgctcggtggggccggcagaggagacctctgccgtctgcggacctttggtccgcaggccccaccgagcgagcgagcgcgcattagagggagtgggcaa

SEQ ID NO: 15 - DNA, 120 bp, Artificial Sequence
  Description of Artificial Sequence: Synthetic polynucleotide
aggaacccctagtgatggagttggccactccctctctgcgcgctcgctcgctcactgaggcgcacgcccgggtttcccgggcggcctcagtgagcgagcgagcgcgcagctgcctgcagg

SEQ ID NO: 16 - DNA, 122 bp, Artificial Sequence
  Description of Artificial Sequence: Synthetic polynucleotide
aggaacccctagtgatggagttggccactccctctctgcgcgctcgctcgctcactgaggccgacgcccgggctttgcccgggcggcctcagtgagcgagcgagcgcgcagctgcctgcagg

SEQ ID NO: 17 - DNA, 129 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggcgcctcagtgagcgagcgagcgcgcagctgcctgcagg

SEQ ID NO: 18 - DNA, 101 bp, Artificial Sequence
  Description of Artificial Sequence: Synthetic polynucleotide
aggaacccctagtgatggagttggccactccctctctgcgcgctcgctcgctcactgaggctttgcctcagtgagcgagcgagcgcgcagctgcctgcagg

SEQ ID NO: 19 - DNA, 139 bp, Artificial Sequence
  Description of Artificial Sequence: Synthetic polynucleotide
aggaacccctagtgatggagttggccactccctctctgcgcgctcgctcgctcactgaggccgggcgacaaagtcgcccgacgcccgggctttgcccgggcggcctcagtgagcgagcgagcgcgcagctgcctgcagg

SEQ ID NO: 20 - DNA, 137 bp, Artificial Sequence
  Description of Artificial Sequence: Synthetic polynucleotide
aggaacccctagtgatggagttggccactccctctctgcgcgctcgctcgctcactgaggccgggcgaaaatcgcccgacgcccgggctttgcccgggcggcctcagtgagcgagcgagcgcgcagctgcctgcagg

SEQ ID NO: 21 - DNA, 135 bp, Artificial Sequence
  Description of Artificial Sequence: Synthetic polynucleotide
aggaacccctagtgatggagttggccactccctctctgcgcgctcgctcgctcactgaggccgggcgaaacgcccgacgcccgggctttgcccgggcggcctcagtgagcgagcgagcgcgcagctgcctgcagg

SEQ ID NO: 22 - DNA, 133 bp, Artificial Sequence
  Description of Artificial Sequence: Synthetic polynucleotide
aggaacccctagtgatggagttggccactccctctctgcgcgctcgctcgctcactgaggccgggcaaagcccgacgcccgggctttgcccgggcggcctcagtgagcgagcgagcgcgcagctgcctgcagg

SEQ ID NO: 23 - DNA, 139 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggtttcccgggcggcctcagtgagcgagcgagcgcgcagctgcctgcagg

SEQ ID NO: 24 - DNA, 137 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccggtttccgggcggcctcagtgagcgagcgagcgcgcagctgcctgcagg

SEQ ID NO: 25 - DNA, 135 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgtttcgggcggcctcagtgagcgagcgagcgcgcagctgcctgcagg

SEQ ID NO: 26 - DNA, 133 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgccctttgggcggcctcagtgagcgagcgagcgcgcagctgcctgcagg

SEQ ID NO: 27 - DNA, 131 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcctttggcggcctcagtgagcgagcgagcgcgcagctgcctgcagg

SEQ ID NO: 28 - DNA, 129 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgctttgcggcctcagtgagcgagcgagcgcgcagctgcctgcagg

SEQ ID NO: 29 - DNA, 127 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgtttcggcctcagtgagcgagcgagcgcgcagctgcctgcagg

SEQ ID NO: 30 - DNA, 122 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacggcctcagtgagcgagcgagcgcgcagctgcctgcagg

SEQ ID NO: 31 - DNA, 130 bp, Artificial Sequence
  Description of Artificial Sequence: Synthetic polynucleotide
aggaacccctagtgatggagttggccactccctctctgcgcgctcgctcgctcactgaggccgggcgaccaaaggtcgcccgacgcccgggcggcctcagtgagcgagcgagcgcgcagctgcctgcagg

SEQ ID NO: 32 - DNA, 120 bp, Artificial Sequence
  Description of Artificial Sequence: Synthetic polynucleotide
cctgcaggcagctgcgcgctcgctcgctcactgaggccgcccgggaaacccgggcgtgcgcctcagtgagcgagcgagcgcgcagagagggagtggccaactccatcactaggggttcct

SEQ ID NO: 33 - DNA, 122 bp, Artificial Sequence
  Description of Artificial Sequence: Synthetic polynucleotide
cctgcaggcagctgcgcgctcgctcgctcactgaggccgtcgggcgacctttggtcgcccggcctcagtgagcgagcgagcgcgcagagagggagtggccaactccatcactaggggttcct

SEQ ID NO: 34 - DNA, 122 bp, Artificial Sequence
  Description of Artificial Sequence: Synthetic polynucleotide
cctgcaggcagctgcgcgctcgctcgctcactgaggccgcccgggcaaagcccgggcgtcggcctcagtgagcgagcgagcgcgcagagagggagtggccaactccatcactaggggttcct

SEQ ID NO: 35 - DNA, 129 bp, Artificial Sequence
  Description of Artificial Sequence: Synthetic polynucleotide
cctgcaggcagctgcgcgctcgctcgctcactgaggcgcccgggcgtcgggcgacctttggtcgcccggcctcagtgagcgagcgagcgcgcagagagggagtggccaactccatcactaggggttcct

SEQ ID NO: 36 - DNA, 101 bp, Artificial Sequence
  Description of Artificial Sequence: Synthetic polynucleotide
cctgcaggcagctgcgcgctcgctcgctcactgaggcaaagcctcagtgagcgagcgagcgcgcagagagggagtggccaactccatcactaggggttcct

SEQ ID NO: 37 - DNA, 139 bp, Artificial Sequence
  Description of Artificial Sequence: Synthetic polynucleotide
cctgcaggcagctgcgcgctcgctcgctcactgaggccgcccgggcaaagcccgggcgtcgggcgactttgtcgcccggcctcagtgagcgagcgagcgcgcagagagggagtggccaactccatcactaggggttcct

SEQ ID NO: 38 - DNA, 137 bp, Artificial Sequence
  Description of Artificial Sequence: Synthetic polynucleotide
cctgcaggcagctgcgcgctcgctcgctcactgaggccgcccgggcaaagcccgggcgtcgggcgattttcgcccggcctcagtgagcgagcgagcgcgcagagagggagtggccaactccatcactaggggttcct

SEQ ID NO: 39 - DNA, 135 bp, Artificial Sequence
  Description of Artificial Sequence: Synthetic polynucleotide
cctgcaggcagctgcgcgctcgctcgctcactgaggccgcccgggcaaagcccgggcgtcgggcgtttcgcccggcctcagtgagcgagcgagcgcgcagagagggagtggccaactccatcactaggggttcct

SEQ ID NO: 40 - DNA, 133 bp, Artificial Sequence
  Description of Artificial Sequence: Synthetic polynucleotide
cctgcaggcagctgcgcgctcgctcgctcactgaggccgcccgggcaaagcccgggcgtcgggctttgcccggcctcagtgagcgagcgagcgcgcagagagggagtggccaactccatcactaggggttcct

SEQ ID NO: 41 - DNA, 139 bp, Artificial Sequence
  Description of Artificial Sequence: Synthetic polynucleotide
cctgcaggcagctgcgcgctcgctcgctcactgaggccgcccgggaaacccgggcgtcgggcgacctttggtcgcccggcctcagtgagcgagcgagcgcgcagagagggagtggccaactccatcactaggggttcct

SEQ ID NO: 42 - DNA, 137 bp, Artificial Sequence
  Description of Artificial Sequence: Synthetic polynucleotide
cctgcaggcagctgcgcgctcgctcgctcactgaggccgcccggaaaccgggcgtcgggcgacctttggtcgcccggcctcagtgagcgagcgagcgcgcagagagggagtggccaactccatcactaggggttcct

SEQ ID NO: 43 - DNA, 135 bp, Artificial Sequence
  Description of Artificial Sequence: Synthetic polynucleotide
cctgcaggcagctgcgcgctcgctcgctcactgaggccgcccgaaacgggcgtcgggcgacctttggtcgcccggcctcagtgagcgagcgagcgcgcagagagggagtggccaactccatcactaggggttcct

SEQ ID NO: 44 - DNA, 133 bp, Artificial Sequence
  Description of Artificial Sequence: Synthetic polynucleotide
cctgcaggcagctgcgcgctcgctcgctcactgaggccgcccaaagggcgtcgggcgacctttggtcgcccggcctcagtgagcgagcgagcgcgcagagagggagtggccaactccatcactaggggttcct

SEQ ID NO: 45 - DNA, 131 bp, Artificial Sequence
  Description of Artificial Sequence: Synthetic polynucleotide
cctgcaggcagctgcgcgctcgctcgctcactgaggccgccaaaggcgtcgggcgacctttggtcgcccggcctcagtgagcgagcgagcgcgcagagagggagtggccaactccatcactaggggttcct

SEQ ID NO: 46 - DNA, 129 bp, Artificial Sequence
  Description of Artificial Sequence: Synthetic polynucleotide
cctgcaggcagctgcgcgctcgctcgctcactgaggccgcaaagcgtcgggcgacctttggtcgcccggcctcagtgagcgagcgagcgcgcagagagggagtggccaactccatcactaggggttcct

SEQ ID NO: 47 - DNA, 127 bp, Artificial Sequence
  Description of Artificial Sequence: Synthetic polynucleotide
cctgcaggcagctgcgcgctcgctcgctcactgaggccgaaacgtcgggcgacctttggtcgcccggcctcagtgagcgagcgagcgcgcagagagggagtggccaactccatcactaggggttcct

SEQ ID NO: 48 - DNA, 122 bp, Artificial Sequence; identical to SEQ ID NO: 30
  Description of Artificial Sequence: Synthetic polynucleotide

SEQ ID NO: 49-59 - skipped (11 entries)

SEQ ID NO: 60 - DNA, 16 bp, Adeno-associated virus 2
gcgcgctcgctcgctc

SEQ ID NO: 61-122 - skipped (62 entries)

SEQ ID NO: 123 - DNA, 8 bp, Artificial Sequence
  Description of Artificial Sequence: Synthetic oligonucleotide
gtttaaac

SEQ ID NO: 124 - DNA, 8 bp, Artificial Sequence
  Description of Artificial Sequence: Synthetic oligonucleotide
ttaattaa

SEQ ID NO: 125 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 126 - DNA, 16 bp, Adeno-associated virus 2
gagcgagcgagcgcgc

SEQ ID NO: 127 - DNA, 12 bp, Artificial Sequence
  Description of Artificial Sequence: Synthetic oligonucleotide
atcgaacgatcg

SEQ ID NO: 128 - DNA, 12 bp, Artificial Sequence
  Description of Artificial Sequence: Synthetic oligonucleotide
cgatcgttcgat

SEQ ID NO: 129 - DNA, 12 bp, Artificial Sequence
  Description of Artificial Sequence: Synthetic oligonucleotide
atcgaaccatcg

SEQ ID NO: 130 - DNA, 165 bp, Adeno-associated virus 2
aggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 131 - DNA, 140 bp, Adeno-associated virus 2
cccctagtgatggagttggccactccctctctgcgcgctcgctcgctcactgaggccgcccgggcaaagcccgggcgtcgggcgacctttggtcgcccggcctcagtgagcgagcgagcgcgcagagagatcactagggg

SEQ ID NO: 132 - DNA, 91 bp, Adeno-associated virus 2
gcgcgctcgctcgctcactgaggccgcccgggcaaagcccgggcgtcgggcgacctttggtcgcccggcctcagtgagcgagcgagcgcgc

SEQ ID NO: 133 - DNA, 80 bp, Artificial Sequence
  Description of Artificial Sequence: Synthetic oligonucleotide
gcgcgctcgctcgctcactgaggccgcccgggcgtcgggcgacctttggtcgcccggcctcagtgagcgagcgagcgcgc

SEQ ID NO: 134 - DNA, 91 bp, Adeno-associated virus 2
gcgcgctcgctcgctcactgaggccgggcgaccaaaggtcgcccgacgcccgggctttgcccgggcggcctcagtgagcgagcgagcgcgc

SEQ ID NO: 135 - DNA, 80 bp, Artificial Sequence
  Description of Artificial Sequence: Synthetic oligonucleotide
gcgcgctcgctcgctcactgaggccgggcgaccaaaggtcgcccgacgcccgggcggcctcagtgagcgagcgagcgcgc